(19) United States
(12) Patent Application Publication
(10) Pub. No.: US 2025/0346907 A1
(43) Pub. Date: Nov. 13, 2025

(54) COMPOUNDS AND METHODS FOR REDUCING GLYCOGEN SYNTHASE 1

(71) Applicant: Ionis Pharmaceuticals, Inc., Carlsbad, CA (US)

(72) Inventors: Susan M. Freier, San Diego, CA (US); Huynh-Hoa Bui, San Diego, CA (US); Bethany Fitzsimmons, Webster, NY (US); Holly Kordasiewicz, San Diego, CA (US); Tamar R. Grossman, San Diego, CA (US)

(73) Assignee: Ionis Pharmaceuticals, Inc., Carlsbad, CA (US)

(21) Appl. No.: 18/722,831

(22) PCT Filed: Dec. 21, 2022
(86) PCT No.: PCT/US2022/082148

§ 371 Date: Jun. 21, 2024

Related U.S. Application Data
(60) Provisional application No. 63/292,883, filed on Dec. 22, 2021.
(60) Provisional application No. 63/317,437, filed on Mar. 7, 2022.

(51) Int. Cl. C12N 15/115; A61K 47/46

(52) U.S. Cl. CPC C12N 15/115; A61K 47/46

(57) ABSTRACT

Provided are compounds, methods, and pharmaceutical compositions for reducing the amount or activity of GYS1 RNA in a cell or subject, and in certain instances reducing the amount of GYS1 protein in a cell or subject. Such compounds, methods, and pharmaceutical compositions are useful to ameliorate at least one symptom or hallmark of a glycogen storage disease. Such glycogen storage diseases include Lafora disease, adult polyglucosan body disease (APBD), Andersen's disease, and Pompe disease.

### SEQUENCE LISTING

The present application is being filed along with a Sequence Listing in electronic format. The Sequence Listing is provided as a file entitled BIOL0447SEQ.xml, created on Dec. 16, 2022, which is 2.91 MB in size. The information in the electronic format of the sequence listing is incorporated herein by reference in its entirety.

### FIELD

Provided are compounds, pharmaceutical compositions, and methods of use for reducing the amount or activity of glycogen synthase 1 (hereinafter referred to as GYS1) RNA in a cell or subject, and in certain instances reducing the amount of GYS1 protein in a cell or subject. Such compounds, methods, and pharmaceutical compositions are useful to ameliorate at least one symptom or hallmark of a polyglucosan disorder in a subject. In certain embodiments, such compounds, methods, and pharmaceutical compositions are useful to ameliorate at least one symptom or hallmark of a glycogen storage disease. In certain embodiments, such compounds, methods, and pharmaceutical compositions are useful to ameliorate at least one symptom or hallmark of Lafora disease in a subject. In certain embodiments, such compounds, methods, and pharmaceutical compositions are useful to ameliorate at least one symptom or hallmark of adult polyglucosan body disease (APBD) in a subject.

### BACKGROUND

Glycogen is a branched polymer of glucose that constitutes the sole carbohydrate reserve for mammals. It is synthesized by glycogen synthase (GYS), the only mammalian enzyme able to polymerize glucose (Bollen M. et al. Biochem. J. 1998 336: 19-31). Glycogen biosynthesis involves chain elongation by glycogen synthase and chain branching by glycogen branching enzyme. If chain elongation outbalances chain branching, glycogen forms starch-like precipitates made up of long, non-branched chains called polyglucosans. The most glycogenic tissues are muscle and liver.

Glycogen synthase 1 (GYS1) is an enzyme involved in converting glucose to glycogen by catalyzing the elongation of short glucose polymers into long glycogen polymers. Mutations in GYS1 are associated with glycogen storage diseases. In the brain, glycogen is normally stored in astrocytes (brown A. M. J. Neurochem. 89: 537-552, 2004) and glycogen synthesis is normally absent in neurons because of tight regulation of GYS1 by laforin and malin (Vilchez et al., Nat. Neurosci. 10: 1407-1413, 2007). Nevertheless, aberrant glycogen accumulation in neurons is a hallmark of patients suffering from Lafora disease, Pompe disease, Andersen's disease, adult polyglucosan body disease, or other GYS1-associated diseases or disorders.

Currently, there is a lack of specific inhibitors for GYS1. It is therefore an objective herein to provide methods for the treatment of such diseases or disorders. It is therefore an objective herein to provide compounds, methods, and pharmaceutical compositions for the treatment of such diseases or disorders.

### SUMMARY OF THE INVENTION

Provided herein are compounds, pharmaceutical compositions, and methods of use for reducing the amount or activity of GYS1 RNA, and in certain embodiments reducing the expression of GYS1 protein in a cell or subject. In certain embodiments, the subject has a disease or disorder associated with GYS1. In certain embodiments, the disease or disorder associated with GYS1 is a glycogen storage disease. In certain embodiments, the subject has a neurogenerative disease characterized by an accumulation of aberrant glycogen, an accumulation of polyglucosan bodies, and/or an accumulation of Lafora bodies. In certain embodiments, compounds useful for reducing the amount or activity of GYS1 RNA are oligomeric compounds. In certain embodiments, compounds useful for reducing the amount or activity of GYS1 RNA are modified oligonucleotides. In certain embodiments, compounds useful for reducing expression of GYS1 protein are oligomeric compounds. In certain embodiments, compounds useful for reducing expression of GYS1 protein are modified oligonucleotides.

Also provided are methods useful for ameliorating at least one symptom of a disease or disorder associated with GYS1. In certain embodiments, the disease or disorder associated with GYS1 is a glycogen storage disease. In certain embodiments, the glycogen storage disease is Lafora disease. In certain embodiments, the glycogen storage disease is adult polyglucosan body disease (APBD). In certain embodiments, the glycogen storage disease is Andersen's disease. In certain embodiments, the glycogen storage disease is Pompe disease. In certain embodiments, at least one symptom or hallmark of the glycogen storage disease is seizures, cognitive deterioration, neuromuscular weakness, myoclonus, dementia, ataxia, cerebellar dysfunction, impaired speech, loss of ambulation, swallowing difficulty, or epileptic episodes. In certain embodiments, a symptom or hallmark of the glycogen storage disease is an increase in glycogen levels, accumulation of polyglucosan bodies, or accumulation of Lafora bodies.

### DETAILED DESCRIPTION OF THE INVENTION

It is to be understood that both the foregoing general description and the following detailed description are exemplary and explanatory only and are not restrictive. Herein, the use of the singular includes the plural unless specifically stated otherwise. As used herein, the use of “or” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited in this application, including, but not limited to, patents, patent applications, articles, books, treatises, and GenBank, ENSEMBL, and NCBI reference sequence records, are hereby expressly incorporated-by-reference for the portions of the document discussed herein, as well as in their entirety.

### Definitions

Unless specific definitions are provided, the nomenclature used in connection with, and the procedures and techniques of, analytical chemistry, synthetic organic chemistry, and medicinal and pharmaceutical chemistry described herein are those well-known and commonly used in the art. Where permitted, all patents, applications, published applications and other publications and other data referred to throughout the disclosure are incorporated by reference herein in their entirety.

Unless otherwise indicated, the following terms have the following meanings:

As used herein, “2′-deoxynucleoside” means a nucleoside comprising a 2′-H(H) deoxyfuranosyl sugar moiety. In certain embodiments, a 2′-deoxynucleoside is a 2′-β-D-deoxynucleoside and comprises a 2′-β-D-deoxyribosyl sugar moiety, which has the β-D ribosyl configuration as found in naturally occurring deoxyribonucleic acids (DNA). In certain embodiments, a 2′-deoxynucleoside may comprise a modified nucleobase or may comprise an RNA nucleobase (uracil).

As used herein, “2′-MOE” means a 2′-OCH₂CH₂OCH₃ group in place of the 2′-OH group of a ribosyl sugar moiety. A “2′-MOE modified sugar moiety” means a sugar moiety with a 2′-OCH₂CH₂OCH₃ group in place of the 2′-OH group of a ribosyl sugar moiety. Unless otherwise indicated, a 2′-MOE modified sugar moiety is in the β-D-ribosyl configuration. “MOE” means O-methoxyethyl.

As used herein, “2′-MOE nucleoside” or “2′-MOE modified nucleoside” or “2′-O(CH₂)₂OCH₃ nucleoside” means a nucleoside comprising a 2′-MOE modified sugar moiety (or 2′-O(CH₂)₂OCH₃ ribosyl sugar moiety).

As used herein, “2′-OMe” means a 2′-OCH₃ group in place of the 2′-OH group of a ribosyl sugar moiety. A “2′-O-methyl sugar moiety” or “2′-OMe modified sugar moiety” means a sugar moiety with a 2′-OCH₃ group in place of the 2′-OH group of a ribosyl sugar moiety. Unless otherwise indicated, a 2′-OMe modified sugar moiety is in the β-D-ribosyl configuration.

As used herein, “2′-OMe nucleoside” or “2′-OMe modified nucleoside” means a nucleoside comprising a 2′-OMe modified sugar moiety.

As used herein, “2′-F” means a 2′-fluoro group in place of the 2′-OH group of a ribosyl sugar moiety. A “2′-F modified sugar moiety” or “2′-fluororibosyl sugar” means a sugar moiety with a 2′—F group in place of the 2′-OH group of a ribosyl sugar moiety. Unless otherwise indicated, a 2′-F modified sugar moiety is in the β-D-ribosyl configuration.

As used herein, “2′-F nucleoside” or “2′-F modified nucleoside” means a nucleoside comprising a 2′-F modified sugar moiety.

As used herein, “2′-substituted nucleoside” means a nucleoside comprising a 2′-substituted furanosyl sugar moiety. As used herein, “2′-substituted” in reference to a sugar moiety means a sugar moiety comprising at least one 2′-substituent group other than H or OH.

As used herein, “5-methylcytosine” means a cytosine modified with a methyl group attached to the 5 position. A 5-methylcytosine is a modified nucleobase.

As used herein, “abasic sugar moiety” means a sugar moiety of a nucleoside that is not attached to a nucleobase. Such abasic sugar moieties are sometimes referred to in the art as “abasic nucleosides.”

As used herein, “about” means within +10% of a value. For example, if it is stated, “the compounds affected about 70% reduction of GYS1”, it is implied that GYS1 levels are reduced within a range of 63% and 77%.

As used herein, “administration” or “administering” means providing a pharmaceutical agent or composition to a subject.

As used herein, “ameliorate” in reference to a treatment means improvement in at least one symptom or hallmark relative to the same symptom or hallmark in the absence of the treatment. In certain embodiments, amelioration is the reduction in the severity or frequency of a symptom or hallmark or the delayed onset of or slowing of progression in the severity or frequency of a symptom or hallmark. In certain embodiments, the symptom or hallmark is seizures, cognitive deterioration, neuromuscular weakness, myoclonus, dementia, ataxia, cerebellar dysfunction, impaired speech, loss of ambulation, swallowing difficulty, or epileptic episodes. The progression or severity of indicators may be determined by subjective or objective measures, which are known to those skilled in the art.

As used herein, “antisense activity” means any detectable and/or measurable change attributable to the hybridization of an antisense compound to its target nucleic acid. In certain embodiments, antisense activity is a decrease in the amount or expression of a target nucleic acid or protein encoded by such target nucleic acid compared to target nucleic acid levels or target protein levels in the absence of the antisense compound.

As used herein, “antisense agent” means an antisense compound and optionally one or more additional features, such as a sense compound.

As used herein, “antisense compound” means an antisense oligonucleotide and optionally one or more additional features, such as a conjugate group.

As used herein, “sense compound” means a sense oligonucleotide and optionally one or more additional features, such as a conjugate group.

As used herein, “antisense oligonucleotide” means an oligonucleotide, including the oligonucleotide portion of an antisense compound, that is capable of hybridizing to a target nucleic acid and is capable of at least one antisense activity. Antisense oligonucleotides include but are not limited to antisense RNAi oligonucleotides and antisense RNase H oligonucleotides.

As used herein, “sense oligonucleotide” means an oligonucleotide, including the oligonucleotide portion of a sense compound, that is capable of hybridizing to an antisense oligonucleotide.

As used herein, “Adult polyglucosan body disease” is characterized by dysfunction of the central and peripheral nervous systems. Associated symptoms and findings may include sensory loss in the legs, progressive muscle weakness of the arms and legs, gait disturbances, urination difficulties, and/or cognitive impairment or dementia.

As used herein, “Andersen's disease”, also known as glycogen storage disease type IV, is caused by deficient activity of the glycogen-branching enzyme, resulting in accumulation of abnormal glycogen in the liver, muscle, and other tissues. The disease course is typically characterized by progressive liver cirrhosis and liver failure. In some case, several neuromuscular variants of Andersen's disease occur that may be evident at birth, late childhood, or adulthood.

As used herein, “ataxia” means impaired motor coordination.

As used herein, “bicyclic nucleoside” or “BNA” means a nucleoside comprising a bicyclic sugar moiety.

As used herein, “bicyclic sugar” or “bicyclic sugar moiety” means a modified sugar moiety comprising two rings, wherein the second ring is formed via a bridge connecting two of the atoms in the first ring thereby forming a bicyclic structure. In certain embodiments, the first ring of the bicyclic sugar moiety is a furanosyl sugar moiety. In certain embodiments, the furanosyl sugar moiety is a ribosyl sugar moiety. In certain embodiments, the bicyclic sugar moiety does not comprise a furanosyl sugar moiety.

As used herein, “cell-targeting moiety” means a conjugate group or portion of a conjugate group that is capable of binding to a particular cell type or particular cell types.

As used herein, “cerebrospinal fluid” or “CSF” means the fluid filling the space around the brain and spinal cord. “Artificial cerebrospinal fluid” or “aCSF” means a prepared or manufactured fluid that has certain properties (e.g., osmolarity, pH, and/or electrolytes) similar to cerebrospinal fluid and is biocompatible with CSF.

As used herein, “chirally enriched population” means a plurality of molecules of identical molecular formula, wherein the number or percentage of molecules within the population that contain a particular stereochemical configuration at a particular chiral center is greater than the number or percentage of molecules expected to contain the same particular stereochemical configuration at the same particular chiral center within the population if the particular chiral center were stereorandom. Chirally enriched populations of molecules having multiple chiral centers within each molecule may contain one or more stereorandom chiral centers. In certain embodiments, the molecules are modified oligonucleotides. In certain embodiments, the molecules are compounds comprising modified oligonucleotides.

As used herein, “chirally controlled” in reference to an internucleoside linkage means chirality at that linkage is enriched for a particular stereochemical configuration.

As used herein, “cleavable moiety” means a bond or group of atoms that is cleaved under physiological conditions, for example, inside a cell, a subject, an animal, or a human.

As used herein, “complementary” in reference to an oligonucleotide means that at least 70% of the nucleobases of the oligonucleotide and the nucleobases of another nucleic acid or one or more portions thereof are capable of hydrogen bonding with one another when the nucleobase sequence of the oligonucleotide and the other nucleic acid are aligned in opposing directions. “Complementary region” in reference to a region of an oligonucleotide means that at least 70% of the nucleobases of that region and the nucleobases of another nucleic acid or one or more regions thereof are capable of hydrogen bonding with one another when the nucleobase sequence of the oligonucleotide and the other nucleic acid are aligned in opposing directions. “Complementary nucleobases” means nucleobases that are capable of forming hydrogen bonds with one another. Complementary nucleobase pairs include adenine (A) and thymine (T), adenine (A) and uracil (U), cytosine (C) and guanine (G), and 5-methylcytosine (^{m}C) and guanine (G). Certain modified nucleobases that pair with natural nucleobases or with other modified nucleobases are known in the art and are not considered complementary nucleobases as define dherein unless indicated otherwise. For example, inosine can pair, but is not considered complementary, with adenosine, cytosine, or uracil. Complementary oligonucleotides and/or target nucleic acids need not have nucleobase complementarity at each nucleoside. Rather, some mismatches are tolerated. As used herein, “fully complementary” or “100% complementary” in reference to an oligonucleotide, or a portion thereof, means that the oligonucleotide, or portion thereof, is complementary to another oligonucleotide or nucleic acid at each nucleobase of the oligonucleotide or nucleic acid.

As used herein, “conjugate group” means a group of atoms that is directly attached to an oligonucleotide. Conjugate groups include a conjugate moiety and a conjugate linker that attaches the conjugate moiety to the oligonucleotide.

As used herein, “conjugate linker” means a single bond or a group of atoms comprising at least one bond that connects a conjugate moiety to an oligonucleotide.

As used herein, “conjugate moiety” means a group of atoms that modifies one or more properties of a molecule compared to the identical molecule lacking the conjugate moiety, including but not limited to pharmacodynamics, pharmacokinetics, stability, binding, absorption, tissue distribution, cellular distribution, cellular uptake, charge and clearance.

As used herein, “contiguous” in the context of an oligonucleotide refers to nucleosides, nucleobases, sugar moieties, or internucleoside linkages that are immediately adjacent to each other. For example, “contiguous nucleobases” means nucleobases that are immediately adjacent to each other in a sequence.

As used herein, “constrained ethyl” or “cEt” or “cEt modified sugar moiety” means a β-D ribosyl bicyclic sugar moiety wherein the second ring of the bicyclic sugar is formed via a bridge connecting the 4′-carbon and the 2′-carbon of the β-D ribosyl sugar moiety, wherein the bridge has the formula 4′-CH(CH₃)—O-2′, and wherein the methyl group of the bridge is in the S configuration.

As used herein, “cEt nucleoside” means a nucleoside comprising a cEt modified sugar moiety.

As used herein, “deoxy region” means a region of 5-12 contiguous nucleotides, wherein at least 70% of the nucleosides comprise a 2′-β-D-deoxyribosyl sugar moiety. In certain embodiments, a deoxy region is the gap of a gapmer.

As used herein, “dementia” means a loss of intellectual function that impairs memory, judgment, or thought.

As used herein, “diluent” means an ingredient in a composition that lacks pharmacological activity, but is pharmaceutically necessary or desirable. For example, the diluent in an injected composition can be a liquid, e.g. aCSF, PBS, or saline solution.

As used herein, “epilepsy” is a central nervous system disorder in which nerve cell activity in the brain becomes chronically hyperexcitable. This predisposes to recurrent episodes of seizures, which may be associated with focal or generalized motor and/or sensory disturbances as well as loss of consciousness. In certain instances, it may also be associated and/or cause other symptoms including myoclonus, cognitive deficits, learning disabilities, or developmental delay in children. In certain instances, it may lead to death in some patients. In certain instances, some forms of epilepsy are associated with progressive neurodegenerative diseases. Many people with epilepsy have more than one symptom.

As used herein, “gapmer” means a modified oligonucleotide comprising an internal region positioned between external regions having one or more nucleosides, wherein the nucleosides comprising the internal region are chemically distinct from the nucleoside or nucleosides comprising the external regions, and wherein the modified oligonucleotide supports RNase H cleavage. The internal region may be referred to as the “gap” and the external regions may be referred to as the “wing.” In certain embodiments, the internal region is a deoxy region. The positions of the internal region or gap refer to the order of the nucleosides of the internal region and are counted starting from the 5′-end of the internal region. Unless otherwise indicated, “gapmer” refers to a sugar motif. In certain embodiments, the sugar moiety of each nucleoside of the gap is a 2′-β-D-deoxyribosyl sugar moiety. In certain embodiments, the gap comprises one 2′-substituted nucleoside at position 1, 2, 3, 4, or 5 of the gap, and the remainder of the nucleosides of the gap are 2′-β-D-deoxynucleosides. As used herein, the term “MOE gapmer” indicates a gapmer having a gap comprising 2′-β-D-deoxynucleosides and wings comprising 2′-MOE nucleosides. As used herein, the term “mixed wing gapmer” indicates a gapmer having wings comprising modified nucleosides comprising at least two different sugar modifications. Unless otherwise indicated, a gapmer may comprise one or more modified internucleoside linkages and/or modified nucleobases and such modifications do not necessarily follow the gapmer pattern of the sugar modifications.

As used herein, “glycogen” is a polysaccharide that is the principal storage form of glucose in animals. Glycogen is found in the form of granules in the cystosol in a variety of tissues, including brain.

As used herein, “GYS1-specific inhibitor” refers to any agent capable of specifically reducing GYS1 expression or activity at the molecular level. For example, GYS1-specific inhibitors include nucleic acids (including antisense compounds), peptides, antibodies, small molecules, and other agents capable of reducing the expression or activity of GYS1.

As used herein, “hotspot region” is a range of nucleobases on a target nucleic acid that is amenable to oligomeric agent or oligomeric compound-mediated reduction of the amount or activity of the target nucleic acid.

As used herein, “hybridization” means the annealing of oligonucleotides and/or nucleic acids. While not limited to a particular mechanism, the most common mechanism of hybridization involves hydrogen bonding, which may be Watson-Crick, Hoogsteen or reversed Hoogsteen hydrogen bonding, between complementary nucleobases. In certain embodiments, complementary nucleic acid molecules include, but are not limited to, an antisense compound and a nucleic acid target. In certain embodiments, complementary nucleic acid molecules include, but are not limited to, an oligonucleotide and a nucleic acid target.

As used herein, “internucleoside linkage” is the covalent linkage between adjacent nucleosides in an oligonucleotide. As used herein, “modified internucleoside linkage” means any internucleoside linkage other than a phosphodiester internucleoside linkage. “Phosphorothioate internucleoside linkage” or “PS internucleoside linkage” is a modified internucleoside linkage in which one of the non-bridging oxygen atoms of a phosphodiester internucleoside linkage is replaced with a sulfur atom.

As used herein, “inverted nucleoside” means a nucleotide having a 3′ to 3′ and/or 5′ to 5′ internucleoside linkage, as shown herein.

As used herein, “inverted sugar moiety” means the sugar moiety of an inverted nucleoside or an abasic sugar moiety having a 3′ to 3′ and/or 5′ to 5′ internucleoside linkage.

As used herein, “Lafora bodies” are neurotoxic inclusions formed as a result of the formation of abnormal glycogen and its precipitation and accumulation to form polyglucosan.

As used herein, “Lafora disease” (LD) is a severe and universally fatal form of adolescence-onset epilepsy resulting from accumulation of Lafora bodies in neurons, muscle, and other tissues. It is characterized by progressive worsening of seizures, myoclonus, cognitive decline, ataxia and speech and swallowing difficulties. Patients ultimately become wheelchair bound, mute, unable to swallow (need gastrostomy feeding), with profound dementia and myoclonic status, and all die usually within 5-10 years from disease onset.

As used herein, “linked nucleosides” are nucleosides that are connected in a contiguous sequence (i.e., no additional nucleosides are presented between those that are linked).

As used herein, “linker-nucleoside” means a nucleoside that links, either directly or indirectly, an oligonucleotide to a conjugate moiety. Linker-nucleosides are located within the conjugate linker of an oligomeric compound. Linker-nucleosides are not considered part of the oligonucleotide portion of an oligomeric compound even if they are contiguous with the oligonucleotide.

As used herein, “mismatch” or “non-complementary” means a nucleobase of a first nucleic acid sequence that is not complementary with the corresponding nucleobase of a second nucleic acid sequence or target nucleic acid when the first and second nucleic acid sequences are aligned in opposing directions.

As used herein, “motif” means the pattern of unmodified and/or modified sugar moieties, nucleobases, and/or internucleoside linkages, in an oligonucleotide.

As used herein, “myoclonus” means episodes of repeated, stereotypic, involuntary muscle jerking or twitching that can affect part of the body or the entire body for variable durations.

As used herein, “non-bicyclic modified sugar moiety” means a modified sugar moiety that comprises a modification, such as a substituent, that does not form a bridge between two atoms of the sugar to form a second ring.

As used herein, “nucleobase” means an unmodified nucleobase or a modified nucleobase. A nucleobase is a heterocyclic moiety. As used herein an “unmodified nucleobase” is adenine (A), thymine (T), cytosine (C), uracil (U), or guanine (G). As used herein, a “modified nucleobase” is a group of atoms other than unmodified A, T, C, U, or G capable of pairing with at least one unmodified nucleobase. A “5-methylcytosine” is a modified nucleobase. A universal base is a modified nucleobase that can pair with any one of the five unmodified nucleobases.

As used herein, “nucleobase sequence” means the order of contiguous nucleobases in a nucleic acid or oligonucleotide independent of any sugar or internucleoside linkage modification.

As used herein, “nucleoside” means a compound, or a fragment of a compound, comprising a nucleobase and a sugar moiety. The nucleobase and sugar moiety are each, independently, unmodified or modified. As used herein, “modified nucleoside” means a nucleoside comprising a modified nucleobase and/or a modified sugar moiety. Modified nucleosides include abasic nucleosides, which lack a nucleobase. “Linked nucleosides” are nucleosides that are connected in a contiguous sequence (i.e., no additional nucleosides are presented between those that are linked).

As used herein, “oligomeric agent” means an oligomeric compound and optionally one or more additional features, such as a second oligomeric compound. An oligomeric agent may be a single-stranded oligomeric compound or may be an oligomeric duplex formed by two complementary oligomeric compounds.

As used herein, “oligomeric compound” means an oligonucleotide and optionally one or more additional features, such as a conjugate group or terminal group. An oligomeric compound may be paired with a second oligomeric compound that is complementary to the first oligomeric compound or may be unpaired. A “singled-stranded oligomeric compound” is an unpaired oligomeric compound.

The term “oligomeric duplex” means a duplex formed by two oligomeric compounds having complementary nucleobase sequences.

As used herein, “oligonucleotide” means a strand of linked nucleosides connected via internucleoside linkages, wherein each nucleoside and internucleoside linkage may be modified or unmodified. Unless otherwise indicated, oligonucleotides consist of 8-50 linked nucleosides. As used herein, “modified oligonucleotide” means an oligonucleotide, wherein at least one nucleoside or internucleoside linkage is modified. As used herein, “unmodified oligonucleotide” means an oligonucleotide that does not comprise any nucleoside modifications or internucleoside modifications. An oligonucleotide may be paired with a second oligonucleotide that is complementary to the oligonucleotide or it may be unpaired. A “single-stranded oligonucleotide” is an unpaired oligonucleotide. A “double-stranded oligonucleotide” is an oligonucleotide that is paired with a second oligonucleotide.

As used herein, “pharmaceutically acceptable carrier or diluent” means any substance suitable for use in administering to an animal. Certain such carriers enable pharmaceutical compositions to be formulated as, for example, tablets, pills, dragees, capsules, liquids, gels, syrups, slurries, suspension and lozenges for the oral ingestion by an animal. In certain embodiments, a pharmaceutically acceptable carrier or diluent is sterile water, sterile saline, sterile buffer solution or sterile artificial cerebrospinal fluid.

As used herein, “pharmaceutically acceptable salts” means physiologically and pharmaceutically acceptable salts of compounds. Pharmaceutically acceptable salts retain the desired biological activity of the parent compound and do not impart undesired toxicological effects thereto.

As used herein, “pharmaceutical composition” means a mixture of substances suitable for administering to a subject. For example, a pharmaceutical composition may comprise an oligomeric compound and a sterile aqueous solution. In certain embodiments, a pharmaceutical composition shows activity in free uptake assay in certain cell lines.

As used herein, “Pompe disease” also called glycogen storage disease type II, is a neuromuscular disorder caused by buildup of glycogen in the body's cells. Pompe disease is a single disease continuum with variable rates of disease progression and different ages of onset. The first symptoms can occur at any age from birth to late adulthood.

As used herein, “prevent” refers to a delaying or forestalling of the onset, development, or progression of a disease, disorder, or condition for a period of time, e.g., from minutes to indefinitely.

As used herein, “prodrug” means a therapeutic agent in a first form outside the body that is converted to a second form within an animal or cells thereof. Typically, conversion of a prodrug within the animal is facilitated by the action of an enzymes (e.g., endogenous or viral enzyme) or chemicals present in cells or tissues and/or by physiologic conditions. In certain embodiments, the first form of the prodrug is less active than the second form.

As used herein, “reducing or inhibiting the amount or activity” refers to a reduction or blockade of the transcriptional expression or activity relative to the transcriptional expression or activity in an untreated or control sample and does not necessarily indicate a total elimination of transcriptional expression or activity.

As used herein, “RNA” means an RNA transcript and includes pre-mRNA and mature mRNA unless otherwise specified.

As used herein, “RNAi agent” means an antisense agent that acts, at least in part, through RISC or Ago2 to modulate a target nucleic acid and/or protein encoded by a target nucleic acid. RNAi agents include, but are not limited to double-stranded siRNA, single-stranded RNA (ssRNAi), and microRNA, including microRNA mimics. RNAi agents may comprise conjugate groups and/or terminal groups. In certain embodiments, an RNAi agent modulates the amount, activity, and/or splicing of a target nucleic acid. The term RNAi agent excludes antisense agents that act through RNase H.

As used herein, “RNase H agent” means an antisense agent that acts through RNase H to modulate a target nucleic acid and/or protein encoded by a target nucleic acid. In certain embodiments, RNase H agents are single-stranded. In certain embodiments, RNase H agents are double-stranded. RNase H agents may comprise conjugate groups and/or terminal groups. In certain embodiments, an RNase H agent modulates the amount and/or activity of a target nucleic acid. The term RNase H agent excludes antisense agents that act principally through RISC/Ago2.

As used herein, “self-complementary” in reference to an oligonucleotide means an oligonucleotide that at least partially hybridizes to itself.

As used herein, “seizures” are a symptom of many different disorders and conditions that can affect the brain. “Seizures” are typically caused by dysfunction in the electric communication between neurons in the brain, resulting from a brain injury or an underlying disease or disorder, such as a genetic condition, for example. In patients with a seizure disorder, neurons are hyperexcitable with a propensity to increased epileptiform discharges and spontaneous firing which can intermittently culminate in a seizure episode. Seizures can take on different forms and affect people in different ways. Common physical changes that may occur during a seizure are difficulty talking, inability to swallow, drooling, repeated blinking of the eyes, staring, lack of movement of muscle tone, slumping tremors, twitching, or jerking movements, rigid or tense muscles, repeated non-purposeful movements, called automatisms, involving the face, arms, or legs, convulsions, loss of control of urine or stool, sweating, change in skin color (paleness or flushing), dilation of pupils, biting of tongue, difficulty breathing, heart palpitations. In some embodiments, seizures are mild. In other embodiments, seizures are completely disabling or may result in death. Abnormal brain activity can often be documented by abnormal findings on an electroencephalogram (EEG) with epileptiform wave forms indicative of the underlying hyperexcitable seizure phenotype.

As used herein, “single-stranded” means a nucleic acid (including but not limited to an oligonucleotide) that is unpaired and is not part of a duplex. Single-stranded compounds are capable of hybridizing with complementary nucleic acids to form duplexes, at which point they are no longer single-stranded.

As used herein, “stabilized phosphate group” means a 5′-phosphate analog that is metabolically more stable than a 5′-phosphate as naturally occurs on DNA or RNA.

As used herein, “standard in vitro assay” means the assays described in Examples 1-3 and reasonable variations thereof.

As used herein, “stereorandom chiral center” in the context of a population of molecules of identical molecular formula means a chiral center having a random stereochemical configuration. For example, in a population of molecules comprising a stereorandom chiral center, the number of molecules having the (S) configuration of the stereorandom chiral center may be but is not necessarily the same as the number of molecules having the (R) configuration of the stereorandom chiral center. The stereochemical configuration of a chiral center is considered random when it is the result of a synthetic method that is not designed to control the stereochemical configuration. In certain embodiments, a stereorandom chiral center is a stereorandom phosphorothioate internucleoside linkage.

As used herein, “subject” means a human or non-human animal. In certain embodiments, the subject is a human.

As used herein, “sugar moiety” means an unmodified sugar moiety or a modified sugar moiety. As used herein, “unmodified sugar moiety” means a 2′-OH(H) β-D-ribosyl moiety, as found in RNA (an “unmodified RNA sugar moiety”), or a 2′-H(H) β-D-deoxyribosyl sugar moiety, as found in DNA (an “unmodified DNA sugar moiety”). Unmodified sugar moieties have one hydrogen at each of the 1′, 3′, and 4′ positions, an oxygen at the 3′ position, and two hydrogens at the 5′ position. As used herein, “modified sugar moiety” or “modified sugar” means a modified furanosyl sugar moiety or a sugar surrogate.

As used herein, “sugar surrogate” means a modified sugar moiety having other than a furanosyl moiety that can link a nucleobase to another group, such as an internucleoside linkage, conjugate group, or terminal group in an oligonucleotide. Modified nucleosides comprising sugar surrogates can be incorporated into one or more positions within an oligonucleotide and such oligonucleotides are capable of hybridizing to complementary oligomeric compounds or target nucleic acids.

As used herein, “symptom or hallmark” means any physical feature or test result that indicates the existence or extent of a disease or disorder. In certain embodiments, a symptom is apparent to a subject or to a medical professional examining or testing the subject. In certain embodiments, a hallmark is apparent upon invasive diagnostic testing, including, but not limited to, post-mortem tests. In certain embodiments, a hallmark is apparent on a brain MRI scan.

As used herein, “target nucleic acid” and “target RNA” mean a nucleic acid that an antisense compound is designed to affect. Target RNA means an RNA transcript and includes pre-mRNA and mRNA unless otherwise specified.

As used herein, “target region” means a portion of a target nucleic acid to which an oligomeric compound is designed to hybridize.

As used herein, “terminal group” means a chemical group or group of atoms that is covalently linked to a terminus of an oligonucleotide.

As used herein, “treating” means improving a subject's disease or condition by administering an oligomeric agent or oligomeric compound described herein. In certain embodiments, treating a subject improves a symptom relative to the same symptom in the absence of the treatment. In certain embodiments, treatment reduces in the severity or frequency of a symptom, or delays the onset of a symptom, slows the progression of a symptom, or slows the severity or frequency of a symptom.

As used herein, “therapeutically effective amount” means an amount of a pharmaceutical agent that provides a therapeutic benefit to a subject. For example, a therapeutically effective amount improves a symptom of a disease.

### CERTAIN EMBODIMENTS

The present disclosure provides the following non-limiting numbered embodiments:

Embodiment 1. An oligomeric compound comprising a modified oligonucleotide consisting of 12 to 30 linked nucleosides wherein the nucleobase sequence of the modified oligonucleotide is at least 80% complementary to an equal length portion of a GYS1 nucleic acid, and wherein the modified oligonucleotide comprises at least one modification selected from a modified sugar moiety and a modified internucleoside linkage.

Embodiment 2. An oligomeric compound comprising a modified oligonucleotide consisting of 12 to 30 linked nucleosides and having a nucleobase sequence comprising at least 12, at least 13, at least 14, at least 15, at least 16, at least 17, at least 18, at least 19, or 20 contiguous nucleobases of any of the nucleobase sequences of SEQ ID NOs: 19-3023, wherein the modified oligonucleotide comprises at least one modification selected from a modified sugar moiety and a modified internucleoside linkage.

Embodiment 3. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or 20 contiguous nucleobases complementary to:
an equal length portion of nucleobases 244-271 of SEQ ID NO: 1;
an equal length portion of nucleobases 289-317 of SEQ ID NO: 1;
an equal length portion of nucleobases 391-434 of SEQ ID NO: 1;
an equal length portion of nucleobases 1191-1230 of SEQ ID NO: 1;
an equal length portion of nucleobases 2633-2686 of SEQ ID NO: 1;
an equal length portion of nucleobases 2727-2764 of SEQ ID NO: 1;
an equal length portion of nucleobases 2809-2851 of SEQ ID NO: 1;
an equal length portion of nucleobases 3277-3326 of SEQ ID NO: 1;
an equal length portion of nucleobases 3341-3385 of SEQ ID NO: 1;
an equal length portion of nucleobases 3565-3591 of SEQ ID NO: 1;
an equal length portion of nucleobases 3239-3298 of SEQ ID NO: 2;
an equal length portion of nucleobases 3324-3389 of SEQ ID NO: 2;
an equal length portion of nucleobases 3576-3612 of SEQ ID NO: 2;
an equal length portion of nucleobases 5310-5350 of SEQ ID NO: 2;
an equal length portion of nucleobases 5706-5762 of SEQ ID NO: 2;
an equal length portion of nucleobases 5914-5948 of SEQ ID NO: 2;
an equal length portion of nucleobases 6189-6221 of SEQ ID NO: 2;
an equal length portion of nucleobases 6986-7034 of SEQ ID NO: 2;
an equal length portion of nucleobases 7803-7844 of SEQ ID NO: 2;
an equal length portion of nucleobases 8371-8400 of SEQ ID NO: 2;
an equal length portion of nucleobases 8514-8555 of SEQ ID NO: 2;
an equal length portion of nucleobases 8826-8857 of SEQ ID NO: 2;
an equal length portion of nucleobases 12100-12138 of SEQ ID NO: 2;
an equal length portion of nucleobases 12350-12385 of SEQ ID NO: 2;
an equal length portion of nucleobases 12612-12659 of SEQ ID NO: 2;
an equal length portion of nucleobases 15653-15688 of SEQ ID NO: 2;
an equal length portion of nucleobases 15801-15845 of SEQ ID NO: 2;
an equal length portion of nucleobases 16745-16800 of SEQ ID NO: 2;
an equal length portion of nucleobases 16828-16864 of SEQ ID NO: 2;
an equal length portion of nucleobases 16856-16910 of SEQ ID NO: 2;
an equal length portion of nucleobases 16915-16947 of SEQ ID NO: 2;
an equal length portion of nucleobases 19917-19980 of SEQ ID NO: 2;
an equal length portion of nucleobases 24606-24665 of SEQ ID NO: 2; or
an equal length portion of SEQ ID NO: 3024,
   wherein the modified oligonucleotide comprises at least one modification selected from a modified sugar moiety and a modified internucleoside linkage.

Embodiment 4. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or 20 contiguous nucleobases of a sequence selected from:
SEQ ID NOs: 102, 1406, 1479, 1556, 1633, 1710, 1787, 1863, and 179;
SEQ ID NOs: 256, 1939, 2015, 2092, 2169, 333, and 2246;
SEQ ID NOs: 792, 26, 869, 946, 1023, 104, and 1100;
SEQ ID NOs: 344, 1181, 421, 1257, 37, and 1334;
SEQ ID NOs: 1724, 56, 1877, 1953, 2029, 2106, 2183, 2260, 2337, 2414, 133, and 2491;
SEQ ID NOs: 2721, 2797, 2874, 210, 500, 577, 654, 730, 806, 883, 960, 287, 364, 1037, 1114, 1191, and 1267;
SEQ ID NOs: 1344, 1421, 1494, 1571, 1648, and 441;
SEQ ID NOs: 2723, 2800, 2877, 503, 61, 580, 657, 138, and 733;
SEQ ID NOs: 1194, 1270, 1347, 446, 1424, 1497, 1574, and 1651;
SEQ ID NOs: 140, 1348, 217, 1498, and 1575;
SEQ ID NOs: 1273, 1350, 1426, 1500, 1577, 1654, 1731, 1807, 372, and 1960;
SEQ ID NOs: 2498, 2575, 2652, 2727, 2804, 2881, 507, 584, 661, 737, 813, 890, 967, 1044, 1121, 65, 1198, 1274, 1351, 1427, 1501, 1578, 1655, 1732, 1808, 1884, 1961, 2037, 2114, 2191, 2268, 2345, 2422, 2499, and 2576;
SEQ ID NOs: 1353, 373, 1503, 1580, 1657, 1734, 1810, 1886, 1963, 2039, 2116, 2193, 2270, 2347, and 450;
SEQ ID NOs: 2044, 2121, 2198, 2275, 2352, 2429, and 2506;
SEQ ID NOs: 2584, 70, and 2736;
SEQ ID NOs: 2508, 2585, 2661, 2737, 2814, 2891, 517, and 594;
SEQ ID NOs: 2201, 2278, 2355, 2432, 2509, 2586, 2662, 2738, 2815, 302, and 2892;
SEQ ID NOs: 1743, 1819, 1895, 1972, 2048, 456, 2125, 2202, 2279, and 2356;
SEQ ID NOs: 1513, 1590, 1667, and 1744;
SEQ ID NOs: 2511, 2588, 2664, 2740, 2817, 2894, 520, 597, 3021, 673, 3022, and 750;
SEQ ID NOs: 1514, 1591, 1668, 1745, 73, 1821, and 1897;
SEQ ID NOs: 2818, 2895, 521, 598, 674, 751, and 827;
SEQ ID NOs: 2674, 2750, 2827, and 2904;
SEQ ID NOs: 914, 991, 1068, 1145, and 1222;
SEQ ID NOs: 532, 2976, 2964, 609, 310, 685, 2972, 2988, 2993, 2959, 762, 2968, 838, 915, 992, 1069, 2973, 2992, and 1146;
SEQ ID NOs: 1303, 83, 1380, 1454, 2954, 2947, 1530, 1607, 1684, 1761, 1837, 2951, 2949, 2944, 2943, 1913, and 1990;
SEQ ID NOs: 314, 843, 920, 997, and 1074;
SEQ ID NOs: 1303, 83, 1380, 1454, 1530, 1607, 1684, 1761, 1837, 1913, and 1990;
SEQ ID NOs: 2954, 2947, 2951, 2949, 2944, and 2943;
SEQ ID NOs: 2682, 2758, 2835, 2912, 538, 615, 691, 767, 844, 921, 998, 1075, 1152, 161, and 1305;
SEQ ID NOs: 1382, 1456, and 1532;
SEQ ID NOs: 1915, 469, 2068, 2979, 2967, 2958, 2938, 2939, 2940, 2997, 2996, 2941, 2995, 2974, 2970, 2980, 2971, 2961, 2984, 2957, and 2145;
SEQ ID NOs: 2376, 2453, 2530, 2607, 2683, 2759, 2836, 2913, 539, and 616;
SEQ ID NOs: 1390, 1464, 1540, 243, 1617, and 1694; or
SEQ ID NOs: 2621, 2697, 2773, 2850, and 2927;
   wherein the modified oligonucleotide comprises at least one modification selected from a modified sugar moiety and a modified internucleoside linkage.

Embodiment 5. The oligomeric compound of any of embodiments 1-4, wherein the modified oligonucleotide has a nucleobase sequence that is at least 85%, at least 90%, at least 95%, or 100% complementary to the nucleobase sequence of any one of SEQ ID NOs: 1-10 and 14 when measured across the entire nucleobase sequence of the modified oligonucleotide.

Embodiment 6. The oligomeric compound of any of embodiments 1-5, wherein the modified oligonucleotide comprises at least one modified nucleoside.

Embodiment 7. The oligomeric compound of any of embodiments 1-6, wherein the modified oligonucleotide comprises at least one modified nucleoside comprising a modified sugar moiety.

Embodiment 8. The oligomeric compound of embodiment 7, wherein the modified oligonucleotide comprises at least one modified nucleoside comprising a bicyclic sugar moiety.

Embodiment 9. The oligomeric compound of embodiment 8, wherein the bicyclic sugar moiety has a 2′-4′ bridge selected from —O—CH₂—; and —O—CH(CH₃)—.

Embodiment 10. The oligomeric compound of any of embodiments 6-9, wherein the modified oligonucleotide comprises at least one modified nucleoside comprising a non-bicyclic modified sugar moiety.

Embodiment 11. The oligomeric compound of embodiment 10, wherein the non-bicyclic modified sugar moiety is a 2′-O(CH₂)₂OCH₃ ribosyl sugar moiety or a 2′-OMe modified sugar moiety.

Embodiment 12. The oligomeric compound of any of embodiments 6-11, wherein the modified oligonucleotide comprises at least one modified nucleoside comprising a sugar surrogate.

Embodiment 13. The oligomeric compound of embodiment 12, wherein the sugar surrogate is any of morpholino, modified morpholino, PNA, THP, and F-HNA.

Embodiment 14. The oligomeric compound of any of embodiments 1-13, wherein the modified oligonucleotide is a gapmer.

Embodiment 15. The oligomeric compound of any of embodiments 1-14, wherein the modified oligonucleotide comprises at least one modified internucleoside linkage.

Embodiment 16. The oligomeric compound of embodiment 15, wherein each internucleoside linkage of the modified oligonucleotide is a modified internucleoside linkage.

Embodiment 17. The oligomeric compound of embodiment 15 or embodiment 16, wherein at least one internucleoside linkage is a phosphorothioate internucleoside linkage.

Embodiment 18. The oligomeric compound of embodiment 15 or embodiment 17, wherein the modified oligonucleotide comprises at least one phosphodiester internucleoside linkage.

Embodiment 19. The oligomeric compound of any of embodiments 15 or 17-18, wherein each internucleoside linkage is independently selected from a phosphodiester internucleoside linkage or a phosphorothioate internucleoside linkage.

Embodiment 20. The oligomeric compound of any of embodiments 15 or 17-19, wherein at least 4, at least 5, at least 6, at least 7, at least 8, at least 9, at least 10, at least 11, at least 12, at least 13, at least 14, at least 15, at least 16, at least 17, at least 18, or at least 19 internucleoside linkages of the modified oligonucleotide are phosphorothioate internucleoside linkages.

Embodiment 21. The oligomeric compound of any of embodiments 1-15 or 17-20, wherein the internucleoside linkage motif of the modified oligonucleotide is selected from: 5′-soooosssssssssssooss-3′, 5′-sososssssssssssooss-3′, 5′-sooosssssssssssooss-3′, 5′-soooossssssssssooss-3′, 5′-sooosssssssssssooos-3′, 5′-sooossssssssssooss-3′, and 5′-sooooossssssssssoss-3′; wherein each “s” represents a phosphorothioate internucleoside linkage and each “o” represents a phosphodiester internucleoside linkage.

Embodiment 22. The oligomeric compound of any of embodiments 1-21, wherein the modified oligonucleotide comprises at least one modified nucleobase.

Embodiment 23. The oligomeric compound of embodiment 22, wherein the modified nucleobase is a 5-methylcytosine.

Embodiment 24. The oligomeric compound of any of embodiments 1-23, wherein the modified oligonucleotide comprises a deoxy region.

Embodiment 25. The oligomeric compound of embodiment 24, wherein each nucleoside of the deoxy region is a 2′-deoxynucleoside.

Embodiment 26. The oligomeric compound of embodiment 24 or embodiment 25, wherein the deoxy region consists of 6, 7, 8, 9, 10, or 6-10 linked nucleosides.

Embodiment 27. The oligomeric compound of any of embodiments 24-26, wherein each nucleoside immediately adjacent to the deoxy region comprises a modified sugar moiety.

Embodiment 28. The oligomeric compound of any of embodiments 24-27, wherein the deoxy region is flanked on the 5′-side by a 5′-region consisting of 1-6 linked 5′-region nucleosides and on the 3′-side by a 3′-region consisting of 1-6 linked 3′-region nucleosides; wherein at least one nucleoside of the 5′-region comprises a modified sugar moiety; and at least one nucleoside of the 3′-region comprises a modified sugar moiety.

Embodiment 29. The oligomeric compound of embodiment 28, wherein each nucleoside of the 5′-region comprises a modified sugar moiety.

Embodiment 30. The oligomeric compound of embodiment 28 or embodiment 29, wherein each nucleoside of the 3′-region comprises a modified sugar moiety.

Embodiment 31. The oligomeric compound of any of embodiments 1-30, wherein the modified oligonucleotide consists of 12-30, 12-22, 12-20,14-18, 14-20, 15-17, 15-25, 16-20, 18-22, or 18-20 linked nucleosides.

Embodiment 32. The oligomeric compound of any of embodiments 1-31, wherein the modified oligonucleotide is a pharmaceutically acceptable salt.

Embodiment 33. The oligomeric compound of embodiment 32, wherein the modified oligonucleotide is a pharmaceutically acceptable salt comprising one or more cations selected from sodium, potassium, calcium, and magnesium.

Embodiment 34. The oligomeric compound of any of embodiments 1-33, wherein the modified oligonucleotide consists of 16, 17, 18, 19, or 20 linked nucleosides.

Embodiment 35. The oligomeric compound of any of embodiments 1-34, wherein the modified oligonucleotide consists of 20 linked nucleosides.

Embodiment 36. The oligomeric compound of any of embodiments 1-35, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides; wherein
each of the 5′-region nucleosides and each of the 3′-region nucleosides comprises a modified sugar moiety and at least one of the central region nucleosides comprises a 2′-β-D-deoxyribosyl sugar moiety.

Embodiment 37. The oligomeric compound of any of embodiments 1-35, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides; wherein
each of the 5′-region nucleosides and each of the 3′-region nucleosides comprises a modified sugar moiety and at least 6 of the central region nucleosides comprises a 2′-β-D-deoxyribosyl sugar moiety.

Embodiment 38. The oligomeric compound of any of embodiments 1-35, wherein the modified oligonucleotide has a sugar motif comprising:
a 5′-region consisting of 1-6 linked 5′-region nucleosides;
a central region consisting of 6-10 linked central region nucleosides; and
a 3′-region consisting of 1-6 linked 3′-region nucleosides; wherein
each of the 5′-region nucleosides and each of the 3′-region nucleosides comprises a modified sugar moiety and each of the central region nucleosides comprises a 2′-β-D-deoxyribosyl sugar moiety.

Embodiment 39. The oligomeric compound of embodiment 38, wherein the modified oligonucleotide has a sugar motif comprising:
a 5′-region consisting of 5 linked 5′-region nucleosides;
a central region consisting of 10 linked central region nucleosides; and
a 3′-region consisting of 5 linked 3′-region nucleosides; wherein
each of the 5′-region nucleosides and each of the 3′-region nucleosides comprises a 2′-O(CH₂)₂OCH₃ ribosyl sugar moiety, and each of the central region nucleosides comprises a 2′-β-D-deoxyribosyl sugar moiety.

Embodiment 40. The oligomeric compound of embodiment 38, wherein the modified oligonucleotide has a sugar motif comprising:
a 5′-region consisting of 6 linked 5′-region nucleosides;
a central region consisting of 10 linked central region nucleosides; and
a 3′-region consisting of 4 linked 3′-region nucleosides; wherein
each of the 5′-region nucleosides and each of the 3′-region nucleosides comprises a 2′-O(CH₂)₂OCH₃ ribosyl sugar moiety, and each of the central region nucleosides comprises a 2′-β-D-deoxyribosyl sugar moiety.

Embodiment 41. The oligomeric compound of embodiment 38, wherein the modified oligonucleotide has a sugar motif comprising:
a 5′-region consisting of 5 linked 5′-region nucleosides;
a central region consisting of 9 linked central region nucleosides; and
a 3′-region consisting of 5 linked 3′-region nucleosides; wherein
each of the 5′-region nucleosides and each of the 3′-region nucleosides comprises a 2′-O(CH₂)₂OCH₃ ribosyl sugar moiety, and each of the central region nucleosides comprises a 2′-β-D-deoxyribosyl sugar moiety.

Embodiment 42. The oligomeric compound of any of embodiments 1-41, consisting of the modified oligonucleotide.

Embodiment 43. The oligomeric compound of any of embodiments 1-41, wherein the oligomeric compound comprises a conjugate group.

Embodiment 44. The oligomeric compound of embodiment 43, wherein the conjugate group comprises a conjugate linker and a conjugate moiety.

Embodiment 45. The oligomeric compound of embodiment 43 or embodiment 44, wherein the conjugate linker consists of a single bond.

Embodiment 46. The oligomeric compound of embodiment 43 or embodiment 44, wherein the conjugate linker comprises 1-3 linker nucleosides.

Embodiment 47. The oligomeric compound of any of embodiments 43-45, wherein the conjugate linker does not comprise any linker nucleosides.

Embodiment 48. The oligomeric compound of any of embodiments 43-47, wherein the conjugate group is attached to the modified oligonucleotide at the 5′-end of the modified oligonucleotide.

Embodiment 49. The oligomeric compound of any of embodiments 43-47, wherein the conjugate group is attached to the modified oligonucleotide at the 3′-end of the modified oligonucleotide.

Embodiment 50. The oligomeric compound of any of embodiments 1-41 or 43-49, wherein the oligomeric compound comprises a terminal group.

Embodiment 51. The oligomeric compound of embodiment 50, wherein the terminal group is an abasic sugar moiety.

Embodiment 52. The oligomeric compound of any of embodiments 1-51, wherein the oligomeric compound is an RNase H agent comprising the oligomeric compound.

Embodiment 53. An oligomeric compound comprising a modified oligonucleotide according to the following chemical notation: ^{m}Cₑₛ^{m}CₑₒGₑₒTₑₒ^{m}CₑₛT_{ds}A_{ds}^{m}C_{ds}A_{ds}G_{ds}G_{ds}A_{ds}T_{ds}T_{ds}T_{ds}Tₑₒ^{m}CₑₒTₑₛAₑₛGₑ (SEQ ID NO: 83), wherein:
A=an adenine nucleobase,
^{m}C=a 5-methylcytosine nucleobase,
G=a guanine nucleobase,
T=a thymine nucleobase,
e=a 2′-O(CH₂)₂OCH₃ ribosyl sugar moiety,
d=a 2′-β-D-deoxyribosyl sugar moiety,
s=a phosphorothioate internucleoside linkage, and
o=a phosphodiester internucleoside linkage.

Embodiment 54. An oligomeric compound comprising a modified oligonucleotide according to the following chemical notation: TₑₛTₑₒ^{m}Cₑₒ^{m}CₑₒGₑₛT_{ds}^{m}C_{ds}T_{ds}A_{ds}^{m}C_{ds}A_{ds}G_{ds}G_{ds}A_{ds}T_{ds}TₑₒTₑₒTₑₛ^{m}CₑₛTₑ (SEQ ID NO: 1454), wherein:
A=an adenine nucleobase,
^{m}C=a 5-methylcytosine nucleobase,
G=a guanine nucleobase,
T=a thymine nucleobase,
e=a 2′-O(CH₂)₂OCH₃ ribosyl sugar moiety,
d=a 2′-β-D-deoxyribosyl sugar moiety,
s=a phosphorothioate internucleoside linkage, and
o=a phosphodiester internucleoside linkage.

Embodiment 55. An oligomeric compound comprising a modified oligonucleotide according to the following chemical notation: TₑₛTₑₒ^{m}Cₑₒ^{m}CₑₒGₑₒTₑₒ^{m}C_{ds}T_{ds}A_{ds}^{m}C_{ds}A_{ds}G_{ds}G_{ds}A_{ds}T_{ds}T_{ds}TₑₒTₑₛ^{m}CₑₛTₑ (SEQ ID NO: 1454), wherein:
A=an adenine nucleobase,
^{m}C=a 5-methylcytosine nucleobase,
G=a guanine nucleobase,
T=a thymine nucleobase,
e=a 2′-O(CH₂)₂OCH₃ ribosyl sugar moiety,
d=a 2′-β-D-deoxyribosyl sugar moiety,
s=a phosphorothioate internucleoside linkage, and
o=a phosphodiester internucleoside linkage.

Embodiment 56. An oligomeric compound comprising a modified oligonucleotide according to the following chemical notation: Gₑₛ^{m}CₑₒAₑₒ^{m}CₑₒAₑₛ^{m}C_{ds}A_{ds}A_{ds}G_{ds}T_{ds}A_{ds}A_{ds}A_{ds}G_{ds}^{m}C_{ds}TₑₒAₑₒGₑₛ^{m}CₑₛAₑ (SEQ ID NO: 921), wherein:
A=an adenine nucleobase,
^{m}C=a 5-methylcytosine nucleobase,
G=a guanine nucleobase,
T=a thymine nucleobase,
e=a 2′-O(CH₂)₂OCH₃ ribosyl sugar moiety,
d=a 2′-β-D-deoxyribosyl sugar moiety,
s=a phosphorothioate internucleoside linkage, and
o=a phosphodiester internucleoside linkage.

Embodiment 57. The oligomeric compound of any of embodiments 53-56, wherein the modified oligonucleotide is a pharmaceutically acceptable salt.

Embodiment 58. The oligomeric compound of embodiment 57, wherein the modified oligonucleotide is a pharmaceutically acceptable salt comprising one or more cations selected from sodium, potassium, calcium, and magnesium.

Embodiment 59. The oligomeric compound of any of embodiments 1-58, wherein the oligomeric compound is a singled-stranded oligomeric compound.

Embodiment 60. An oligomeric duplex, comprising a first oligomeric compound and a second oligomeric compound comprising a second modified oligonucleotide, wherein the first oligomeric compound is an oligomeric compound of any of embodiments 1-59.

Embodiment 61. The oligomeric duplex of embodiment 60, wherein the second modified oligonucleotide consists of 12 to 50 linked nucleosides, and wherein the nucleobase sequence of the second modified oligonucleotide comprises a complementary region of at least 8 nucleobases that is at least 90% complementary to an equal length portion of the first modified oligonucleotide.

Embodiment 62. The oligomeric duplex of embodiment 60 or embodiment 61, wherein the modified oligonucleotide of the first oligomeric compound comprises a 5′-stabilized phosphate group.

Embodiment 63. The oligomeric duplex of embodiment 62, wherein the stabilized phosphate group comprises a cyclopropyl phosphonate or a vinyl phosphonate.

Embodiment 64. The oligomeric duplex of any of embodiments 60-63, wherein at least one nucleoside of the second modified oligonucleotide comprises a modified sugar moiety.

Embodiment 65. The oligomeric duplex of embodiment 64, wherein the modified sugar moiety of the second modified oligonucleotide comprises a bicyclic sugar moiety.

Embodiment 66. The oligomeric duplex of embodiment 65, wherein the bicyclic sugar moiety of the second modified oligonucleotide comprises a 2′-4′ bridge selected from —O—CH₂—; and —O—CH(CH₃)—.

Embodiment 67. The oligomeric duplex of embodiment 64, wherein the modified sugar moiety of the second modified oligonucleotide comprises a non-bicyclic modified sugar moiety.

Embodiment 68. The oligomeric duplex of embodiment 67, wherein the non-bicyclic modified sugar moiety of the second modified oligonucleotide is a 2′-O(CH₂)₂OCH₃ ribosyl sugar moiety, a 2′-F modified sugar moiety, or 2′-OMe modified sugar moiety.

Embodiment 69. The oligomeric duplex of any one of embodiments 60-68, wherein at least one internucleoside linkage of the second modified oligonucleotide is a modified internucleoside linkage.

Embodiment 70. The oligomeric duplex of embodiment 69, wherein at least one modified internucleoside linkage of the second modified oligonucleotide is a phosphorothioate internucleoside linkage.

Embodiment 71. The oligomeric duplex of any of embodiments 60-70, wherein at least one internucleoside linkage of the second modified oligonucleotide is a phosphodiester internucleoside linkage.

Embodiment 72. The oligomeric duplex of any of embodiments 60-71, wherein each internucleoside linkage of the second modified oligonucleotide is independently selected from a phosphodiester or a phosphorothioate internucleoside linkage.

Embodiment 73. The oligomeric duplex of any of embodiments 60-72, wherein the second modified oligonucleotide comprises at least one modified nucleobase.

Embodiment 74. The oligomeric duplex of embodiment 73, wherein the at least one modified nucleobase of the second modified oligonucleotide is 5-methylcytosine.

Embodiment 75. The oligomeric duplex of any of embodiments 60-74, wherein the second modified oligonucleotide comprises a conjugate group.

Embodiment 76. The oligomeric duplex of embodiment 75, wherein the conjugate group comprises a conjugate linker and a conjugate moiety.

Embodiment 77. The oligomeric duplex of embodiment 75 or embodiment 76, wherein the conjugate group is attached to the 5′-end of the second modified oligonucleotide.

Embodiment 78. The oligomeric duplex of embodiment 75 or embodiment 76, wherein the conjugate group is attached to the 3′-end of the second modified oligonucleotide.

Embodiment 79. The oligomeric duplex of any of embodiments 75-78, wherein the conjugate group comprises a lipid.

Embodiment 80. The oligomeric duplex of any of embodiments 60-79, wherein the second modified oligonucleotide comprises a terminal group.

Embodiment 81. The oligomeric duplex of embodiment 80, wherein the terminal group is an abasic sugar moiety.

Embodiment 82. The oligomeric duplex of any of embodiments 60-81, wherein the second modified oligonucleotide consists of 12 to 20, 12 to 25, 12 to 30, 12 to 50, 13 to 20, 13 to 25, 13 to 30, 13 to 50, 14 to 20, 14 to 25, 14 to 30, 14 to 50, 15 to 20, 15 to 25, 15 to 30, 15 to 50, 16 to 18, 16 to 20, 16 to 25, 16 to 30, 16 to 50, 17 to 20, 17 to 25, 17 to 30, 17 to 50, 18 to 20, 18 to 22, 18 to 25, 18 to 30, 18 to 50, 19 to 20, 19 to 25, 19 to 30, 19 to 50, 20 to 25, 20 to 30, 20 to 50, 21 to 25, 21 to 30, 21 to 50, 22 to 25, 22 to 30, 22 to 50, 23 to 25, 23 to 30, or 23 to 50 linked nucleosides.

Embodiment 83. An antisense agent comprising an antisense compound, wherein the antisense compound is an oligomeric compound of any of embodiments 1-59.

Embodiment 84. An antisense agent comprising an antisense compound, wherein the antisense compound is an oligomeric duplex of any of embodiments 60-82.

Embodiment 85. The antisense agent of embodiment 83 or embodiment 84, wherein the antisense agent comprises a conjugate group, wherein the conjugate group comprises a cell-targeting moiety.

Embodiment 86. A modified oligonucleotide according to the following chemical structure:

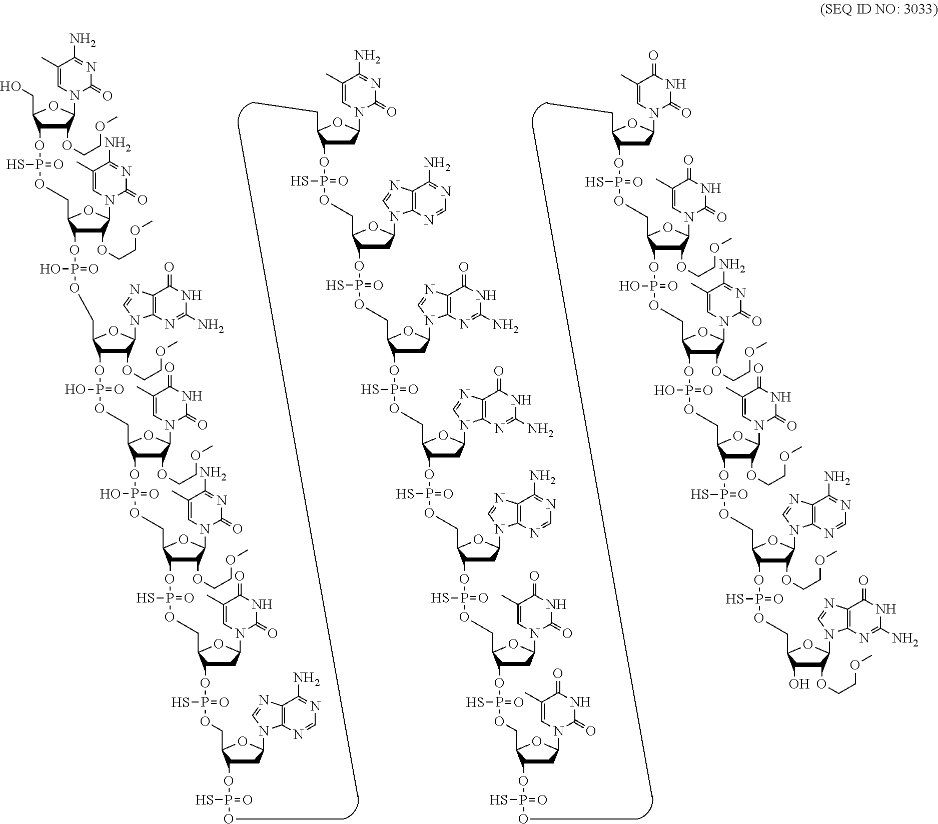

or a pharmaceutically acceptable salt thereof.

Embodiment 87. A modified oligonucleotide according to the following chemical structure:

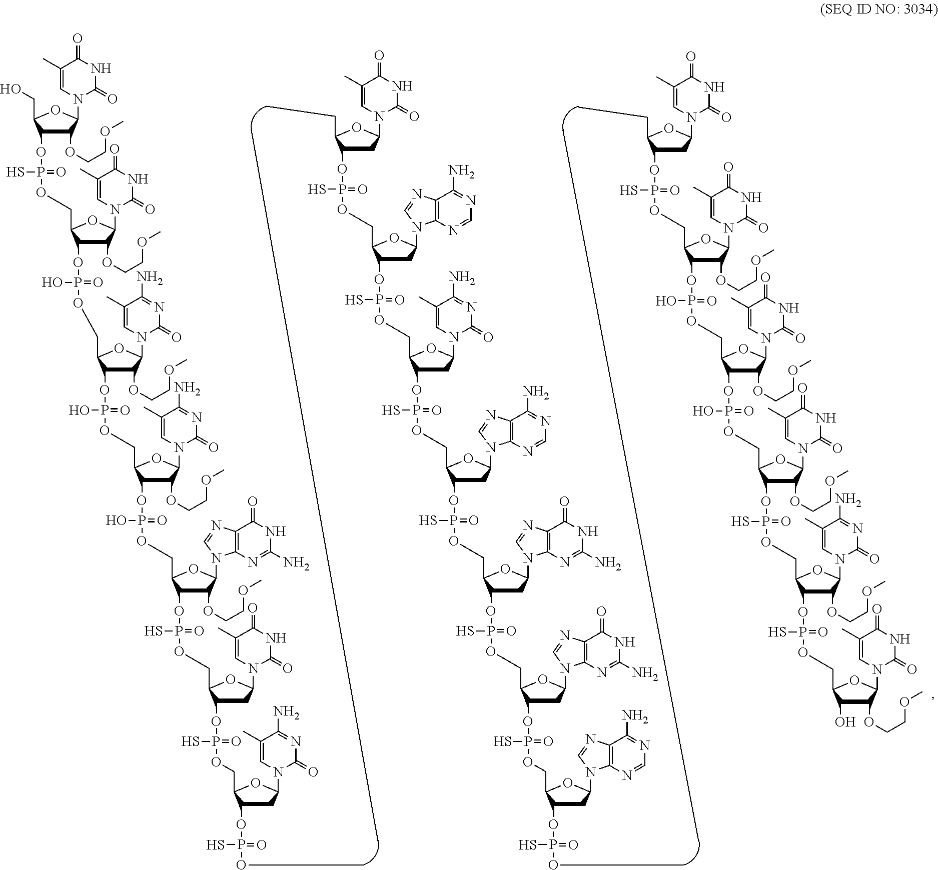

or a pharmaceutically acceptable salt thereof.

Embodiment 88. A modified oligonucleotide according to the following chemical structure:

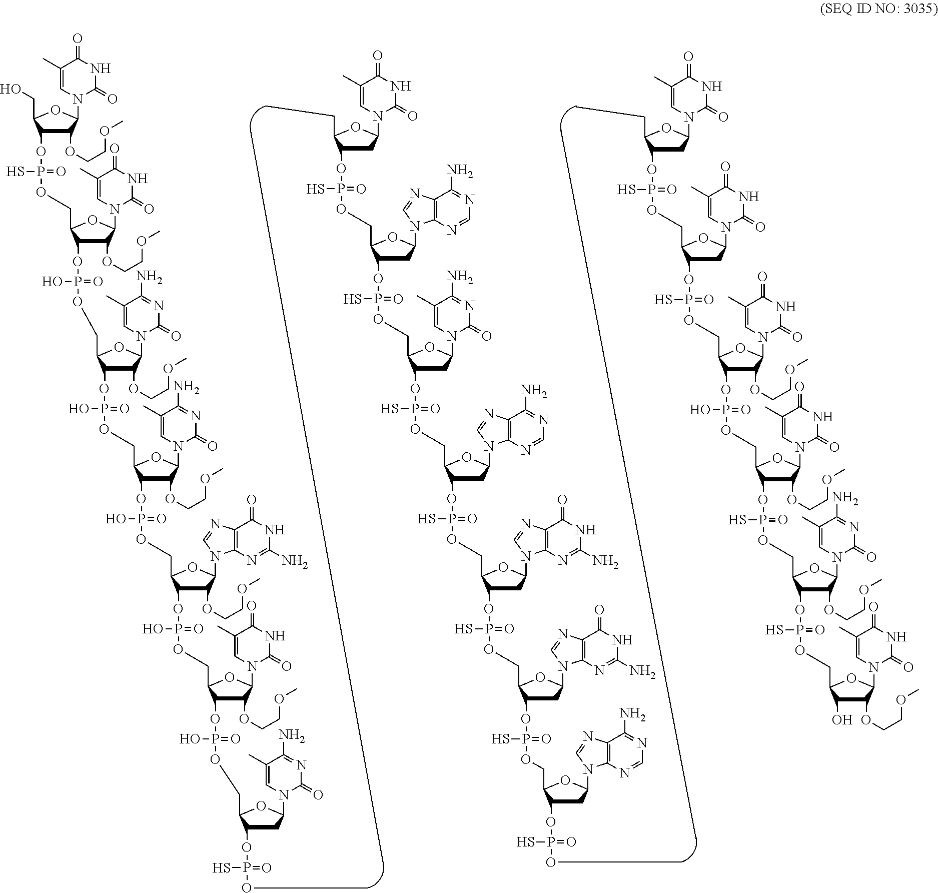

or a pharmaceutically acceptable salt thereof.

Embodiment 89. A modified oligonucleotide according to the following chemical structure:

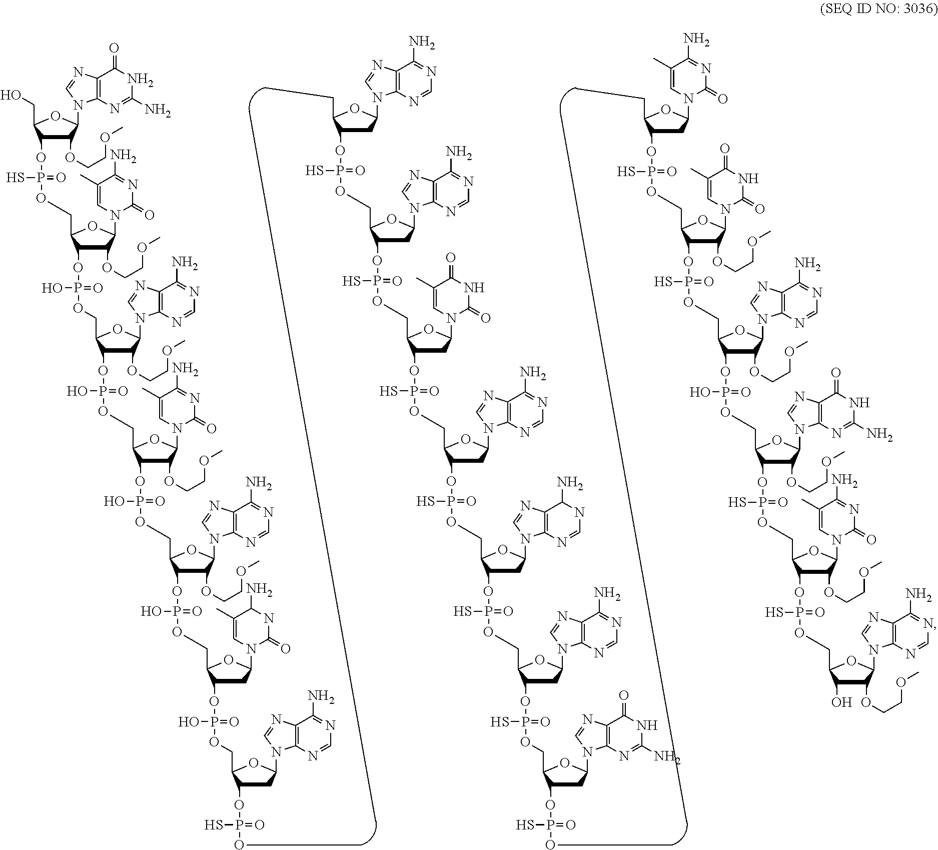

or a pharmaceutically acceptable salt thereof.

Embodiment 90. The modified oligonucleotide of any one of embodiments 86-89, which is a pharmaceutically acceptable salt comprising one or more cations selected from sodium, potassium, calcium, and magnesium.

Embodiment 91. A modified oligonucleotide according to the following chemical structure:

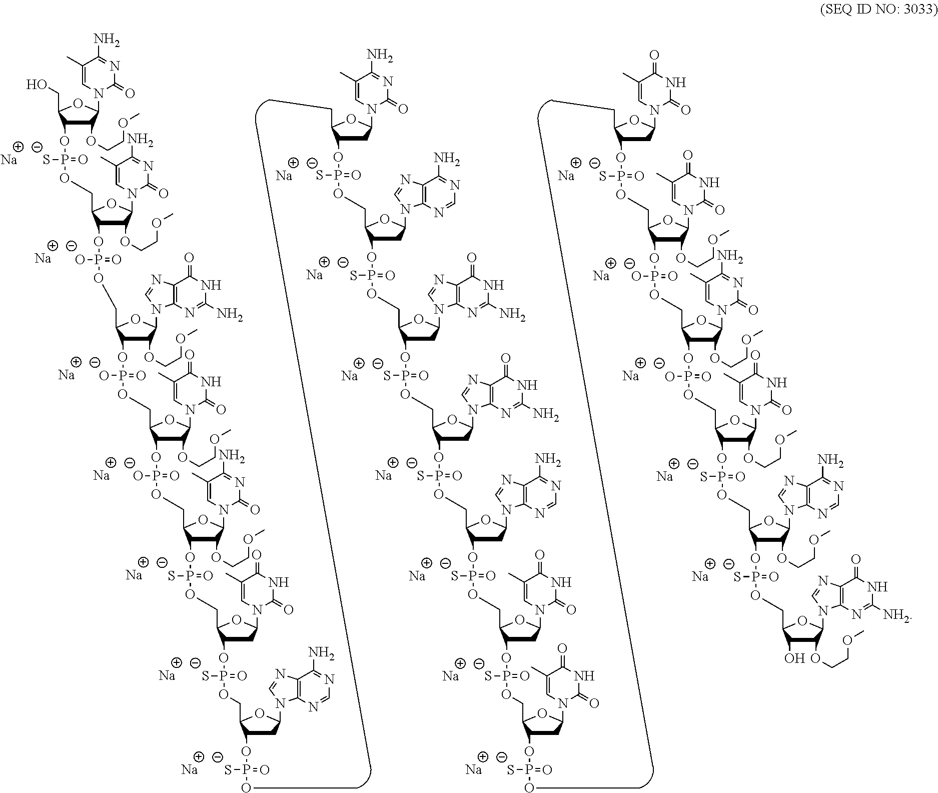

Embodiment 92. A modified oligonucleotide according to the following chemical structure:

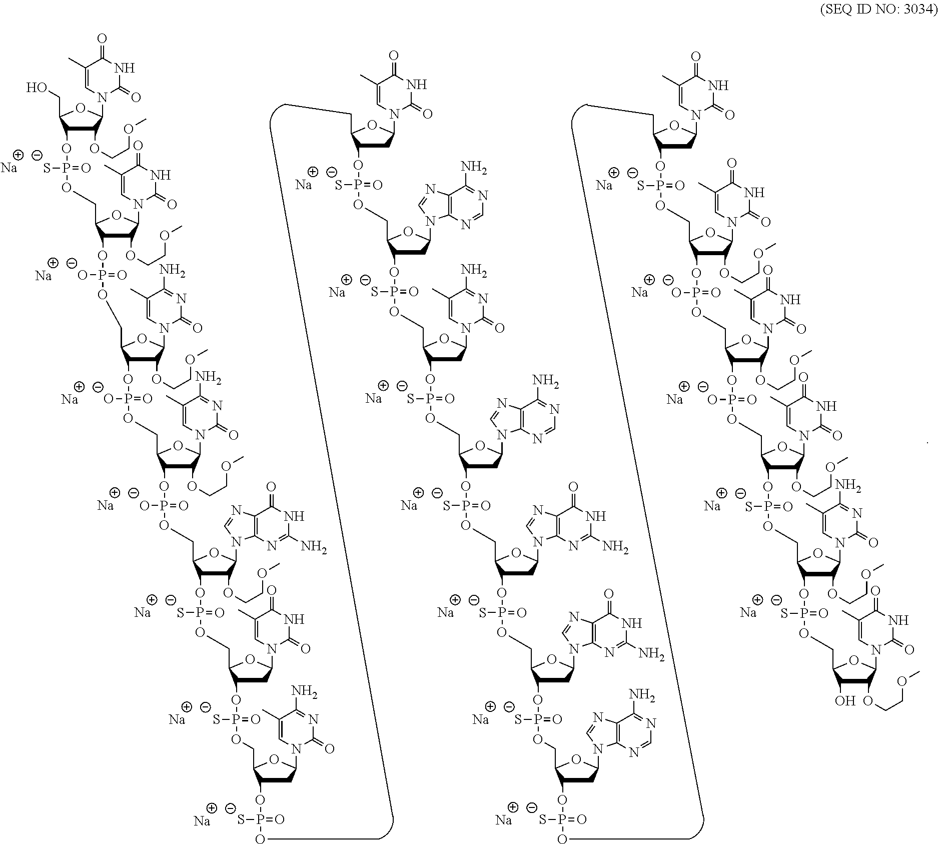

Embodiment 93. A modified oligonucleotide according to the following chemical structure:

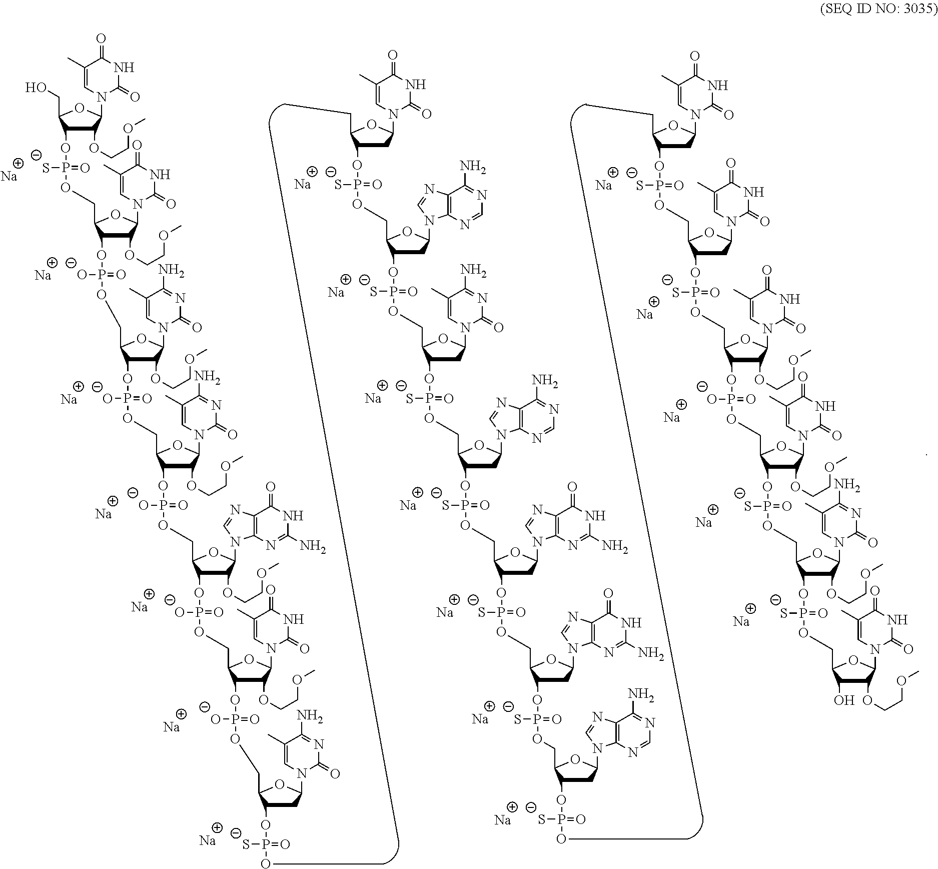

Embodiment 94. A modified oligonucleotide according to the following chemical structure:

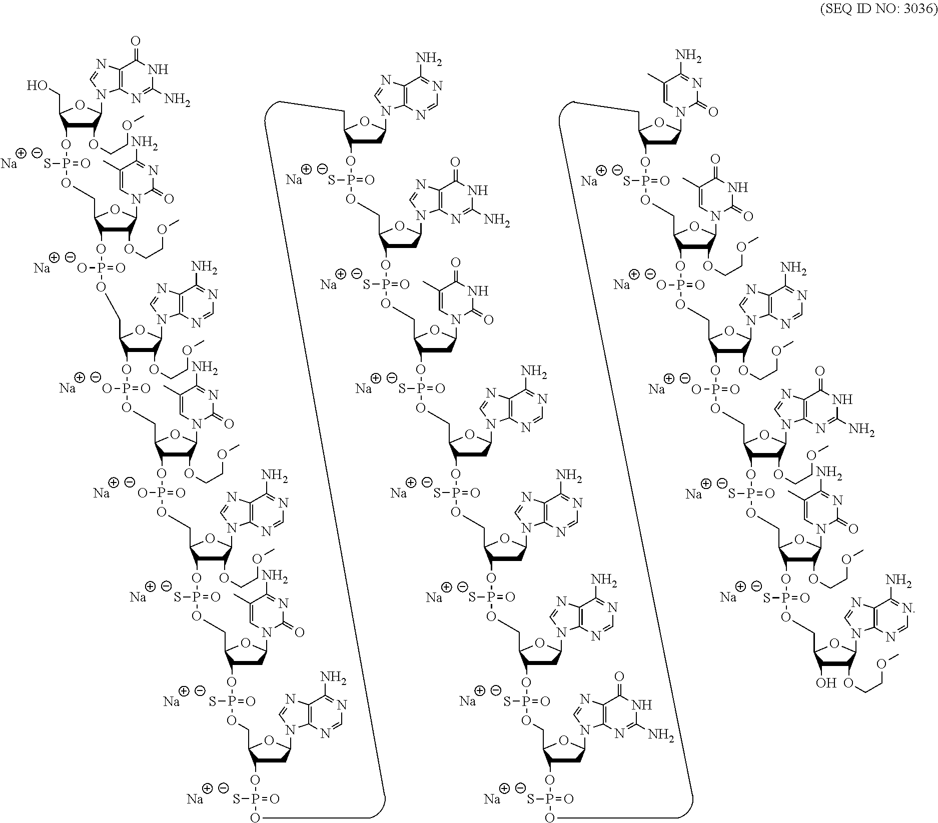

Embodiment 95. A chirally enriched population of oligomeric compounds of any of embodiments 1-59 or modified oligonucleotides of any of embodiments 86-94, wherein the population is enriched for modified oligonucleotides comprising at least one particular phosphorothioate internucleoside linkage having a particular stereochemical configuration.

Embodiment 96. The chirally enriched population of embodiment 95, wherein the population is enriched for modified oligonucleotides comprising at least one particular phosphorothioate internucleoside linkage having the (Sp) configuration.

Embodiment 97. The chirally enriched population of embodiment 95, wherein the population is enriched for modified oligonucleotides comprising at least one particular phosphorothioate internucleoside linkage having the (Rp) configuration.

Embodiment 98. The chirally enriched population of embodiment 95, wherein the population is enriched for modified oligonucleotides having a particular, independently selected stereochemical configuration at each phosphorothioate internucleoside linkage.

Embodiment 99. The chirally enriched population of embodiment 95, wherein the population is enriched for modified oligonucleotides having the (Sp) configuration at each phosphorothioate internucleoside linkage or for modified oligonucleotides having the (Rp) configuration at each phosphorothioate internucleoside linkage.

Embodiment 100. The chirally enriched population of embodiment 95, wherein the population is enriched for modified oligonucleotides having the (Rp) configuration at one particular phosphorothioate internucleoside linkage and the (Sp) configuration at each of the remaining phosphorothioate internucleoside linkages.

Embodiment 101. The chirally enriched population of embodiment 95, wherein the population is enriched for modified oligonucleotides having at least 3 contiguous phosphorothioate internucleoside linkages in the Sp, Sp, and Rp configurations, in the 5′ to 3′ direction.

Embodiment 102. A population of oligomeric compounds of any of embodiments 1-59 or modified oligonucleotides of any of embodiments 86-94, wherein all of the phosphorothioate internucleoside linkages of the modified oligonucleotide are stereorandom.

Embodiment 103. A pharmaceutical composition comprising an oligomeric compound of any of embodiments 1-59, a modified oligonucleotide of any of embodiments 86-94, an oligomeric duplex of any of embodiments 60-82, an antisense agent of any of embodiments 83-85, or a population of any of embodiments 95-102, and a pharmaceutically acceptable diluent.

Embodiment 104. The pharmaceutical composition of embodiment 103, wherein the pharmaceutically acceptable diluent is artificial cerebrospinal fluid (aCSF) or phosphate-buffered saline (PBS).

Embodiment 105. The pharmaceutical composition of embodiment 104, wherein the pharmaceutical composition consists essentially of the oligomeric compound of any of embodiments 1-59, the modified oligonucleotide of any of embodiments 86-94, the oligomeric duplex of any of embodiments 60-82, the antisense agent of any of embodiments 83-85, or the population of any of embodiments 95-102, and aCSF.

Embodiment 106. The pharmaceutical composition of embodiment 104, wherein the pharmaceutical composition consists essentially of the oligomeric compound of any of embodiments 1-59, the modified oligonucleotide of any of embodiments 86-94, the oligomeric duplex of any of embodiments 60-82, the antisense agent of any of embodiments 83-85, or the population of any of embodiments 95-102, and PBS.

Embodiment 107. A method comprising administering to a subject an oligomeric compound of any of embodiments 1-59, a modified oligonucleotide of any of embodiments 86-94, an oligomeric duplex of any of embodiments 60-82, an antisense agent of any of embodiments 83-85, a population of any of embodiments 95-102, or a pharmaceutical composition of any of embodiments 103-106.

Embodiment 108. A method of treating a glycogen storage disease comprising administering to a subject having or at risk of developing a glycogen storage disease a therapeutically effective amount of an oligomeric compound of any of embodiments 1-59, a modified oligonucleotide of any of embodiments 86-94, an oligomeric duplex of any of embodiments 60-82, an antisense agent of any of embodiments 83-85, a population of any of embodiments 95-102, or a pharmaceutical composition of any of embodiments 103-106.

Embodiment 109. The method of embodiment 108, wherein the glycogen storage disease is Lafora disease, adult polyglucosan body disease (APBD), Andersen's disease, or Pompe disease.

Embodiment 110. The method of embodiment 108, wherein the glycogen storage disease is Lafora disease.

Embodiment 111. The method of any of embodiments 108-110, wherein at least one symptom or hallmark of the glycogen storage disease is ameliorated.

Embodiment 112. The method of embodiment 111, wherein the at least one symptom or hallmark is seizures, cognitive deterioration, neuromuscular weakness, myoclonus, dementia, ataxia, cerebellar dysfunction, impaired speech, loss of ambulation, swallowing difficulty, or epileptic episode.

Embodiment 113. The method of embodiment 111 or embodiment 112, wherein administering the oligomeric compound of any of embodiments 1-59, the modified oligonucleotide of any of embodiments 86-94, the oligomeric duplex of any of embodiments 60-82, the antisense agent of any of embodiments 83-85, the population of any of embodiments 95-102, or the pharmaceutical composition of any of embodiments 103-106 reduces or delays the onset or progression of seizures, neuromuscular weakness, myoclonus, dementia, ataxia, cerebellar dysfunction, impaired speech, loss of ambulation, swallowing difficulty, or epileptic episode, or slows cognitive deterioration in the subject.

Embodiment 114. The method of any of embodiments 107-113, wherein the oligomeric compound of any of embodiments 1-59, the modified oligonucleotide of any of embodiments 86-94, the oligomeric duplex of any of embodiments 60-82, the antisense agent of any of embodiments 83-85, the population of any of embodiments 95-102, or the pharmaceutical composition of any of embodiments 103-106 is administered to the central nervous system or systemically.

Embodiment 115. The method of any of embodiments 107-114, wherein the oligomeric compound of any of embodiments 1-59, the modified oligonucleotide of any of embodiments 86-94, the oligomeric duplex of any of embodiments 60-82, the antisense agent of any of embodiments 83-85, the population of any of embodiments 95-102, or the pharmaceutical composition of any of embodiments 103-106 is administered intrathecally.

Embodiment 116. The method of any of embodiments 107-115, wherein the subject is a human.

Embodiment 117. A method of reducing expression of GYS1 in a cell comprising contacting the cell with an oligomeric compound of any of embodiments 1-59, a modified oligonucleotide of any of embodiments 86-94, an oligomeric duplex of any of embodiments 60-82, an antisense agent of any of embodiments 83-85, a population of any of embodiments 95-102, or a pharmaceutical composition of any of embodiments 103-106.

Embodiment 118. The method of embodiment 117, wherein the cell is a neuron.

Embodiment 119. The method of embodiment 117 or embodiment 118, wherein the cell is a human cell.

Embodiment 120. Use of an oligomeric compound of any of embodiments 1-59, a modified oligonucleotide of any of embodiments 86-94, an oligomeric duplex of any of embodiments 60-82, an antisense agent of any of embodiments 83-85, a population of any of embodiments 95-102, or a pharmaceutical composition of any of embodiments 103-106 for treating a glycogen storage disease.

Embodiment 121. Use of an oligomeric compound of any of embodiments 1-59, a modified oligonucleotide of any of embodiments 86-94, an oligomeric duplex of any of embodiments 60-82, an antisense agent of any of embodiments 83-85, a population of any of embodiments 95-102, or a pharmaceutical composition of any of embodiments 103-106 in the manufacture of a medicament for treating a glycogen storage disease.

Embodiment 122. The use of embodiment 120 or embodiment 121, wherein the glycogen storage disease is Lafora disease, adult polyglucosan body disease (APBD), Andersen's disease, or Pompe disease.

Embodiment 123. The use of embodiment 120 or embodiment 121, wherein the glycogen storage disease is Lafora disease.

Embodiment 124. The method of embodiment 107, wherein the subject has a glycogen storage disease.

Embodiment 125. The method of embodiment 107, wherein the subject has Lafora disease.

### I. Certain Oligonucleotides

In certain embodiments, provided herein are oligomeric compounds comprising oligonucleotides, which consist of linked nucleosides. Oligonucleotides may be unmodified oligonucleotides (RNA or DNA) or may be modified oligonucleotides. Modified oligonucleotides comprise at least one modification relative to unmodified RNA or DNA. That is, modified oligonucleotides comprise at least one modified nucleoside (comprising a modified sugar moiety and/or a modified nucleobase) and/or at least one modified internucleoside linkage. Certain modified nucleosides and modified internucleoside linkages suitable for use in modified oligonucleotides are described below.

### A. Certain Modified Nucleosides

Modified nucleosides comprise a modified sugar moiety or a modified nucleobase or both a modifed sugar moiety and a modified nucleobase. In certain embodiments, modified nucleosides comprising the following modified sugar moieties and/or the following modified nucleobases may be incorporated into modified oligonucleotides.

### 1. Certain Sugar Moieties

In certain embodiments, modified sugar moieties are non-bicyclic modified sugar moieties. In certain embodiments, modified sugar moieties are bicyclic or tricyclic sugar moieties. In certain embodiments, modified sugar moieties are sugar surrogates. Such sugar surrogates may comprise one or more substitutions corresponding to those of other types of modified sugar moieties.

In certain embodiments, modified sugar moieties are non-bicyclic modified furanosyl sugar moieties comprising one or more acyclic substituent, including, but not limited, to substituents at the 2′, 3′, 4′, and/or 5′ positions. In certain embodiments, the furanosyl sugar moiety is a ribosyl sugar moiety. In certain embodiments, one or more acyclic substituent of non-bicyclic modified sugar moieties is branched.

In certain embodiments, non-bicyclic modifed sugar moieties comprise a substituent group at the 2′-position. Examples of substituent groups suitable for the 2′-position of modified sugar moieties include but are not limited to: —F, —OCH₃ (“OMe” or “O-methyl”), and —O(CH₂)₂OCH₃ (“MOE”). In certain embodiments, 2′-substituent groups are selected from among: halo, allyl, amino, azido, SH, CN, OCN, CF₃, OCF₃, O—C₁-C₁₀ alkoxy, O—C₁-C₁₀ substituted alkoxy, O—C₁-C₁₀ alkyl, O—C₁-C₁₀ substituted alkyl, S-alkyl, N(Rₘ)-alkyl, O-alkenyl, S-alkenyl, N(Rₘ)-alkenyl, O-alkynyl, S-alkynyl, N(Rₘ)-alkynyl, O-alkylenyl-O-alkyl, alkynyl, alkaryl, aralkyl, O-alkaryl, O-aralkyl, O(CH₂)₂SCH₃, O(CH₂)₂ON(Rₘ)(Rₙ) or OCH₂C(═O)—N(Rₘ)(Rₙ), where each Rₘ and Rₙ is, independently, H, an amino protecting group, or substituted or unsubstituted C₁-C₁₀ alkyl, —O(CH₂)₂ON(CH₃)₂ (“DMAOE”), 2′-O(CH₂)₂O(CH₂)₂N(CH₃)₂ (“DMAEOE”), and the 2′-substituent groups described in Cook et al., U.S. Pat. No. 6,531,584; Cook et al., U.S. Pat. No. 5,859,221; and Cook et al., U.S. Pat. No. 6,005,087. Certain embodiments of these 2′-substituent groups can be further substituted with one or more substituent groups independently selected from among: hydroxyl, amino, alkoxy, carboxy, benzyl, phenyl, nitro (NO₂), thiol, thioalkoxy, thioalkyl, halogen, alkyl, aryl, alkenyl and alkynyl.

In certain embodiments, a 2′-substituted non-bicyclic modified nucleoside comprises a sugar moiety comprising a non-bridging 2′-substituent group selected from: F, NH₂, N₃, OCF₃, OCH₃, O(CH₂)₃NH₂, CH₂CH═CH₂, OCH₂CH═CH₂, OCH₂CH₂OCH₃, O(CH₂)₂SCH₃, O(CH₂)₂ON(Rₘ)(Rₙ), O(CH₂)₂O(CH₂)₂N(CH₃)₂, and N-substituted acetamide (OCH₂C(═O)—N(Rₘ)(Rₙ)), where each Rₘ and Rₙ is, independently, H, an amino protecting group, or substituted or unsubstituted C₁-C₁₀ alkyl.

In certain embodiments, a 2′-substituted non-bicyclic modified nucleoside comprises a sugar moiety comprising a non-bridging 2′-substituent group selected from: F, OCF₃, OCH₃, OCH₂CH₂OCH₃, O(CH₂)₂SCH₃, O(CH₂)₂ON(CH₃)₂, O(CH₂)₂O(CH₂)₂N(CH₃)₂, and OCH₂C(═O)—N(H)CH₃ (“NMA”).

In certain embodiments, a 2′-substituted non-bicyclic modified nucleoside comprises a sugar moiety comprising a non-bridging 2′-substituent group selected from: F, OCH₃, and OCH₂CH₂OCH₃.

In certain embodiments, modified furanosyl sugar moieties and nucleosides incorporating such modified furanosyl sugar moieties are further defined by isomeric configuration. For example, a 2′-deoxyfuranosyl sugar moiety may be in seven isomeric configurations other than the naturally occurring β-D-deoxyribosyl configuration. Such modified sugar moieties are described in, e.g., WO 2019/157531, incorporated by reference herein. A 2′-modified sugar moiety has an additional stereocenter at the 2′-position relative to a 2′-deoxyfuranosyl sugar moiety; therefore, such sugar moieties have a total of sixteen possible isomeric configurations. 2′-modified sugar moieties described herein are in the β-D-ribosyl isomeric configuration unless otherwise specified.

In certain embodiments, non-bicyclic modifed sugar moieties comprise a substituent group at the 4′-position. Examples of substituent groups suitable for the 4′-position of modified sugar moieties include but are not limited to alkoxy (e.g., methoxy), alkyl, and those described in Manoharan et al., WO 2015/106128.

In certain embodiments, non-bicyclic modifed sugar moieties comprise a substituent group at the 3′-position. Examples of substituent groups suitable for the 3′-position of modified sugar moieties include but are not limited to alkoxy (e.g., methoxy), alkyl (e.g., methyl, ethyl).

In certain embodiments, non-bicyclic modifed sugar moieties comprise a substituent group at the 5′-position. Examples of substituent groups suitable for the 5′-position of modified sugar moieties include but are not limited to vinyl, alkoxy (e.g., methoxy), alkyl (e.g., methyl (R or S), ethyl).

In certain embodiments, non-bicyclic modified sugar moieties comprise more than one non-bridging sugar substituent, for example, 2′-F-5′-methyl sugar moieties and the modified sugar moieties and modified nucleosides described in Migawa et al., WO 2008/101157 and Rajeev et al., US2013/0203836).

In naturally occurring nucleic acids, sugars are linked to one another 3′ to 5′. In certain embodiments, oligonucleotides include one or more nucleoside or sugar moiety linked at an alternative position, for example at the 2′ position or inverted 5′ to 3′. For example, where the linkage is at the 2′ position, the 2′-substituent groups may instead be at the 3′-position.

Certain modifed sugar moieties comprise a substituent that bridges two atoms of the furanosyl ring to form a second ring, resulting in a bicyclic sugar moiety. In certain such embodiments, the bicyclic sugar moiety comprises a bridge between the 4′ and the 2′ furanose ring atoms. Examples of such 4′ to 2′ bridging sugar substituents include but are not limited to: 4′-CH₂-2′, 4′-(CH₂)₂-2′, 4′-(CH₂)₃-2′, 4′-CH₂—O-2′ (“LNA”), 4′-CH₂—S-2′, 4′-(CH₂)₂—O-2′ (“ENA”), 4′-CH(CH₃)—O-2′ (referred to as “constrained ethyl” or “cEt”), 4′-CH₂—O—CH₂-2′, 4′-CH₂—N(R)-2′, 4′-CH(CH₂OCH₃)—O-2′ (“constrained MOE” or “cMOE”) and analogs thereof (see, e.g., Seth et al., U.S. Pat. No. 7,399,845, Bhat et al., U.S. Pat. No. 7,569,686, Swayze et al., U.S. Pat. No. 7,741,457, and Swayze et al., U.S. Pat. No. 8,022,193), 4′-C(CH₃)(CH₃)—O-2′ and analogs thereof (see, e.g., Seth et al., U.S. Pat. No. 8,278,283), 4′-CH₂—N(OCH₃)-2′ and analogs thereof (see, e.g., Prakash et al., U.S. Pat. No. 8,278,425), 4′-CH₂—O—N(CH₃)-2′ (see, e.g., Allerson et al., U.S. Pat. No. 7,696,345 and Allerson et al., U.S. Pat. No. 8,124,745), 4′-CH₂—C(H)(CH₃)-2′ (see, e.g., Zhou, et al., J. Org. Chem., 2009, 74, 118-134), 4′-CH₂—C(═CH₂)-2′ and analogs thereof (see e.g., Seth et al., U.S. Pat. No. 8,278,426), 4′-C(RₐR_{b})—N(R)—O-2′, 4′-C(RₐR_{b})—O—N(R)-2′, 4′-CH₂—O—N(R)-2′, and 4′-CH₂—N(R)—O-2′, wherein each R, Rₐ, and R_{b} is, independently, H, a protecting group, or C₁-C₁₂ alkyl (see, e.g. Imanishi et al., U.S. Pat. No. 7,427,672).

In certain embodiments, such 4′ to 2′ bridges independently comprise from 1 to 4 linked groups independently selected from: —[C(Rₐ)(R_{b})]ₙ—, —[C(Rₐ)(R_{b})]—O—, —C(Rₐ)═C(R_{b})—, —C(Rₐ)═N—, —C(═NRₐ)—, —C(═O)—, —C(═S)—, —O—, —Si(Rₐ)₂—, —S(═O)ₓ—, and —N(Rₐ)—;
wherein:
x is 0, 1, or 2;
n is 1, 2, 3, or 4;
each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H), substituted acyl, CN, sulfonyl (S(═O)₂-J₁), or sulfoxyl (S(═O)-J₁); and
each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H), substituted acyl, a heterocycle radical, a substituted heterocycle radical, C₁-C₁₂ aminoalkyl, substituted C₁-C₁₂ aminoalkyl, or a protecting group.

Additional bicyclic sugar moieties are known in the art, see, for example: Freier et al., Nucleic Acids Research, 1997, 25(22), 4429-4443, Albaek et al., J. Org. Chem., 2006, 71, 7731-7740, Singh et al., Chem. Commun., 1998, 4, 455-456; Koshkin et al., Tetrahedron, 1998, 54, 3607-3630; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129, 8362-8379; Wengel et al., U.S. Pat. No. 7,053,207; Imanishi et al., U.S. Pat. No. 6,268,490; Imanishi et al. U.S. Pat. No. 6,770,748; Imanishi et al., U.S. RE44,779; Wengel et al., U.S. Pat. No. 6,794,499; Wengel et al., U.S. Pat. No. 6,670,461; Wengel et al., U.S. Pat. No. 7,034,133; Wengel et al., U.S. Pat. No. 8,080,644; Wengel et al., U.S. Pat. No. 8,034,909; Wengel et al., U.S. Pat. No. 8,153,365; Wengel et al., U.S. Pat. No. 7,572,582; Ramasamy et al., U.S. Pat. No. 6,525,191; Torsten et al., WO 2004/106356; Wengel et al., WO 1999/014226; Seth et al., WO 2007/134181; Seth et al., U.S. Pat. No. 7,547,684; Seth et al., U.S. Pat. No. 7,666,854; Seth et al., U.S. Pat. No. 8,088,746; Seth et al., U.S. Pat. No. 7,750,131; Seth et al., U.S. Pat. No. 8,030,467; Seth et al., U.S. Pat. No. 8,268,980; Seth et al., U.S. Pat. No. 8,546,556; Seth et al., U.S. Pat. No. 8,530,640; Migawa et al., U.S. Pat. No. 9,012,421; Seth et al., U.S. Pat. No. 8,501,805; and U.S. Patent Publication Nos. Allerson et al., US2008/0039618 and Migawa et al., US2015/0191727.

In certain embodiments, bicyclic sugar moieties and nucleosides incorporating such bicyclic sugar moieties are further defined by isomeric configuration. For example, an LNA nucleoside (described herein) may be in the α-L configuration or in the β-D configuration.

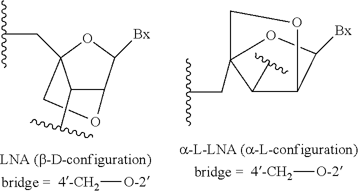

α-L-methyleneoxy (4′-CH₂—O-2′) or α-L-LNA bicyclic nucleosides have been incorporated into oligonucleotides that showed antisense activity (Frieden et al., Nucleic Acids Research, 2003, 21, 6365-6372). The addition of locked nucleic acids to siRNAs has been shown to increase siRNA stability in serum, and to reduce off-target effects (Elmen, J. et al., (2005) Nucleic Acids Research 33(1):439-447; Mook, OR. et al., (2007) Mal Cane Ther 6(3):833-843; Grunweller, A. et al., (2003) Nucleic Acids Research 31(12):3185-3193). Herein, general descriptions of bicyclic nucleosides include both isomeric configurations. When the positions of specific bicyclic nucleosides (e.g., LNA or cEt) are identified in exemplified embodiments herein, they are in the β-D configuration, unless otherwise specified.

In certain embodiments, modified sugar moieties comprise one or more non-bridging sugar substituent and one or more bridging sugar substituent (e.g., 5′-substituted and 4′-2′ bridged sugars).

In certain embodiments, modified sugar moieties are sugar surrogates. In certain such embodiments, the oxygen atom of the sugar moiety is replaced, e.g., with a sulfur, carbon or nitrogen atom. In certain such embodiments, such modified sugar moieties also comprise bridging and/or non-bridging substituents as described herein. For example, certain sugar surrogates comprise a 4′-sulfur atom and a substitution at the 2′-position (see, e.g., Bhat et al., U.S. Pat. No. 7,875,733 and Bhat et al., U.S. Pat. No. 7,939,677) and/or the 5′ position.

In certain embodiments, sugar surrogates comprise rings having other than 5 atoms. For example, in certain embodiments, a sugar surrogate comprises a six-membered tetrahydropyran (“THP”). Such tetrahydropyrans may be further modified or substituted. Nucleosides comprising such modified tetrahydropyrans include but are not limited to hexitol nucleic acid (“HNA”), anitol nucleic acid (“ANA”), manitol nucleic acid (“MNA”) (see, e.g., Leumann, C J. Bioorg. & Med. Chem. 2002, 10, 841-854), fluoro HNA:

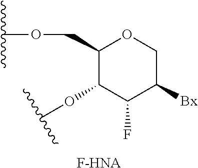

(“F-HNA”, see e.g. Swayze et al., U.S. Pat. No. 8,088,904; Swayze et al., U.S. Pat. No. 8,440,803; Swayze et al., U.S. Pat. No. 8,796,437; and Swayze et al., U.S. Pat. No. 9,005,906; F-HNA can also be referred to as a F-THP or 3′-fluoro tetrahydropyran), and nucleosides comprising additional modified THP compounds having the formula:

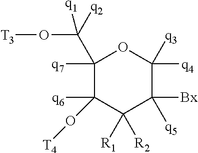

wherein, independently, for each of the modified THP nucleosides:
Bx is a nucleobase moiety;
T₃ and T₄ are each, independently, an internucleoside linking group linking the modified THP nucleoside to the remainder of an oligonucleotide or one of T₃ and T₄ is an internucleoside linking group linking the modified THP nucleoside to the remainder of an oligonucleotide and the other of T₃ and T₄ is H, a hydroxyl protecting group, a linked conjugate group, or a 5′ or 3′-terminal group;
q₁, q₂, q₃, q₄, q₅, q₆ and q₇ are each, independently, H, C₁-C₆ alkyl, substituted C₁-C₆ alkyl, C₂-C₆ alkenyl, substituted C₂-C₆ alkenyl, C₂-C₆ alkynyl, or substituted C₂-C₆ alkynyl; and
each of R₁ and R₂ is independently selected from among: hydrogen, halogen, substituted or unsubstituted alkoxy, NJ₁J₂, SJ₁, N₃, OC(═X)J₁, OC(═X)NJ₁J₂, NJ₃C(═X)NJ₁J₂, and CN, wherein X is O, S or NJ₁, and each J₁, J₂, and J₃ is, independently, H or C₁-C₆ alkyl.

In certain embodiments, modified THP nucleosides are provided wherein q₁, q₂, q₃, q₄, q₅, q₆ and q₇ are each H. In certain embodiments, at least one of q₁, q₂, q₃, q₄, q₅, q₆ and q₇ is other than H. In certain embodiments, at least one of q₁, q₂, q₃, q₄, q₅, q₆ and q₇ is methyl. In certain embodiments, modified THP nucleosides are provided wherein one of R₁ and R₂ is F. In certain embodiments, R₁ is F and R₂ is H, in certain embodiments, R₁ is methoxy and R₂ is H, and in certain embodiments, R₁ is methoxyethoxy and R₂ is H.

In certain embodiments, sugar surrogates comprise rings having more than 5 atoms and more than one heteroatom. For example, nucleosides comprising morpholino sugar moieties and their use in oligonucleotides have been reported (see, e.g., Braasch et al., Biochemistry, 2002, 41, 4503-4510 and Summerton et al., U.S. Pat. No. 5,698,685; Summerton et al., U.S. Pat. No. 5,166,315; Summerton et al., U.S. Pat. No. 5,185,444; and Summerton et al., U.S. Pat. No. 5,034,506). As used here, the term “morpholino” means a sugar surrogate having the following structure:

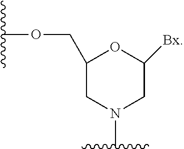

In certain embodiments, morpholinos may be modified, for example by adding or altering various substituent groups from the above morpholino structure. Such sugar surrogates are referred to herein as “modifed morpholinos.”

In certain embodiments, sugar surrogates comprise acyclic moieites. Examples of nucleosides and oligonucleotides comprising such acyclic sugar surrogates include but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., WO2011/133876. In certain embodiments, sugar surrogates comprise acyclic moieties. Examples of nucleosides and oligonucleotides comprising such acyclic sugar surrogates include, but are not limited to: peptide nucleic acid (“PNA”), acyclic butyl nucleic acid (see, e.g., Kumar et al., Org. Biomol. Chem., 2013, 11, 5853-5865), and nucleosides and oligonucleotides described in Manoharan et al., US2013/130378. Representative U.S. patents that teach the preparation of PNA compounds include, but are not limited to, U.S. Pat. Nos. 5,539,082; 5,714,331; and 5,719,262. Additional PNA compounds suitable for use in the oligonucleotides of the invention are described in, for example, in Nielsen et al., Science, 1991, 254, 1497-1500.

In certain embodiments, sugar surrogates are the “unlocked” sugar structure of UNA (unlocked nucleic acid) nucleosides. UNA is an unlocked acyclic nucleic acid, wherein any of the bonds of the sugar has been removed, forming an unlocked sugar surrogate. Representative U.S. publications that teach the preparation of UNA include, but are not limited to, U.S. Pat. No. 8,314,227; and US Patent Publication Nos. 2013/0096289; 2013/0011922; and 2011/0313020, the entire contents of each of which are hereby incorporated herein by reference.

In certain embodiments, sugar surrogates are the glycerol as found in GNA (glycol nucleic acid) nucleosides as depicted below:

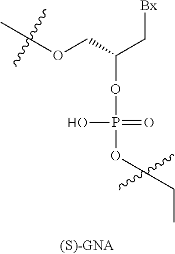

where Bx represents any nucleobase.

Many other bicyclic and tricyclic sugar and sugar surrogate ring systems are known in the art that can be used in modified nucleosides.

### 2. Certain Modified Nucleobases

In certain embodiments, modified oligonucleotides comprise one or more nucleosides comprising an unmodified nucleobase. In certain embodiments, modified oligonucleotides comprise one or more nucleoside comprising a modified nucleobase. In certain embodiments, modified oligonucleotides comprise one or more nucleoside that does not comprise a nucleobase, referred to as an abasic nucleoside. In certain embodiments, modified oligonucleotides comprise one or more inosine nucleosides (i.e., nucleosides comprising a hypoxanthine nucleobase).

In certain embodiments, modified nucleobases are selected from: 5-substituted pyrimidines, 6-azapyrimidines, alkyl or alkynyl substituted pyrimidines, alkyl substituted purines, and N-2, N-6 and 0-6 substituted purines. In certain embodiments, modified nucleobases are selected from: 2-aminopropyladenine, 5-hydroxymethyl cytosine, xanthine, hypoxanthine, 2-aminoadenine, 6-N-methylguanine, 6-N-methyladenine, 2-propyladenine, 2-thiouracil, 2-thiothymine and 2-thiocytosine, 5-propynyl (—C≡C—CH₃) uracil, 5-propynylcytosine, 6-azouracil, 6-azocytosine, 6-azothymine, 5-ribosyluracil (pseudouracil), 4-thiouracil, 8-halo, 8-amino, 8-thiol, 8-thioalkyl, 8-hydroxyl, 8-aza and other 8-substituted purines, 5-halo, particularly 5-bromo, 5-trifluoromethyl, 5-halouracil, and 5-halocytosine, 7-methylguanine, 7-methyladenine, 2-F-adenine, 2-aminoadenine, 7-deazaguanine, 7-deazaadenine, 3-deazaguanine, 3-deazaadenine, 6-N-benzoyladenine, 2-N-isobutyrylguanine, 4-N-benzoylcytosine, 4-N-benzoyluracil, 5-methyl 4-N-benzoylcytosine, 5-methyl 4-N-benzoyluracil, universal bases, hydrophobic bases, promiscuous bases, size-expanded bases, and fluorinated bases. Further modified nucleobases include tricyclic pyrimidines, such as 1,3-diazaphenoxazine-2-one, 1,3-diazaphenothiazine-2-one and 9-(2-aminoethoxy)-1,3-diazaphenoxazine-2-one (G-clamp). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in Merigan et al., U.S. Pat. No. 3,687,808, those disclosed in The Concise Encyclopedia Of Polymer Science And Engineering, Kroschwitz, J. I., Ed., John Wiley & Sons, 1990, 858-859; Englisch et al., Angewandte Chemie, International Edition, 1991, 30, 613; Sanghvi, Y. S., Chapter 15, Antisense Research and Applications, Crooke, S. T. and Lebleu, B., Eds., CRC Press, 1993, 273-288; and those disclosed in Chapters 6 and 15, Antisense Drug Technology, Crooke S. T., Ed., CRC Press, 2008, 163-166 and 442-443.

Publications that teach the preparation of certain of the above noted modified nucleobases as well as other modified nucleobases include without limitation, Manoharan et al., US2003/0158403; Manoharan et al., US2003/0175906; Dinh et al., U.S. Pat. No. 4,845,205; Spielvogel et al., U.S. Pat. No. 5,130,302; Rogers et al., U.S. Pat. No. 5,134,066; Bischofberger et al., U.S. Pat. No. 5,175,273; Urdea et al., U.S. Pat. No. 5,367,066; Benner et al., U.S. Pat. No. 5,432,272; Matteucci et al., U.S. Pat. No. 5,434,257; Gmeiner et al., U.S. Pat. No. 5,457,187; Cook et al., U.S. Pat. No. 5,459,255; Froehler et al., U.S. Pat. No. 5,484,908; Matteucci et al., U.S. Pat. No. 5,502,177; Hawkins et al., U.S. Pat. No. 5,525,711; Haralambidis et al., U.S. Pat. No. 5,552,540; Cook et al., U.S. Pat. No. 5,587,469; Froehler et al., U.S. Pat. No. 5,594,121; Switzer et al., U.S. Pat. No. 5,596,091; Cook et al., U.S. Pat. No. 5,614,617; Froehler et al., U.S. Pat. No. 5,645,985; Cook et al., U.S. Pat. No. 5,681,941; Cook et al., U.S. Pat. No. 5,811,534; Cook et al., U.S. Pat. No. 5,750,692; Cook et al., U.S. Pat. No. 5,948,903; Cook et al., U.S. Pat. No. 5,587,470; Cook et al., U.S. Pat. No. 5,457,191; Matteucci et al., U.S. Pat. No. 5,763,588; Froehler et al., U.S. Pat. No. 5,830,653; Cook et al., U.S. Pat. No. 5,808,027; Cook et al., 6,166,199; and Matteucci et al., U.S. Pat. No. 6,005,096.

### 3. Certain Modified Internucleoside Linkages

The naturally occurring internucleoside linkage of RNA and DNA is a 3′ to 5′ phosphodiester linkage. In certain embodiments, nucleosides of modified oligonucleotides may be linked together using one or more modified internucleoside linkages. The two main classes of internucleoside linking groups are defined by the presence or absence of a phosphorus atom. Representative phosphorus-containing internucleoside linkages include but are not limited to phosphodiesters, which contain a phosphodiester bond (“P(O₂)═O”) (also referred to as unmodified or naturally occurring linkages), phosphotriesters, methylphosphonates, phosphoramidates, phosphorothioates (“P(O₂)═S”), and phosphorodithioates (“HS—P═S”). Representative non-phosphorus containing internucleoside linking groups include but are not limited to methylenemethylimino (—CH₂—N(CH₃)—O—CH₂—), thiodiester, thionocarbamate (—O—C(═O)(NH)—S—); siloxane (—O—SiH₂—O—); and N,N′-dimethylhydrazine (—CH₂—N(CH₃)—N(CH₃)—). Modified internucleoside linkages, compared to naturally occurring phosphodiester internucleoside linkages, can be used to alter, typically increase, nuclease resistance of the oligonucleotide. In certain embodiments, internucleoside linkages having a chiral atom can be prepared as a racemic mixture, or as separate enantiomers. Methods of preparation of phosphorous-containing and non-phosphorous-containing internucleoside linkages are well known to those skilled in the art.

In certain embodiments, a modified internucleoside linkage is any of those described in WO/2021/030778, incorporated by reference herein. In certain embodiments, a modified internucleoside linkage comprises the formula:

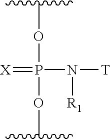

wherein independently for each internucleoside linking group of the modified oligonucleotide:
X is selected from O or S;
R₁ is selected from H, C₁-C₆ alkyl, and substituted C₁-C₆ alkyl; and
T is selected from SO₂R₂, C(═O)R₃, and P(═O)R₄R₅, wherein:
R₂ is selected from an aryl, a substituted aryl, a heterocycle, a substituted heterocycle, an aromatic heterocycle, a substituted aromatic heterocycle, a diazole, a substituted diazole, a C₁-C₆ alkoxy, C₁-C₆ alkyl, C₁-C₆ alkenyl, C₁-C₆ alkynyl, substituted C₁-C₆ alkyl, substituted C₁-C₆ alkenyl substituted C₁-C₆ alkynyl, and a conjugate group;
R₃ is selected from an aryl, a substituted aryl, CH₃, N(CH₃)₂, OCH₃ and a conjugate group;
R₄ is selected from OCH₃, OH, C₁-C₆ alkyl, substituted C₁-C₆ alkyl and a conjugate group; and
R₅ is selected from OCH₃, OH, C₁-C₆ alkyl, and substituted C₁-C₆ alkyl.
   In certain embodiments, a modified internucleoside linkage comprises a mesyl phosphoramidate linking group having a formula:

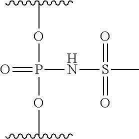 [SMILES: *OP(=O)(NS(C)(=O)=O)O*]

In certain embodiments, a mesyl phosphoramidate internucleoside linkage may comprise a chiral center. In certain embodiments, modified oligonucleotides comprising (Rp) and/or (Sp) mesyl phosphoramidates comprise one or more of the following formulas, respectively, wherein “B” indicates a nucleobase:

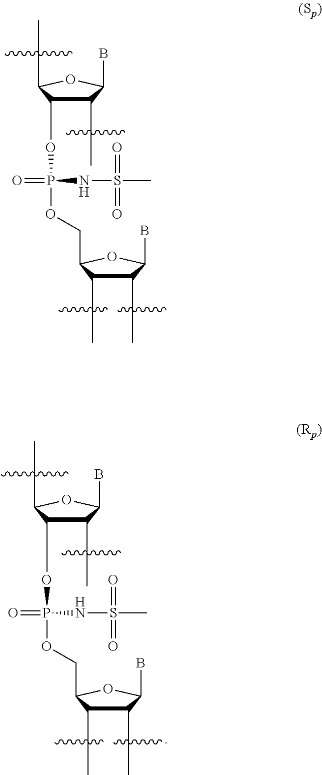

Representative internucleoside linkages having a chiral center include but are not limited to alkylphosphonates and phosphorothioates. Modified oligonucleotides comprising internucleoside linkages having a chiral center can be prepared as populations of modified oligonucleotides comprising stereorandom internucleoside linkages, or as populations of modified oligonucleotides comprising phosphorothioate internucleoside linkages in particular stereochemical configurations. In certain embodiments, populations of modified oligonucleotides comprise phosphorothioate internucleoside linkages wherein all of the phosphorothioate internucleoside linkages are stereorandom. Such modified oligonucleotides can be generated using synthetic methods that result in random selection of the stereochemical configuration of each phosphorothioate internucleoside linkage. Nonetheless, as is well understood by those of skill in the art, each individual phosphorothioate of each individual oligonucleotide molecule has a defined stereoconfiguration. In certain embodiments, populations of modified oligonucleotides are enriched for modified oligonucleotides comprising one or more particular phosphorothioate internucleoside linkage in a particular, independently selected stereochemical configuration. In certain embodiments, the particular configuration of the particular phosphorothioate internucleoside linkage is present in at least 65% of the molecules in the population. In certain embodiments, the particular configuration of the particular phosphorothioate internucleoside linkage is present in at least 70% of the molecules in the population. In certain embodiments, the particular configuration of the particular phosphorothioate internucleoside linkage is present in at least 80% of the molecules in the population. In certain embodiments, the particular configuration of the particular phosphorothioate internucleoside linkage is present in at least 90% of the molecules in the population. In certain embodiments, the particular configuration of the particular phosphorothioate internucleoside linkage is present in at least 99% of the molecules in the population. Such chirally enriched populations of modified oligonucleotides can be generated using synthetic methods known in the art, e.g., methods described in Oka et al., JACS, 2003, 125, 8307, Wan et al., Nuc. Acid. Res., 2014, 42, 13456, and WO 2017/015555. In certain embodiments, a population of modified oligonucleotides is enriched for modified oligonucleotides having at least one indicated phosphorothioate in the (Sp) configuration. In certain embodiments, a population of modified oligonucleotides is enriched for modified oligonucleotides having at least one phosphorothioate in the (Rp) configuration. In certain embodiments, modified oligonucleotides comprising (Rp) and/or (Sp) phosphorothioates comprise one or more of the following formulas, respectively, wherein “B” indicates a nucleobase:

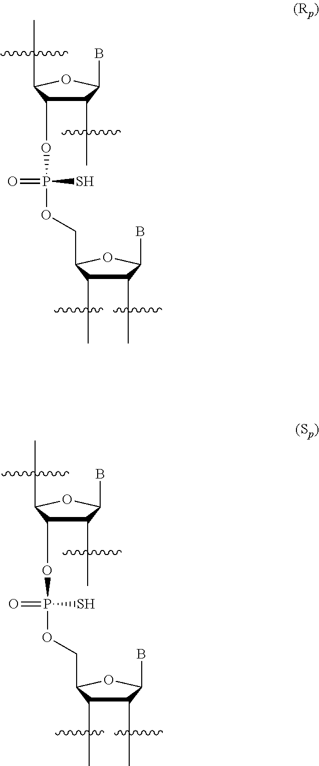

Unless otherwise indicated, chiral internucleoside linkages of modified oligonucleotides described herein can be stereorandom or in a particular stereochemical configuration.

Neutral internucleoside linkages include, without limitation, phosphotriesters, methylphosphonates, MMI (3′-CH₂—N(CH₃)—O-5′), amide-3 (3′-CH₂—C(═O)—N(H)-5′), amide-4 (3′-CH₂—N(H)—C(═O)-5′), formacetal (3′-O—CH₂—O-5′), methoxypropyl (MOP), and thioformacetal (3′-S—CH₂—O-5′). Further neutral internucleoside linkages include nonionic linkages comprising siloxane (dialkylsiloxane), carboxylate ester, carboxamide, sulfide, sulfonate ester and amides (see e.g., Carbohydrate Modifications in Antisense Research; Y. S. Sanghvi and P. D. Cook, Eds., ACS Symposium Series 580; Chapters 3 and 4, 40-65). Further neutral internucleoside linkages include nonionic linkages comprising mixed N, O, S and CH₂ component parts.

In certain embodiments, modified oligonucleotides comprise one or more inverted nucleoside, as shown below:

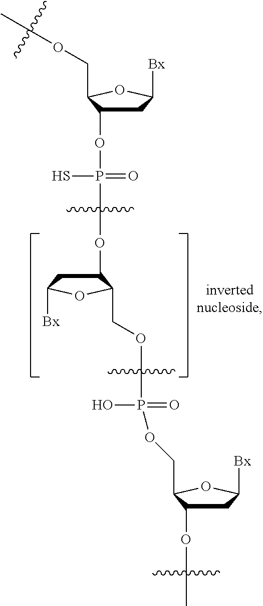

wherein each Bx independently represents any nucleobase.

In certain embodiments, an inverted nucleoside is terminal (i.e., the last nucleoside on one end of an oligonucleotide) and so only one internucleoside linkage depicted above will be present. In certain such embodiments, additional features (such as a conjugate group) may be attached to the inverted nucleoside. Such terminal inverted nucleosides can be attached to either or both ends of an oligonucleotide.

In certain embodiments, such groups lack a nucleobase and are referred to herein as inverted sugar moieties. In certain embodiments, an inverted sugar moiety is terminal (i.e., attached to the last nucleoside on one end of an oligonucleotide) and so only one internucleoside linkage above will be present. In certain such embodiments, additional features (such as a conjugate group) may be attached to the inverted sugar moiety. Such terminal inverted sugar moieties can be attached to either or both ends of an oligonucleotide.

In certain embodiments, nucleic acids can be linked 2′ to 5′ rather than the standard 3′ to 5′ linkage. Such a linkage is illustrated below.

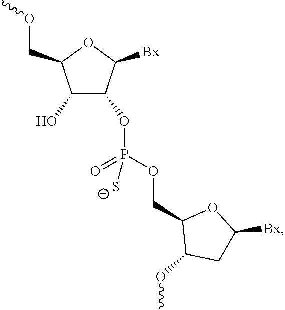

wherein each Bx represents any nucleobase.

### B. Certain Motifs

In certain embodiments, modified oligonucleotides comprise one or more modified nucleosides comprising a modified sugar moiety. In certain embodiments, modified oligonucleotides comprise one or more modified nucleosides comprising a modified nucleobase. In certain embodiments, modified oligonucleotides comprise one or more modified internucleoside linkage. In such embodiments, the modified, unmodified, and differently modified sugar moieties, nucleobases, and/or internucleoside linkages of a modified oligonucleotide define a pattern or motif. In certain embodiments, the patterns of sugar moieties, nucleobases, and internucleoside linkages are each independent of one another. Thus, a modified oligonucleotide may be described by its sugar motif, nucleobase motif and/or internucleoside linkage motif (as used herein, nucleobase motif describes the modifications to the nucleobases independent of the sequence of nucleobases).

### 1. Certain Sugar Motifs

In certain embodiments, oligonucleotides comprise one or more type of modified sugar and/or unmodified sugar moiety arranged along the oligonucleotide or portion thereof in a defined pattern or sugar motif. In certain instances, such sugar motifs include but are not limited to any of the sugar modifications discussed herein.

### Gapmer Oligonucleotides

In certain embodiments, modified oligonucleotides comprise or consist of a region having a gapmer motif, which is defined by two external regions or “wings” and a central or internal region or “gap.” The three regions of a gapmer motif (the 5′-wing, the gap, and the 3′-wing) form a contiguous sequence of nucleosides wherein at least some of the sugar moieties of the nucleosides of each of the wings differ from at least some of the sugar moieties of the nucleosides of the gap. Specifically, at least the sugar moieties of the nucleosides of each wing that are closest to the gap (the 3′-most nucleoside of the 5′-wing and the 5′-most nucleoside of the 3′-wing) differ from the sugar moiety of the neighboring gap nucleosides, thus defining the boundary between the wings and the gap (i.e., the wing/gap junction). In certain embodiments, the sugar moieties within the gap are the same as one another. In certain embodiments, the gap includes one or more nucleoside having a sugar moiety that differs from the sugar moiety of one or more other nucleosides of the gap. In certain embodiments, the sugar motifs of the two wings are the same as one another (symmetric gapmer). In certain embodiments, the sugar motif of the 5′-wing differs from the sugar motif of the 3′-wing (asymmetric gapmer).

In certain embodiments, the wings of a gapmer comprise 1-6 nucleosides. In certain embodiments, each nucleoside of each wing of a gapmer comprises a modified sugar moiety. In certain embodiments, at least one nucleoside of each wing of a gapmer comprises a modified sugar moiety. In certain embodiments, at least two nucleosides of each wing of a gapmer comprises a modified sugar moiety. In certain embodiments, at least three nucleosides of each wing of a gapmer comprises a modified sugar moiety. In certain embodiments, at least four nucleosides of each wing of a gapmer comprises a modified sugar moiety. In certain embodiments, at least five nucleosides of each wing of a gapmer comprises a modified sugar moiety.

In certain embodiments, the gap of a gapmer comprises 7-12 nucleosides. In certain embodiments, each nucleoside of the gap of a gapmer comprises a 2′-β-D-deoxyribosyl sugar moiety. In certain embodiments, at least six nucleosides of the gap of a gapmer comprise a 2′-β-D-deoxyribosyl sugar moiety. In certain embodiments, each nucleoside of the gap of a gapmer comprises a 2′-β-D-deoxyribosyl sugar moiety. In certain embodiments, at least one nucleoside of the gap of a gapmer comprises a modified sugar moiety. In certain embodiments, at least one nucleoside of the gap of a gapmer comprises a 2′-OMe modified sugar moiety.

In certain embodiments, the gapmer is a deoxy gapmer. In certain embodiments, the nucleosides on the gap side of each wing/gap junction comprise 2′-β-D-deoxyribosyl sugar moieties and the nucleosides on the wing sides of each wing/gap junction comprise modified sugar moieties. In certain embodiments, at least six nucleosides of the gap of a gapmer comprise a 2′-β-D-deoxyribosyl sugar moiety. In certain embodiments, each nucleoside of the gap comprises a 2′-β-D-deoxyribosyl sugar moiety. In certain embodiments, each nucleoside of each wing of a gapmer comprises a modified sugar moiety. In certain embodiments, one nucleoside of the gap comprises a modified sugar moiety and each remaining nucleoside of the gap comprises a 2′-β-D-deoxyribosyl sugar moiety.

In certain embodiments, modified oligonucleotides comprise or consist of a portion having a fully modified sugar motif. In such embodiments, each nucleoside of the fully modified portion of the modified oligonucleotide comprises a modified sugar moiety. In certain embodiments, each nucleoside of the entire modified oligonucleotide comprises a modified sugar moiety. In certain embodiments, modified oligonucleotides comprise or consist of a portion having a fully modified sugar motif, wherein each nucleoside within the fully modified portion comprises the same modified sugar moiety, referred to herein as a uniformly modified sugar motif. In certain embodiments, a fully modified oligonucleotide is a uniformly modified oligonucleotide. In certain embodiments, each nucleoside of a uniformly modified oligonucleotide comprises the same 2′-modification.

Herein, the lengths (number of nucleosides) of the three regions of a gapmer may be provided using the notation [#of nucleosides in the 5′-wing]-[#of nucleosides in the gap]-[#of nucleosides in the 3′-wing]. Thus, a 5-10-5 gapmer consists of 5 linked nucleosides in each wing and 10 linked nucleosides in the gap. Where such nomenclature is followed by a specific modification, that modification is the modification in each sugar moiety of each wing and the gap nucleosides comprises a 2′-β-D-deoxyribosyl sugar moiety. Thus, a 5-10-5 MOE gapmer consists of 5 linked 2′-MOE nucleosides in the 5′-wing, 10 linked a 2′-β-D-deoxynucleosides in the gap, and 5 linked 2′-MOE nucleosides in the 3′-wing. A 3-10-3 cEt gapmer consists of 3 linked cEt nucleosides in the 5′-wing, 10 linked 2′-β-D-deoxynucleosides in the gap, and 3 linked cEt nucleosides in the 3′-wing. A 5-8-5 gapmer consists of 5 linked nucleosides comprising a modified sugar moiety in the 5′-wing, 8 linked a 2′-β-D-deoxynucleosides in the gap, and 5 linked nucleosides comprising a modified sugar moiety in the 3′-wing. A mixed wing gapmer has at least two different modified sugars in the 5′ and/or 3′ wing. A 5-8-5 or 5-8-4 mixed wing gapmer has at least two different modified sugar moieties in the 5′- and/or the 3′-wing.

In certain embodiments, modified oligonucleotides are 5-10-5 MOE gapmers. In certain embodiments, modified oligonucleotides are 6-10-4 MOE gapmers. In certain embodiments, modified oligonucleotides are 4-10-6 MOE gapmers. In certain embodiments, modified oligonucleotides are 5-8-4 MOE gapmers. In certain embodiments, modified oligonucleotides are 3-10-7 MOE gapmers. In certain embodiments, modified oligonucleotides are 7-10-3 MOE gapmers. In certain embodiments, modified oligonucleotides are 5-8-5 MOE gapmers. In certain embodiments, modified oligonucleotides are 5-9-5 MOE gapmers. In certain embodiments, modified oligonucleotides are X-Y-Z MOE gapmers, wherein X and Z are independently selected from 1, 2, 3, 4, 5, 6, or 7 linked 2′-MOE nucleosides and Y is selected from 7, 8, 9, 10, or 11 linked deoxynucleosides.

In certain embodiments, modified oligonucleotides have a sugar motif selected from the following (5′ to 3′): eeeeeddddddddddeeeee, wherein ‘d’ represents a 2′-β-D-deoxyribosyl sugar moiety, and ‘e’ represents a 2′-O(CH₂)₂OCH₃ ribosyl sugar moiety. In certain embodiments, modified oligonucleotides have a sugar motif selected from the following (5′ to 3′): eeeeeeddddddddddeeee, wherein ‘d’ represents a 2′-β-D-deoxyribosyl sugar moiety, and ‘e’ represents a 2′-O(CH₂)₂OCH₃ ribosyl sugar moiety. In certain embodiments, modified oligonucleotides have a sugar motif selected from the following (5′ to 3′): eeeeedddddddddeeeee, wherein ‘d’ represents a 2′-β-D-deoxyribosyl sugar moiety, and ‘e’ represents a 2′-O(CH₂)₂OCH₃ ribosyl sugar moiety.

### 2. Certain Nucleobase Motifs

In certain embodiments, oligonucleotides comprise modified and/or unmodified nucleobases arranged along the oligonucleotide or portion thereof in a defined pattern or motif. In certain embodiments, each nucleobase is modified. In certain embodiments, none of the nucleobases are modified. In certain embodiments, each purine or each pyrimidine is modified. In certain embodiments, each adenine is modified. In certain embodiments, each guanine is modified. In certain embodiments, each thymine is modified. In certain embodiments, each uracil is modified. In certain embodiments, each cytosine is modified. In certain embodiments, some or all of the cytosine nucleobases in a modified oligonucleotide are 5-methylcytosines. In certain embodiments, all of the cytosine nucleobases are 5-methylcytosines and all of the other nucleobases of the modified oligonucleotide are unmodified nucleobases.

In certain embodiments, modified oligonucleotides comprise a block of modified nucleobases. In certain such embodiments, the block is at the 3′-end of the oligonucleotide. In certain embodiments the block is within 3 nucleosides of the 3′-end of the oligonucleotide. In certain embodiments, the block is at the 5′-end of the oligonucleotide. In certain embodiments, the block is within 3 nucleosides of the 5′-end of the oligonucleotide.

In certain embodiments, oligonucleotides having a gapmer motif comprise a nucleoside comprising a modified nucleobase. In certain such embodiments, one nucleoside comprising a modified nucleobase is in the central gap of an oligonucleotide having a gapmer motif. In certain such embodiments, the sugar moiety of the nucleoside is a 2′-β-D-deoxyribosyl sugar moiety. In certain embodiments, the modified nucleobase is selected from: a 2-thiopyrimidine and a 5-propynepyrimidine.

### 3. Certain Internucleoside Linkage Motifs

In certain embodiments, oligonucleotides comprise modified and/or unmodified internucleoside linkages arranged along the oligonucleotide or portion thereof in a defined pattern or motif. In certain embodiments, each internucleoside linking group is a phosphodiester internucleoside linkage (P(O₂)═O). In certain embodiments, each internucleoside linking group of a modified oligonucleotide is a phosphorothioate internucleoside linkage (P(O₂)═S). In certain embodiments, each internucleoside linkage of a modified oligonucleotide is independently selected from a phosphorothioate internucleoside linkage and phosphodiester internucleoside linkage. In certain embodiments, each phosphorothioate internucleoside linkage is independently selected from a stereorandom phosphorothioate, a (Sp) phosphorothioate, and a (Rp) phosphorothioate.

In certain embodiments, the sugar motif of a modified oligonucleotide is a gapmer and the internucleoside linkages within the gap are all modified. In certain embodiments, some or all of the internucleoside linkages in the wings are unmodified phosphodiester internucleoside linkages. In certain embodiments, the terminal internucleoside linkages are modified. In certain embodiments, the sugar motif of a modified oligonucleotide is a gapmer, and the internucleoside linkage motif comprises at least one phosphodiester internucleoside linkage in at least one wing, wherein the at least one phosphodiester internucleoside linkage is not a terminal internucleoside linkage, and the remaining internucleoside linkages are phosphorothioate internucleoside linkages. In certain embodiments, all of the phosphorothioate internucleoside linkages are stereorandom. In certain embodiments, all of the phosphorothioate internucleoside linkages in the wings are (Sp) phosphorothioates, and the gap comprises at least one Sp, Sp, or Rp motif. In certain embodiments, populations of modified oligonucleotides are enriched for modified oligonucleotides comprising such internucleoside linkage motifs.

In certain embodiments, all of the internucleoside linkages are either phosphodiester internucleoside linkages or phosphorothioate internucleoside linkages, and the chiral motif is (5′ to 3′): Sp-o-o-o-Sp-Sp-Sp-Rp-Sp-Sp-Rp-Sp-Sp-Sp-Sp-Sp-Sp-Sp-Sp or Sp-o-o-o-Sp-Sp-Sp-Rp-Sp-Sp-Sp-Sp-Sp-Sp-Sp-Sp-Sp-Sp-Sp, wherein each ‘Sp’ represents a (Sp) phosphorothioate internucleoside linkage, each ‘Rp’ is a Rp internucleoside linkage, and each ‘o’ represents a phosphodiester internucleoside linkage. In certain embodiments, populations of modified oligonucleotides are enriched for modified oligonucleotides comprising such internucleoside linkage motifs.

In certain embodiments, modified oligonucleotides have an internucleoside linkage motif of soooosssssssssssooss, wherein each “s” represents a phosphorothioate internucleoside linkage and each “o” represents a phosphodiester internucleoside linkage. In certain embodiments, modified oligonucleotides have an internucleoside linkage motif of sososssssssssssooss, wherein each “s” represents a phosphorothioate internucleoside linkage and each “o” represents a phosphodiester internucleoside linkage. In certain embodiments, modified oligonucleotides have an internucleoside linkage motif of sooosssssssssssooss, wherein each “s” represents a phosphorothioate internucleoside linkage and each “o” represents a phosphodiester internucleoside linkage. In certain embodiments, modified oligonucleotides have an internucleoside linkage motif of soooossssssssssooss, wherein each “s” represents a phosphorothioate internucleoside linkage and each “o” represents a phosphodiester internucleoside linkage. In certain embodiments, modified oligonucleotides have an internucleoside linkage motif of sooosssssssssssooos, wherein each “s” represents a phosphorothioate internucleoside linkage and each “o” represents a phosphodiester internucleoside linkage. In certain embodiments, modified oligonucleotides have an internucleoside linkage motif of sooossssssssssooss, wherein each “s” represents a phosphorothioate internucleoside linkage and each “o” represents a phosphodiester internucleoside linkage. In certain embodiments, modified oligonucleotides have an internucleoside linkage motif of sooooossssssssssoss, wherein each “s” represents a phosphorothioate internucleoside linkage and each “o” represents a phosphodiester internucleoside linkage.

In certain embodiments, modified oligonucleotides have an internucleoside linkage motif comprising one or more mesyl phosphoramidate linking groups. In certain embodiments, one or more phosphorothioate internucleoside linkages or one or more phosphodiester internucleoside linkages of the internucleoside linkage motifs herein is substituted with a mesyl phosphoramidates linking group.

### C. Certain Lengths

It is possible to increase or decrease the length of an oligonucleotide without eliminating activity. For example, in Woolf et al., Proc. Natl. Acad. Sci. USA, 1992, 89, 7305-7309, 1992), a series of oligonucleotides 13-25 nucleobases in length were tested for their ability to induce cleavage of a target nucleic acid in an oocyte injection model. Oligonucleotides 25 nucleobases in length with 8 or 11 mismatch bases near the ends of the oligonucleotides were able to direct specific cleavage of the target nucleic acid, albeit to a lesser extent than the oligonucleotides that contained no mismatches. Similarly, target specific cleavage was achieved using 13 nucleobase oligonucleotides, including those with 1 or 3 mismatches.

In certain embodiments, oligonucleotides (including modified oligonucleotides) can have any of a variety of ranges of lengths. In certain embodiments, oligonucleotides consist of X to Y linked nucleosides, where X represents the fewest number of nucleosides in the range and Y represents the largest number nucleosides in the range. In certain such embodiments, X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X≤Y. For example, in certain embodiments, oligonucleotides consist of 12 to 13, 12 to 14, 12 to 15, 12 to 16, 12 to 17, 12 to 18, 12 to 19, 12 to 20, 12 to 21, 12 to 22, 12 to 23, 12 to 24, 12 to 25, 12 to 26, 12 to 27, 12 to 28, 12 to 29, 12 to 30, 13 to 14, 13 to 15, 13 to 16, 13 to 17, 13 to 18, 13 to 19, 13 to 20, 13 to 21, 13 to 22, 13 to 23, 13 to 24, 13 to 25, 13 to 26, 13 to 27, 13 to 28, 13 to 29, 13 to 30, 14 to 15, 14 to 16, 14 to 17, 14 to 18, 14 to 19, 14 to 20, 14 to 21, 14 to 22, 14 to 23, 14 to 24, 14 to 25, 14 to 26, 14 to 27, 14 to 28, 14 to 29, 14 to 30, 15 to 16, 15 to 17, 15 to 18, 15 to 19, 15 to 20, 15 to 21, 15 to 22, 15 to 23, 15 to 24, 15 to 25, 15 to 26, 15 to 27, 15 to 28, 15 to 29, 15 to 30, 16 to 17, 16 to 18, 16 to 19, 16 to 20, 16 to 21, 16 to 22, 16 to 23, 16 to 24, 16 to 25, 16 to 26, 16 to 27, 16 to 28, 16 to 29, 16 to 30, 17 to 18, 17 to 19, 17 to 20, 17 to 21, 17 to 22, 17 to 23, 17 to 24, 17 to 25, 17 to 26, 17 to 27, 17 to 28, 17 to 29, 17 to 30, 18 to 19, 18 to 20, 18 to 21, 18 to 22, 18 to 23, 18 to 24, 18 to 25, 18 to 26, 18 to 27, 18 to 28, 18 to 29, 18 to 30, 19 to 20, 19 to 21, 19 to 22, 19 to 23, 19 to 24, 19 to 25, 19 to 26, 19 to 27, 19 to 28, 19 to 29, 19 to 30, 20 to 21, 20 to 22, 20 to 23, 20 to 24, 20 to 25, 20 to 26, 20 to 27, 20 to 28, 20 to 29, 20 to 30, 21 to 22, 21 to 23, 21 to 24, 21 to 25, 21 to 26, 21 to 27, 21 to 28, 21 to 29, 21 to 30, 22 to 23, 22 to 24, 22 to 25, 22 to 26, 22 to 27, 22 to 28, 22 to 29, 22 to 30, 23 to 24, 23 to 25, 23 to 26, 23 to 27, 23 to 28, 23 to 29, 23 to 30, 24 to 25, 24 to 26, 24 to 27, 24 to 28, 24 to 29, 24 to 30, 25 to 26, 25 to 27, 25 to 28, 25 to 29, 25 to 30, 26 to 27, 26 to 28, 26 to 29, 26 to 30, 27 to 28, 27 to 29, 27 to 30, 28 to 29, 28 to 30, or 29 to 30 linked nucleosides.

In certain embodiments, oligonucleotides consist of 16 linked nucleosides. In certain embodiments, oligonucleotides consist of 17 linked nucleosides. In certain embodiments, oligonucleotides consist of 18 linked nucleosides. In certain embodiments, oligonucleotides consist of 19 linked nucleosides. In certain embodiments, oligonucleotides consist of 20 linked nucleosides.

### D. Certain Modified Oligonucleotides

In certain embodiments, the above modifications (sugar, nucleobase, internucleoside linkage) are incorporated into a modified oligonucleotide. In certain embodiments, modified oligonucleotides are characterized by their modification motifs and overall lengths. In certain embodiments, such parameters are each independent of one another. Thus, unless otherwise indicated, each internucleoside linkage of an oligonucleotide having a gapmer sugar motif may be modified or unmodified and may or may not follow the gapmer modification pattern of the sugar modifications. For example, the internucleoside linkages within the wing regions of a sugar gapmer may be the same or different from one another and may be the same or different from the internucleoside linkages of the gap region of the sugar motif. Likewise, such sugar gapmer oligonucleotides may comprise one or more modified nucleobase independent of the gapmer pattern of the sugar modifications. Unless otherwise indicated, all modifications are independent of nucleobase sequence.

### E. Certain Populations of Modified Oligonucleotides

Populations of modified oligonucleotides in which all of the modified oligonucleotides of the population have the same molecular formula can be stereorandom populations or chirally enriched populations. All of the chiral centers of all of the modified oligonucleotides are stereorandom in a stereorandom population. In a chirally enriched population, at least one particular chiral center is not stereorandom in the modified oligonucleotides of the population. In certain embodiments, the modified oligonucleotides of a chirally enriched population are enriched for β-D ribosyl sugar moieties, and all of the phosphorothioate internucleoside linkages are stereorandom. In certain embodiments, the modified oligonucleotides of a chirally enriched population are enriched for both β-D ribosyl sugar moieties and at least one, particular phosphorothioate internucleoside linkage in a particular stereochemical configuration.

### F. Nucleobase Sequence

In certain embodiments, oligonucleotides (unmodified or modified oligonucleotides) are further described by their nucleobase sequence. In certain embodiments oligonucleotides have a nucleobase sequence that is complementary to a second oligonucleotide or an identified reference nucleic acid, such as a target nucleic acid. In certain such embodiments, a portion of an oligonucleotide has a nucleobase sequence that is complementary to a second oligonucleotide or an identified reference nucleic acid, such as a target nucleic acid. In certain embodiments, the nucleobase sequence of a portion or entire length of an oligonucleotide is at least 50%, at least 60%, at least 70%, at least 80%, at least 85%, at least 90%, at least 95%, or 100% complementary to the second oligonucleotide or nucleic acid, such as a target nucleic acid.

### II. Certain Oligomeric Compounds

In certain embodiments, provided herein are oligomeric compounds, which consist of an oligonucleotide (modified or unmodified) and optionally one or more conjugate groups and/or terminal groups. Conjugate groups consist of one or more conjugate moiety and a conjugate linker which links the conjugate moiety to the oligonucleotide. Conjugate groups may be attached to either or both ends of an oligonucleotide and/or at any internal position. In certain embodiments, conjugate groups are attached to the 2-position of a nucleoside of a modified oligonucleotide. In certain embodiments, conjugate groups that are attached to either or both ends of an oligonucleotide are terminal groups. In certain such embodiments, conjugate groups or terminal groups are attached at the 3′ and/or 5′-end of oligonucleotides. In certain such embodiments, conjugate groups (or terminal groups) are attached at the 3′-end of oligonucleotides. In certain embodiments, conjugate groups are attached near the 3′-end of oligonucleotides. In certain embodiments, conjugate groups (or terminal groups) are attached at the 5′-end of oligonucleotides. In certain embodiments, conjugate groups are attached near the 5′-end of oligonucleotides.

Examples of terminal groups include but are not limited to conjugate groups, capping groups, phosphate moieties, protecting groups, abasic nucleosides, modified or unmodified nucleosides, and two or more nucleosides that are independently modified or unmodified.

### A. Certain Conjugate Groups

In certain embodiments, oligonucleotides are covalently attached to one or more conjugate groups. In certain embodiments, conjugate groups modify one or more properties of the attached oligonucleotide, including but not limited to pharmacodynamics, pharmacokinetics, stability, binding, absorption, tissue distribution, cellular distribution, cellular uptake, charge and clearance.

In certain embodiments, conjugation of one or more carbohydrate moieties to a modified oligonucleotide can optimize one or more properties of the modified oligonucleotide. In certain embodiments, the carbohydrate moiety is attached to a modified subunit of the modified oligonucleotide. For example, the ribose sugar of one or more ribonucleotide subunits of a modified oligonucleotide can be replaced with another moiety, e.g. a non-carbohydrate (preferably cyclic) carrier to which is attached a carbohydrate ligand. A ribonucleotide subunit in which the ribose sugar of the subunit has been so replaced is referred to herein as a ribose replacement modification subunit (RRMS), which is a modified sugar moiety. A cyclic carrier may be a carbocyclic ring system, i.e., one or more ring atoms may be a heteroatom, e.g., nitrogen, oxygen, sulphur. The cyclic carrier may be a monocyclic ring system, or may contain two or more rings, e.g. fused rings. The cyclic carrier may be a fully saturated ring system, or it may contain one or more double bonds. In certain embodiments, the modified oligonucleotide is a gapmer.

In certain embodiments, conjugate groups impart a new property on the attached oligonucleotide, e.g., fluorophores or reporter groups that enable detection of the oligonucleotide. Certain conjugate groups and conjugate moieties have been described previously, for example: cholesterol moiety (Letsinger et al., Proc. Natl. Acad. Sci. USA, 1989, 86, 6553-6556), cholic acid (Manoharan et al., Bioorg. Med. Chem. Lett., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Lett., 1993, 3, 2765-2770), a thiocholesterol (Oberhauser et al., Nucl. Acids Res., 1992, 20, 533-538), an aliphatic chain, e.g., do-decan-diol or undecyl residues (Saison-Behmoaras et al., EMBO J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14, 969-973), or adamantane acetic acid a palmityl moiety (Mishra et al., Biochim. Biophys. Acta, 1995, 1264, 229-237), an octadecylamine or hexylamino-carbonyl-oxycholesterol moiety (Crooke et al., J. Pharmacol. Exp. Ther., 1996, 277, 923-937), a tocopherol group (Nishina et al., Molecular Therapy Nucleic Acids, 2015, 4, e220; and Nishina et al., Molecular Therapy, 2008, 16, 734-740), or a GalNAc cluster (e.g., WO2014/179620).

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, C5 alkyl, C22 alkenyl, C20 alkenyl, C16 alkenyl, C10 alkenyl, C21 alkenyl, C19 alkenyl, C18 alkenyl, C15 alkenyl, C14 alkenyl, C13 alkenyl, C12 alkenyl, C11 alkenyl, C9 alkenyl, C8 alkenyl, C7 alkenyl, C6 alkenyl, or C5 alkenyl.

In certain embodiments, the conjugate group may comprise a conjugate moiety selected from any of a C22 alkyl, C20 alkyl, C16 alkyl, C10 alkyl, C21 alkyl, C19 alkyl, C18 alkyl, C15 alkyl, C14 alkyl, C13 alkyl, C12 alkyl, C11 alkyl, C9 alkyl, C8 alkyl, C7 alkyl, C6 alkyl, or C5 alkyl, where the alkyl chain has one or more unsaturated bonds.

In certain embodiments, a conjugate group is a lipid having the following structure:

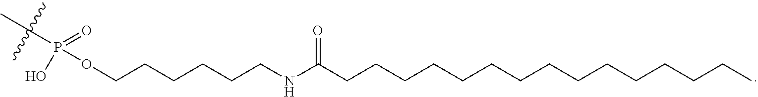 [SMILES: *P(=O)(O)OCCCCCCNC(=O)CCCCCCCCCCCCCCC]

### 1. Conjugate Moieties

Conjugate moieties include, without limitation, intercalators, reporter molecules, polyamines, polyamides, peptides, carbohydrates, antibodies, vitamin moieties, polyethylene glycols, thioethers, polyethers, cholesterols, thiocholesterols, cholic acid moieties, folate, lipids, lipophilic groups, phospholipids, biotin, phenazine, phenanthridine, anthraquinone, adamantane, acridine, fluoresceins, rhodamines, coumarins, fluorophores, and dyes.

In certain embodiments, a conjugate moiety comprises an active drug substance, for example, aspirin, warfarin, phenylbutazone, ibuprofen, suprofen, fen-bufen, ketoprofen, (S)-(+)-pranoprofen, carprofen, dansylsarcosine, 2,3,5-triiodobenzoic acid, fingolimod, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic.

### 2. Conjugate Linkers

Conjugate moieties are attached to oligonucleotides through conjugate linkers. In certain oligomeric compounds, the conjugate linker is a single chemical bond (i.e., the conjugate moiety is attached directly to an oligonucleotide through a single bond). In certain oligomeric compounds, a conjugate moiety is attached to an oligonucleotide via a more complex conjugate linker comprising one or more conjugate linker moieties, which are sub-units making up a conjugate linker. In certain embodiments, the conjugate linker comprises a chain structure, such as a hydrocarbyl chain, or an oligomer of repeating units such as ethylene glycol, nucleosides, or amino acid units.

In certain embodiments, a conjugate linker comprises pyrrolidine.

In certain embodiments, a conjugate linker comprises one or more groups selected from alkyl, amino, oxo, amide, disulfide, polyethylene glycol, ether, thioether, and hydroxylamino. In certain such embodiments, the conjugate linker comprises groups selected from alkyl, amino, oxo, amide and ether groups. In certain embodiments, the conjugate linker comprises groups selected from alkyl and amide groups. In certain embodiments, the conjugate linker comprises groups selected from alkyl and ether groups. In certain embodiments, the conjugate linker comprises at least one phosphorus moiety. In certain embodiments, the conjugate linker comprises at least one phosphate group. In certain embodiments, the conjugate linker includes at least one neutral linking group.

In certain embodiments, conjugate linkers, including the conjugate linkers described above, are bifunctional linking moieties, e.g., those known in the art to be useful for attaching conjugate groups to parent compounds, such as the oligonucleotides provided herein. In general, a bifunctional linking moiety comprises at least two functional groups. One of the functional groups is selected to bind to a particular site on a parent compound and the other is selected to bind to a conjugate group. Examples of functional groups used in a bifunctional linking moiety include but are not limited to electrophiles for reacting with nucleophilic groups and nucleophiles for reacting with electrophilic groups. In certain embodiments, bifunctional linking moieties comprise one or more groups selected from amino, hydroxyl, carboxylic acid, thiol, alkyl, alkenyl, and alkynyl.

Examples of conjugate linkers include but are not limited to pyrrolidine, 8-amino-3,6-dioxaoctanoic acid (ADO), succinimidyl 4-(N-maleimidomethyl) cyclohexane-1-carboxylate (SMCC) and 6-aminohexanoic acid (AHEX or AHA). Other conjugate linkers include but are not limited to substituted or unsubstituted C₁-C₁₀ alkyl, substituted or unsubstituted C₂-C₁₀ alkenyl or substituted or unsubstituted C₂-C₁₀ alkynyl, wherein a nonlimiting list of preferred substituent groups includes hydroxyl, amino, alkoxy, carboxy, benzyl, phenyl, nitro, thiol, thioalkoxy, halogen, alkyl, aryl, alkenyl and alkynyl.

In certain embodiments, conjugate linkers comprise 1-10 linker-nucleosides. In certain embodiments, conjugate linkers comprise 2-5 linker-nucleosides. In certain embodiments, conjugate linkers comprise exactly 3 linker-nucleosides. In certain embodiments, conjugate linkers comprise the TCA motif. In certain embodiments, such linker-nucleosides are modified nucleosides. In certain embodiments such linker-nucleosides comprise a modified sugar moiety. In certain embodiments, linker-nucleosides are unmodified. In certain embodiments, linker-nucleosides comprise an optionally protected heterocyclic base selected from a purine, substituted purine, pyrimidine or substituted pyrimidine. In certain embodiments, a cleavable moiety is a nucleoside selected from uracil, thymine, cytosine, 4-N-benzoylcytosine, 5-methylcytosine, 4-N-benzoyl-5-methylcytosine, adenine, 6-N-benzoyladenine, guanine and 2-N-isobutyrylguanine. It is typically desirable for linker-nucleosides to be cleaved from the oligomeric compound after it reaches a target tissue. Accordingly, linker-nucleosides are typically linked to one another and to the remainder of the oligomeric compound through cleavable bonds. In certain embodiments, such cleavable bonds are phosphodiester bonds.

Herein, linker-nucleosides are not considered to be part of the oligonucleotide. Accordingly, in embodiments in which an oligomeric compound comprises an oligonucleotide consisting of a specified number or range of linked nucleosides and/or a specified percent complementarity to a reference nucleic acid and the oligomeric compound also comprises a conjugate group comprising a conjugate linker comprising linker-nucleosides, those linker-nucleosides are not counted toward the length of the oligonucleotide and are not used in determining the percent complementarity of the oligonucleotide for the reference nucleic acid. For example, an oligomeric compound may comprise (1) a modified oligonucleotide consisting of 8-30 nucleosides and (2) a conjugate group comprising 1-10 linker-nucleosides that are contiguous with the nucleosides of the modified oligonucleotide. The total number of contiguous linked nucleosides in such an oligomeric compound is more than 30. Alternatively, an oligomeric compound may comprise a modified oligonucleotide consisting of 8-30 nucleosides and no conjugate group. The total number of contiguous linked nucleosides in such an oligomeric compound is no more than 30. Unless otherwise indicated conjugate linkers comprise no more than 10 linker-nucleosides. In certain embodiments, conjugate linkers comprise no more than 5 linker-nucleosides. In certain embodiments, conjugate linkers comprise no more than 3 linker-nucleosides. In certain embodiments, conjugate linkers comprise no more than 2 linker-nucleosides. In certain embodiments, conjugate linkers comprise no more than 1 linker-nucleoside.

In certain embodiments, it is desirable for a conjugate group to be cleaved from the oligonucleotide. For example, in certain circumstances oligomeric compounds comprising a particular conjugate moiety are better taken up by a particular cell type, but once the oligomeric compound has been taken up, it is desirable that the conjugate group be cleaved to release the unconjugated or parent oligonucleotide. Thus, certain conjugate linkers may comprise one or more cleavable moieties. In certain embodiments, a cleavable moiety is a cleavable bond. In certain embodiments, a cleavable moiety is a group of atoms comprising at least one cleavable bond. In certain embodiments, a cleavable moiety comprises a group of atoms having one, two, three, four, or more than four cleavable bonds. In certain embodiments, a cleavable moiety is selectively cleaved inside a cell or subcellular compartment, such as a lysosome. In certain embodiments, a cleavable moiety is selectively cleaved by endogenous enzymes, such as nucleases.

In certain embodiments, a cleavable bond is selected from among: an amide, an ester, an ether, one or both esters of a phosphodiester, a phosphate ester, a carbamate, or a disulfide. In certain embodiments, a cleavable bond is one or both of the esters of a phosphodiester. In certain embodiments, a cleavable moiety comprises a phosphate or phosphodiester. In certain embodiments, the cleavable moiety is a phosphate or phosphodiester linkage between an oligonucleotide and a conjugate moiety or conjugate group.

In certain embodiments, a cleavable moiety comprises or consists of one or more linker-nucleosides. In certain such embodiments, the one or more linker-nucleosides are linked to one another and/or to the remainder of the oligomeric compound through cleavable bonds. In certain embodiments, such cleavable bonds are unmodified phosphodiester bonds. In certain embodiments, a cleavable moiety is 2′-deoxynucleoside that is attached to either the 3′ or 5′-terminal nucleoside of an oligonucleotide by a phosphodiester internucleoside linkage and covalently attached to the remainder of the conjugate linker or conjugate moiety by a phosphate or phosphorothioate internucleoside linkage. In certain such embodiments, the cleavable moiety is 2′-deoxyadenosine.

### 3. Cell-Targeting Moieties

In certain embodiments, a conjugate group comprises a cell-targeting moiety. In certain embodiments, a conjugate group has the general formula:

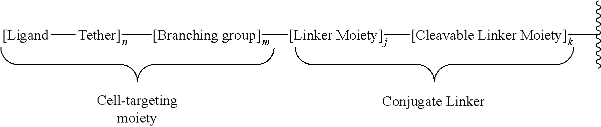

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, each ligand of a cell-targeting moiety has an affinity for at least one type of receptor on a target cell. In certain embodiments, each ligand has an affinity for at least one type of receptor on the surface of a mammalian liver cell. In certain embodiments, each ligand has an affinity for the hepatic asialoglycoprotein receptor (ASGP-R). In certain embodiments, each ligand is a carbohydrate.

In certain embodiments, a conjugate group comprises a cell-targeting conjugate moiety. In certain embodiments, a conjugate group has the general formula:

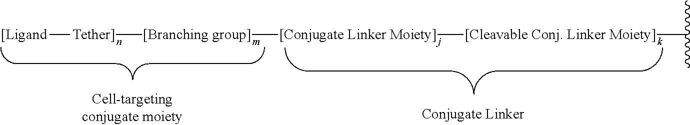

wherein n is from 1 to about 3, m is 0 when n is 1, m is 1 when n is 2 or greater, j is 1 or 0, and k is 1 or 0.

In certain embodiments, n is 1, j is 1 and k is 0. In certain embodiments, n is 1, j is 0 and k is 1. In certain embodiments, n is 1, j is 1 and k is 1. In certain embodiments, n is 2, j is 1 and k is 0. In certain embodiments, n is 2, j is 0 and k is 1. In certain embodiments, n is 2, j is 1 and k is 1. In certain embodiments, n is 3, j is 1 and k is 0. In certain embodiments, n is 3, j is 0 and k is 1. In certain embodiments, n is 3, j is 1 and k is 1.

In certain embodiments, conjugate groups comprise cell-targeting moieties that have at least one tethered ligand. In certain embodiments, cell-targeting moieties comprise two tethered ligands covalently attached to a branching group. In certain embodiments, cell-targeting moieties comprise three tethered ligands covalently attached to a branching group.

In certain embodiments, conjugate groups comprise cell-targeting moieties that have affinities for transferrin receptor (TfR) (also referred to herein as TfR1 and CD71). In certain embodiments, a conjugate group described herein comprises an anti-TfR1 antibody or fragment thereof. In certain embodiments, the conjugate group comprises a protein or peptide capable of binding TfR1. In certain embodiments, the conjugate group comprises an aptamer capable of binding TfR1. In certain embodiments, the anti-TfR1 antibody or fragment thereof can be any known in the art including but not limited to those described in WO1991/004753; WO2013/103800; WO2014/144060; WO2016/081643; WO2016/179257; WO2016/207240; WO2017/221883; WO2018/129384; WO2018/124121; WO2019/151539; WO2020/132584; WO2020/028864; U.S. Pat. Nos. 7,208,174; 9,034,329; and 10,550,188. In certain embodiments, a fragment of an anti-TfR1 antibody is F(ab′)₂, Fab, Fab′, Fv, or scFv.

In certain embodiments, the conjugate group comprises a protein or peptide capable of binding TfR1. In certain embodiments, the protein or peptide capable of binding TfR1 can be any known in the art including but not limited to those described in WO2019/140050; WO2020/037150; WO2020/124032; and U.S. Pat. No. 10,138,483.

In certain embodiments, the conjugate group comprises an aptamer capable of binding TfR1. In certain embodiments, the aptamer capable of binding TfR1 can be any known in the art including but not limited to those described in WO2013/163303; WO2019/033051; and WO2020/245198.

### B. Certain Terminal Groups

In certain embodiments, oligomeric compounds comprise one or more terminal groups. In certain such embodiments, oligomeric compounds comprise a stabilized 5′-phosphate. Stabilized 5′-phosphates include, but are not limited to 5′-phosphoanates, including, but not limited to 5′-vinylphosphonates. In certain embodiments, terminal groups comprise one or more abasic nucleosides and/or inverted nucleosides. In certain embodiments, terminal groups comprise one or more 2′-linked nucleosides. In certain such embodiments, the 2′-linked nucleoside is an abasic nucleoside.

### III. Antisense Activity

In certain embodiments, oligomeric compounds and oligomeric duplexes are capable of hybridizing to a target nucleic acid, resulting in at least one antisense activity; such oligomeric compounds and oligomeric duplexes are antisense compounds. In certain embodiments, antisense compounds have antisense activity when they reduce the amount or activity of a target nucleic acid by 25% or more in the standard in vitro assay. In certain embodiments, antisense compounds selectively affect one or more target nucleic acid. Such antisense compounds comprise a nucleobase sequence that hybridizes to one or more target nucleic acid, resulting in one or more desired antisense activity and does not hybridize to one or more non-target nucleic acid or does not hybridize to one or more non-target nucleic acid in such a way that results in significant undesired antisense activity.

In certain antisense activities, hybridization of an antisense compound to a target nucleic acid results in recruitment of a protein that cleaves the target nucleic acid. For example, certain antisense compounds result in RNase H mediated cleavage of the target nucleic acid. RNase H is a cellular endonuclease that cleaves the RNA strand of an RNA:DNA duplex. The DNA in such an RNA:DNA duplex need not be unmodified DNA. In certain embodiments, described herein are antisense compounds that are sufficiently “DNA-like” to elicit RNase H activity. In certain embodiments, one or more non-DNA-like nucleoside in the gap of a gapmer is tolerated.

In certain antisense activities, an antisense compound or a portion of an antisense compound is loaded into an RNA-induced silencing complex (RISC), ultimately resulting in cleavage of the target nucleic acid. For example, certain antisense compounds result in cleavage of the target nucleic acid by Argonaute. Antisense compounds that are loaded into RISC are RNAi agents. RNAi agents may be double-stranded (siRNA) or single-stranded (ssRNA).

In certain embodiments, hybridization of an antisense compound to a target nucleic acid does not result in recruitment of a protein that cleaves that target nucleic acid. In certain embodiments, hybridization of the antisense compound to the target nucleic acid results in alteration of splicing of the target nucleic acid. In certain embodiments, hybridization of an antisense compound to a target nucleic acid results in inhibition of a binding interaction between the target nucleic acid and a protein or other nucleic acid. In certain embodiments, hybridization of an antisense compound to a target nucleic acid results in alteration of translation of the target nucleic acid.

Antisense activities may be observed directly or indirectly. In certain embodiments, observation or detection of an antisense activity involves observation or detection of a change in an amount of a target nucleic acid or protein encoded by such target nucleic acid, a change in the ratio of splice variants of a nucleic acid or protein and/or a phenotypic change in a cell or subject.

### IV. Certain Target Nucleic Acids

In certain embodiments, oligomeric compounds comprise or consist of an oligonucleotide comprising a portion that is complementary to a target nucleic acid. In certain embodiments, the target nucleic acid is an endogenous RNA molecule. In certain embodiments, the target nucleic acid encodes a protein. In certain such embodiments, the target nucleic acid is selected from: a mature mRNA and a pre-mRNA, including intronic, exonic and untranslated regions. In certain embodiments, the target nucleic acid is a mature mRNA. In certain embodiments, the target nucleic acid is a pre-mRNA. In certain embodiments, the target region is entirely within an intron. In certain embodiments, the target region spans an intron/exon junction. In certain embodiments, the target region is at least 50% within an intron.

### A. Complementarity/Mismatches to the Target Nucleic Acid

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a region that is 100% or fully complementary to a target nucleic acid. In certain embodiments, the region of full complementarity is from 6 to 20, 10 to 18, or 18 to 20 nucleobases in length.

It is possible to introduce mismatch bases without eliminating activity. For example, Gautschi et al (J. Natl. Cancer Inst. 93:463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo. Maher and Dolnick (Nuc. Acid. Res. 16:3341-3358, 1988) tested a series of tandem 14 nucleobase oligonucleotides, and a 28 and 42 nucleobase oligonucleotides comprised of the sequence of two or three of the tandem oligonucleotides, respectively, for their ability to arrest translation of human DHFR in a rabbit reticulocyte assay. Each of the three 14 nucleobase oligonucleotides alone was able to inhibit translation, albeit at a more modest level than the 28 or 42 nucleobase oligonucleotides.

In certain embodiments, oligonucleotides are complementary to the target nucleic acid over the entire length of the oligonucleotide. In certain embodiments, oligonucleotides are 99%, 95%, 90%, 85%, or 80% complementary to the target nucleic acid. In certain embodiments, oligonucleotides are at least 80% complementary to the target nucleic acid over the entire length of the oligonucleotide and comprise a portion that is 100% or fully complementary to a target nucleic acid. In certain embodiments, the portion of full complementarity is 6, 7, 8, 9, 10, 11, 12, 13, 14, 15, 16, 17, 18, 19, 20, 21, 22, 23, or 24 nucleobases in length.

In certain embodiments, oligonucleotides comprise one or more mismatched nucleobases relative to the target nucleic acid. In certain embodiments, antisense activity against the target is reduced by such mismatch, but activity against a non-target is reduced by a greater amount. Thus, in certain embodiments selectivity of the oligonucleotide is improved. In certain embodiments, the mismatch is specifically positioned within an oligonucleotide having a gapmer motif. In certain embodiments, the mismatch is at position 1, 2, 3, 4, 5, 6, 7, 8, 9, 10, or 11 from the 5′-end of the gap region. In certain embodiments, the mismatch is at position 1, 2, 3, 4, 5, or 6 from the 5′-end of the 5′ wing region or the 3′ wing region.

### B. GYS1

In certain embodiments, oligomeric compounds comprise or consist of an oligonucleotide that is complementary to a target nucleic acid, wherein the target nucleic acid is a glycogen synthase 1 (GYS1) nucleic acid. In certain embodiments, the GYS1 nucleic acid includes a DNA sequence encoding GYS1, or an RNA sequence transcribed from DNA encoding GYS1 (including genomic DNA comprising introns and exons). In certain embodiments, the GYS1 nucleic acid has the sequence set forth in SEQ ID NO: 1 (GENBANK Accession No. NM_002103.4), SEQ ID NO: 2 (the complement of GENBANK Accession No. NC_000019.10 truncated from nucleotides 48965001 to 48996000), SEQ ID NO: 3 (HG19_CHR19:49468258-49499257(−)), SEQ ID NO: 4 (GENBANK Accession No. NG_012923.1), SEQ ID NO: 5 (UCSC ID: UC0002PLP.3), SEQ ID NO: 6 (UCSC ID: UC010EMM.3), SEQ ID NO: 7 (UCSC ID: UC010XZZ.2), SEQ ID NO: 8 (GENBANK Accession No. NM_001161587.1), SEQ ID NO: 9 (GENBANK Accession No. NR_027763.1), SEQ ID NO: 10 (GENBANK Accession No. AK303712.1), or SEQ ID NO: 14 (ENSEMBL GENE ID: ENSG00000104812.15 from Enesmbl Release 108 (October 2022)).

In certain embodiments, contacting a cell with an oligomeric compound complementary to any one of SEQ ID NOs: 1-10 and 14 reduces the amount of GYS1 RNA in a cell. In certain embodiments, contacting a cell with an oligomeric compound complementary to any one of SEQ ID NOs: 1-10 and 14 reduces the amount of GYS1 protein in a cell. In certain embodiments, the cell is in vitro. In certain embodiments, the cell is in a subject. In certain embodiments, the oligomeric compound consists of a modified oligonucleotide. In certain embodiments, contacting a cell in a subject with an oligomeric compound complementary to any one of SEQ ID NOs: 1-10 and 14 ameliorates one or more symptoms or hallmarks of a polyglucosan disease. In certain embodiments, the polyglucosan disease is Lafora disease. In certain embodiments, the polyglucosan disease is adult polyglucosan body disease (APBD). In certain embodiments, the polyglucosan disease is Pompe disease or Andersen's disease.

In certain embodiments, an oligomeric compound complementary to any one of SEQ ID NOs: 1-10 and 14 is capable of reducing the amount of GYS1 RNA in vitro by at least 10%, at least 20%, at least 30%, at least 40%, at least 50%, at least 60%, at least 70%, at least 80%, or at least 90% when administered according to the standard in vitro assay. In certain embodiments, an oligomeric compound complementary to any one of SEQ ID NOs: 1-10 and 14 is capable of reducing the amount of GYS1 RNA in vivo by at least 10%, at least 20%, at least 30%, at least 40%, at least 50%, at least 60%, at least 70%, at least 80%, or at least 90% when administered according to the standard in vivo assay. In certain embodiments, an oligomeric compound complementary to any one of SEQ ID NOs: 1-10 and 14 is capable of reducing the amount of GYS1 protein in vitro by at least 10%, at least 20%, at least 30%, at least 40%, at least 50%, at least 60%, at least 70%, at least 80%, or at least 90% when administered according to the standard in vitro assay. In certain embodiments, an oligomeric compound complementary to any one of SEQ ID NOs: 1-10 and 14 is capable of reducing the amount of GYS1 protein in vivo by at least 10%, at least 20%, at least 30%, at least 40%, at least 50%, at least 60%, at least 70%, at least 80%, or at least 90% when administered according to the standard in vivo assay. In certain embodiments, an oligomeric compound complementary to any one of SEQ ID NOs: 1-10 and 14 is capable of reducing the amount of GYS1 in the CSF of a subject by at least 10%, at least 20%, at least 30%, at least 40%, at least 50%, at least 60%, at least 70%, at least 80%, or at least 90%. In certain embodiments, an oligomeric compound complementary to any one of SEQ ID NOs: 1-10 and 14 is capable of reducing the amount of GYS1 protein in the CSF of a subject by at least 10%, at least 20%, at least 30%, at least 40%, at least 50%, at least 60%, at least 70%, at least 80%, or at least 90%.

### C. Certain Target Nucleic Acids in Certain Tissues

In certain embodiments, oligomeric compounds comprise or consist of an oligonucleotide comprising a portion that is complementary to a target nucleic acid, wherein the target nucleic acid is expressed in a pharmacologically relevant tissue. In certain embodiments, the pharmacologically relevant tissues are the cells and tissues that comprise the central nervous system (CNS). Such tissues include the brain and spinal cord. In certain embodiments, the pharmacologically relevant tissues include white matter tracts, such tissues include the corpus callosum, cerebellum, striatum, hippocampus, and brainstem.

### V. Certain Methods and Uses

Certain embodiments provided herein relate to methods of reducing or inhibiting GYS1 expression or activity, which can be useful for treating, preventing, or ameliorating a disease or disorder associated with GYS1. In certain embodiments, the disease or disorder associated with GYS1 is a neurogenerative disease characterized by an accumulation of aberrant glycogen, an accumulation of polyglucosan bodies, and/or an accumulation of Lafora bodies. In certain embodiments, the disease or disorder associated with GYS1 is a glycogen storage disease. In certain embodiments, the glycogen storage disease is Lafora disease, adult polyglucosan body disease (APBD), Andersen's disease, or Pompe disease. In certain embodiments, the glycogen storage disease is Lafora disease.

In certain embodiments, a method comprises administering to a subject an oligomeric compound, a modified oligonucleotide, an oligomeric duplex, or an antisense agent, any of which having a nucleobase sequence complementary to a GYS1 nucleic acid. In certain embodiments, the subject has or is at risk for developing a disease or disorder associated with GYS1. In certain embodiments, the subject has a glycogen storage disease. In certain embodiments, the subject has a neurogenerative disease characterized by an accumulation of aberrant glycogen, an accumulation of polyglucosan bodies, and/or an accumulation of Lafora bodies. In certain embodiments, the subject has Lafora disease. In certain embodiments, the subject has adult polyglucosan body disease (APBD). In certain embodiments, the subject has Andersen's disease. In certain embodiments, the subject has Pompe disease.

In certain embodiments, a method of treating a disease or disorder associated with GYS1 comprises administering to a subject an oligomeric compound, a modified oligonucleotide, an oligomeric duplex, or an antisense agent, any of which having a nucleobase sequence complementary to a GYS1 nucleic acid. In certain embodiments, the subject has or is at risk for developing a disease or disorder associated with GYS1. In certain embodiments, the subject has a neurogenerative disease characterized by an accumulation of aberrant glycogen, an accumulation of polyglucosan bodies, and/or an accumulation of Lafora bodies.

In certain embodiments, a method of treating a glycogen storage disease comprises administering to a subject an oligomeric compound, a modified oligonucleotide, an oligomeric duplex, or an antisense agent, any of which having a nucleobase sequence complementary to a GYS1 nucleic acid. In certain embodiments, the subject has Lafora disease. In certain embodiments, the subject has adult polyglucosan body disease (APBD). In certain embodiments, the subject has Andersen's disease. In certain embodiments, the subject has Pompe disease. In certain embodiments, at least one symptom or hallmark of the glycogen storage disease is ameliorated. In certain embodiments, the at least one symptom or hallmark is seizures, cognitive deterioration, neuromuscular weakness, myoclonus, dementia, ataxia, cerebellar dysfunction, impaired speech, loss of ambulation, swallowing difficulty, or epileptic episode. In certain embodiments, administration of the oligomeric compound, the modified oligonucleotide, the oligomeric duplex, or the antisense agent to the subject reduces or delays the onset or progression of seizures, neuromuscular weakness, myoclonus, dementia, ataxia, cerebellar dysfunction, impaired speech, a loss of ambulation, swallowing difficulty, or epileptic episode, or slows cognitive deterioration in the subject.

In certain embodiments, a method of reducing expression of GYS1 nucleic acid, for example RNA, or reducing expression of GYS1 protein in a cell comprises contacting the cell with an oligomeric compound, a modified oligonucleotide, an oligomeric duplex, or an antisense agent, any of which having a nucleobase sequence complementary to a GYS1 nucleic acid. In certain embodiments, the subject has or is at risk for developing a disease or disorder associated with GYS1. In certain embodiments, the subject has or is at risk for developing a glycogen storage disease. In certain embodiments, the cell is a neuron. In certain embodiments, the cell is a human cell.

Certain embodiments are drawn to an oligomeric compound, a modified oligonucleotide, an oligomeric duplex, or an antisense agent, any of which having a nucleobase sequence complementary to a GYS1 nucleic acid, for use in treating a disease or disorder associated with GYS1 or for use in the manufacture of a medicament for treating a disease or disorder associated with GYS1. In certain embodiments, the disease or disorder associated with GYS1 is a neurogenerative disease characterized by an accumulation of aberrant glycogen, an accumulation of polyglucosan bodies, and/or an accumulation of Lafora bodies.

Certain embodiments are drawn to an oligomeric compound, a modified oligonucleotide, an oligomeric duplex, or an antisense agent, any of which having a nucleobase sequence complementary to a GYS1 nucleic acid, for use in treating a glycogen storage disease or for use in the manufacture of a medicament for treating a glycogen storage disease. In certain embodiments, the glycogen storage disease is Lafora disease, adult polyglucosan body disease (APBD), Andersen's disease, or Pompe disease. In certain embodiments, the glycogen storage disease is Lafora disease.

In any of the methods or uses described herein, the oligomeric compound, the modified oligonucleotide, the oligomeric duplex, or the antisense agent can be any described herein.

### VI. Certain Pharmaceutical Compositions

In certain embodiments, described herein are pharmaceutical compositions comprising one or more oligomeric compounds. In certain embodiments, the one or more oligomeric compounds each consists of a modified oligonucleotide. In certain embodiments, the pharmaceutical composition comprises a pharmaceutically acceptable diluent or carrier. In certain embodiments, a pharmaceutical composition comprises or consists of a sterile saline solution and one or more oligomeric compound. In certain embodiments, the sterile saline is pharmaceutical grade saline. In certain embodiments, a pharmaceutical composition comprises or consists of one or more oligomeric compound and sterile water. In certain embodiments, the sterile water is pharmaceutical grade water. In certain embodiments, a pharmaceutical composition comprises or consists of one or more oligomeric compound and phosphate-buffered saline (PBS). In certain embodiments, the sterile PBS is pharmaceutical grade PBS. In certain embodiments, a pharmaceutical composition comprises or consists of one or more oligomeric compound and artificial cerebrospinal fluid (“artificial CSF” or “aCSF”). In certain embodiments, the artificial cerebrospinal fluid is pharmaceutical grade.

In certain embodiments, a pharmaceutical composition comprises a modified oligonucleotide and artificial cerebrospinal fluid (aCSF). In certain embodiments, a pharmaceutical composition consists of a modified oligonucleotide and artificial cerebrospinal fluid. In certain embodiments, a pharmaceutical composition consists essentially of a modified oligonucleotide and artificial cerebrospinal fluid. In certain embodiments, the artificial cerebrospinal fluid is pharmaceutical grade.

In certain embodiments, aCSF comprises sodium chloride, potassium chloride, sodium dihydrogen phosphate dihydrate, sodium phosphate dibasic anhydrous, calcium chloride dihydrate, and magnesium chloride hexahydrate. In certain embodiments, the pH of an aCSF solution is modulated with a suitable pH-adjusting agent, for example, with acids such as hydrochloric acid and alkalis such as sodium hydroxide, to a range of from about 7.1-7.3, or to about 7.2.

In certain embodiments, pharmaceutical compositions comprise one or more oligomeric compound and one or more excipients. In certain embodiments, excipients are selected from water, salt solutions, alcohol, polyethylene glycols, gelatin, lactose, amylase, magnesium stearate, talc, silicic acid, viscous paraffin, hydroxymethylcellulose and polyvinylpyrrolidone.

In certain embodiments, oligomeric compounds may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered.

In certain embodiments, pharmaceutical compositions comprising an oligomeric compound encompass any pharmaceutically acceptable salts of the oligomeric compound, esters of the oligomeric compound, or salts of such esters. In certain embodiments, pharmaceutical compositions comprising oligomeric compounds comprising one or more oligonucleotide, upon administration to a subject, including a human, are capable of providing (directly or indirectly) the biologically active metabolite or residue thereof. Accordingly, for example, the disclosure is also drawn to pharmaceutically acceptable salts of oligomeric compounds, prodrugs, pharmaceutically acceptable salts of such prodrugs, and other bioequivalents. In certain embodiments, pharmaceutically acceptable salts comprise inorganic salts, such as monovalent or divalent inorganic salts. Suitable pharmaceutically acceptable salts include, but are not limited to, sodium, potassium, calcium, and magnesium salts. In certain embodiments, prodrugs comprise one or more conjugate group attached to an oligonucleotide, wherein the conjugate group is cleaved by endogenous nucleases within the body.

In certain embodiments, oligomeric compounds are lyophilized and isolated as sodium salts. In certain embodiments, the sodium salt of an oligomeric compound is mixed with a pharmaceutically acceptable diluent. In certain embodiments, the pharmaceutically acceptable diluent comprises sterile saline, sterile water, PBS, or aCSF. In certain embodiments, the sodium salt of an oligomeric compound is mixed with PBS. In certain embodiments, the sodium salt of an oligomeric compound is mixed with aCSF.

Lipid moieties have been used in nucleic acid therapies in a variety of methods. In certain such methods, the nucleic acid, such as an oligomeric compound, is introduced into preformed liposomes or lipoplexes made of mixtures of cationic lipids and neutral lipids. In certain methods, DNA complexes with mono- or poly-cationic lipids are formed without the presence of a neutral lipid. In certain embodiments, a lipid moiety is selected to increase distribution of a pharmaceutical agent to a particular cell or tissue. In certain embodiments, a lipid moiety is selected to increase distribution of a pharmaceutical agent to fat tissue. In certain embodiments, a lipid moiety is selected to increase distribution of a pharmaceutical agent to muscle tissue.

In certain embodiments, pharmaceutical compositions comprise a delivery system. Examples of delivery systems include, but are not limited to, liposomes and emulsions. Certain delivery systems are useful for preparing certain pharmaceutical compositions including those comprising hydrophobic compounds. In certain embodiments, certain organic solvents such as dimethylsulfoxide are used.

In certain embodiments, pharmaceutical compositions comprise one or more tissue-specific delivery molecules designed to deliver the one or more pharmaceutical agents comprising an oligomeric compound provided herein to specific tissues or cell types. For example, in certain embodiments, pharmaceutical compositions include liposomes coated with a tissue-specific antibody.

In certain embodiments, pharmaceutical compositions comprise a co-solvent system. Certain of such co-solvent systems comprise, for example, benzyl alcohol, a nonpolar surfactant, a water-miscible organic polymer, and an aqueous phase. In certain embodiments, such co-solvent systems are used for hydrophobic compounds. A non-limiting example of such a co-solvent system is the VPD co-solvent system, which is a solution of absolute ethanol comprising 3% w/v benzyl alcohol, 8% w/v of the nonpolar surfactant Polysorbate 80™ and 65% w/v polyethylene glycol 300. The proportions of such co-solvent systems may be varied considerably without significantly altering their solubility and toxicity characteristics. Furthermore, the identity of co-solvent components may be varied: for example, other surfactants may be used instead of Polysorbate 80™; the fraction size of polyethylene glycol may be varied; other biocompatible polymers may replace polyethylene glycol, e.g., polyvinyl pyrrolidone; and other sugars or polysaccharides may substitute for dextrose.

In certain embodiments, pharmaceutical compositions are prepared for oral administration. In certain embodiments, pharmaceutical compositions are prepared for buccal administration. In certain embodiments, a pharmaceutical composition is prepared for administration by injection (e.g., intravenous, subcutaneous, intramuscular, intrathecal (IT), intracerebroventricular (ICV), etc.). In certain of such embodiments, a pharmaceutical composition comprises a carrier and is formulated in aqueous solution, such as water or physiologically compatible buffers such as Hanks's solution, Ringer's solution, or physiological saline buffer. In certain embodiments, other ingredients are included (e.g., ingredients that aid in solubility or serve as preservatives). In certain embodiments, injectable suspensions are prepared using appropriate liquid carriers, suspending agents and the like. Certain pharmaceutical compositions for injection are presented in unit dosage form, e.g., in ampoules or in multi-dose containers. Certain pharmaceutical compositions for injection are suspensions, solutions or emulsions in oily or aqueous vehicles, and may contain formulatory agents such as suspending, stabilizing and/or dispersing agents. Certain solvents suitable for use in pharmaceutical compositions for injection include, but are not limited to, lipophilic solvents and fatty oils, such as sesame oil, synthetic fatty acid esters, such as ethyl oleate or triglycerides, and liposomes.

Under certain conditions, certain compounds disclosed herein act as acids. Although such compounds may be drawn or described in protonated (free acid) form, or ionized and in association with a cation (salt) form, aqueous solutions of such compounds exist in equilibrium among such forms. For example, a phosphodiester linkage of an oligonucleotide in aqueous solution exists in equilibrium among free acid, anion and salt forms. Unless otherwise indicated, compounds described herein are intended to include all such forms. Moreover, certain oligonucleotides have several such linkages, each of which is in equilibrium. Thus, oligonucleotides in solution exist in an ensemble of forms at multiple positions all at equilibrium. The term “oligonucleotide” is intended to include all such forms. Drawn structures necessarily depict a single form. Nevertheless, unless otherwise indicated, such drawings are likewise intended to include corresponding forms. Herein, a structure depicting the free acid of a compound followed by the term “or a pharmaceutically acceptable salt thereof” expressly includes all such forms that may be fully or partially protonated/de-protonated/in association with a cation or a combination of cations. In certain embodiments, one or more specific cation is identified. The cations include, but are not limited to, sodium, potassium, calcium, and magnesium. In certain embodiments, a structure depicting the free acid of a compound followed by the term “or a pharmaceutically acceptable salt thereof” expressly includes all such forms that may be fully or partially protonated/de-protonated/in association with one or more cations selected from sodium, potassium, calcium, and magnesium.

In certain embodiments, modified oligonucleotides or oligomeric compounds are in aqueous solution with sodium. In certain embodiments, modified oligonucleotides or oligomeric compounds are in aqueous solution with potassium. In certain embodiments, modified oligonucleotides or oligomeric compounds are in PBS. In certain embodiments, modified oligonucleotides or oligomeric compounds are in water. In certain such embodiments, the pH of the solution is adjusted with NaOH and/or HCl to achieve a desired pH.

Herein, certain specific doses are described. A dose may be in the form of a dosage unit. For clarity, a dose (or dosage unit) of a modified oligonucleotide or an oligomeric compound in milligrams indicates the mass of the free acid form of the modified oligonucleotide or oligomeric compound. As described above, in aqueous solution, the free acid is in equilibrium with anionic and salt forms. However, for the purpose of calculating dose, it is assumed that the modified oligonucleotide or oligomeric compound exists as a solvent-free, sodium-acetate free, anhydrous, free acid.

In certain embodiments, where a modified oligonucleotide or an oligomeric compound is in solution comprising sodium (e.g., saline), the modified oligonucleotide or oligomeric compound may be partially or fully de-protonated and in association with sodium ions. However, the mass of the protons is nevertheless counted toward the weight of the dose, and the mass of the sodium ions is not counted toward the weight of the dose. Thus, for example, a dose, or dosage unit, of 10 mg of Compound No. 1127954, equals the number of fully protonated molecules that weighs 10 mg. This would be equivalent to 10.47 mg of solvent-free, sodium acetate-free, anhydrous sodiated Compound No. 1127954.

In certain embodiments, where a modified oligonucleotide or oligomeric compound is in a solution, such as aCSF, comprising sodium, potassium, calcium, and magnesium, the modified oligonucleotide or oligomeric compound may be partially or fully de-protonated and in association with sodium, potassium, calcium, and/or magnesium. However, the mass of the protons is nevertheless counted toward the weight of the dose, and the mass of the sodium, potassium, calcium, and magnesium ions is not counted toward the weight of the dose.

In certain embodiments, when an oligomeric compound comprises a conjugate group, the mass of the conjugate group may be included in calculating the dose of such oligomeric compound. If the conjugate group also has an acid, the conjugate group is likewise assumed to be fully protonated for the purpose of calculating dose.

### VII. Certain Compositions

### 1. Compound No. 1127954

In certain embodiments, Compound No. 1127954 is characterized as a 5-10-5 MOE gapmer having a nucleobase sequence (from 5′ to 3′) of CCGTCTACAGGATTTTCTAG (SEQ ID NO: 83), wherein each of nucleosides 1-5 and 16-20 (from 5′ to 3′) are 2′-O(CH₂)₂OCH₃ nucleosides and each of nucleosides 6-15 are 2′-β-D-deoxynucleosides, wherein the internucleoside linkages between nucleosides 2 to 3, 3 to 4, 4 to 5, 16 to 17, and 17 to 18 are phosphodiester internucleoside linkages, the internucleoside linkages between nucleosides 1 to 2, 5 to 6, 6 to 7, 7 to 8, 8 to 9, 9 to 10, 10 to 11, 11 to 12, 12 to 13, 13 to 14, 14 to 15, 15 to 16, 18 to 19, and 19 to 20 are phosphorothioate internucleoside linkages, and wherein each cytosine is a 5-methylcytosine.

In certain embodiments, Compound No. 1127954 is represented by the following chemical notation: ^{m}Cₑₛ^{m}CₑₒGₑₒTₑₒ^{m}CₑₛT_{ds}A_{ds}^{m}C_{ds}A_{ds}G_{ds}G_{ds}A_{ds}T_{ds}T_{ds}T_{ds}Tₑₒ^{m}CₑₒTₑₛAₑₛGₑ (SEQ ID NO: 83), wherein:
A=an adenine nucleobase,
^{m}C=a 5-methylcytosine nucleobase,
G=a guanine nucleobase,
T=a thymine nucleobase,
e=a 2′-O(CH₂)₂OCH₃ ribosyl sugar moiety,
d=a 2′-β-D-deoxyribosyl sugar moiety,
s=a phosphorothioate internucleoside linkage, and
o=a phosphodiester internucleoside linkage.

In certain embodiments, Compound No. 1127954 is represented by the following chemical structure:

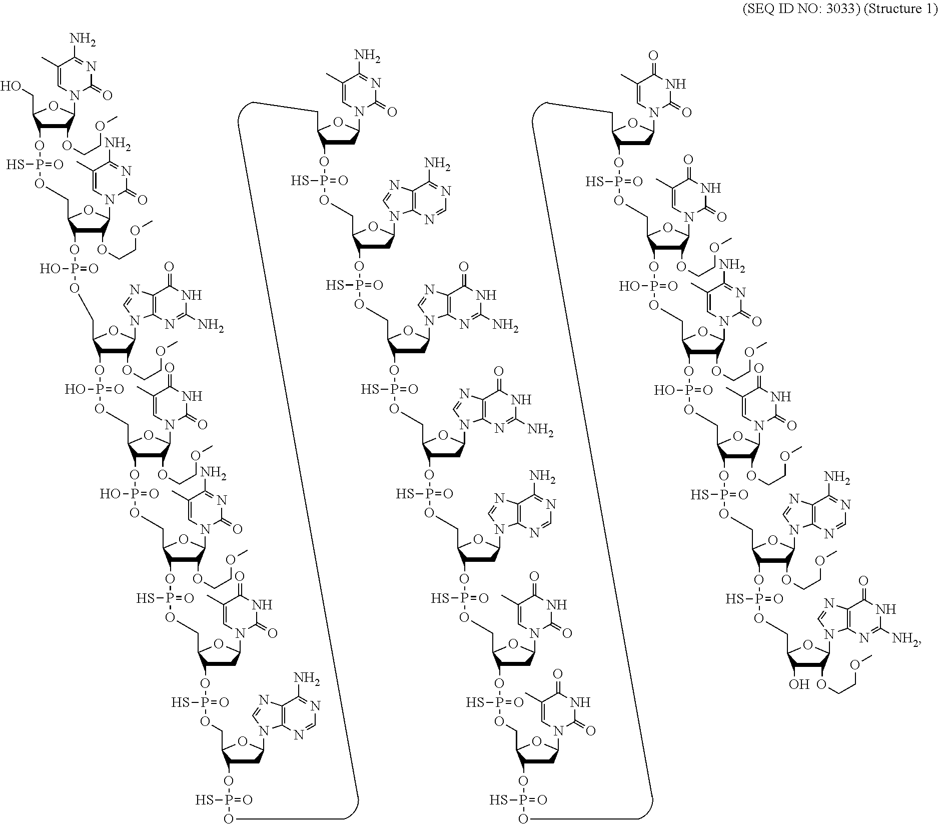

or a pharmaceutically acceptable salt thereof. In certain embodiments, the pharmaceutically acceptable salt of Compound No. 1127954 comprises one or more cations selected from sodium, potassium, calcium, and magnesium.

In certain embodiments, the sodium salt of Compound No. 1127954 is represented by the following chemical structure:

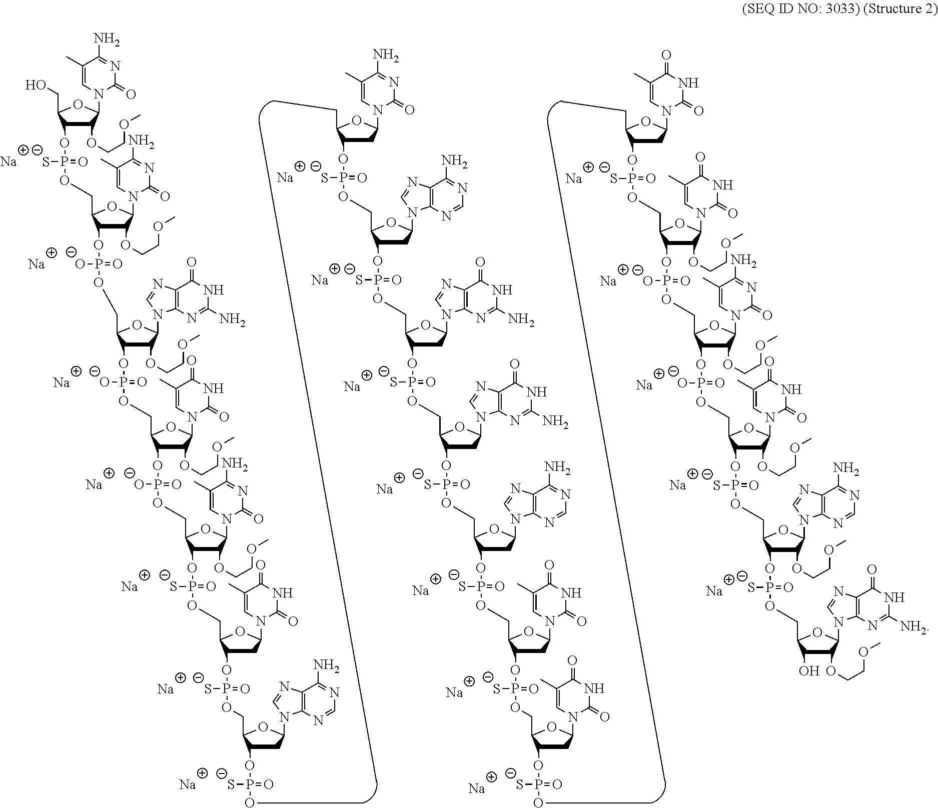

### 2. Compound No. 1127956

In certain embodiments, Compound No. 1127956 is characterized as a 5-10-5 MOE gapmer having a nucleobase sequence (from 5′ to 3′) of TTCCGTCTACAGGATTTTCT (SEQ ID NO: 1454), wherein each of nucleosides 1-5 and 16-20 (from 5′ to 3′) are 2′-O(CH₂)₂OCH₃ nucleosides and each of nucleosides 6-15 are 2′-β-D-deoxynucleosides, wherein the internucleoside linkages between nucleosides 2 to 3, 3 to 4, 4 to 5, 16 to 17, and 17 to 18 are phosphodiester internucleoside linkages, the internucleoside linkages between nucleosides 1 to 2, 5 to 6, 6 to 7, 7 to 8, 8 to 9, 9 to 10, 10 to 11, 11 to 12, 12 to 13, 13 to 14, 14 to 15, 15 to 16, 18 to 19, and 19 to 20 are phosphorothioate internucleoside linkages, and wherein each cytosine is a 5-methylcytosine.

In certain embodiments, Compound No. 1127956 is represented by the following chemical notation: TₑₛTₑₒ^{m}Cₑₒ^{m}CₑₒGₑₛT_{ds}^{m}C_{ds}T_{ds}A_{ds}^{m}C_{ds}A_{ds}G_{ds}G_{ds}A_{ds}T_{ds}TₑₒTₑₒTₑₛ^{m}CₑₛTₑ (SEQ ID NO: 1454), wherein:
A=an adenine nucleobase,
^{m}C=a 5-methylcytosine nucleobase,
G=a guanine nucleobase,
T=a thymine nucleobase,
e=a 2′-O(CH₂)₂OCH₃ ribosyl sugar moiety,
d=a 2′-β-D-deoxyribosyl sugar moiety,
s=a phosphorothioate internucleoside linkage, and
o=a phosphodiester internucleoside linkage.

In certain embodiments, Compound No. 1127956 is represented by the following chemical structure:

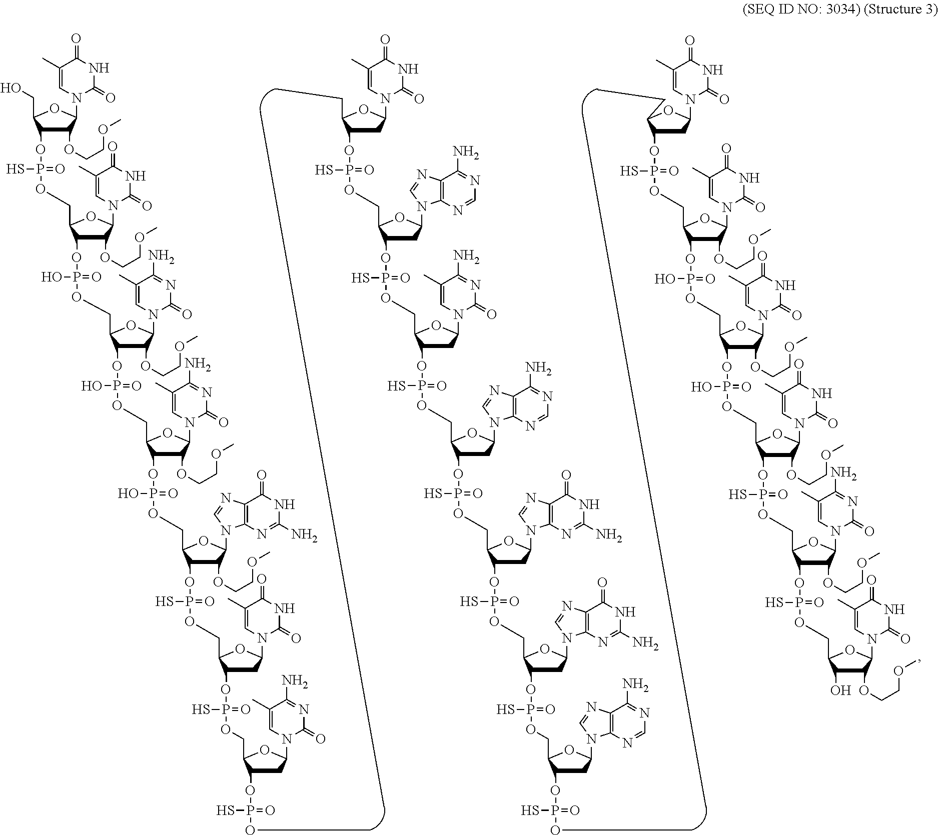

or a pharmaceutically acceptable salt thereof. In certain embodiments, the pharmaceutically acceptable salt of Compound No. 1127956 comprises one or more cations selected from sodium, potassium, calcium, and magnesium.

In certain embodiments, the sodium salt of Compound No. 1127956 is represented by the following chemical structure:

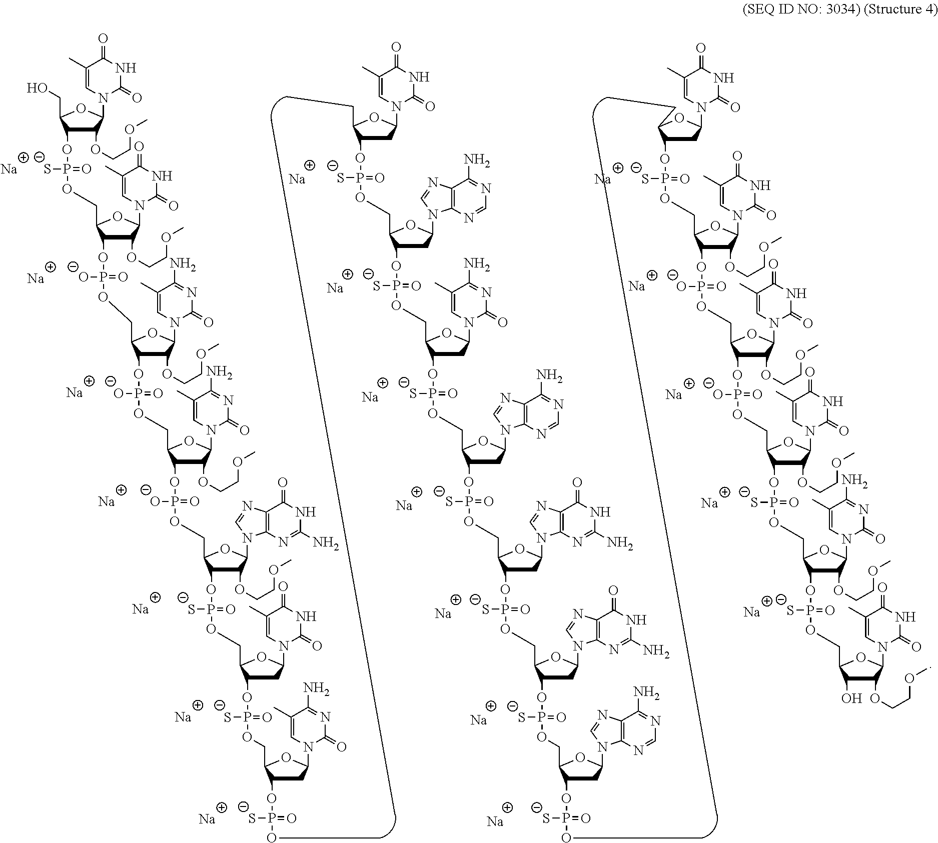

### 3. Compound No. 1311856

In certain embodiments, Compound No. 1311856 is characterized as a 6-10-4 MOE gapmer having a nucleobase sequence (from 5′ to 3′) of TTCCGTCTACAGGATTTTCT (SEQ ID NO: 1454), wherein each of nucleosides 1-6 and 17-20 (from 5′ to 3′) are 2′-O(CH₂)₂OCH₃ nucleosides and each of nucleosides 7-16 are 2′-β-D-deoxynucleosides, wherein the internucleoside linkages between nucleosides 2 to 3, 3 to 4, 4 to 5, 5 to 6, 6 to 7, and 17 to 18 are phosphodiester internucleoside linkages, the internucleoside linkages between nucleosides 1 to 2, 7 to 8, 8 to 9, 9 to 10, 10 to 11, 11 to 12, 12 to 13, 13 to 14, 14 to 15, 15 to 16, 16 to 17, 18 to 19, and 19 to 20 are phosphorothioate internucleoside linkages, and wherein each cytosine is a 5-methylcytosine.

In certain embodiments, Compound No. 1311856 is represented by the following chemical notation: TₑₛTₑₒ^{m}Cₑₒ^{m}CₑₒGₑₒTₑₒ^{m}C_{ds}T_{ds}A_{ds}^{m}C_{ds}A_{ds}G_{ds}G_{ds}A_{ds}T_{ds}T_{ds}TₑₒTₑₛ^{m}CₑₛTₑ (SEQ ID NO: 1454), wherein:
A=an adenine nucleobase,
^{m}C=a 5-methylcytosine nucleobase,
G=a guanine nucleobase,
T=a thymine nucleobase,
e=a 2′-O(CH₂)₂OCH₃ ribosyl sugar moiety,
d=a 2′-β-D-deoxyribosyl sugar moiety,
s=a phosphorothioate internucleoside linkage, and
o=a phosphodiester internucleoside linkage.

In certain embodiments, Compound No. 1311856 is represented by the following chemical structure:

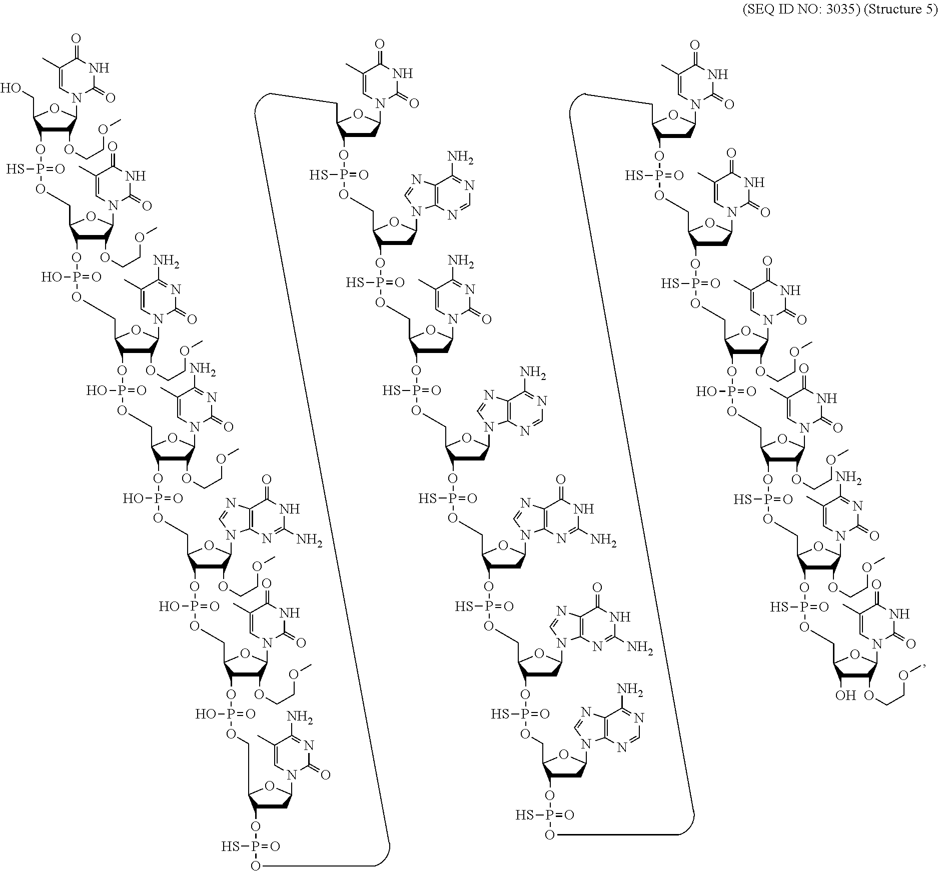

or a pharmaceutically acceptable salt thereof. In certain embodiments, the pharmaceutically acceptable salt of Compound No. 1311856 comprises one or more cations selected from sodium, potassium, calcium, and magnesium.

In certain embodiments, the sodium salt of Compound No. 1311856 is represented by the following chemical structure:

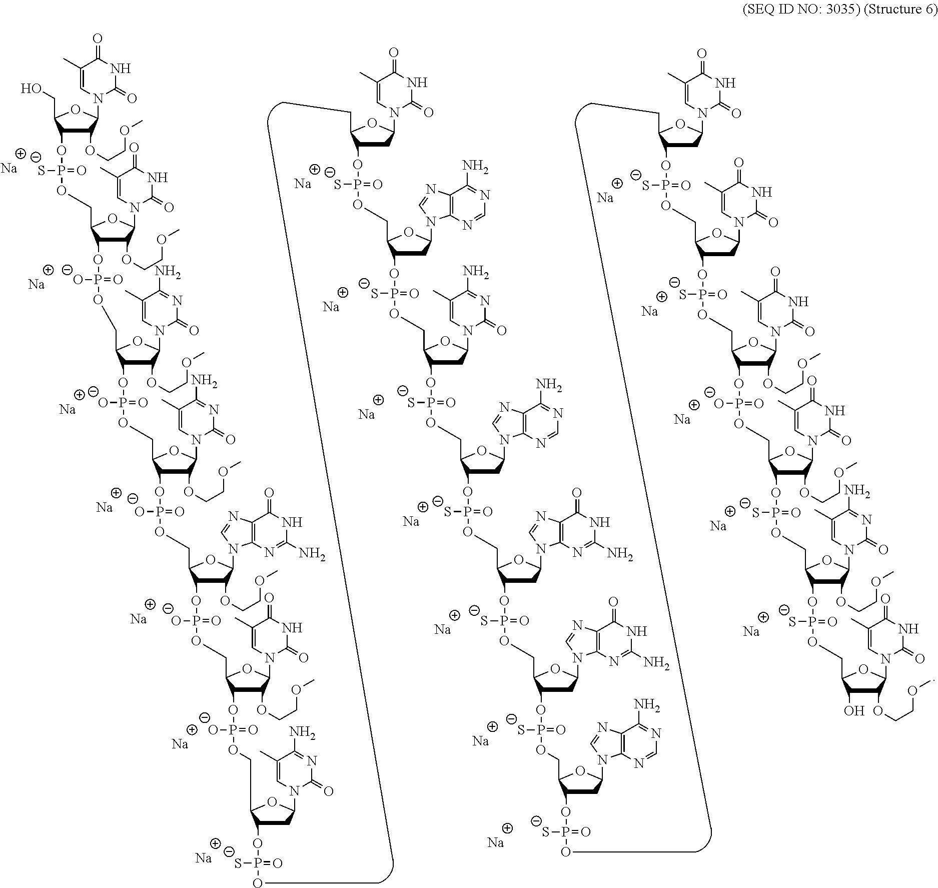

### 4. Compound No. 1128013

In certain embodiments, Compound No. 1128013 is characterized as a 5-10-5 MOE gapmer having a nucleobase sequence (from 5′ to 3′) of GCACACAAGTAAAGCTAGCA (SEQ ID NO: 921), wherein each of nucleosides 1-5 and 16-20 (from 5′ to 3′) are 2′-O(CH₂)₂OCH₃ nucleosides and each of nucleosides 6-15 are 2′-β-D-deoxynucleosides, wherein the internucleoside linkages between nucleosides 2 to 3, 3 to 4, 4 to 5, 16 to 17, and 17 to 18 are phosphodiester internucleoside linkages, the internucleoside linkages between nucleosides 1 to 2, 5 to 6, 6 to 7, 7 to 8, 8 to 9, 9 to 10, 10 to 11, 11 to 12, 12 to 13, 13 to 14, 14 to 15, 15 to 16, 18 to 19, and 19 to 20 are phosphorothioate internucleoside linkages, and wherein each cytosine is a 5-methylcytosine.

In certain embodiments, Compound No. 1128013 is represented by the following chemical notation: Gₑₛ^{m}CₑₒAₑₒ^{m}CₑₒAₑₛ^{m}C_{ds}A_{ds}A_{ds}G_{ds}T_{ds}A_{ds}A_{ds}A_{ds}G_{ds}^{m}C_{ds}TₑₒAₑₒGₑₛ^{m}CₑₛAₑ (SEQ ID NO: 921), wherein:
A=an adenine nucleobase,
^{m}C=a 5-methylcytosine nucleobase,
G=a guanine nucleobase,
T=a thymine nucleobase,
e=a 2′-O(CH₂)₂OCH₃ ribosyl sugar moiety,
d=a 2′-β-D-deoxyribosyl sugar moiety,
s=a phosphorothioate internucleoside linkage, and
o=a phosphodiester internucleoside linkage.

In certain embodiments, Compound No. 1128013 is represented by the following chemical structure:

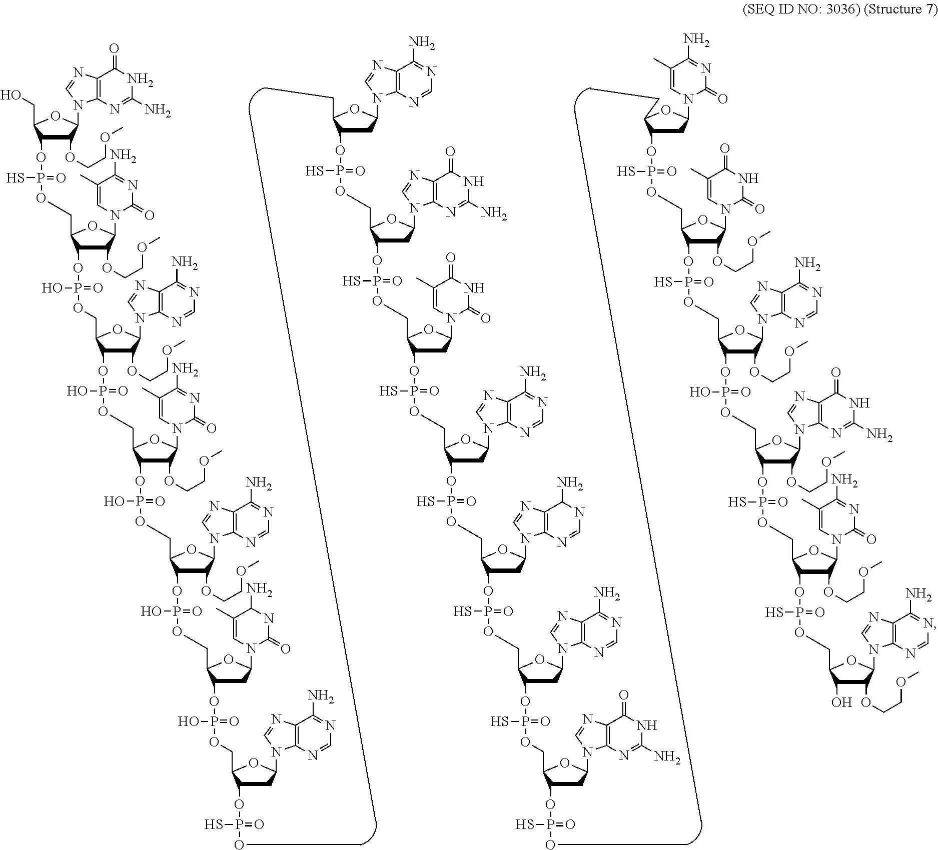

or a pharmaceutically acceptable salt thereof. In certain embodiments, the pharmaceutically acceptable salt of Compound No. 1128013 comprises one or more cations selected from sodium, potassium, calcium, and magnesium.

In certain embodiments, the sodium salt of Compound No. 1128013 is represented by the following chemical structure:

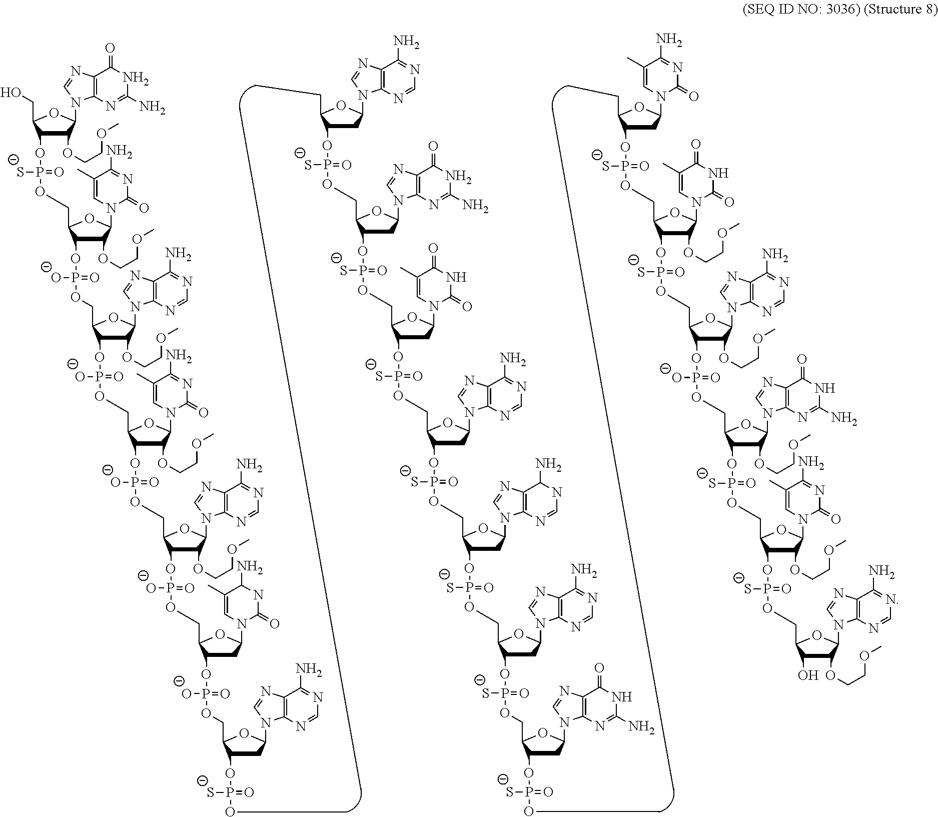

### VIII. Certain Hotspot Regions

In certain embodiments, nucleobases in the ranges specified below comprise a hotspot region of a GYS1 nucleic acid. In certain embodiments, modified oligonucleotides that are complementary to a hotspot region of GYS1 nucleic acid achieve an average of more than 50% reduction of GYS1 RNA in vitro in the standard cell assay. In certain embodiments, modified oligonucleotides that are complementary to a hotspot region of GYS1 nucleic acid achieve an average of 50% or greater reduction of GYS1 RNA in vivo in the standard in vivo assay.

### 1. Nucleobases 3341-3385 of SEQ ID NO: 1

In certain embodiments, nucleobases 3341-3385 of SEQ ID NO: 1 comprise a hotspot region. In certain embodiments, modified oligonucleotides are complementary to a portion of nucleobases 3341-3385 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages.

The nucleobase sequences of SEQ ID NOs: 1194, 1270, 1347, 446, 1424, 1497, 1574, and 1651 are complementary to a portion of nucleobases 3341-3385 of SEQ ID NO: 1.

The nucleobase sequences of Compound Nos.: 1126896, 1126897, 1126898, 941588, 1126899, 1126900, 1126901, and 1126902 are complementary to a portion of nucleobases 3341-3385 of SEQ ID NO: 1.

In certain embodiments, modified oligonucleotides complementary to a portion of nucleobases 3341-3385 of SEQ ID NO: 1 achieve at least 62% reduction of GYS1 RNA in the standard in vitro assay.

In certain embodiments, modified oligonucleotides complementary to a portion of nucleobases 3341-3385 of SEQ ID NO: 1 achieve an average of 81.8% reduction of GYS1 RNA in the standard in vitro assay.

### 2. Nucleobases 3565-3591 of SEQ ID NO: 1

In certain embodiments, nucleobases 3565-3591 of SEQ ID NO: 1 comprise a hotspot region. In certain embodiments, modified oligonucleotides are complementary to a portion of nucleobases 3565-3591 of SEQ ID NO: 1. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages.

The nucleobase sequences of SEQ ID NOs: 140, 1348, 217, 1498, and 1575 are complementary to a portion of nucleobases 3565-3591 of SEQ ID NO: 1.

The nucleobase sequences of Compound Nos.: 941596, 1126930, 941597, 1126931, 1126932, and 1126933 are complementary to a portion of nucleobases 3565-3591 of SEQ ID NO: 1.

In certain embodiments, modified oligonucleotides complementary to a portion of nucleobases 3565-3591 of SEQ ID NO: 1 achieve at least 70% reduction of GYS1 RNA in the standard in vitro assay.

In certain embodiments, modified oligonucleotides complementary to a portion of nucleobases 3565-3591 of SEQ ID NO: 1 achieve an average of 79.5% reduction of GYS1 RNA in the standard in vitro assay.

### 3. Nucleobases 5914-5948 of SEQ ID NO: 2

In certain embodiments, nucleobases 5914-5948 of SEQ ID NO: 2 comprise a hotspot region. In certain embodiments, modified oligonucleotides are complementary to a portion of nucleobases 5914-5948 of SEQ ID NO: 2. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: sooosssssssssssooss, wherein each “s” represents a phosphorothioate internucleoside linkage and each “o” represents a phosphodiester internucleoside linkage.

The nucleobase sequences of SEQ ID NOs: 2508, 2585, 2661, 2737, 2814, 2891, 517, and 594 are complementary to a portion of nucleobases 5914-5948 of SEQ ID NO: 2.

The nucleobase sequences of Compound Nos.: 1127329, 1127330, 1127331, 1127332, 1127333, 1127334, 1127335, and 1127336 are complementary to a portion of nucleobases 5914-5948 of SEQ ID NO: 2.

In certain embodiments, modified oligonucleotides complementary to a portion of nucleobases 5914-5948 of SEQ ID NO: 2 achieve at least 60% reduction of GYS1 RNA in the standard in vitro assay. In certain embodiments, modified oligonucleotides complementary to a portion of nucleobases 5914-5948 of SEQ ID NO: 2 achieve an average of 76.8% reduction of GYS1 RNA in the standard in vitro assay.

### 4. Nucleobases 7803-7844 of SEQ ID NO: 2

In certain embodiments, nucleobases 7803-7844 of SEQ ID NO: 2 comprise a hotspot region. In certain embodiments, modified oligonucleotides are complementary to a portion of nucleobases 7803-7844 of SEQ ID NO: 2. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: sooosssssssssssooss, wherein each “s” represents a phosphorothioate internucleoside linkage and each “o” represents a phosphodiester internucleoside linkage.

The nucleobase sequences of SEQ ID NOs: 1513, 1590, 1667, and 1744 are complementary to a portion of nucleobases 7803-7844 of SEQ ID NO: 2.

The nucleobase sequences of Compound Nos.: 1127412, 1127413, 1127414, and 1127415 are complementary to a portion of nucleobases 7803-7844 of SEQ ID NO: 2.

In certain embodiments, modified oligonucleotides complementary to a portion of nucleobases 7803-7844 of SEQ ID NO: 2 achieve at least 74% reduction of GYS1 RNA in the standard in vitro assay.

In certain embodiments, modified oligonucleotides complementary to a portion of nucleobases 7803-7844 of SEQ ID NO: 2 achieve an average of 81.8% reduction of GYS1 RNA in the standard in vitro assay.

### 5. Nucleobases 12350-12385 of SEQ ID NO: 2

In certain embodiments, nucleobases 12350-12385 of SEQ ID NO: 2 comprise a hotspot region. In certain embodiments, modified oligonucleotides are complementary to a portion of nucleobases 12350-12385 of SEQ ID NO: 2. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: sooosssssssssssooss, wherein each “s” represents a phosphorothioate internucleoside linkage and each “o” represents a phosphodiester internucleoside linkage.

The nucleobase sequences of SEQ ID NOs: 914, 991, 1068, 1145, and 1222 are complementary to a portion of nucleobases 12350-12385 of SEQ ID NO: 2.

The nucleobase sequences of Compound Nos.: 1127788, 1127789, 1127790, 1127791, and 1127792 are complementary to a portion of nucleobases 12350-12385 of SEQ ID NO: 2.

In certain embodiments, modified oligonucleotides complementary to a portion of nucleobases 12350-12385 of SEQ ID NO: 2 achieve at least 65% reduction of GYS1 RNA in the standard in vitro assay. In certain embodiments, modified oligonucleotides complementary to a portion of nucleobases 12350-12385 of SEQ ID NO: 2 achieve an average of 72.4% reduction of GYS1 RNA in the standard in vitro assay.

### 6. Nucleobases: 15653-15688 of SEQ ID NO: 2

In certain embodiments, nucleobases 15653-15688 of SEQ ID NO: 2 comprise a hotspot region. In certain embodiments, modified oligonucleotides are complementary to a portion of nucleobases 15653-15688 of SEQ ID NO: 2. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages.

The nucleobase sequences of SEQ ID NOs: 1303, 83, 1380, 1454, 2954, 2947, 1530, 1607, 1684, 1761, 1837, 2951, 2949, 2944, 2943, 1913, and 1990 are complementary to a portion of nucleobases 15653-15688 of SEQ ID NO: 2. The nucleobase sequences of SEQ ID NOs: 1303, 83, 1380, 1454, 1530, 1607, 1684, 1761, 1837, 1913, and 1990 are complementary to a portion of nucleobases 15653-15688 of SEQ ID NO: 2. The nucleobase sequences of SEQ ID NOs: 2954, 2947, 2951, 2949, 2944, and 2943 are complementary to a portion of nucleobases 15653-15688 of SEQ ID NO: 2.

The nucleobase sequences of Compound Nos.: 1127953, 941715, 1127954, 1311857, 1311858, 1127955, 1311856, 1127956, 1251622, 1251615, 1127957, 1127958, 1127959, 1127960, 1127961, 1251619, 1251617, 1251612, 1251611, 1127962, and 1127963 are complementary to a portion of nucleobases 15653-15688 of SEQ ID NO: 2. The nucleobase sequence of Compound Nos.: 1127953, 941715, 1127954, 1127955, 1127956, 1127957, 1127958, 1127959, 1127960, 1127961, 1127962, and 1127963 are complementary to a portion of nucleobases 15653-15688 of SEQ ID NO: 2. The nucleobase sequence of Compound Nos.: 1311857, 1311858, 1311856, 1251622, 1251615, 1251619, 1251617, 1251612, and 1251611 are complementary to a portion of nucleobases 15653-15688 of SEQ ID NO: 2.

In certain embodiments, modified oligonucleotides complementary to a portion of nucleobases 15653-15688 of SEQ ID NO: 2 achieve at least 30% reduction of GYS1 RNA in the standard in vitro assay. In certain embodiments, modified oligonucleotides complementary to a portion of nucleobases 15653-15688 of SEQ ID NO: 2 achieve an average of 77.7% reduction of GYS1 RNA in the standard in vitro assay.

### 7. Nucleobases: 15801-15845 of SEQ ID NO: 2

In certain embodiments, nucleobases 15801-15845 of SEQ ID NO: 2 comprise a hotspot region. In certain embodiments, modified oligonucleotides are complementary to a portion of nucleobases 15801-15845 of SEQ ID NO: 2. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages.

The nucleobase sequences of SEQ ID NOs: 314, 843, 920, 997, and 1074 are complementary to a portion of nucleobases 15801-15845 of SEQ ID NO: 2.

The nucleobase sequences of Compound Nos.: 941718, 1127979, 1127980, 1127981, 1127982, and 1127983 are complementary to a portion of nucleobases 15801-15845 of SEQ ID NO: 2.

In certain embodiments, modified oligonucleotides complementary to a portion of nucleobases 15801-15845 of SEQ ID NO: 2 achieve at least 88% reduction of GYS1 RNA in the standard in vitro assay. In certain embodiments, modified oligonucleotides complementary to a portion of nucleobases 15801-15845 of SEQ ID NO: 2 achieve an average of 93% reduction of GYS1 RNA in the standard in vitro assay.

### 8. Nucleobases: 16745-16800 of SEQ ID NO: 2

In certain embodiments, nucleobases 16745-16800 of SEQ ID NO: 2 comprise a hotspot region. In certain embodiments, modified oligonucleotides are complementary to a portion of nucleobases 16745-16800 of SEQ ID NO: 2. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages.

The nucleobase sequences of SEQ ID NOs: 2682, 2758, 2835, 2912, 538, 615, 691, 767, 844, 921, 998, 1075, 1152, 161, and 1305 are complementary to a portion of nucleobases 16745-16800 of SEQ ID NO: 2.

The nucleobase sequences of Compound Nos.: 1128004, 1128005, 1128006, 1128007, 1128008, 1128009, 1128010, 1128011, 1128012, 1128013, 1128014, 1128015, 1128016, 941722, 1128017, and 1128018 are complementary to a portion of nucleobases 16745-16800 of SEQ ID NO: 2.

In certain embodiments, modified oligonucleotides complementary to a portion of nucleobases 16745-16800 of SEQ ID NO: 2 achieve at least 25% reduction of GYS1 RNA in the standard in vitro assay. In certain embodiments, modified oligonucleotides complementary to a portion of nucleobases 16745-16800 of SEQ ID NO: 2 achieve an average of 68.5% reduction of GYS1 RNA in the standard in vitro assay.

### 9. Nucleobases: 16828-16864 of SEQ ID NO: 2

In certain embodiments, nucleobases 16828-16864 of SEQ ID NO: 2 comprise a hotspot region. In certain embodiments, modified oligonucleotides are complementary to a portion of nucleobases 16828-16864 of SEQ ID NO: 2. In certain embodiments, modified oligonucleotides are 20 nucleobases in length. In certain embodiments, modified oligonucleotides are gapmers. In certain embodiments, the gapmers are MOE gapmers. In certain embodiments, the gapmers are 5-10-5 MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: sooosssssssssssooss, wherein each “s” represents a phosphorothioate internucleoside linkage and each “o” represents a phosphodiester internucleoside linkage.

The nucleobase sequences of SEQ ID NOs: 1382, 1456, and 1532 are complementary to a portion of nucleobases 16828-16864 of SEQ ID NO: 2.

The nucleobase sequences of Compound Nos.: 1128019, 1128020, and 1128021 are complementary to a portion of nucleobases 16828-16864 of SEQ ID NO: 2.

In certain embodiments, modified oligonucleotides complementary to a portion of nucleobases 16828-16864 of SEQ ID NO: 2 achieve at least 71% reduction of GYS1 RNA in the standard in vitro assay. In certain embodiments, modified oligonucleotides complementary to a portion of nucleobases 16828-16864 of SEQ ID NO: 2 achieve an average of 84.7% reduction of GYS1 RNA in the standard in vitro assay.

### 10. Nucleobases: 16915-16947 of SEQ ID NO: 2

In certain embodiments, nucleobases 16915-16947 of SEQ ID NO: 2 comprise a hotspot region. In certain embodiments, modified oligonucleotides are complementary to a portion of nucleobases 16915-16947 of SEQ ID NO: 2. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: sooosssssssssssooss, wherein each “s” represents a phosphorothioate internucleoside linkage and each “o” represents a phosphodiester internucleoside linkage.

The nucleobase sequences of SEQ ID NOs: 2376, 2453, 2530, 2607, 2683, 2759, 2836, 2913, 539, and 616 are complementary to a portion of nucleobases 16915-16947 of SEQ ID NO: 2.

The nucleobase sequences of Compound Nos.: 1128032, 1128033, 1128034, 1128035, 1128036, 1128037, 1128038, 1128039, 1128040, and 1128041 are complementary to a portion of nucleobases 16915-16947 of SEQ ID NO: 2.

In certain embodiments, modified oligonucleotides complementary to a portion of nucleobases 16915-16947 of SEQ ID NO: 2 achieve at least 67% reduction of GYS1 RNA in the standard in vitro assay. In certain embodiments, modified oligonucleotides complementary to a portion of nucleobases 16915-16947 of SEQ ID NO: 2 achieve an average of 82.9% reduction of GYS1 RNA in the standard in vitro assay.

### 11. Additional Hotspot Regions

In certain embodiments, the ranges described in the Table below comprise hotspot regions. Each hotspot region begins with the nucleobase of SEQ ID NO: 1 or 2 identified in the ‘Target SEQ ID NO” column. The target region starts at the nucleobase identified in the “Target Start Site” column and ends with the nucleobase identified in the “Target Stop Site” column. In certain embodiments, modified oligonucleotides are complementary within any of the hotspot regions 1-23, as defined in the table below. In certain embodiments, modified oligonucleotides are 20 nucleobases in length. In certain embodiments, modified oligonucleotides are gapmers. In certain embodiments, the gapmers are MOE gapmers. In certain embodiments, all of the internucleoside linkages of the modified oligonucleotides are phosphorothioate internucleoside linkages. In certain embodiments, the internucleoside linkages of the modified oligonucleotides are phosphorothioate internucleoside linkages and phosphodiester internucleoside linkages. In certain embodiments, the phosphodiester (“o”) and phosphorothioate (“s”) internucleoside linkages are arranged in the order from 5′ to 3′: sooosssssssssssooss, wherein each “s” represents a phosphorothioate internucleoside linkage and each “o” represents a phosphodiester internucleoside linkage.

The nucleobase sequence of compounds listed in the “Compound No. in range” column in the table below are complementary to SEQ ID NO: 1 or 2 within the specified hotspot region. The nucleobase sequence of the oligonucleotides listed in the “SEQ ID NO: in range” column in the table below are complementary to the target sequence, SEQ ID NO: 1 or 2, within the specified hotspot region.

In certain embodiments, modified oligonucleotides complementary to nucleobases within the hotspot region achieve at least “Min. % Red. in vitro” (minimum % reduction, relative to untreated control cells) of GYS1 RNA in vitro in the standard cell assay, as indicated in the table below. In certain embodiments, modified oligonucleotides complementary to nucleobases within the hotspot region achieve an average of “Avg. % Red. in vitro” (average % reduction, relative to untreated control cells) of GYS1 RNA in vitro in the standard cell assay, as indicated in the table below.

TABLE 1
GYS1 Hotspots

| | Target | Target | Target | Min. % | Avg. % | | |
|---|---|---|---|---|---|---|---|
| Hotspot | SEQ | Start | Stop | Red. in | Red. in | | SEQ ID NOs in |
| ID | ID NO | Site | Site | vitro | vitro | Compound No. in Range | Range |
| 1 | 1 | 244 | 271 | 46 | 63 | 941367, 1126323, | 102, 1406, 1479, |
| | | | | | | 1126324, 1126325, | 1556, 1633, |
| | | | | | | 1126326, 1126327, | 1710, 1787, |
| | | | | | | 1126328, 1126329, and | 1863, and 179 |
| | | | | | | 941368 | |
| 2 | 1 | 289 | 317 | 48 | 62 | 1311870, 941369, | 256, 1939, 2015, |
| | | | | | | 1126330, 648138, | 2092, 2169, 333, |
| | | | | | | 1311878, 1311840, | and 2246 |
| | | | | | | 1126331, 1311872, | |
| | | | | | | 1126332, 1311877, | |
| | | | | | | 1126333, 1311841, | |
| | | | | | | 941370, and 1126334 | |
| 3 | 1 | 391 | 434 | 46 | 60 | 1126347, 941378, | 792, 26, 869, |
| | | | | | | 1126348, 1126349, | 946, 1023, 104, |
| | | | | | | 1126350, 941379, and | and 1100 |
| | | | | | | 1126351 | |
| 4 | 1 | 1191 | 1230 | 53 | 66 | 941437, 1126480, 941438, | 344, 1181, 421, |
| | | | | | | 1126481, 941439, and | 1257, 37, and |
| | | | | | | 1126482 | 1334 |
| 5 | 1 | 2633 | 2686 | 50 | 76 | 1126775, 941553, | 1724, 56, 1877, |
| | | | | | | 1126776, 1126777, | 1953, 2029, |
| | | | | | | 1126778, 1126779, | 2106, 2183, |
| | | | | | | 1126780, 1126781, | 2260, 2337, |
| | | | | | | 1126782, 1126783, | 2414, 133, and |
| | | | | | | 1126784, 941554, and | 2491 |
| | | | | | | 1126785 | |
| 6 | 1 | 2727 | 2764 | 44 | 65 | 1126788, 1126789, | 2721, 2797, |
| | | | | | | 1126790, 941555, | 2874, 210, 500, |
| | | | | | | 1126791, 1126792, | 577, 654, 730, |
| | | | | | | 1126793, 1126794, | 806, 883, 960, |
| | | | | | | 1126795, 1126796, | 287, 364, 1037, |
| | | | | | | 1126797, 941556, 941557, | 1114, 1191, and |
| | | | | | | 1126798, 1126799, | 1267 |
| | | | | | | 1126800, and 1126801 | |
| 7 | 1 | 2809 | 2851 | 42 | 70 | 1126802, 1126803, | 1344, 1421, |
| | | | | | | 1126804, 1126805, | 1494, 1571, |
| | | | | | | 1126806, and 941558 | 1648, and 441 |
| 8 | 1 | 3277 | 3326 | 54 | 74 | 1126884, 1126885, | 2723, 2800, |
| | | | | | | 1126886, 1126887, | 2877, 503, 61, |
| | | | | | | 941583, 1126888, | 580, 657, 138, |
| | | | | | | 1126889, 941584, and | and 733 |
| | | | | | | 1126890 | |
| 9 | 2 | 3239 | 3298 | 55 | 78 | 1126993, 1126994, | 1273, 1350, |
| | | | | | | 1126995, 1126996, | 1426, 1500, |
| | | | | | | 1126997, 1311846, | 1577, 1654, |
| | | | | | | 1126998, 1126999, | 1731, 1807, 372, |
| | | | | | | 1127000, 941605, | and 1960 |
| | | | | | | 1127001, and 1127002 | |
| 10 | 2 | 3324 | 3389 | 16 | 64 | 1311874, 1127009, | 2498, 2575, |
| | | | | | | 1127010, 1127011, | 2652, 2727, |
| | | | | | | 1127012, 1127013, | 2804, 2881, 507, |
| | | | | | | 1127014, 1127015, | 584, 661, 737, |
| | | | | | | 1127016, 1127017, | 813, 890, 967, |
| | | | | | | 1127018, 1127019, | 1044, 1121, 65, |
| | | | | | | 1127020, 1127021, | 1198, 1274, |
| | | | | | | 1127022, 1127023, | 1351, 1427, |
| | | | | | | 941607, 1127024, | 1501, 1578, |
| | | | | | | 1127025, 1127026, | 1655, 1732, |
| | | | | | | 1127027, 1127028, | 1808, 1884, |
| | | | | | | 1127029, 1127030, | 1961, 2037, |
| | | | | | | 1127031, 1127032, | 2114, 2191, |
| | | | | | | 1127033, 1127034, | 2268, 2345, |
| | | | | | | 1127035, 1127036, | 2422, 2499, and |
| | | | | | | 1127037, 1127038, | 2576 |
| | | | | | | 1127039, 1127040, | |
| | | | | | | 1127041, and 1127042 | |
| 11 | 2 | 3576 | 3612 | 49 | 73 | 1127090, 941611, | 1353, 373, 1503, |
| | | | | | | 1127091, 1127092, | 1580, 1657, |
| | | | | | | 1127093, 1127094, | 1734, 1810, |
| | | | | | | 1127095, 1127096, | 1886, 1963, |
| | | | | | | 1127097, 1127098, | 2039, 2116, |
| | | | | | | 1127099, 1127100, | 2193, 2270, |
| | | | | | | 1127101, 1127102, | 2347, and 450 |
| | | | | | | 1127103, and 941612 | |
| 12 | 2 | 5310 | 5350 | 51 | 68 | 1127259, 1311848, | 2044, 2121, |
| | | | | | | 1127260, 1127261, | 2198, 2275, |
| | | | | | | 1127262, 1127263, | 2352, 2429, and |
| | | | | | | 1127264, and 1127265 | 2506 |
| 13 | 2 | 5706 | 5762 | 56 | 78 | 1127298, 941637, | 2584, 70, and |
| | | | | | | 1127299, 1311867, | 2736 |
| | | | | | | 1311839, and 1127300 | |
| 14 | 2 | 6189 | 6221 | 51 | 69 | 1127357, 1127358, | 2201, 2278, |
| | | | | | | 1127359, 1127360, | 2355, 2432, |
| | | | | | | 1127361, 1127362, | 2509, 2586, |
| | | | | | | 1127363, 1127364, | 2662, 2738, |
| | | | | | | 1127365, 941646, and | 2815, 302, and |
| | | | | | | 1127366 | 2892 |
| 15 | 2 | 6986 | 7034 | 56 | 71 | 1127383, 1311860, | 1743, 1819, |
| | | | | | | 1127384, 1311861, | 1895, 1972, |
| | | | | | | 1127385, 1311859, | 2048, 456, 2125, |
| | | | | | | 1127386, 1127387, | 2202, 2279, and |
| | | | | | | 941648, 1311863, | 2356 |
| | | | | | | 1127388, 1311862, | |
| | | | | | | 1127389, 1311864, | |
| | | | | | | 1127390, and 1127391 | |
| 16 | 2 | 8371 | 8400 | 53 | 83 | 1127425, 1127426, | 2511, 2588, |
| | | | | | | 1127427, 1311851, | 2664, 2740, |
| | | | | | | 1127428, 1311852, | 2817, 2894, 520, |
| | | | | | | 1127429, 1311853, | 597, 3021, 673, |
| | | | | | | 1127430, 1311855, | 3022, and 750 |
| | | | | | | 1127431, 1311854, | |
| | | | | | | 1127432, 1251625, | |
| | | | | | | 1127433, 1251627, | |
| | | | | | | 1251628, 1251626, and | |
| | | | | | | 1127434 | |
| 17 | 2 | 8514 | 8555 | 49 | 66 | 127444, 127445, 127446, | 1514, 1591, |
| | | | | | | 127447, 941655, 127448, | 1668, 1745, 73, |
| | | | | | | and 127449 | 1821, and 1897 |
| 18 | 2 | 8826 | 8857 | 56 | 78 | 1127461, 1127462, | 2818, 2895, 521, |
| | | | | | | 1127463, 1127464, | 598, 674, 751, |
| | | | | | | 1127465, 1127466, and | and 827 |
| | | | | | | 1127467 | |
| 19 | 2 | 12100 | 12138 | 50 | 69 | 1127747, 1127748, | 2674, 2750, |
| | | | | | | 1127749, and 1127750 | 2827, and 2904 |
| 20 | 2 | 12612 | 12659 | 51 | 74 | 1127815, 1270775, | 532, 2976, 2964, |
| | | | | | | 1270762, 1127816, | 609, 310, 685, |
| | | | | | | 1270764, 941694, | 2972, 2988, |
| | | | | | | 1127817, 1270771, | 2993, 2959, 762, |
| | | | | | | 1270789, 1270795, | 2968, 838, 915, |
| | | | | | | 1270757, 1127818, | 992, 1069, 2973, |
| | | | | | | 1270767, 1127819, | 2992, and 1146 |
| | | | | | | 1127820, 1127821, | |
| | | | | | | 1127822, 1270772, | |
| | | | | | | 1270794, and 1127823 | |
| 21 | 2 | 16856 | 16910 | 54 | 76 | 1128026, 941726, | 1915, 469, 2068, |
| | | | | | | 1128027, 1128028, | 2979, 2967, |
| | | | | | | 1270778, 1270766, | 2958, 2938, |
| | | | | | | 1270756, 648408, 648409, | 2939, 2940, |
| | | | | | | 648410, 1311837, | 2997, 2996, |
| | | | | | | 1318984, 1311836, | 2941, 2995, |
| | | | | | | 1318985, 648411, | 2974, 2970, |
| | | | | | | 1311869, 1311835, | 2980, 2971, |
| | | | | | | 1270773, 1270769, | 2961, 2984, |
| | | | | | | 1270779, 1270770, | 2957, and 2145 |
| | | | | | | 1270759, 1270785, | |
| | | | | | | 1270755, and 1128029 | |
| 22 | 2 | 19917 | 19980 | 59 | 72 | 1128275, 1128276, | 1390, 1464, |
| | | | | | | 1128277, 941753, | 1540, 243, 1617, |
| | | | | | | 1128278, and 1128279 | and 1694 |
| 23 | 2 | 24606 | 24665 | 41 | 61 | 1128483, 1128484, | 2621, 2697, |
| | | | | | | 1128485, 1128486, and | 2773, 2850, and |
| | | | | | | 1128487 | 2927 |

### NONLIMITING DISCLOSURE AND INCORPORATION BY REFERENCE

Each of the literature and patent publications listed herein is incorporated by reference in its entirety.

While certain compounds, compositions and methods described herein have been described with specificity in accordance with certain embodiments, the following examples serve only to illustrate the compounds described herein and are not intended to limit the same. Each of the references, GenBank accession numbers, and the like recited in the present application is incorporated herein by reference in its entirety.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in certain instances, arbitrary. For example, an oligonucleotide comprising a nucleoside comprising a 2′-OH sugar moiety and a thymine base could be described as a DNA having a modified sugar moiety (2′-OH in place of one 2′-H of DNA) or as an RNA having a modified base (thymine (methylated uracil) in place of a uracil of RNA). Accordingly, nucleic acid sequences provided herein, including, but not limited to those in the sequence listing, are intended to encompass nucleic acids containing any combination of natural or modified RNA and/or DNA, unless otherwise stated, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to, such compounds comprising RNA bases, such as those having sequence “AUCGAUCG” and those having some DNA bases and some RNA bases such as “AUCGATCG” and oligomeric compounds having other modified nucleobases, such as “AT^{m}CGAUCG,” wherein ^{m}C indicates a cytosine base comprising a methyl group at the 5-position. Finally, for clarity, unless otherwise indicated, the phrase “nucleobase sequence of SEQ ID NO: X”, refers only to the sequence of nucleobases in that SEQ ID NO: X, independent of any sugar or internucleoside linkage modifications also described in such SEQ ID.

While effort has been made to accurately describe compounds in the accompanying sequence listing, should there be any discrepancies between a description in this specification and in the accompanying sequence listing, the description in the specification and not in the sequence listing is the accurate description.

Certain compounds described herein (e.g., modified oligonucleotides) have one or more asymmetric center and thus give rise to enantiomers, diastereomers, and other stereoisomeric configurations that may be defined, in terms of absolute stereochemistry, as (R) or (S), as α or β such as for sugar anomers, or as (D) or (L), such as for amino acids, etc. Compounds provided herein that are drawn or described as having certain stereoisomeric configurations include only the indicated compounds. Compounds provided herein that are drawn or described with undefined stereochemistry include all such possible isomers, including their stereorandom and optically pure forms, unless specified otherwise. Likewise, all cis- and trans-isomers and tautomeric forms of the compounds herein are also included unless otherwise indicated. Oligomeric compounds described herein include chirally pure or enriched mixtures as well as racemic mixtures. For example, oligomeric compounds having a plurality of phosphorothioate internucleoside linkages include such compounds in which chirality of the phosphorothioate internucleoside linkages is controlled or is random. Unless otherwise indicated, compounds described herein are intended to include corresponding salt forms.

The compounds described herein include variations in which one or more atoms are replaced with a non-radioactive isotope or radioactive isotope of the indicated element. For example, compounds herein that comprise hydrogen atoms encompass all possible deuterium substitutions for each of the ¹H hydrogen atoms. Isotopic substitutions encompassed by the compounds herein include but are not limited to: ²H or ³H in place of ¹H, ¹³C or ¹⁴C in place of ¹²C, ¹⁵N in place of ¹⁴N, ¹⁷O or ¹⁸O in place of ¹⁶O, and ³³S, ³⁴S, ³⁵S, or ³⁶S in place of ³²S. In certain embodiments, non-radioactive isotopic substitutions may impart new properties on the oligomeric compound that are beneficial for use as a therapeutic or research tool. In certain embodiments, radioactive isotopic substitutions may make the compound suitable for research or diagnostic purposes such as imaging.

### EXAMPLES

The following examples illustrate certain embodiments of the present disclosure and are not limiting. Moreover, where specific embodiments are provided, the inventors have contemplated generic application of those specific embodiments.

### Example 1: Effect of 5-10-5 MOE Gapmer Modified Oligonucleotides on Human GYS1 RNA In Vitro, Single Dose

Modified oligonucleotides complementary to human GYS1 nucleic acid were designed and tested for their single dose effects on GYS1 RNA in vitro. The modified oligonucleotides were tested in a series of experiments that had the same culture conditions.

The modified oligonucleotides in the tables below are 5-10-5 MOE gapmers. The gapmers are 20 nucleosides in length, wherein the central gap segment consists of ten 2′-β-D-deoxynucleosides and the 5′ and 3′ wing segments each consists of five 2′-MOE modified nucleosides. The sugar motif for the gapmers is (from 5′ to 3′): eeeeeddddddddddeeeee; wherein ‘d’ represents a 2′-β-D-deoxyribosyl sugar moiety, and ‘e’ represents a 2′-O(CH₂)₂OCH₃ ribosyl sugar moiety. The internucleoside linkage motif for the gapmers is (from 5′ to 3′): sososssssssssssooss; wherein each ‘o’ represents a phosphodiester internucleoside linkage and each ‘s’ represents a phosphorothioate internucleoside linkage. Each cytosine residue is a 5-methylcytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the Tables below is 100% complementary to either SEQ ID NO: 1 (GENBANK Accession No. NM_002103.4), or SEQ ID NO: 2 (the complement of GENBANK Accession No. NC_000019.10 truncated from nucleotides 48965001 to 48996000) or both. ‘N/A’ indicates that the modified oligonucleotide is not 100% complementary to that particular target nucleic acid sequence.

Cultured A431 cells were treated with modified oligonucleotide at a concentration of 4,000 nM using free uptake at a density of 10,000 cells per well. After a treatment period of approximately 48 hours, total RNA was isolated from the cells and GYS1 RNA levels were measured by quantitative real-time RTPCR. GYS1 RNA levels were measured by Human GYS1 primer probe set RTS36346 (forward sequence CACTACTGTGTCCCAGATCAC, designated herein as SEQ ID NO: 11; reverse sequence CTGAGCATGGAGGTTCTGG, designated herein as SEQ ID NO: 12; probe sequence AAGAGGAAACCAGATATTGTGACCCCC, designated herein as SEQ ID NO: 13). GYS1 RNA levels were normalized to total RNA content, as measured by RIBOGREEN®. Results are presented as percent reduction of GYS1 RNA relative to the amount of GYS1 RNA in untreated control cells (% reduction). As used herein, a value of ‘0’ indicates that treatment with the modified oligonucleotide did not reduce GYS1 RNA levels. Each table represents results from an individual assay plate. The values marked with the symbol “†” indicate that the modified oligonucleotide is complementary to the amplicon region of the primer probe set.

TABLE 2
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 941342 | 2 | 21 | 2649 | 2668 | GAAACCTCGCAGCCGCCAGG | 2 | 20 |
| 941348 | 83 | 102 | 2730 | 2749 | GACCGCACCCCTGCCCCGAA | 64 | 21 |
| 941354 | 128 | 147 | 2775 | 2794 | GGTGCCCGACGGGAAGCTTG | 4 | 22 |
| 941360 | 176 | 195 | 2823 | 2842 | CGGAGGTGTCTAGGGAATGC | 47 | 23 |
| 941366 | 238 | 257 | 2885 | 2904 | TGCGGTTTAAAGGCATGGCT | 86 | 24 |
| 941372 | 320 | 339 | 2967 | 2986 | GCCACTTCGAAGAGCACTGC | 5 | 25 |
| 941378 | 409 | 428 | 4567 | 4586 | AGTAGTTGTCGCCCCATTCG | 73 | 26 |
| 941384 | 427 | 446 | 4585 | 4604 | TGTACGGCCCCACCAGGAAG | 26 | 27 |
| 941390 | 440 | 459 | 4598 | 4617 | ACGCCCTGCTCCGTGTACGG | 52 | 28 |
| 941396 | 553 | 572 | 8627 | 8646 | CTCCCTCGATCAGCCAGCGC | 25 | 29 |
| 941398 | 558 | 577 | 8632 | 8651 | AGGGCCTCCCTCGATCAGCC | 36 | 30 |
| 941403 | 645 | 664 | 8719 | 8738 | CACTCCGATGTTGCAGGTAT | 33 | 31 |
| 941409 | 807 | 826 | 10039 | 10058 | TCGCCGGGCACGACACAGGC | 0 | 32 |
| 941415 | 873 | 892 | 10105 | 10124 | GGCACCGGCACACAGGTAGC | 16 | 33 |
| 941421 | 973 | 992 | 10449 | 10468 | AGTGGGCTGCCGCCCTTTCC | 0 | 34 |
| 941427 | 1021 | 1040 | 10497 | 10516 | GTGCCTCGATGGCGGTGATC | 87† | 35 |
| 941433 | 1133 | 1152 | 13232 | 13251 | ATTCGAGCCTTGCTCTGAGC | 12† | 36 |
| 941439 | 1210 | 1229 | 13653 | 13672 | CGGCGATAAAGAAGTATAAG | 60 | 37 |
| 941445 | 1222 | 1241 | 13665 | 13684 | ACTCATAGCGGCCGGCGATA | 11 | 38 |
| 941451 | 1233 | 1252 | 13676 | 13695 | CTTGTTGGAGAACTCATAGC | 61 | 39 |
| 941457 | 1314 | 1333 | 14375 | 14394 | GGCAACCACTGTCTGCTCGC | 31 | 40 |
| 941463 | 1471 | 1490 | 17999 | 18018 | TCATGTCGGGAAGGCTCCCA | 25 | 41 |
| 941469 | 1620 | 1639 | 21338 | 21357 | GAAGAGGCCGATTCGGCGGA | 19 | 42 |
| 941475 | 1723 | 1742 | 21721 | 21740 | GACAGCCACGGACAAACTCC | 32 | 43 |
| 941481 | 1795 | 1814 | 24982 | 25001 | GGATTCCCATAACCGTGCAC | 0 | 44 |
| 941487 | 1890 | 1909 | 25295 | 25314 | CCGCCGGTCAAGAATGTAGA | 2 | 45 |
| 941493 | 1911 | 1930 | 25316 | 25335 | GGAATCATCCAGGCTGCGGA | 14 | 46 |
| 941499 | 1968 | 1987 | 25373 | 25392 | ACGCTGCCGCCGGCTCTGCT | 0 | 47 |
| 941505 | 2011 | 2030 | 25416 | 25435 | CCAGAAGGTCGGAGAGGCGC | 0 | 48 |
| 941511 | 2092 | 2111 | 26187 | 26206 | CGTAGGTGAAGTGCTCTGGA | 62 | 49 |
| 941517 | 2159 | 2178 | 26417 | 26436 | GGCGACGGTGGCACCGAGGC | 11 | 50 |
| 941523 | 2200 | 2219 | 26458 | 26477 | CGTCCTCACTCTGGTGCGGG | 61 | 51 |
| 941529 | 2290 | 2309 | 26548 | 26567 | CACGGATGTTGCGCCGGTCC | 22 | 52 |
| 941535 | 2346 | 2365 | 26604 | 26623 | GCGCTTGCTGCCGCTGGTGG | 44 | 53 |
| 941541 | 2364 | 2383 | 26622 | 26641 | GGCCGTGTCCACAGAGTTGC | 13 | 54 |
| 941547 | 2444 | 2463 | 26702 | 26721 | GGGCGGACTTAGTTACGCTC | 0 | 55 |
| 941553 | 2634 | 2653 | 26892 | 26911 | GGCACGGCTTTGTGGATTCT | 93 | 56 |
| 941559 | 2850 | 2869 | 27108 | 27127 | CCGCATGCCGGGCCTGAGCG | 9 | 57 |
| 941565 | 2898 | 2917 | 27156 | 27175 | GATCGCCCCATTCGCAGGGA | 56 | 58 |
| 941571 | 2922 | 2941 | 27180 | 27199 | TGAAAGTGCCCCGGCTCTGG | 30 | 59 |
| 941577 | 3053 | 3072 | 27311 | 27330 | GGAGCCGGATGGAGGGATCC | 4 | 60 |
| 941583 | 3285 | 3304 | 27543 | 27562 | AGAGCAGTTGGGAATAAGCC | 80 | 61 |
| 941589 | 3403 | 3422 | 27661 | 27680 | GGTTCTAGAACCCAGTGACC | 28 | 62 |
| 941595 | 3560 | 3579 | 27818 | 27837 | TCCGGACACACTCCAATCAC | 37 | 63 |
| 941601 | N/A | N/A | 3049 | 3068 | GTTCCGGGCCCCCATCCACT | 59 | 64 |
| 941607 | N/A | N/A | 3346 | 3365 | TGAAACCCCGGCAGCTTCCC | 44 | 65 |
| 941613 | N/A | N/A | 3616 | 3635 | TGACTAACTTAGGACTTCCC | 55 | 66 |
| 941619 | N/A | N/A | 4057 | 4076 | ACCCCCTCATGGCCAATCCA | 18 | 67 |
| 941625 | N/A | N/A | 4474 | 4493 | AGGCCCCGGCCGAGGTCCAT | 0 | 68 |
| 941631 | N/A | N/A | 5334 | 5353 | TCCCAGCACCGCTAGAAGGT | 9 | 69 |
| 941637 | N/A | N/A | 5731 | 5750 | TTTCTCCTTGGACAACAGCG | 96 | 70 |
| | | | 5765 | 5784 | | | |
| 941643 | N/A | N/A | 5902 | 5921 | CCCTTAGGGACACAGTCGGA | 63 | 71 |
| 941649 | N/A | N/A | 7221 | 7240 | CAAGGTATCACATCCCGGCT | 68 | 72 |
| 941655 | N/A | N/A | 8533 | 8552 | TCCGTGGTTCTCCCATTTGC | 72 | 73 |
| 941661 | N/A | N/A | 8814 | 8833 | GGTGGAATGTGTCAGACGGG | 47 | 74 |
| 941667 | N/A | N/A | 9673 | 9692 | GTTAGATTGGACAGACTGAA | 73 | 75 |
| 941673 | N/A | N/A | 10168 | 10187 | ACTCGCAGTCCCCCATCTGC | 18 | 76 |
| 941679 | N/A | N/A | 10772 | 10791 | GGCGAATATTTTAGACAGGG | 93 | 77 |
| 941685 | N/A | N/A | 12096 | 12115 | GCCTCAAATACAGGTCACCA | 51 | 78 |
| 941691 | N/A | N/A | 12472 | 12491 | CCTAGGATATATCCTCTACT | 18 | 79 |
| 941697 | N/A | N/A | 13133 | 13152 | GGTGAGGGTCCCATGTTTTA | 47 | 80 |
| 941703 | N/A | N/A | 13616 | 13635 | GTCCAGATGCCTAAAGAACC | 18 | 81 |
| 941709 | N/A | N/A | 14462 | 14481 | GGCTGCTAACCAAAGCTGTT | 25 | 82 |
| 941715 | N/A | N/A | 15654 | 15673 | CCGTCTACAGGATTTTCTAG | 97 | 83 |
| 941721 | N/A | N/A | 16731 | 16750 | CCACGGTCACTCCTCCACTT | 80 | 84 |
| 941727 | N/A | N/A | 16930 | 16949 | GAGGTTTGCGATGAGGACCC | 55 | 85 |
| 941733 | N/A | N/A | 17894 | 17913 | GGGCACTCACACCAGTAAGG | 15 | 86 |
| 941739 | N/A | N/A | 19197 | 19216 | GTGTCCGCCTCGCTCTGTTG | 21 | 87 |
| 941745 | N/A | N/A | 19427 | 19446 | ACCGCAGCTGCTGAGAGTTT | 8 | 88 |
| 941751 | N/A | N/A | 19769 | 19788 | GTTAGCCAAAAGCAGTTCAC | 30 | 89 |
| 941757 | N/A | N/A | 20673 | 20692 | AGCGATGCATCCGTTTTGGC | 50 | 90 |
| 941763 | N/A | N/A | 21770 | 21789 | CCCGGTGTGTAGCCCCAAGG | 21 | 91 |
| 941769 | N/A | N/A | 22144 | 22163 | GGCTAATGTGAACCAACAAG | 63 | 92 |
| 941775 | N/A | N/A | 22884 | 22903 | GGTGGTTCCCCCGCCGCTGT | 0 | 93 |
| 941781 | N/A | N/A | 24699 | 24718 | TATTGAGCATCCCAATATCC | 0 | 94 |
| 941787 | N/A | N/A | 24970 | 24989 | CCGTGCACTCAGCTGCGGGA | 0 | 95 |
| 941793 | N/A | N/A | 25566 | 25585 | ATCGGCCTGGTTCATTGCTT | 16 | 96 |
| 941799 | N/A | N/A | 25992 | 26011 | GACTCCACCCCTTATGTAGA | 5 | 97 |

TABLE 3
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 941343 | 6 | 25 | 2653 | 2672 | CAGTGAAACCTCGCAGCCGC | 4 | 98 |
| 941349 | 90 | 109 | 2737 | 2756 | ATTGCAAGACCGCACCCCTG | 35 | 99 |
| 941355 | 131 | 150 | 2778 | 2797 | GCTGGTGCCCGACGGGAAGC | 28 | 100 |
| 941361 | 179 | 198 | 2826 | 2845 | CCCCGGAGGTGTCTAGGGAA | 18 | 101 |
| 941367 | 244 | 263 | 2891 | 2910 | ACAAAGTGCGGTTTAAAGGC | 84 | 102 |
| 941373 | 323 | 342 | 2970 | 2989 | CAGGCCACTTCGAAGAGCAC | 9 | 103 |
| 941379 | 414 | 433 | 4572 | 4591 | CAGGAAGTAGTTGTCGCCCC | 56 | 104 |
| 941385 | 430 | 449 | 4588 | 4607 | CCGTGTACGGCCCCACCAGG | 25 | 105 |
| 941391 | 442 | 461 | 4600 | 4619 | TCACGCCCTGCTCCGTGTAC | 21 | 106 |
| 941397 | 554 | 573 | 8628 | 8647 | CCTCCCTCGATCAGCCAGCG | 51 | 107 |
| 941398 | 558 | 577 | 8632 | 8651 | AGGGCCTCCCTCGATCAGCC | 34 | 30 |
| 941404 | 651 | 670 | 8725 | 8744 | CCACGGCACTCCGATGTTGC | 46 | 108 |
| 941410 | 812 | 831 | 10044 | 10063 | GGCAGTCGCCGGGCACGACA | 14 | 109 |
| 941416 | 876 | 895 | 10108 | 10127 | CACGGCACCGGCACACAGGT | 43 | 110 |
| 941422 | 977 | 996 | 10453 | 10472 | GCGCAGTGGGCTGCCGCCCT | 0 | 111 |
| 941428 | 1024 | 1043 | 10500 | 10519 | GCTGTGCCTCGATGGCGGTG | 93† | 112 |
| 941434 | 1151 | 1170 | 13250 | 13269 | CCCCGCACAAACTCCTGGAT | 7 | 113 |
| 941440 | 1212 | 1231 | 13655 | 13674 | GCCGGCGATAAAGAAGTATA | 0 | 114 |
| 941446 | 1224 | 1243 | 13667 | 13686 | GAACTCATAGCGGCCGGCGA | 5 | 115 |
| 941452 | 1243 | 1262 | 13686 | 13705 | CGTCAGCACCCTTGTTGGAG | 71 | 116 |
| 941458 | 1336 | 1355 | 14397 | 14416 | TCCGCGCTGGCATGATGAAG | 29 | 117 |
| 941464 | 1476 | 1495 | 18004 | 18023 | CTTGTTCATGTCGGGAAGGC | 21 | 118 |
| 941470 | 1631 | 1650 | 21349 | 21368 | GCACTGCTATTGAAGAGGCC | 0 | 119 |
| 941476 | 1761 | 1780 | 21759 | 21778 | GCCCCAAGGCTCATAGTAGG | 6 | 120 |
| 941482 | 1822 | 1841 | 25009 | 25028 | AGCCGGAGAGATTGGTGGAG | 38 | 121 |
| 941488 | 1897 | 1916 | 25302 | 25321 | TGCGGAACCGCCGGTCAAGA | 6 | 122 |
| 941494 | 1917 | 1936 | 25322 | 25341 | CGAGCAGGAATCATCCAGGC | 0 | 123 |
| 941500 | 1972 | 1991 | 25377 | 25396 | TGATACGCTGCCGCCGGCTC | 0 | 124 |
| 941506 | 2018 | 2037 | 25423 | 25442 | TTCCAGTCCAGAAGGTCGGA | 0 | 125 |
| 941512 | 2131 | 2150 | 26389 | 26408 | GGTAGCGGTACCCCTGGGCC | 3 | 126 |
| 941518 | 2164 | 2183 | 26422 | 26441 | GCGAGGGCGACGGTGGCACC | 30 | 127 |
| 941524 | 2230 | 2249 | 26488 | 26507 | CCTCCAGCGGCCCGTTCCGG | 33 | 128 |
| 941530 | 2297 | 2316 | 26555 | 26574 | TCTGGTGCACGGATGTTGCG | 34 | 129 |
| 941536 | 2349 | 2368 | 26607 | 26626 | GTTGCGCTTGCTGCCGCTGG | 47 | 130 |
| 941542 | 2430 | 2449 | 26688 | 26707 | ACGCTCCTCGCCCAGGGAGC | 16 | 131 |
| 941548 | 2515 | 2534 | 26773 | 26792 | GGGTTTAGGAGCAGCACCCC | 0 | 132 |
| 941554 | 2666 | 2685 | 26924 | 26943 | CGCTGATTATGCATATTCTG | 82 | 133 |
| 941560 | 2856 | 2875 | 27114 | 27133 | GGTGGCCCGCATGCCGGGCC | 0 | 134 |
| 941566 | 2903 | 2922 | 27161 | 27180 | GACTTGATCGCCCCATTCGC | 63 | 135 |
| 941572 | 2925 | 2944 | 27183 | 27202 | CTCTGAAAGTGCCCCGGCTC | 43 | 136 |
| 941578 | 3061 | 3080 | 27319 | 27338 | GGATGACAGGAGCCGGATGG | 45 | 137 |
| 941584 | 3306 | 3325 | 27564 | 27583 | CCTAGTGGTTTCACAGTGGG | 66 | 138 |
| 941590 | 3423 | 3442 | 27681 | 27700 | AGCCTCGAGGTAAATGTGGG | 41 | 139 |
| 941596 | 3565 | 3584 | 27823 | 27842 | TCATCTCCGGACACACTCCA | 70 | 140 |
| 941602 | N/A | N/A | 3087 | 3106 | AGGGTTCCCCTAGTAGCCCC | 20 | 141 |
| 941608 | N/A | N/A | 3374 | 3393 | CTAGACCATGCTGTTAGGGT | 60 | 142 |
| 941614 | N/A | N/A | 3624 | 3643 | CCAGCCTCTGACTAACTTAG | 58 | 143 |
| 941620 | N/A | N/A | 4093 | 4112 | TAAATCGCAGCCAGGCTCCA | 31 | 144 |
| 941626 | N/A | N/A | 4741 | 4760 | GGCTCCCACCCCGATGGCAG | 19 | 145 |
| 941632 | N/A | N/A | 5408 | 5427 | CACCGCCCACACAATGTGCT | 22 | 146 |
| 941638 | N/A | N/A | 5744 | 5763 | GCCGCCTGATGGCTTTCTCC | 11 | 147 |
| | | | 5778 | 5797 | | | |
| 941644 | N/A | N/A | 5971 | 5990 | GGGCTATTCTTAGGCCCCCA | 4 | 148 |
| 941650 | N/A | N/A | 7382 | 7401 | GAGCCACCTAAAGCAGGCCC | 6 | 149 |
| 941656 | N/A | N/A | 8539 | 8558 | CACTCATCCGTGGTTCTCCC | 66 | 150 |
| 941662 | N/A | N/A | 8899 | 8918 | TAAAGGACCCTATGACTCCC | 42 | 151 |
| 941668 | N/A | N/A | 9682 | 9701 | CCTGGCAGGGTTAGATTGGA | 53 | 152 |
| 941674 | N/A | N/A | 10309 | 10328 | GGGTCCCAAGAGAAATTGGT | 15 | 153 |
| 941680 | N/A | N/A | 10773 | 10792 | TGGCGAATATTTTAGACAGG | 63 | 154 |
| 941686 | N/A | N/A | 12195 | 12214 | GACCAATCTACATTAGCCAA | 87 | 155 |
| 941692 | N/A | N/A | 12523 | 12542 | TTAGCCCACCCTGAGATGGA | 30 | 156 |
| 941698 | N/A | N/A | 13264 | 13283 | ACCCATAAAAATGGCCCCGC | 57 | 157 |
| 941704 | N/A | N/A | 13793 | 13812 | GGCTGTTCTCCCTAGTTATA | 31 | 158 |
| 941710 | N/A | N/A | 14467 | 14486 | GCGAGGGCTGCTAACCAAAG | 65 | 159 |
| 941716 | N/A | N/A | 15712 | 15731 | GTCCTGACAACCCCGTCTCA | 41 | 160 |
| 941722 | N/A | N/A | 16778 | 16797 | GGACCACTTAGGCACACAAG | 96 | 161 |
| 941728 | N/A | N/A | 17047 | 17066 | TGGAAGGCTTAGGACCCACG | 47 | 162 |
| 941734 | N/A | N/A | 17995 | 18014 | GTCGGGAAGGCTCCCACTGC | 5 | 163 |
| 941740 | N/A | N/A | 19204 | 19223 | TGTGTATGTGTCCGCCTCGC | 42 | 164 |
| 941746 | N/A | N/A | 19498 | 19517 | GTTAGGGATCTAAAGCAGCT | 47 | 165 |
| 941752 | N/A | N/A | 19841 | 19860 | AACTCCCAGCATCCCGATGG | 8 | 166 |
| 941758 | N/A | N/A | 21112 | 21131 | ACCAGGAGTTGTGGATTCCC | 59 | 167 |
| 941764 | N/A | N/A | 21846 | 21865 | GCCAGTGCCTCGCCCCAAGA | 0 | 168 |
| 941770 | N/A | N/A | 22427 | 22446 | GCACAGCACTGCCGGAGTGC | 5 | 169 |
| 941776 | N/A | N/A | 23980 | 23999 | CGAGCACACAACACTACGCA | 19 | 170 |
| 941782 | N/A | N/A | 24728 | 24747 | TCCATAGTTGGGAGATAAGG | 13 | 171 |
| 941788 | N/A | N/A | 25062 | 25081 | GCGCTGACCGTAAGCTGAGG | 39 | 172 |
| 941794 | N/A | N/A | 25579 | 25598 | AGCCACCATCGCGATCGGCC | 3 | 173 |
| 941800 | N/A | N/A | 26056 | 26075 | CTTGGCCACACCCTTTATGC | 32 | 174 |

TABLE 4
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 941344 | 12 | 31 | 2659 | 2678 | CCCCTGCAGTGAAACCTCGC | 10 | 175 |
| 941350 | 97 | 116 | 2744 | 2763 | GCTTCCTATTGCAAGACCGC | 18 | 176 |
| 941356 | 136 | 155 | 2783 | 2802 | GAGTAGCTGGTGCCCGACGG | 56 | 177 |
| 941362 | 182 | 201 | 2829 | 2848 | GGACCCCGGAGGTGTCTAGG | 0 | 178 |
| 941368 | 252 | 271 | 2899 | 2918 | GGACATGGACAAAGTGCGGT | 69 | 179 |
| 941374 | 329 | 348 | 2976 | 2995 | ACCTCCCAGGCCACTTCGAA | 31 | 180 |
| 941380 | 417 | 436 | 4575 | 4594 | CACCAGGAAGTAGTTGTCGC | 47 | 181 |
| 941386 | 432 | 451 | 4590 | 4609 | CTCCGTGTACGGCCCCACCA | 65 | 182 |
| 941392 | 444 | 463 | 4602 | 4621 | CCTCACGCCCTGCTCCGTGT | 32 | 183 |
| 941398 | 558 | 577 | 8632 | 8651 | AGGGCCTCCCTCGATCAGCC | 36 | 30 |
| 941399 | 561 | 580 | 8635 | 8654 | CAGAGGGCCTCCCTCGATCA | 9 | 184 |
| 941405 | 655 | 674 | 8729 | 8748 | CGTACCACGGCACTCCGATG | 72 | 185 |
| 941411 | 818 | 837 | 10050 | 10069 | GCTACAGGCAGTCGCCGGGC | 11 | 186 |
| 941417 | 879 | 898 | 10111 | 10130 | GTCCACGGCACCGGCACACA | 20 | 187 |
| 941423 | 980 | 999 | 10456 | 10475 | TGAGCGCAGTGGGCTGCCGC | 0 | 188 |
| 941429 | 1026 | 1045 | 10502 | 10521 | GTGCTGTGCCTCGATGGCGG | 84† | 189 |
| 941435 | 1155 | 1174 | 13254 | 13273 | ATGGCCCCGCACAAACTCCT | 19 | 190 |
| 941441 | 1215 | 1234 | 13658 | 13677 | GCGGCCGGCGATAAAGAAGT | 0 | 191 |
| 941447 | 1225 | 1244 | 13668 | 13687 | AGAACTCATAGCGGCCGGCG | 0 | 192 |
| 941453 | 1272 | 1291 | 13715 | 13734 | GTTGAGCCGAGCCAATGCCT | 42 | 193 |
| 941459 | 1341 | 1360 | 14402 | 14421 | GTTGGTCCGCGCTGGCATGA | 76 | 194 |
| 941465 | 1570 | 1589 | 21288 | 21307 | CCAGCATATTGTGGGTGCAC | 39 | 195 |
| 941471 | 1639 | 1658 | 21357 | 21376 | CCCTGTCGGCACTGCTATTG | 23 | 196 |
| 941477 | 1764 | 1783 | 21762 | 21781 | GTAGCCCCAAGGCTCATAGT | 21 | 197 |
| 941483 | 1837 | 1856 | 25024 | 25043 | CCATGAAGCAGCCGAAGCCG | 9 | 198 |
| 941489 | 1900 | 1919 | 25305 | 25324 | GGCTGCGGAACCGCCGGTCA | 0 | 199 |
| 941495 | 1921 | 1940 | 25326 | 25345 | GCTGCGAGCAGGAATCATCC | 0 | 200 |
| 941501 | 1980 | 1999 | 25385 | 25404 | CCGCTGGATGATACGCTGCC | 0 | 201 |
| 941507 | 2029 | 2048 | 25434 | 25453 | GGCCTAGGTATTTCCAGTCC | 27 | 202 |
| 941513 | 2137 | 2156 | 26395 | 26414 | GCCGTGGGTAGCGGTACCCC | 5 | 203 |
| 941519 | 2168 | 2187 | 26426 | 26445 | GACAGCGAGGGCGACGGTGG | 30 | 204 |
| 941525 | 2236 | 2255 | 26494 | 26513 | CGTCTTCCTCCAGCGGCCCG | 0 | 205 |
| 941531 | 2301 | 2320 | 26559 | 26578 | CCACTCTGGTGCACGGATGT | 55 | 206 |
| 941537 | 2351 | 2370 | 26609 | 26628 | GAGTTGCGCTTGCTGCCGCT | 35 | 207 |
| 941543 | 2433 | 2452 | 26691 | 26710 | GTTACGCTCCTCGCCCAGGG | 68 | 208 |
| 941549 | 2536 | 2555 | 26794 | 26813 | CCCAGTGCAGATCTGGAGCG | 14 | 209 |
| 941555 | 2730 | 2749 | 26988 | 27007 | CACCGCAGAGTAATGGCAGA | 78 | 210 |
| 941561 | 2859 | 2878 | 27117 | 27136 | CTTGGTGGCCCGCATGCCGG | 0 | 211 |
| 941567 | 2909 | 2928 | 27167 | 27186 | GCTCTGGACTTGATCGCCCC | 45 | 212 |
| 941573 | 2928 | 2947 | 27186 | 27205 | AAACTCTGAAAGTGCCCCGG | 90 | 213 |
| 941579 | 3097 | 3116 | 27355 | 27374 | GTGGTTCTACCACCTCTTGC | 5 | 214 |
| 941585 | 3317 | 3336 | 27575 | 27594 | AGGACCTAGAACCTAGTGGT | 59 | 215 |
| 941591 | 3432 | 3451 | 27690 | 27709 | GGATGGAAGAGCCTCGAGGT | 15 | 216 |
| 941597 | 3570 | 3589 | 27828 | 27847 | TCATCTCATCTCCGGACACA | 94 | 217 |
| 941603 | N/A | N/A | 3168 | 3187 | GTCAAACGAACCATCCCTCT | 46 | 218 |
| 941609 | N/A | N/A | 3434 | 3453 | ACCGCCGCTCAGGCCTCCTA | 1 | 219 |
| 941615 | N/A | N/A | 3742 | 3761 | GTGTGAGATTGGCTTGTTCC | 89 | 220 |
| 941621 | N/A | N/A | 4138 | 4157 | ACCCGGACTCAGGTTCCCGA | 43 | 221 |
| 941627 | N/A | N/A | 4801 | 4820 | GTGTCCAAGCCTGCCTCGCT | 61 | 222 |
| 941633 | N/A | N/A | 5460 | 5479 | TCCAAGCTTGGCTGTTAGTA | 70 | 223 |
| 941639 | N/A | N/A | 5745 | 5764 | TGCCGCCTGATGGCTTTCTC | 21 | 224 |
| | | | 5779 | 5798 | | | |
| 941645 | N/A | N/A | 6067 | 6086 | CGCTCCTCGCCGACCTGGCT | 24 | 225 |
| 941651 | N/A | N/A | 7663 | 7682 | AATTCCTTCCACGGGATGCT | 74 | 226 |
| 941657 | N/A | N/A | 8544 | 8563 | ATCTTCACTCATCCGTGGTT | 48 | 227 |
| 941663 | N/A | N/A | 8963 | 8982 | ATCGAGAGTAGAGCCTGGGC | 31 | 228 |
| 941669 | N/A | N/A | 9834 | 9853 | AGAGTGGGCAGCGGCCCACT | 20 | 229 |
| 941675 | N/A | N/A | 10356 | 10375 | GTCCGGTTAGAAGGATTGGG | 28 | 230 |
| 941681 | N/A | N/A | 10892 | 10911 | GGCCACCCAAATCATGCAAT | 14 | 231 |
| 941687 | N/A | N/A | 12258 | 12277 | GCCCAGTTCTGACCTAACCT | 26 | 232 |
| 941693 | N/A | N/A | 12577 | 12596 | GGGATCTGATCCACACTGGT | 58 | 233 |
| 941699 | N/A | N/A | 13302 | 13321 | CGCCCTCCTCTCTTAAGACC | 15 | 234 |
| 941705 | N/A | N/A | 14173 | 14192 | ACGGCCCCCCACCATACTCA | 26 | 235 |
| 941711 | N/A | N/A | 14471 | 14490 | CGGAGCGAGGGCTGCTAACC | 0 | 236 |
| 941717 | N/A | N/A | 15747 | 15766 | CGTCTCTGCCCAATAACACT | 76 | 237 |
| 941723 | N/A | N/A | 16851 | 16870 | GGACTTGTACTTGTCCTGCA | 32 | 238 |
| 941729 | N/A | N/A | 17161 | 17180 | TGCCGCGCCCAGCTACCCTT | 32 | 239 |
| 941735 | N/A | N/A | 18104 | 18123 | GGCTGCCAGGAACTGCTATC | 0 | 240 |
| 941741 | N/A | N/A | 19299 | 19318 | CCGTGTGCAGCAAGGCCCAA | 0 | 241 |
| 941747 | N/A | N/A | 19571 | 19590 | GTAGTTTTTTGGGCCCCTAG | 28 | 242 |
| 941753 | N/A | N/A | 19932 | 19951 | GGGTGTATAGTTCTTAGCAA | 66 | 243 |
| 941759 | N/A | N/A | 21187 | 21206 | ATGAGCCATGCGGACCCTGG | 35 | 244 |
| 941765 | N/A | N/A | 21914 | 21933 | CGCGCTGTAGCAGAACAGGT | 32 | 245 |
| 941771 | N/A | N/A | 22539 | 22558 | GCACAGTTTGCAGGACTTGT | 64 | 246 |
| 941777 | N/A | N/A | 24525 | 24544 | CCTGTGTACAGAGCATCTAG | 33 | 247 |
| 941783 | N/A | N/A | 24731 | 24750 | AAGTCCATAGTTGGGAGATA | 47 | 248 |
| 941789 | N/A | N/A | 25180 | 25199 | CGGCCCGAGAGCCCCCCCAT | 22 | 249 |
| 941795 | N/A | N/A | 25627 | 25646 | TGCGATCCTTCCTGTTAGGC | 57 | 250 |
| 941801 | N/A | N/A | 26144 | 26163 | GCGCGCAGACATATAGTACT | 0 | 251 |

TABLE 5
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 941345 | 32 | 51 | 2679 | 2698 | CGTCACTGAGCCCACTGGCG | 0 | 252 |
| 941351 | 104 | 123 | 2751 | 2770 | ACGCTCGGCTTCCTATTGCA | 24 | 253 |
| 941357 | 160 | 179 | 2807 | 2826 | ATGCACCAGGTAGGGTGCGG | 0 | 254 |
| 941363 | 187 | 206 | 2834 | 2853 | GGTAGGGACCCCGGAGGTGT | 17 | 255 |
| 941369 | 289 | 308 | 2936 | 2955 | CGAATTCATCCTCCCAGTCC | 48 | 256 |
| 941375 | 361 | 380 | 4519 | 4538 | GCACCGTGTAGATGCCACCC | 34 | 257 |
| 941381 | 419 | 438 | 4577 | 4596 | CCCACCAGGAAGTAGTTGTC | 41 | 258 |
| 941387 | 433 | 452 | 4591 | 4610 | GCTCCGTGTACGGCCCCACC | 28 | 259 |
| 941393 | 447 | 466 | 4605 | 4624 | GGTCCTCACGCCCTGCTCCG | 29 | 260 |
| 941398 | 558 | 577 | 8632 | 8651 | AGGGCCTCCCTCGATCAGCC | 38 | 30 |
| 941400 | 564 | 583 | 8638 | 8657 | CACCAGAGGGCCTCCCTCGA | 0 | 261 |
| 941406 | 773 | 792 | 10005 | 10024 | GCCAACCACTCATGGAAGTG | 59 | 262 |
| 941412 | 821 | 840 | 10053 | 10072 | GTTGCTACAGGCAGTCGCCG | 0 | 263 |
| 941418 | 887 | 906 | 10119 | 10138 | TTGTAGAAGTCCACGGCACC | 8 | 264 |
| 941424 | 1011 | 1030 | 10487 | 10506 | GGCGGTGATCTGGGACACAG | 97† | 265 |
| 941430 | 1028 | 1047 | 10504 | 10523 | AAGTGCTGTGCCTCGATGGC | 82† | 266 |
| 941436 | 1164 | 1183 | 13263 | 13282 | CCCATAAAAATGGCCCCGCA | 26 | 267 |
| 941442 | 1216 | 1235 | 13659 | 13678 | AGCGGCCGGCGATAAAGAAG | 0 | 268 |
| 941448 | 1226 | 1245 | 13669 | 13688 | GAGAACTCATAGCGGCCGGC | 0 | 269 |
| 941454 | 1279 | 1298 | 13722 | 13741 | GCAGATAGTTGAGCCGAGCC | 51 | 270 |
| 941460 | 1345 | 1364 | 14406 | 14425 | AATTGTTGGTCCGCGCTGGC | 36 | 271 |
| 941466 | 1575 | 1594 | 21293 | 21312 | GTCATCCAGCATATTGTGGG | 74 | 272 |
| 941472 | 1642 | 1661 | 21360 | 21379 | TCACCCTGTCGGCACTGCTA | 5 | 273 |
| 941478 | 1768 | 1787 | 21766 | 21785 | GTGTGTAGCCCCAAGGCTCA | 3 | 274 |
| 941484 | 1852 | 1871 | 25039 | 25058 | CTGCGATGTGTTCCTCCATG | 72 | 275 |
| 941490 | 1903 | 1922 | 25308 | 25327 | CCAGGCTGCGGAACCGCCGG | 44 | 276 |
| 941496 | 1925 | 1944 | 25330 | 25349 | GTGAGCTGCGAGCAGGAATC | 0 | 277 |
| 941502 | 1984 | 2003 | 25389 | 25408 | GGTTCCGCTGGATGATACGC | 11 | 278 |
| 941508 | 2052 | 2071 | 26147 | 26166 | GTGGCGCGCAGACATATAGT | 0 | 279 |
| 941514 | 2142 | 2161 | 26400 | 26419 | GGCTGGCCGTGGGTAGCGGT | 14 | 280 |
| 941520 | 2171 | 2190 | 26429 | 26448 | CGTGACAGCGAGGGCGACGG | 18 | 281 |
| 941526 | 2239 | 2258 | 26497 | 26516 | CGCCGTCTTCCTCCAGCGGC | 9 | 282 |
| 941532 | 2305 | 2324 | 26563 | 26582 | GCGGCCACTCTGGTGCACGG | 0 | 283 |
| 941538 | 2355 | 2374 | 26613 | 26632 | CACAGAGTTGCGCTTGCTGC | 36 | 284 |
| 941544 | 2437 | 2456 | 26695 | 26714 | CTTAGTTACGCTCCTCGCCC | 18 | 285 |
| 941550 | 2574 | 2593 | 26832 | 26851 | GTGTTTGGCGGACTGGGTGG | 9 | 286 |
| 941556 | 2738 | 2757 | 26996 | 27015 | TCTGGCACCACCGCAGAGTA | 48 | 287 |
| 941562 | 2865 | 2884 | 27123 | 27142 | CCAGAACTTGGTGGCCCGCA | 42 | 288 |
| 941568 | 2910 | 2929 | 27168 | 27187 | GGCTCTGGACTTGATCGCCC | 23 | 289 |
| 941574 | 2959 | 2978 | 27217 | 27236 | AATGGAGGACCATCTGCTCT | 71 | 290 |
| 941580 | 3105 | 3124 | 27363 | 27382 | GCAGCCAAGTGGTTCTACCA | 28 | 291 |
| 941586 | 3320 | 3339 | 27578 | 27597 | GCCAGGACCTAGAACCTAGT | 51 | 292 |
| 941592 | 3511 | 3530 | 27769 | 27788 | AACCAAGCGGTGCAGACACA | 59 | 293 |
| 941598 | N/A | N/A | 2989 | 3008 | ACCCTTGTTAGCCACCTCCC | 55 | 294 |
| 941604 | N/A | N/A | 3222 | 3241 | GCGCCCTCTTCCCTAGGACC | 31 | 295 |
| 941610 | N/A | N/A | 3492 | 3511 | TACAGTCTCCAGCCCGTCCC | 30 | 296 |
| 941616 | N/A | N/A | 3854 | 3873 | GGCCCTGGCGATACCCGGCT | 17 | 297 |
| 941622 | N/A | N/A | 4209 | 4228 | GGTGCCGGGACCCCTTGGTC | 0 | 298 |
| 941628 | N/A | N/A | 4875 | 4894 | CGCCCATGTCTGGGATCCAC | 0 | 299 |
| 941634 | N/A | N/A | 5529 | 5548 | CCCCAGTCTATTACTCTTGC | 72 | 300 |
| 941640 | N/A | N/A | 5746 | 5765 | GTGCCGCCTGATGGCTTTCT | 37 | 301 |
| 941646 | N/A | N/A | 6201 | 6220 | TCTAGCCACTGCCGTGTTTC | 66 | 302 |
| 941652 | N/A | N/A | 7667 | 7686 | TGCTAATTCCTTCCACGGGA | 77 | 303 |
| 941658 | N/A | N/A | 8607 | 8626 | CCGAAATACACCTGGGATGG | 52 | 304 |
| 941664 | N/A | N/A | 9044 | 9063 | CTCGGGCAAGCCCGCCAGCC | 0 | 305 |
| 941670 | N/A | N/A | 9885 | 9904 | CCAGTAGGGACAAGGACTCG | 28 | 306 |
| 941676 | N/A | N/A | 10364 | 10383 | GGACAGCAGTCCGGTTAGAA | 10 | 307 |
| 941682 | N/A | N/A | 11039 | 11058 | CGTAGGCTAACATAAAGTGT | 44 | 308 |
| 941688 | N/A | N/A | 12316 | 12335 | GGAGTTGTAGTTCTCACCTT | 48 | 309 |
| 941694 | N/A | N/A | 12618 | 12637 | GCACAAGTATAAGTCTCCTC | 97 | 310 |
| 941700 | N/A | N/A | 13390 | 13409 | CCGAATACCCAGGTGCCCCC | 2 | 311 |
| 941706 | N/A | N/A | 14265 | 14284 | GGGATCCTGCCATACCCATT | 3 | 312 |
| 941712 | N/A | N/A | 14473 | 14492 | GCCGGAGCGAGGGCTGCTAA | 2 | 313 |
| 941718 | N/A | N/A | 15801 | 15820 | CGAGTTTGCTGTTCCTCAGA | 95 | 314 |
| 941724 | N/A | N/A | 16852 | 16871 | AGGACTTGTACTTGTCCTGC | 82 | 315 |
| 941730 | N/A | N/A | 17215 | 17234 | GGCTGGTAATTGGCCACCTC | 29 | 316 |
| 941736 | N/A | N/A | 18200 | 18219 | GGACCTCAGAATAGATCCCT | 16 | 317 |
| 941742 | N/A | N/A | 19311 | 19330 | GTAGAGGTGGAGCCGTGTGC | 7 | 318 |
| 941748 | N/A | N/A | 19574 | 19593 | GTTGTAGTTTTTTGGGCCCC | 0 | 319 |
| 941754 | N/A | N/A | 19964 | 19983 | GCTAGTGTGATAGGAAGTCC | 30 | 320 |
| 941760 | N/A | N/A | 21535 | 21554 | GTGTTTGGCAGGACCAATGT | 34 | 321 |
| 941766 | N/A | N/A | 21918 | 21937 | AGCACGCGCTGTAGCAGAAC | 36 | 322 |
| 941772 | N/A | N/A | 22564 | 22583 | ACCGCGACTCCTAATTATCA | 77 | 323 |
| 941778 | N/A | N/A | 24582 | 24601 | GTACGGACTTAGTCTATTTT | 47 | 324 |
| 941784 | N/A | N/A | 24737 | 24756 | GCGGGAAAGTCCATAGTTGG | 18 | 325 |
| 941790 | N/A | N/A | 25437 | 25456 | CCCGGCCTAGGTATTTCCAG | 13 | 326 |
| 941796 | N/A | N/A | 25823 | 25842 | GTTACTCTAGGATGGAGTGC | 36 | 327 |
| 941802 | N/A | N/A | 26218 | 26237 | GGTCCACTCACCGCATCCGC | 10 | 328 |

TABLE 6
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 941346 | 62 | 81 | 2709 | 2728 | CGTTGGGACCTAGGCAGAAG | 71 | 329 |
| 941352 | 120 | 139 | 2767 | 2786 | ACGGGAAGCTTGCAAGACGC | 48 | 330 |
| 941358 | 166 | 185 | 2813 | 2832 | TAGGGAATGCACCAGGTAGG | 76 | 331 |
| 941364 | 195 | 214 | 2842 | 2861 | GATCTCCAGGTAGGGACCCC | 41 | 332 |
| 941370 | 297 | 316 | 2944 | 2963 | CTCCAGGTCGAATTCATCCT | 77 | 333 |
| 941376 | 366 | 385 | 4524 | 4543 | CTGCAGCACCGTGTAGATGC | 37 | 334 |
| 941382 | 423 | 442 | 4581 | 4600 | CGGCCCCACCAGGAAGTAGT | 3 | 335 |
| 941388 | 436 | 455 | 4594 | 4613 | CCTGCTCCGTGTACGGCCCC | 25 | 336 |
| 941394 | 457 | 476 | 4615 | 4634 | GTTCCACCTGGGTCCTCACG | 60 | 337 |
| 941398 | 558 | 577 | 8632 | 8651 | AGGGCCTCCCTCGATCAGCC | 40 | 30 |
| 941401 | 641 | 660 | 8715 | 8734 | CCGATGTTGCAGGTATCCCA | 56 | 338 |
| 941407 | 778 | 797 | 10010 | 10029 | CGCCTGCCAACCACTCATGG | 0 | 339 |
| 941413 | 824 | 843 | 10056 | 10075 | ATGGTTGCTACAGGCAGTCG | 13 | 340 |
| 941419 | 891 | 910 | 10123 | 10142 | GTTGTTGTAGAAGTCCACGG | 42 | 341 |
| 941425 | 1015 | 1034 | 10491 | 10510 | CGATGGCGGTGATCTGGGAC | 71† | 342 |
| 941431 | 1033 | 1052 | 10509 | 10528 | TGAGCAAGTGCTGTGCCTCG | 67† | 343 |
| 941437 | 1191 | 1210 | 13634 | 13653 | GGTCTTGTCCAAGTTGAAGT | 74 | 344 |
| 941443 | 1219 | 1238 | 13662 | 13681 | CATAGCGGCCGGCGATAAAG | 38 | 345 |
| 941449 | 1228 | 1247 | 13671 | 13690 | TGGAGAACTCATAGCGGCCG | 0 | 346 |
| 941455 | 1304 | 1323 | 14365 | 14384 | GTCTGCTCGCTGCCGTTCAC | 65 | 347 |
| 941461 | 1380 | 1399 | 14441 | 14460 | GCGCACAGCTTGGCCTTTGA | 69 | 348 |
| 941467 | 1608 | 1627 | 21326 | 21345 | TCGGCGGATGGTGGTCAGGA | 36 | 349 |
| 941473 | 1647 | 1666 | 21365 | 21384 | CACCTTCACCCTGTCGGCAC | 10 | 350 |
| 941479 | 1771 | 1790 | 21769 | 21788 | CCGGTGTGTAGCCCCAAGGC | 26 | 351 |
| 941485 | 1854 | 1873 | 25041 | 25060 | GTCTGCGATGTGTTCCTCCA | 79 | 352 |
| 941491 | 1905 | 1924 | 25310 | 25329 | ATCCAGGCTGCGGAACCGCC | 0 | 353 |
| 941497 | 1961 | 1980 | 25366 | 25385 | CGCCGGCTCTGCTGACAGAA | 30 | 354 |
| 941503 | 1990 | 2009 | 25395 | 25414 | CCGTGCGGTTCCGCTGGATG | 0 | 355 |
| 941509 | 2059 | 2078 | 26154 | 26173 | GCGCCATGTGGCGCGCAGAC | 0 | 356 |
| 941515 | 2146 | 2165 | 26404 | 26423 | CCGAGGCTGGCCGTGGGTAG | 30 | 357 |
| 941521 | 2189 | 2208 | 26447 | 26466 | TGGTGCGGGCTGGAGTGTCG | 18 | 358 |
| 941527 | 2277 | 2296 | 26535 | 26554 | CCGGTCCTTGGCGGCCTCCT | 0 | 359 |
| 941533 | 2309 | 2328 | 26567 | 26586 | CGGCGCGGCCACTCTGGTGC | 20 | 360 |
| 941539 | 2358 | 2377 | 26616 | 26635 | GTCCACAGAGTTGCGCTTGC | 56 | 361 |
| 941545 | 2440 | 2459 | 26698 | 26717 | GGACTTAGTTACGCTCCTCG | 50 | 362 |
| 941551 | 2577 | 2596 | 26835 | 26854 | GGAGTGTTTGGCGGACTGGG | 55 | 363 |
| 941557 | 2741 | 2760 | 26999 | 27018 | ACCTCTGGCACCACCGCAGA | 70 | 364 |
| 941563 | 2873 | 2892 | 27131 | 27150 | CGTGGTTTCCAGAACTTGGT | 91 | 365 |
| 941569 | 2914 | 2933 | 27172 | 27191 | CCCCGGCTCTGGACTTGATC | 28 | 366 |
| 941575 | 3002 | 3021 | 27260 | 27279 | GAGGGCTAGAACATCCCTCC | 8 | 367 |
| 941581 | 3222 | 3241 | 27480 | 27499 | CGGATTCCCTGGAGGGAGAT | 74 | 368 |
| 941587 | 3329 | 3348 | 27587 | 27606 | GATCTAGAAGCCAGGACCTA | 39 | 369 |
| 941593 | 3514 | 3533 | 27772 | 27791 | GCAAACCAAGCGGTGCAGAC | 60 | 370 |
| 941599 | N/A | N/A | 2993 | 3012 | GCTCACCCTTGTTAGCCACC | 0 | 371 |
| 941605 | N/A | N/A | 3278 | 3297 | ACCCAGGTATCTAGTCTCTC | 96 | 372 |
| 941611 | N/A | N/A | 3577 | 3596 | ACGCATGTGTCCCAGGCCAT | 89 | 373 |
| 941617 | N/A | N/A | 3950 | 3969 | GACTAGGGACACCAGCATGC | 77 | 374 |
| 941623 | N/A | N/A | 4370 | 4389 | GGGAGCCCATAGTTTGGAGT | 37 | 375 |
| 941629 | N/A | N/A | 4932 | 4951 | TTGGCCTCTTGGATCTCCGT | 75 | 376 |
| 941635 | N/A | N/A | 5599 | 5618 | CCCCGATATTCCATGCTTCT | 84 | 377 |
| 941641 | N/A | N/A | 5747 | 5766 | CGTGCCGCCTGATGGCTTTC | 22 | 378 |
| 941647 | N/A | N/A | 6270 | 6289 | ACCGCCCTCGGCCACGCCAC | 38 | 379 |
| 941653 | N/A | N/A | 8366 | 8385 | TTCCGTGCCCCCTGCTTACT | 86 | 380 |
| 941659 | N/A | N/A | 8610 | 8629 | CGCCCGAAATACACCTGGGA | 21 | 381 |
| 941665 | N/A | N/A | 9550 | 9569 | GCCATAGGAGCAGCCAGTTC | 38 | 382 |
| 941671 | N/A | N/A | 9927 | 9946 | GGCCACTGTCTCCACGAGTG | 25 | 383 |
| 941677 | N/A | N/A | 10576 | 10595 | GGCATAGACAGCTGCCTACC | 4 | 384 |
| 941683 | N/A | N/A | 12006 | 12025 | CTTAGGACTTAGGATGGTCC | 39 | 385 |
| 941689 | N/A | N/A | 12386 | 12405 | ACTACAACCCCCATCAGCGC | 35 | 386 |
| 941695 | N/A | N/A | 12654 | 12673 | GCCTTCTTGCCTAATATTAC | 89 | 387 |
| 941701 | N/A | N/A | 13497 | 13516 | TAATACAGAGGCATCACGGG | 49 | 388 |
| 941707 | N/A | N/A | 14361 | 14380 | GCTCGCTGCCGTTCACCTGC | 0 | 389 |
| 941713 | N/A | N/A | 14474 | 14493 | GGCCGGAGCGAGGGCTGCTA | 0 | 390 |
| 941719 | N/A | N/A | 15846 | 15865 | CCCCATGGGACTCCCCACCG | 49 | 391 |
| 941725 | N/A | N/A | 16855 | 16874 | ATTAGGACTTGTACTTGTCC | 39 | 392 |
| 941731 | N/A | N/A | 17784 | 17803 | GCCATTTACTGTTAGGCTTC | 39 | 393 |
| 941737 | N/A | N/A | 18262 | 18281 | CCCACCCTAAGTGCCTCGCC | 0 | 394 |
| 941743 | N/A | N/A | 19377 | 19396 | TTGCTCACCTCCTGACTGCG | 0 | 395 |
| 941749 | N/A | N/A | 19577 | 19596 | GGAGTTGTAGTTTTTTGGGC | 46 | 396 |
| 941755 | N/A | N/A | 20410 | 20429 | GACTGTGCCAATATGGCAGC | 41 | 397 |
| 941761 | N/A | N/A | 21618 | 21637 | GCCCAAGTGCCTTATTTAGC | 27 | 398 |
| 941767 | N/A | N/A | 21986 | 22005 | CATCCAGTTCACTAGTCCTC | 41 | 399 |
| 941773 | N/A | N/A | 22569 | 22588 | GGCTGACCGCGACTCCTAAT | 46 | 400 |
| 941779 | N/A | N/A | 24594 | 24613 | TGCCAGTATCTTGTACGGAC | 59 | 401 |
| 941785 | N/A | N/A | 24835 | 24854 | GTCGCTGAGCCCCCACAACC | 41 | 402 |
| 941791 | N/A | N/A | 25445 | 25464 | GGGTCCTACCCGGCCTAGGT | 0 | 403 |
| 941797 | N/A | N/A | 25899 | 25918 | TCTTTGGCCACACCCTTATG | 49 | 404 |
| 941803 | N/A | N/A | 26386 | 26405 | AGCGGTACCCCTGGGCCTGC | 22 | 405 |

TABLE 7
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 941347 | 80 | 99 | 2727 | 2746 | CGCACCCCTGCCCCGAAGCG | 0 | 406 |
| 941353 | 124 | 143 | 2771 | 2790 | CCCGACGGGAAGCTTGCAAG | 0 | 407 |
| 941359 | 169 | 188 | 2816 | 2835 | GTCTAGGGAATGCACCAGGT | 95 | 408 |
| 941365 | 231 | 250 | 2878 | 2897 | TAAAGGCATGGCTGGCGCAG | 42 | 409 |
| 941371 | 301 | 320 | 2948 | 2967 | CGTTCTCCAGGTCGAATTCA | 87 | 410 |
| 941377 | 369 | 388 | 4527 | 4546 | CGTCTGCAGCACCGTGTAGA | 42 | 411 |
| 941383 | 426 | 445 | 4584 | 4603 | GTACGGCCCCACCAGGAAGT | 32 | 412 |
| 941389 | 437 | 456 | 4595 | 4614 | CCCTGCTCCGTGTACGGCCC | 16 | 413 |
| 941395 | 507 | 526 | 4665 | 4684 | GTTCATGGAATCCAGTGTCC | 68 | 414 |
| 941398 | 558 | 577 | 8632 | 8651 | AGGGCCTCCCTCGATCAGCC | 40 | 30 |
| 941402 | 642 | 661 | 8716 | 8735 | TCCGATGTTGCAGGTATCCC | 58 | 415 |
| 941408 | 804 | 823 | 10036 | 10055 | CCGGGCACGACACAGGCAGA | 47 | 416 |
| 941414 | 843 | 862 | 10075 | 10094 | CGTGGCATGGGTGGTGAAGA | 41 | 417 |
| 941420 | 964 | 983 | 10440 | 10459 | CCGCCCTTTCCATGCAGTAT | 55 | 418 |
| 941426 | 1018 | 1037 | 10494 | 10513 | CCTCGATGGCGGTGATCTGG | 88† | 419 |
| 941432 | 1130 | 1149 | 13229 | 13248 | CGAGCCTTGCTCTGAGCATG | 73† | 420 |
| 941438 | 1197 | 1216 | 13640 | 13659 | GTATAAGGTCTTGTCCAAGT | 91 | 421 |
| 941444 | 1220 | 1239 | 13663 | 13682 | TCATAGCGGCCGGCGATAAA | 23 | 422 |
| 941450 | 1231 | 1250 | 13674 | 13693 | TGTTGGAGAACTCATAGCGG | 79 | 423 |
| 941456 | 1309 | 1328 | 14370 | 14389 | CCACTGTCTGCTCGCTGCCG | 66 | 424 |
| 941462 | 1410 | 1429 | 17843 | 17862 | CACCGTGTTGGCCGTGTCCC | 79 | 425 |
| 941468 | 1614 | 1633 | 21332 | 21351 | GCCGATTCGGCGGATGGTGG | 4 | 426 |
| 941474 | 1671 | 1690 | 21669 | 21688 | GGAGAGGAACTCCGGGTGGA | 45 | 427 |
| 941480 | 1791 | 1810 | 24978 | 24997 | TCCCATAACCGTGCACTCAG | 43 | 428 |
| 941486 | 1855 | 1874 | 25042 | 25061 | GGTCTGCGATGTGTTCCTCC | 49 | 429 |
| 941492 | 1906 | 1925 | 25311 | 25330 | CATCCAGGCTGCGGAACCGC | 0 | 430 |
| 941498 | 1964 | 1983 | 25369 | 25388 | TGCCGCCGGCTCTGCTGACA | 21 | 431 |
| 941504 | 1995 | 2014 | 25400 | 25419 | GCGCTCCGTGCGGTTCCGCT | 0 | 432 |
| 941510 | 2075 | 2094 | 26170 | 26189 | GGAAAGGCCTTGGACAGCGC | 32 | 433 |
| 941516 | 2152 | 2171 | 26410 | 26429 | GTGGCACCGAGGCTGGCCGT | 29 | 434 |
| 941522 | 2199 | 2218 | 26457 | 26476 | GTCCTCACTCTGGTGCGGGC | 17 | 435 |
| 941528 | 2280 | 2299 | 26538 | 26557 | GCGCCGGTCCTTGGCGGCCT | 21 | 436 |
| 941534 | 2312 | 2331 | 26570 | 26589 | GCTCGGCGCGGCCACTCTGG | 55 | 437 |
| 941540 | 2361 | 2380 | 26619 | 26638 | CGTGTCCACAGAGTTGCGCT | 0 | 438 |
| 941546 | 2443 | 2462 | 26701 | 26720 | GGCGGACTTAGTTACGCTCC | 0 | 439 |
| 941552 | 2581 | 2600 | 26839 | 26858 | GGGTGGAGTGTTTGGCGGAC | 63 | 440 |
| 941558 | 2832 | 2851 | 27090 | 27109 | CGTGGCCTGCTCTGTATGCT | 80 | 441 |
| 941564 | 2894 | 2913 | 27152 | 27171 | GCCCCATTCGCAGGGACACC | 65 | 442 |
| 941570 | 2917 | 2936 | 27175 | 27194 | GTGCCCCGGCTCTGGACTTG | 40 | 443 |
| 941576 | 3050 | 3069 | 27308 | 27327 | GCCGGATGGAGGGATCCTCC | 39 | 444 |
| 941582 | 3233 | 3252 | 27491 | 27510 | TCCTCTGCAGGCGGATTCCC | 31 | 445 |
| 941588 | 3344 | 3363 | 27602 | 27621 | CGTGGTAAGGTTCCAGATCT | 79 | 446 |
| 941594 | 3521 | 3540 | 27779 | 27798 | CTCAACTGCAAACCAAGCGG | 67 | 447 |
| 941600 | N/A | N/A | 2995 | 3014 | GTGCTCACCCTTGTTAGCCA | 37 | 448 |
| 941606 | N/A | N/A | 3288 | 3307 | CCTGTTCAGGACCCAGGTAT | 6 | 449 |
| 941612 | N/A | N/A | 3593 | 3612 | CTTCCCCTATTCCAGTACGC | 92 | 450 |
| 941618 | N/A | N/A | 4015 | 4034 | AAGCCTGCATGGTCATTCCC | 36 | 451 |
| 941624 | N/A | N/A | 4466 | 4485 | GCCGAGGTCCATAGTTCTGG | 51 | 452 |
| 941630 | N/A | N/A | 4982 | 5001 | GACCAGGCTGTCTTTCGATC | 97 | 453 |
| 941636 | N/A | N/A | 5600 | 5619 | TCCCCGATATTCCATGCTTC | 77 | 454 |
| 941642 | N/A | N/A | 5789 | 5808 | CCCTTAGCCCTGCCGCCTGA | 50 | 455 |
| 941648 | N/A | N/A | 7007 | 7026 | CTGTCACCTGCGACCTCTTT | 85 | 456 |
| 941654 | N/A | N/A | 8503 | 8522 | CTATAGACTTAAGCCTCAGC | 85 | 457 |
| 941660 | N/A | N/A | 8611 | 8630 | GCGCCCGAAATACACCTGGG | 2 | 458 |
| 941666 | N/A | N/A | 9601 | 9620 | GGGTACGCTGTCTGCACCCA | 57 | 459 |
| 941672 | N/A | N/A | 10161 | 10180 | GTCCCCCATCTGCCACGGTC | 29 | 460 |
| 941678 | N/A | N/A | 10639 | 10658 | CGTTTTCGGTTTATGCTATT | 80 | 461 |
| 941684 | N/A | N/A | 12014 | 12033 | GTCCCCAGCTTAGGACTTAG | 32 | 462 |
| 941690 | N/A | N/A | 12448 | 12467 | AGCTTCTTACAGGGACCCAA | 81 | 463 |
| 941696 | N/A | N/A | 13029 | 13048 | CGTTCCTATGAGGTCCCCCC | 50 | 464 |
| 941702 | N/A | N/A | 13575 | 13594 | AAGCCCTCACCCGCTAGCCC | 0 | 465 |
| 941708 | N/A | N/A | 14457 | 14476 | CTAACCAAAGCTGTTTGCGC | 27 | 466 |
| 941714 | N/A | N/A | 15444 | 15463 | CTCTGCTAATGAATGAACGG | 93 | 467 |
| 941720 | N/A | N/A | 16573 | 16592 | GCGCAATCTCGGCCACTTTC | 56 | 468 |
| 941726 | N/A | N/A | 16861 | 16880 | GCTATTATTAGGACTTGTAC | 95 | 469 |
| 941732 | N/A | N/A | 17841 | 17860 | CCGTGTTGGCCGTGTCCCTG | 69 | 470 |
| 941738 | N/A | N/A | 18862 | 18881 | GGCTAGTGTGGGATCTGGCT | 61 | 471 |
| 941744 | N/A | N/A | 19426 | 19445 | CCGCAGCTGCTGAGAGTTTG | 6 | 472 |
| 941750 | N/A | N/A | 19678 | 19697 | TACTGAAGTCCCTGGACCCC | 9 | 473 |
| 941756 | N/A | N/A | 20537 | 20556 | AGCCGTGTTAGCAACTCCCT | 47 | 474 |
| 941762 | N/A | N/A | 21657 | 21676 | CGGGTGGAAAATCACCTGGT | 13 | 475 |
| 941768 | N/A | N/A | 22082 | 22101 | GACTTGGCCACACTCGGCTG | 23 | 476 |
| 941774 | N/A | N/A | 22629 | 22648 | CAGACTAGTACTATCTATCA | 37 | 477 |
| 941780 | N/A | N/A | 24653 | 24672 | GGATCGGGCCAAGGTCCATC | 21 | 478 |
| 941786 | N/A | N/A | 24892 | 24911 | CGCCTCAACACCGCCCTCTA | 0 | 479 |
| 941792 | N/A | N/A | 25496 | 25515 | AAGACCCCTAGCCAGGAGCT | 25 | 480 |
| 941798 | N/A | N/A | 25942 | 25961 | GCCATACCCCTCAAACAGGG | 38 | 481 |
| 941804 | N/A | N/A | 27870 | 27889 | GTCTGACCACACCTGGGATT | 64 | 482 |

### Example 2: Effect of 5-10-5 MOE Gapmer Modified Oligonucleotides on Human GYS1 RNA In Vitro, Single Dose

Modified oligonucleotides complementary to human GYS1 nucleic acid were designed and tested for their single dose effects on GYS1 RNA in vitro. The modified oligonucleotides were tested in a series of experiments that had the same culture conditions.

The modified oligonucleotides in the tables below are 5-10-5 MOE gapmers. The gapmers are 20 nucleosides in length, wherein the central gap segment consists of ten 2′-β-D-deoxynucleosides and the 5′ and 3′ wing segments each consists of five 2′-MOE modified nucleosides. The sugar motif for the gapmers is (from 5′ to 3′): ceeeeeddddddddddeeeee; wherein ‘d’ represents a 2′-β-D-deoxyribosyl sugar moiety, and ‘e’ represents a 2′-O(CH₂)₂OCH₃ ribosyl sugar moiety. The internucleoside linkage motif for the gapmers is (from 5′ to 3′): sooosssssssssssooss; wherein each ‘o’ represents a phosphodiester internucleoside linkage and each ‘s’ represents a phosphorothioate internucleoside linkage. Each cytosine residue is a 5-methylcytosine.

“Start site” indicates the 5′-most nucleoside to which the modified oligonucleotide is complementary in the target nucleic acid sequence. “Stop site” indicates the 3′-most nucleoside to which the modified oligonucleotide is complementary in the target nucleic acid sequence. Each modified oligonucleotide listed in the Tables below is 100% complementary to SEQ ID NO: 1 (described herein above), or SEQ ID NO: 2 (described herein above). ‘N/A’ indicates that the modified oligonucleotide is not 100% complementary to that particular target nucleic acid sequence.

Cultured A431 cells were treated with modified oligonucleotide at a concentration of 4,000 nM using free uptake at a density of 10,000 cells per well. After a treatment period of approximately 48 hours, total RNA was isolated from the cells and GYS1 RNA levels were measured by quantitative real-time RTPCR. GYS1 RNA levels were measured by Human GYS1 primer probe set RTS36346 (described herein above). GYS1 RNA levels were normalized to total RNA content, as measured by RIBOGREEN®. Results are presented as percent reduction of GYS1 relative to the amount of GYS1 RNA in untreated control cells (% reduction). As used herein, a value of ‘0’ indicates that treatment with the modified oligonucleotide did not reduce GYS1 RNA levels. Each table represents results from an individual assay plate. The values marked with the symbol “†” indicate that the modified oligonucleotide is complementary to the amplicon region of the primer probe set.

TABLE 8
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126247 | 1 | 20 | 2648 | 2667 | AAACCTCGCAGCCGCCAGGA | 0 | 483 |
| 1126279 | 127 | 146 | 2774 | 2793 | GTGCCCGACGGGAAGCTTGC | 0 | 484 |
| 1126311 | 193 | 212 | 2840 | 2859 | TCTCCAGGTAGGGACCCCGG | 5 | 485 |
| 1126343 | 364 | 383 | 4522 | 4541 | GCAGCACCGTGTAGATGCCA | 0 | 486 |
| 1126375 | 640 | 659 | 8714 | 8733 | CGATGTTGCAGGTATCCCAG | 37 | 487 |
| 1126407 | 822 | 841 | 10054 | 10073 | GGTTGCTACAGGCAGTCGCC | 0 | 488 |
| 1126439 | 970 | 989 | 10446 | 10465 | GGGCTGCCGCCCTTTCCATG | 0 | 489 |
| 1126471 | 1150 | 1169 | 13249 | 13268 | CCCGCACAAACTCCTGGATT | 0 | 490 |
| 1126503 | 1284 | 1303 | 13727 | 13746 | TCTGAGCAGATAGTTGAGCC | 21 | 491 |
| 1126535 | 1434 | 1453 | 17867 | 17886 | AAGCTTCCTCCCGAACTTTT | 30 | 492 |
| 1126567 | 1611 | 1630 | 21329 | 21348 | GATTCGGCGGATGGTGGTCA | 13 | 493 |
| 1126599 | 1717 | 1736 | 21715 | 21734 | CACGGACAAACTCCTCATAG | 3 | 494 |
| 1126631 | 1901 | 1920 | 25306 | 25325 | AGGCTGCGGAACCGCCGGTC | 0 | 495 |
| 1126663 | 1987 | 2006 | 25392 | 25411 | TGCGGTTCCGCTGGATGATA | 0 | 496 |
| 1126695 | 2160 | 2179 | 26418 | 26437 | GGGCGACGGTGGCACCGAGG | 0 | 497 |
| 1126727 | 2295 | 2314 | 26553 | 26572 | TGGTGCACGGATGTTGCGCC | 0 | 498 |
| 1126759 | 2436 | 2455 | 26694 | 26713 | TTAGTTACGCTCCTCGCCCA | 0 | 499 |
| 1126791 | 2731 | 2750 | 26989 | 27008 | CCACCGCAGAGTAATGGCAG | 50 | 500 |
| 1126823 | 2893 | 2912 | 27151 | 27170 | CCCCATTCGCAGGGACACCA | 1 | 501 |
| 1126855 | 2964 | 2983 | 27222 | 27241 | GTTGAAATGGAGGACCATCT | 57 | 502 |
| 1126887 | 3284 | 3303 | 27542 | 27561 | GAGCAGTTGGGAATAAGCCA | 54 | 503 |
| 1126919 | 3516 | 3535 | 27774 | 27793 | CTGCAAACCAAGCGGTGCAG | 0 | 504 |
| 1126951 | N/A | N/A | 3060 | 3079 | TACAACTCAGAGTTCCGGGC | 37 | 505 |
| 1126983 | N/A | N/A | 3172 | 3191 | TTAAGTCAAACGAACCATCC | 0 | 506 |
| 1127015 | N/A | N/A | 3330 | 3349 | TCCCAGGTTTGCACGCAGAA | 90 | 507 |
| 1127047 | N/A | N/A | 3376 | 3395 | CCCTAGACCATGCTGTTAGG | 9 | 508 |
| 1127079 | N/A | N/A | 3537 | 3556 | ACTAATAGCTCAGGCCCTCA | 20 | 509 |
| 1127111 | N/A | N/A | 3622 | 3641 | AGCCTCTGACTAACTTAGGA | 47 | 510 |
| 1127143 | N/A | N/A | 4066 | 4085 | GTGAGGGAGACCCCCTCATG | 0 | 511 |
| 1127175 | N/A | N/A | 4204 | 4223 | CGGGACCCCTTGGTCTGAAG | 3 | 512 |
| 1127207 | N/A | N/A | 4731 | 4750 | CCGATGGCAGGCTGTCCACC | 6 | 513 |
| 1127239 | N/A | N/A | 4922 | 4941 | GGATCTCCGTCTCAGTCTGT | 70 | 514 |
| 1127271 | N/A | N/A | 5406 | 5425 | CCGCCCACACAATGTGCTAC | 11 | 515 |
| 1127303 | N/A | N/A | 5782 | 5801 | CCCTGCCGCCTGATGGCTTT | 8 | 516 |
| 1127335 | N/A | N/A | 5928 | 5947 | AGGAAGCGACTGGGCTGTTC | 80 | 517 |
| 1127367 | N/A | N/A | 6222 | 6241 | CGTGCCCCAGGGCTGCATTT | 0 | 518 |
| 1127399 | N/A | N/A | 7225 | 7244 | GGCACAAGGTATCACATCCC | 88 | 519 |
| 1127431 | N/A | N/A | 8377 | 8396 | GTTGCAATATTTTCCGTGCC | 86 | 520 |
| 1127463 | N/A | N/A | 8831 | 8850 | TTGTAGTTTCAGGTATGGGT | 85 | 521 |
| 1127495 | N/A | N/A | 8966 | 8985 | GGCATCGAGAGTAGAGCCTG | 0 | 522 |
| 1127527 | N/A | N/A | 9055 | 9074 | TCCTAGAATTCCTCGGGCAA | 32 | 523 |
| 1127559 | N/A | N/A | 9606 | 9625 | CTCAAGGGTACGCTGTCTGC | 45 | 524 |
| 1127591 | N/A | N/A | 9691 | 9710 | CATCTAAGTCCTGGCAGGGT | 22 | 525 |
| 1127623 | N/A | N/A | 9816 | 9835 | CTGCAGCAATCCCAACCGGA | 6 | 526 |
| 1127655 | N/A | N/A | 9908 | 9927 | GTTGGGAAAAGAATCGGCAA | 14 | 527 |
| 1127687 | N/A | N/A | 10182 | 10201 | GGTTTTCCTGGCATACTCGC | 39 | 528 |
| 1127719 | N/A | N/A | 11634 | 11653 | TACATCCACATGAGTCCAAT | 72 | 529 |
| 1127751 | N/A | N/A | 12120 | 12139 | GCCTCCAGAGTATATAGTCA | 38 | 530 |
| 1127783 | N/A | N/A | 12343 | 12362 | TAGAGCTAACCAGTCAAAGG | 56 | 531 |
| 1127815 | N/A | N/A | 12612 | 12631 | GTATAAGTCTCCTCAGCTAC | 57 | 532 |
| 1127847 | N/A | N/A | 13323 | 13342 | CCTAGATGGTTAGGCTCCCA | 53 | 533 |
| 1127879 | N/A | N/A | 13511 | 13530 | GAGGATGTCTTAGGTAATAC | 83 | 534 |
| 1127911 | N/A | N/A | 13787 | 13806 | TCTCCCTAGTTATAAACTGC | 31 | 535 |
| 1127943 | N/A | N/A | 14363 | 14382 | CTGCTCGCTGCCGTTCACCT | 31 | 536 |
| 1127954 | N/A | N/A | 15654 | 15673 | CCGTCTACAGGATTTTCTAG | 95 | 83 |
| 1127976 | N/A | N/A | 15713 | 15732 | TGTCCTGACAACCCCGTCTC | 14 | 537 |
| 1128008 | N/A | N/A | 16755 | 16774 | AGCTAGCAGTGTCACACAAC | 40 | 538 |
| 1128040 | N/A | N/A | 16927 | 16946 | GTTTGCGATGAGGACCCAAT | 93 | 539 |
| 1128072 | N/A | N/A | 17220 | 17239 | CCTCTGGCTGGTAATTGGCC | 14 | 540 |
| 1128104 | N/A | N/A | 18075 | 18094 | AGAGGTCCAATCCATACCTG | 18 | 541 |
| 1128136 | N/A | N/A | 18258 | 18277 | CCCTAAGTGCCTCGCCTCCA | 14 | 542 |
| 1128168 | N/A | N/A | 19347 | 19366 | GGCCCGATAAAACTACCACT | 25 | 543 |
| 1128200 | N/A | N/A | 19556 | 19575 | CCTAGACTCAATAGAATGAC | 20 | 544 |
| 1128232 | N/A | N/A | 19735 | 19754 | ACTTAGCTGTCTGGGTTTCC | 32 | 545 |
| 1128264 | N/A | N/A | 19843 | 19862 | ACAACTCCCAGCATCCCGAT | 17 | 546 |
| 1128296 | N/A | N/A | 21098 | 21117 | ATTCCCACTAGTACAACCTG | 19 | 547 |
| 1128328 | N/A | N/A | 21560 | 21579 | AAGCTTGTCTAGCATCACAC | 33 | 548 |
| 1128360 | N/A | N/A | 21810 | 21829 | AGGATGCCATGACCACGCTG | 37 | 549 |
| 1128392 | N/A | N/A | 21915 | 21934 | ACGCGCTGTAGCAGAACAGG | 27 | 550 |
| 1128424 | N/A | N/A | 22081 | 22100 | ACTTGGCCACACTCGGCTGT | 7 | 551 |
| 1128456 | N/A | N/A | 22881 | 22900 | GGTTCCCCCGCCGCTGTTCT | 2 | 552 |
| 1128488 | N/A | N/A | 24647 | 24666 | GGCCAAGGTCCATCATTCTA | 14 | 553 |
| 1128520 | N/A | N/A | 24745 | 24764 | TCCAAGATGCGGGAAAGTCC | 28 | 554 |
| 1128552 | N/A | N/A | 24925 | 24944 | TCCCTCATCGCCTACCGTCT | 5 | 555 |
| 1128584 | N/A | N/A | 25190 | 25209 | CCTCTCCCAGCGGCCCGAGA | 15 | 556 |
| 1128616 | N/A | N/A | 25564 | 25583 | CGGCCTGGTTCATTGCTTAA | 14 | 557 |
| 1128648 | N/A | N/A | 25944 | 25963 | GAGCCATACCCCTCAAACAG | 21 | 558 |
| 1128680 | N/A | N/A | 26247 | 26266 | CTTTAGCTCCTGGCTAAGCA | 3 | 559 |

TABLE 9
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126248 | 3 | 22 | 2650 | 2669 | TGAAACCTCGCAGCCGCCAG | 0 | 560 |
| 1126280 | 129 | 148 | 2776 | 2795 | TGGTGCCCGACGGGAAGCTT | 2 | 561 |
| 1126312 | 194 | 213 | 2841 | 2860 | ATCTCCAGGTAGGGACCCCG | 15 | 562 |
| 1126344 | 365 | 384 | 4523 | 4542 | TGCAGCACCGTGTAGATGCC | 0 | 563 |
| 1126376 | 643 | 662 | 8717 | 8736 | CTCCGATGTTGCAGGTATCC | 28 | 564 |
| 1126408 | 823 | 842 | 10055 | 10074 | TGGTTGCTACAGGCAGTCGC | 0 | 565 |
| 1126440 | 971 | 990 | 10447 | 10466 | TGGGCTGCCGCCCTTTCCAT | 0 | 566 |
| 1126472 | 1152 | 1171 | 13251 | 13270 | GCCCCGCACAAACTCCTGGA | 0 | 567 |
| 1126504 | 1285 | 1304 | 13728 | 13747 | CTCTGAGCAGATAGTTGAGC | 9 | 568 |
| 1126536 | 1435 | 1454 | 17868 | 17887 | AAAGCTTCCTCCCGAACTTT | 20 | 569 |
| 1126568 | 1612 | 1631 | 21330 | 21349 | CGATTCGGCGGATGGTGGTC | 21 | 570 |
| 1126600 | 1718 | 1737 | 21716 | 21735 | CCACGGACAAACTCCTCATA | 1 | 571 |
| 1126632 | 1902 | 1921 | 25307 | 25326 | CAGGCTGCGGAACCGCCGGT | 0 | 572 |
| 1126664 | 1988 | 2007 | 25393 | 25412 | GTGCGGTTCCGCTGGATGAT | 0 | 573 |
| 1126696 | 2161 | 2180 | 26419 | 26438 | AGGGCGACGGTGGCACCGAG | 0 | 574 |
| 1126728 | 2296 | 2315 | 26554 | 26573 | CTGGTGCACGGATGTTGCGC | 0 | 575 |
| 1126760 | 2438 | 2457 | 26696 | 26715 | ACTTAGTTACGCTCCTCGCC | 24 | 576 |
| 1126792 | 2732 | 2751 | 26990 | 27009 | ACCACCGCAGAGTAATGGCA | 64 | 577 |
| 1126824 | 2896 | 2915 | 27154 | 27173 | TCGCCCCATTCGCAGGGACA | 0 | 578 |
| 1126856 | 2965 | 2984 | 27223 | 27242 | AGTTGAAATGGAGGACCATC | 59 | 579 |
| 1126888 | 3304 | 3323 | 27562 | 27581 | TAGTGGTTTCACAGTGGGCA | 90 | 580 |
| 1126920 | 3517 | 3536 | 27775 | 27794 | ACTGCAAACCAAGCGGTGCA | 6 | 581 |
| 1126952 | N/A | N/A | 3061 | 3080 | CTACAACTCAGAGTTCCGGG | 40 | 582 |
| 1126984 | N/A | N/A | 3173 | 3192 | CTTAAGTCAAACGAACCATC | 30 | 583 |
| 1127016 | N/A | N/A | 3331 | 3350 | TTCCCAGGTTTGCACGCAGA | 87 | 584 |
| 1127048 | N/A | N/A | 3377 | 3396 | CCCCTAGACCATGCTGTTAG | 32 | 585 |
| 1127080 | N/A | N/A | 3538 | 3557 | AACTAATAGCTCAGGCCCTC | 35 | 586 |
| 1127112 | N/A | N/A | 3623 | 3642 | CAGCCTCTGACTAACTTAGG | 52 | 587 |
| 1127144 | N/A | N/A | 4067 | 4086 | AGTGAGGGAGACCCCCTCAT | 6 | 588 |
| 1127176 | N/A | N/A | 4205 | 4224 | CCGGGACCCCTTGGTCTGAA | 21 | 589 |
| 1127208 | N/A | N/A | 4732 | 4751 | CCCGATGGCAGGCTGTCCAC | 14 | 590 |
| 1127240 | N/A | N/A | 4923 | 4942 | TGGATCTCCGTCTCAGTCTG | 70 | 591 |
| 1127272 | N/A | N/A | 5407 | 5426 | ACCGCCCACACAATGTGCTA | 20 | 592 |
| 1127304 | N/A | N/A | 5785 | 5804 | TAGCCCTGCCGCCTGATGGC | 43 | 593 |
| 1127336 | N/A | N/A | 5929 | 5948 | CAGGAAGCGACTGGGCTGTT | 65 | 594 |
| 1127368 | N/A | N/A | 6232 | 6251 | ATCCTCTGTCCGTGCCCCAG | 56 | 595 |
| 1127400 | N/A | N/A | 7380 | 7399 | GCCACCTAAAGCAGGCCCTT | 34 | 596 |
| 1127432 | N/A | N/A | 8378 | 8397 | TGTTGCAATATTTTCCGTGC | 97 | 597 |
| 1127464 | N/A | N/A | 8832 | 8851 | ATTGTAGTTTCAGGTATGGG | 89 | 598 |
| 1127496 | N/A | N/A | 8967 | 8986 | TGGCATCGAGAGTAGAGCCT | 15 | 599 |
| 1127528 | N/A | N/A | 9056 | 9075 | CTCCTAGAATTCCTCGGGCA | 18 | 600 |
| 1127560 | N/A | N/A | 9607 | 9626 | TCTCAAGGGTACGCTGTCTG | 58 | 601 |
| 1127592 | N/A | N/A | 9694 | 9713 | AACCATCTAAGTCCTGGCAG | 48 | 602 |
| 1127624 | N/A | N/A | 9817 | 9836 | ACTGCAGCAATCCCAACCGG | 14 | 603 |
| 1127656 | N/A | N/A | 9909 | 9928 | TGTTGGGAAAAGAATCGGCA | 25 | 604 |
| 1127688 | N/A | N/A | 10183 | 10202 | GGGTTTTCCTGGCATACTCG | 29 | 605 |
| 1127720 | N/A | N/A | 11995 | 12014 | GGATGGTCCAAGTTATGATT | 85 | 606 |
| 1127752 | N/A | N/A | 12121 | 12140 | AGCCTCCAGAGTATATAGTC | 42 | 607 |
| 1127784 | N/A | N/A | 12344 | 12363 | ATAGAGCTAACCAGTCAAAG | 69 | 608 |
| 1127816 | N/A | N/A | 12616 | 12635 | ACAAGTATAAGTCTCCTCAG | 72 | 609 |
| 1127848 | N/A | N/A | 13324 | 13343 | ACCTAGATGGTTAGGCTCCC | 27 | 610 |
| 1127880 | N/A | N/A | 13513 | 13532 | CAGAGGATGTCTTAGGTAAT | 60 | 611 |
| 1127912 | N/A | N/A | 13789 | 13808 | GTTCTCCCTAGTTATAAACT | 38 | 612 |
| 1127944 | N/A | N/A | 14364 | 14383 | TCTGCTCGCTGCCGTTCACC | 42 | 613 |
| 1127954 | N/A | N/A | 15654 | 15673 | CCGTCTACAGGATTTTCTAG | 95 | 83 |
| 1127977 | N/A | N/A | 15716 | 15735 | GCCTGTCCTGACAACCCCGT | 37 | 614 |
| 1128009 | N/A | N/A | 16759 | 16778 | GTAAAGCTAGCAGTGTCACA | 83 | 615 |
| 1128041 | N/A | N/A | 16928 | 16947 | GGTTTGCGATGAGGACCCAA | 75 | 616 |
| 1128073 | N/A | N/A | 17221 | 17240 | ACCTCTGGCTGGTAATTGGC | 42 | 617 |
| 1128105 | N/A | N/A | 18078 | 18097 | CACAGAGGTCCAATCCATAC | 19 | 618 |
| 1128137 | N/A | N/A | 18259 | 18278 | ACCCTAAGTGCCTCGCCTCC | 18 | 619 |
| 1128169 | N/A | N/A | 19348 | 19367 | TGGCCCGATAAAACTACCAC | 8 | 620 |
| 1128201 | N/A | N/A | 19557 | 19576 | CCCTAGACTCAATAGAATGA | 11 | 621 |
| 1128233 | N/A | N/A | 19738 | 19757 | ACAACTTAGCTGTCTGGGTT | 21 | 622 |
| 1128265 | N/A | N/A | 19844 | 19863 | TACAACTCCCAGCATCCCGA | 17 | 623 |
| 1128297 | N/A | N/A | 21099 | 21118 | GATTCCCACTAGTACAACCT | 26 | 624 |
| 1128329 | N/A | N/A | 21566 | 21585 | GGTAAAAAGCTTGTCTAGCA | 39 | 625 |
| 1128361 | N/A | N/A | 21811 | 21830 | TAGGATGCCATGACCACGCT | 37 | 626 |
| 1128393 | N/A | N/A | 21916 | 21935 | CACGCGCTGTAGCAGAACAG | 20 | 627 |
| 1128425 | N/A | N/A | 22083 | 22102 | AGACTTGGCCACACTCGGCT | 0 | 628 |
| 1128457 | N/A | N/A | 22882 | 22901 | TGGTTCCCCCGCCGCTGTTC | 13 | 629 |
| 1128489 | N/A | N/A | 24649 | 24668 | CGGGCCAAGGTCCATCATTC | 12 | 630 |
| 1128521 | N/A | N/A | 24746 | 24765 | CTCCAAGATGCGGGAAAGTC | 31 | 631 |
| 1128553 | N/A | N/A | 24926 | 24945 | TTCCCTCATCGCCTACCGTC | 0 | 632 |
| 1128585 | N/A | N/A | 25235 | 25254 | GGGAGATCTTCATGGTCTCC | 12 | 633 |
| 1128617 | N/A | N/A | 25565 | 25584 | TCGGCCTGGTTCATTGCTTA | 19 | 634 |
| 1128649 | N/A | N/A | 25946 | 25965 | CTGAGCCATACCCCTCAAAC | 30 | 635 |
| 1128681 | N/A | N/A | 26248 | 26267 | CCTTTAGCTCCTGGCTAAGC | 0 | 636 |

TABLE 10
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126249 | 4 | 23 | 2651 | 2670 | GTGAAACCTCGCAGCCGCCA | 0 | 637 |
| 1126281 | 130 | 149 | 2777 | 2796 | CTGGTGCCCGACGGGAAGCT | 6 | 638 |
| 1126313 | 196 | 215 | 2843 | 2862 | GGATCTCCAGGTAGGGACCC | 0 | 639 |
| 1126345 | 367 | 386 | 4525 | 4544 | TCTGCAGCACCGTGTAGATG | 0 | 640 |
| 1126377 | 644 | 663 | 8718 | 8737 | ACTCCGATGTTGCAGGTATC | 34 | 641 |
| 1126409 | 825 | 844 | 10057 | 10076 | GATGGTTGCTACAGGCAGTC | 0 | 642 |
| 1126441 | 972 | 991 | 10448 | 10467 | GTGGGCTGCCGCCCTTTCCA | 0 | 643 |
| 1126473 | 1156 | 1175 | 13255 | 13274 | AATGGCCCCGCACAAACTCC | 0 | 644 |
| 1126505 | 1286 | 1305 | 13729 | 13748 | ACTCTGAGCAGATAGTTGAG | 0 | 645 |
| 1126537 | 1436 | 1455 | 17869 | 17888 | TAAAGCTTCCTCCCGAACTT | 4 | 646 |
| 1126569 | 1613 | 1632 | 21331 | 21350 | CCGATTCGGCGGATGGTGGT | 0 | 647 |
| 1126601 | 1719 | 1738 | 21717 | 21736 | GCCACGGACAAACTCCTCAT | 13 | 648 |
| 1126633 | 1904 | 1923 | 25309 | 25328 | TCCAGGCTGCGGAACCGCCG | 0 | 649 |
| 1126665 | 1989 | 2008 | 25394 | 25413 | CGTGCGGTTCCGCTGGATGA | 0 | 650 |
| 1126697 | 2162 | 2181 | 26420 | 26439 | GAGGGCGACGGTGGCACCGA | 0 | 651 |
| 1126729 | 2298 | 2317 | 26556 | 26575 | CTCTGGTGCACGGATGTTGC | 16 | 652 |
| 1126761 | 2439 | 2458 | 26697 | 26716 | GACTTAGTTACGCTCCTCGC | 32 | 653 |
| 1126793 | 2733 | 2752 | 26991 | 27010 | CACCACCGCAGAGTAATGGC | 51 | 654 |
| 1126825 | 2897 | 2916 | 27155 | 27174 | ATCGCCCCATTCGCAGGGAC | 0 | 655 |
| 1126857 | 2966 | 2985 | 27224 | 27243 | GAGTTGAAATGGAGGACCAT | 15 | 656 |
| 1126889 | 3305 | 3324 | 27563 | 27582 | CTAGTGGTTTCACAGTGGGC | 76 | 657 |
| 1126921 | 3518 | 3537 | 27776 | 27795 | AACTGCAAACCAAGCGGTGC | 0 | 658 |
| 1126953 | N/A | N/A | 3062 | 3081 | CCTACAACTCAGAGTTCCGG | 42 | 659 |
| 1126985 | N/A | N/A | 3174 | 3193 | TCTTAAGTCAAACGAACCAT | 17 | 660 |
| 1127017 | N/A | N/A | 3332 | 3351 | CTTCCCAGGTTTGCACGCAG | 87 | 661 |
| 1127049 | N/A | N/A | 3378 | 3397 | TCCCCTAGACCATGCTGTTA | 4 | 662 |
| 1127081 | N/A | N/A | 3539 | 3558 | GAACTAATAGCTCAGGCCCT | 38 | 663 |
| 1127113 | N/A | N/A | 3742 | 3761 | GTGTGAGATTGGCTTGTTCC | 92 | 220 |
| 1127145 | N/A | N/A | 4089 | 4108 | TCGCAGCCAGGCTCCAAAAC | 42 | 664 |
| 1127177 | N/A | N/A | 4206 | 4225 | GCCGGGACCCCTTGGTCTGA | 9 | 665 |
| 1127209 | N/A | N/A | 4736 | 4755 | CCACCCCGATGGCAGGCTGT | 0 | 666 |
| 1127241 | N/A | N/A | 4924 | 4943 | TTGGATCTCCGTCTCAGTCT | 79 | 667 |
| 1127273 | N/A | N/A | 5409 | 5428 | TCACCGCCCACACAATGTGC | 3 | 668 |
| 1127305 | N/A | N/A | 5786 | 5805 | TTAGCCCTGCCGCCTGATGG | 43 | 669 |
| 1127337 | N/A | N/A | 5930 | 5949 | TCAGGAAGCGACTGGGCTGT | 29 | 670 |
| 1127369 | N/A | N/A | 6238 | 6257 | TAGCCCATCCTCTGTCCGTG | 83 | 671 |
| 1127401 | N/A | N/A | 7537 | 7556 | CACCTCACCTCCTACAGGCG | 0 | 672 |
| 1127433 | N/A | N/A | 8379 | 8398 | CTGTTGCAATATTTTCCGTG | 95 | 673 |
| 1127465 | N/A | N/A | 8833 | 8852 | CATTGTAGTTTCAGGTATGG | 61 | 674 |
| 1127497 | N/A | N/A | 8968 | 8987 | TTGGCATCGAGAGTAGAGCC | 21 | 675 |
| 1127529 | N/A | N/A | 9057 | 9076 | ACTCCTAGAATTCCTCGGGC | 32 | 676 |
| 1127561 | N/A | N/A | 9608 | 9627 | GTCTCAAGGGTACGCTGTCT | 64 | 677 |
| 1127593 | N/A | N/A | 9695 | 9714 | CAACCATCTAAGTCCTGGCA | 38 | 678 |
| 1127625 | N/A | N/A | 9818 | 9837 | CACTGCAGCAATCCCAACCG | 12 | 679 |
| 1127657 | N/A | N/A | 9910 | 9929 | GTGTTGGGAAAAGAATCGGC | 38 | 680 |
| 1127689 | N/A | N/A | 10202 | 10221 | TTGGGCCCCCCAGAGCTTTG | 0 | 681 |
| 1127721 | N/A | N/A | 11996 | 12015 | AGGATGGTCCAAGTTATGAT | 84 | 682 |
| 1127753 | N/A | N/A | 12194 | 12213 | ACCAATCTACATTAGCCAAT | 60 | 683 |
| 1127785 | N/A | N/A | 12345 | 12364 | TATAGAGCTAACCAGTCAAA | 29 | 684 |
| 1127817 | N/A | N/A | 12619 | 12638 | AGCACAAGTATAAGTCTCCT | 88 | 685 |
| 1127849 | N/A | N/A | 13325 | 13344 | GACCTAGATGGTTAGGCTCC | 17 | 686 |
| 1127881 | N/A | N/A | 13514 | 13533 | GCAGAGGATGTCTTAGGTAA | 66 | 687 |
| 1127913 | N/A | N/A | 14175 | 14194 | TCACGGCCCCCCACCATACT | 0 | 688 |
| 1127945 | N/A | N/A | 14463 | 14482 | GGGCTGCTAACCAAAGCTGT | 0 | 689 |
| 1127954 | N/A | N/A | 15654 | 15673 | CCGTCTACAGGATTTTCTAG | 94 | 83 |
| 1127978 | N/A | N/A | 15717 | 15736 | GGCCTGTCCTGACAACCCCG | 15 | 690 |
| 1128010 | N/A | N/A | 16762 | 16781 | CAAGTAAAGCTAGCAGTGTC | 58 | 691 |
| 1128042 | N/A | N/A | 16929 | 16948 | AGGTTTGCGATGAGGACCCA | 40 | 692 |
| 1128074 | N/A | N/A | 17223 | 17242 | TGACCTCTGGCTGGTAATTG | 25 | 693 |
| 1128106 | N/A | N/A | 18080 | 18099 | TGCACAGAGGTCCAATCCAT | 21 | 694 |
| 1128138 | N/A | N/A | 18260 | 18279 | CACCCTAAGTGCCTCGCCTC | 3 | 695 |
| 1128170 | N/A | N/A | 19350 | 19369 | AGTGGCCCGATAAAACTACC | 4 | 696 |
| 1128202 | N/A | N/A | 19558 | 19577 | CCCCTAGACTCAATAGAATG | 0 | 697 |
| 1128234 | N/A | N/A | 19739 | 19758 | TACAACTTAGCTGTCTGGGT | 31 | 698 |
| 1128266 | N/A | N/A | 19854 | 19873 | CCCGAGAAACTACAACTCCC | 8 | 699 |
| 1128298 | N/A | N/A | 21100 | 21119 | GGATTCCCACTAGTACAACC | 35 | 700 |
| 1128330 | N/A | N/A | 21567 | 21586 | GGGTAAAAAGCTTGTCTAGC | 30 | 701 |
| 1128362 | N/A | N/A | 21812 | 21831 | CTAGGATGCCATGACCACGC | 31 | 702 |
| 1128394 | N/A | N/A | 21917 | 21936 | GCACGCGCTGTAGCAGAACA | 16 | 703 |
| 1128426 | N/A | N/A | 22084 | 22103 | GAGACTTGGCCACACTCGGC | 10 | 704 |
| 1128458 | N/A | N/A | 22883 | 22902 | GTGGTTCCCCCGCCGCTGTT | 0 | 705 |
| 1128490 | N/A | N/A | 24650 | 24669 | TCGGGCCAAGGTCCATCATT | 5 | 706 |
| 1128522 | N/A | N/A | 24747 | 24766 | GCTCCAAGATGCGGGAAAGT | 20 | 707 |
| 1128554 | N/A | N/A | 24927 | 24946 | CTTCCCTCATCGCCTACCGT | 7 | 708 |
| 1128586 | N/A | N/A | 25279 | 25298 | TAGATACCTGTGGAGGCCAG | 5 | 709 |
| 1128618 | N/A | N/A | 25567 | 25586 | GATCGGCCTGGTTCATTGCT | 17 | 710 |
| 1128650 | N/A | N/A | 25950 | 25969 | GTTTCTGAGCCATACCCCTC | 30 | 711 |
| 1128682 | N/A | N/A | 26249 | 26268 | CCCTTTAGCTCCTGGCTAAG | 7 | 712 |

TABLE 11
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126250 | 5 | 24 | 2652 | 2671 | AGTGAAACCTCGCAGCCGCC | 0 | 713 |
| 1126282 | 132 | 151 | 2779 | 2798 | AGCTGGTGCCCGACGGGAAG | 52 | 714 |
| 1126314 | 232 | 251 | 2879 | 2898 | TTAAAGGCATGGCTGGCGCA | 31 | 715 |
| 1126346 | 368 | 387 | 4526 | 4545 | GTCTGCAGCACCGTGTAGAT | 25 | 716 |
| 1126378 | 646 | 665 | 8720 | 8739 | GCACTCCGATGTTGCAGGTA | 33 | 717 |
| 1126410 | 826 | 845 | 10058 | 10077 | AGATGGTTGCTACAGGCAGT | 13 | 718 |
| 1126442 | 974 | 993 | 10450 | 10469 | CAGTGGGCTGCCGCCCTTTC | 0 | 719 |
| 1126474 | 1157 | 1176 | 13256 | 13275 | AAATGGCCCCGCACAAACTC | 18 | 720 |
| 1126506 | 1287 | 1306 | 13730 | 13749 | CACTCTGAGCAGATAGTTGA | 0 | 721 |
| 1126538 | 1437 | 1456 | 17870 | 17889 | ATAAAGCTTCCTCCCGAACT | 20 | 722 |
| 1126570 | 1615 | 1634 | 21333 | 21352 | GGCCGATTCGGCGGATGGTG | 12 | 723 |
| 1126602 | 1721 | 1740 | 21719 | 21738 | CAGCCACGGACAAACTCCTC | 8 | 724 |
| 1126634 | 1907 | 1926 | 25312 | 25331 | TCATCCAGGCTGCGGAACCG | 14 | 725 |
| 1126666 | 1991 | 2010 | 25396 | 25415 | TCCGTGCGGTTCCGCTGGAT | 0 | 726 |
| 1126698 | 2163 | 2182 | 26421 | 26440 | CGAGGGCGACGGTGGCACCG | 0 | 727 |
| 1126730 | 2299 | 2318 | 26557 | 26576 | ACTCTGGTGCACGGATGTTG | 26 | 728 |
| 1126762 | 2441 | 2460 | 26699 | 26718 | CGGACTTAGTTACGCTCCTC | 17 | 729 |
| 1126794 | 2734 | 2753 | 26992 | 27011 | GCACCACCGCAGAGTAATGG | 69 | 730 |
| 1126826 | 2899 | 2918 | 27157 | 27176 | TGATCGCCCCATTCGCAGGG | 18 | 731 |
| 1126858 | 3001 | 3020 | 27259 | 27278 | AGGGCTAGAACATCCCTCCC | 0 | 732 |
| 1126890 | 3307 | 3326 | 27565 | 27584 | ACCTAGTGGTTTCACAGTGG | 55 | 733 |
| 1126922 | 3519 | 3538 | 27777 | 27796 | CAACTGCAAACCAAGCGGTG | 9 | 734 |
| 1126954 | N/A | N/A | 3063 | 3082 | TCCTACAACTCAGAGTTCCG | 48 | 735 |
| 1126986 | N/A | N/A | 3176 | 3195 | CATCTTAAGTCAAACGAACC | 14 | 736 |
| 1127018 | N/A | N/A | 3333 | 3352 | GCTTCCCAGGTTTGCACGCA | 79 | 737 |
| 1127050 | N/A | N/A | 3379 | 3398 | ATCCCCTAGACCATGCTGTT | 46 | 738 |
| 1127082 | N/A | N/A | 3540 | 3559 | GGAACTAATAGCTCAGGCCC | 2 | 739 |
| 1127114 | N/A | N/A | 3743 | 3762 | GGTGTGAGATTGGCTTGTTC | 91 | 740 |
| 1127146 | N/A | N/A | 4090 | 4109 | ATCGCAGCCAGGCTCCAAAA | 72 | 741 |
| 1127178 | N/A | N/A | 4207 | 4226 | TGCCGGGACCCCTTGGTCTG | 30 | 742 |
| 1127210 | N/A | N/A | 4742 | 4761 | TGGCTCCCACCCCGATGGCA | 45 | 743 |
| 1127242 | N/A | N/A | 4925 | 4944 | CTTGGATCTCCGTCTCAGTC | 65 | 744 |
| 1127274 | N/A | N/A | 5410 | 5429 | ATCACCGCCCACACAATGTG | 21 | 745 |
| 1127306 | N/A | N/A | 5787 | 5806 | CTTAGCCCTGCCGCCTGATG | 36 | 746 |
| 1127338 | N/A | N/A | 5966 | 5985 | ATTCTTAGGCCCCCAGCACG | 15 | 747 |
| 1127370 | N/A | N/A | 6244 | 6263 | CTAACATAGCCCATCCTCTG | 21 | 748 |
| 1127402 | N/A | N/A | 7654 | 7673 | CACGGGATGCTTCTTTTTTG | 89 | 749 |
| 1127434 | N/A | N/A | 8381 | 8400 | CTCTGTTGCAATATTTTCCG | 87 | 750 |
| 1127466 | N/A | N/A | 8836 | 8855 | GGTCATTGTAGTTTCAGGTA | 97 | 751 |
| 1127498 | N/A | N/A | 8969 | 8988 | GTTGGCATCGAGAGTAGAGC | 89 | 752 |
| 1127530 | N/A | N/A | 9058 | 9077 | AACTCCTAGAATTCCTCGGG | 50 | 753 |
| 1127562 | N/A | N/A | 9609 | 9628 | TGTCTCAAGGGTACGCTGTC | 41 | 754 |
| 1127594 | N/A | N/A | 9696 | 9715 | CCAACCATCTAAGTCCTGGC | 67 | 755 |
| 1127626 | N/A | N/A | 9823 | 9842 | CGGCCCACTGCAGCAATCCC | 13 | 756 |
| 1127658 | N/A | N/A | 9911 | 9930 | AGTGTTGGGAAAAGAATCGG | 14 | 757 |
| 1127690 | N/A | N/A | 10203 | 10222 | CTTGGGCCCCCCAGAGCTTT | 10 | 758 |
| 1127722 | N/A | N/A | 11997 | 12016 | TAGGATGGTCCAAGTTATGA | 50 | 759 |
| 1127754 | N/A | N/A | 12196 | 12215 | AGACCAATCTACATTAGCCA | 66 | 760 |
| 1127786 | N/A | N/A | 12346 | 12365 | ATATAGAGCTAACCAGTCAA | 44 | 761 |
| 1127818 | N/A | N/A | 12627 | 12646 | GGTTCACAAGCACAAGTATA | 72 | 762 |
| 1127850 | N/A | N/A | 13326 | 13345 | AGACCTAGATGGTTAGGCTC | 23 | 763 |
| 1127882 | N/A | N/A | 13515 | 13534 | GGCAGAGGATGTCTTAGGTA | 86 | 764 |
| 1127914 | N/A | N/A | 14184 | 14203 | ATACTCCCCTCACGGCCCCC | 6 | 765 |
| 1127946 | N/A | N/A | 14465 | 14484 | GAGGGCTGCTAACCAAAGCT | 24 | 766 |
| 1127954 | N/A | N/A | 15654 | 15673 | CCGTCTACAGGATTTTCTAG | 93 | 83 |
| 1127979 | N/A | N/A | 15801 | 15820 | CGAGTTTGCTGTTCCTCAGA | 94 | 314 |
| 1128011 | N/A | N/A | 16763 | 16782 | ACAAGTAAAGCTAGCAGTGT | 61 | 767 |
| 1128043 | N/A | N/A | 16937 | 16956 | AACCTCAGAGGTTTGCGATG | 40 | 768 |
| 1128075 | N/A | N/A | 17774 | 17793 | GTTAGGCTTCTTTAGTTAGT | 70 | 769 |
| 1128107 | N/A | N/A | 18082 | 18101 | TCTGCACAGAGGTCCAATCC | 0 | 770 |
| 1128139 | N/A | N/A | 18261 | 18280 | CCACCCTAAGTGCCTCGCCT | 3 | 771 |
| 1128171 | N/A | N/A | 19351 | 19370 | AAGTGGCCCGATAAAACTAC | 4 | 772 |
| 1128203 | N/A | N/A | 19559 | 19578 | GCCCCTAGACTCAATAGAAT | 0 | 773 |
| 1128235 | N/A | N/A | 19744 | 19763 | GTTTCTACAACTTAGCTGTC | 49 | 774 |
| 1128267 | N/A | N/A | 19855 | 19874 | GCCCGAGAAACTACAACTCC | 20 | 775 |
| 1128299 | N/A | N/A | 21101 | 21120 | TGGATTCCCACTAGTACAAC | 40 | 776 |
| 1128331 | N/A | N/A | 21591 | 21610 | TGAGATCCCAAGGTGGCCCA | 0 | 777 |
| 1128363 | N/A | N/A | 21813 | 21832 | ACTAGGATGCCATGACCACG | 17 | 778 |
| 1128395 | N/A | N/A | 21919 | 21938 | AAGCACGCGCTGTAGCAGAA | 28 | 779 |
| 1128427 | N/A | N/A | 22085 | 22104 | GGAGACTTGGCCACACTCGG | 0 | 780 |
| 1128459 | N/A | N/A | 22885 | 22904 | GGGTGGTTCCCCCGCCGCTG | 9 | 781 |
| 1128491 | N/A | N/A | 24651 | 24670 | ATCGGGCCAAGGTCCATCAT | 0 | 782 |
| 1128523 | N/A | N/A | 24748 | 24767 | TGCTCCAAGATGCGGGAAAG | 16 | 783 |
| 1128555 | N/A | N/A | 24929 | 24948 | AGCTTCCCTCATCGCCTACC | 6 | 784 |
| 1128587 | N/A | N/A | 25280 | 25299 | GTAGATACCTGTGGAGGCCA | 16 | 785 |
| 1128619 | N/A | N/A | 25568 | 25587 | CGATCGGCCTGGTTCATTGC | 10 | 786 |
| 1128651 | N/A | N/A | 25951 | 25970 | AGTTTCTGAGCCATACCCCT | 26 | 787 |
| 1128683 | N/A | N/A | 26250 | 26269 | GCCCTTTAGCTCCTGGCTAA | 0 | 788 |

TABLE 12
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126251 | 7 | 26 | 2654 | 2673 | GCAGTGAAACCTCGCAGCCG | 0 | 789 |
| 1126283 | 133 | 152 | 2780 | 2799 | TAGCTGGTGCCCGACGGGAA | 48 | 790 |
| 1126315 | 233 | 252 | 2880 | 2899 | TTTAAAGGCATGGCTGGCGC | 33 | 791 |
| 1126347 | 391 | 410 | 4549 | 4568 | CGTCCCCTGTCACCTTCGCC | 62 | 792 |
| 1126379 | 647 | 666 | 8721 | 8740 | GGCACTCCGATGTTGCAGGT | 34 | 793 |
| 1126411 | 829 | 848 | 10061 | 10080 | TGAAGATGGTTGCTACAGGC | 42 | 794 |
| 1126443 | 975 | 994 | 10451 | 10470 | GCAGTGGGCTGCCGCCCTTT | 0 | 795 |
| 1126475 | 1158 | 1177 | 13257 | 13276 | AAAATGGCCCCGCACAAACT | 0 | 796 |
| 1126507 | 1288 | 1307 | 13731 | 13750 | TCACTCTGAGCAGATAGTTG | 31 | 797 |
| 1126539 | 1438 | 1457 | 17871 | 17890 | CATAAAGCTTCCTCCCGAAC | 6 | 798 |
| 1126571 | 1616 | 1635 | 21334 | 21353 | AGGCCGATTCGGCGGATGGT | 8 | 799 |
| 1126603 | 1722 | 1741 | 21720 | 21739 | ACAGCCACGGACAAACTCCT | 0 | 800 |
| 1126635 | 1908 | 1927 | 25313 | 25332 | ATCATCCAGGCTGCGGAACC | 0 | 801 |
| 1126667 | 1992 | 2011 | 25397 | 25416 | CTCCGTGCGGTTCCGCTGGA | 5 | 802 |
| 1126699 | 2165 | 2184 | 26423 | 26442 | AGCGAGGGCGACGGTGGCAC | 25 | 803 |
| 1126731 | 2300 | 2319 | 26558 | 26577 | CACTCTGGTGCACGGATGTT | 33 | 804 |
| 1126763 | 2442 | 2461 | 26700 | 26719 | GCGGACTTAGTTACGCTCCT | 1 | 805 |
| 1126795 | 2735 | 2754 | 26993 | 27012 | GGCACCACCGCAGAGTAATG | 77 | 806 |
| 1126827 | 2900 | 2919 | 27158 | 27177 | TTGATCGCCCCATTCGCAGG | 25 | 807 |
| 1126859 | 3003 | 3022 | 27261 | 27280 | GGAGGGCTAGAACATCCCTC | 5 | 808 |
| 1126891 | 3308 | 3327 | 27566 | 27585 | AACCTAGTGGTTTCACAGTG | 47 | 809 |
| 1126923 | 3520 | 3539 | 27778 | 27797 | TCAACTGCAAACCAAGCGGT | 14 | 810 |
| 1126955 | N/A | N/A | 3068 | 3087 | CGTCCTCCTACAACTCAGAG | 61 | 811 |
| 1126987 | N/A | N/A | 3177 | 3196 | TCATCTTAAGTCAAACGAAC | 31 | 812 |
| 1127019 | N/A | N/A | 3334 | 3353 | AGCTTCCCAGGTTTGCACGC | 84 | 813 |
| 1127051 | N/A | N/A | 3380 | 3399 | TATCCCCTAGACCATGCTGT | 30 | 814 |
| 1127083 | N/A | N/A | 3541 | 3560 | AGGAACTAATAGCTCAGGCC | 64 | 815 |
| 1127115 | N/A | N/A | 3749 | 3768 | CTTTGAGGTGTGAGATTGGC | 72 | 816 |
| 1127147 | N/A | N/A | 4092 | 4111 | AAATCGCAGCCAGGCTCCAA | 19 | 817 |
| 1127179 | N/A | N/A | 4208 | 4227 | GTGCCGGGACCCCTTGGTCT | 2 | 818 |
| 1127211 | N/A | N/A | 4758 | 4777 | GCACCCTCTCCGTCTGTGGC | 57 | 819 |
| 1127243 | N/A | N/A | 4927 | 4946 | CTCTTGGATCTCCGTCTCAG | 71 | 820 |
| 1127275 | N/A | N/A | 5411 | 5430 | AATCACCGCCCACACAATGT | 13 | 821 |
| 1127307 | N/A | N/A | 5788 | 5807 | CCTTAGCCCTGCCGCCTGAT | 44 | 822 |
| 1127339 | N/A | N/A | 5967 | 5986 | TATTCTTAGGCCCCCAGCAC | 14 | 823 |
| 1127371 | N/A | N/A | 6258 | 6277 | CACGCCACACATTTCTAACA | 17 | 824 |
| 1127403 | N/A | N/A | 7656 | 7675 | TCCACGGGATGCTTCTTTTT | 83 | 825 |
| 1127435 | N/A | N/A | 8440 | 8459 | GCTGCCTTAGCCTTACTTCT | 43 | 826 |
| 1127467 | N/A | N/A | 8838 | 8857 | ATGGTCATTGTAGTTTCAGG | 89 | 827 |
| 1127499 | N/A | N/A | 8970 | 8989 | AGTTGGCATCGAGAGTAGAG | 89 | 828 |
| 1127531 | N/A | N/A | 9059 | 9078 | CAACTCCTAGAATTCCTCGG | 39 | 829 |
| 1127563 | N/A | N/A | 9610 | 9629 | TTGTCTCAAGGGTACGCTGT | 40 | 830 |
| 1127595 | N/A | N/A | 9697 | 9716 | GCCAACCATCTAAGTCCTGG | 70 | 831 |
| 1127627 | N/A | N/A | 9824 | 9843 | GCGGCCCACTGCAGCAATCC | 1 | 832 |
| 1127659 | N/A | N/A | 9912 | 9931 | GAGTGTTGGGAAAAGAATCG | 12 | 833 |
| 1127691 | N/A | N/A | 10307 | 10326 | GTCCCAAGAGAAATTGGTGC | 17 | 834 |
| 1127723 | N/A | N/A | 11998 | 12017 | TTAGGATGGTCCAAGTTATG | 47 | 835 |
| 1127755 | N/A | N/A | 12197 | 12216 | AAGACCAATCTACATTAGCC | 52 | 836 |
| 1127787 | N/A | N/A | 12347 | 12366 | CATATAGAGCTAACCAGTCA | 34 | 837 |
| 1127819 | N/A | N/A | 12629 | 12648 | TAGGTTCACAAGCACAAGTA | 69 | 838 |
| 1127851 | N/A | N/A | 13327 | 13346 | CAGACCTAGATGGTTAGGCT | 37 | 839 |
| 1127883 | N/A | N/A | 13566 | 13585 | CCCGCTAGCCCTGGCCTCAA | 3 | 840 |
| 1127915 | N/A | N/A | 14185 | 14204 | TATACTCCCCTCACGGCCCC | 0 | 841 |
| 1127947 | N/A | N/A | 14466 | 14485 | CGAGGGCTGCTAACCAAAGC | 33 | 842 |
| 1127954 | N/A | N/A | 15654 | 15673 | CCGTCTACAGGATTTTCTAG | 94 | 83 |
| 1127980 | N/A | N/A | 15802 | 15821 | TCGAGTTTGCTGTTCCTCAG | 88 | 843 |
| 1128012 | N/A | N/A | 16764 | 16783 | CACAAGTAAAGCTAGCAGTG | 46 | 844 |
| 1128044 | N/A | N/A | 16938 | 16957 | CAACCTCAGAGGTTTGCGAT | 25 | 845 |
| 1128076 | N/A | N/A | 17775 | 17794 | TGTTAGGCTTCTTTAGTTAG | 49 | 846 |
| 1128108 | N/A | N/A | 18186 | 18205 | ATCCCTTCATAAAGGGCTGC | 0 | 847 |
| 1128140 | N/A | N/A | 18263 | 18282 | GCCCACCCTAAGTGCCTCGC | 0 | 848 |
| 1128172 | N/A | N/A | 19352 | 19371 | AAAGTGGCCCGATAAAACTA | 16 | 849 |
| 1128204 | N/A | N/A | 19560 | 19579 | GGCCCCTAGACTCAATAGAA | 0 | 850 |
| 1128236 | N/A | N/A | 19768 | 19787 | TTAGCCAAAAGCAGTTCACG | 31 | 851 |
| 1128268 | N/A | N/A | 19856 | 19875 | GGCCCGAGAAACTACAACTC | 0 | 852 |
| 1128300 | N/A | N/A | 21102 | 21121 | GTGGATTCCCACTAGTACAA | 6 | 853 |
| 1128332 | N/A | N/A | 21604 | 21623 | TTTAGCTAAGCCCTGAGATC | 0 | 854 |
| 1128364 | N/A | N/A | 21816 | 21835 | CTGACTAGGATGCCATGACC | 18 | 855 |
| 1128396 | N/A | N/A | 21920 | 21939 | AAAGCACGCGCTGTAGCAGA | 28 | 856 |
| 1128428 | N/A | N/A | 22086 | 22105 | AGGAGACTTGGCCACACTCG | 4 | 857 |
| 1128460 | N/A | N/A | 23979 | 23998 | GAGCACACAACACTACGCAT | 12 | 858 |
| 1128492 | N/A | N/A | 24652 | 24671 | GATCGGGCCAAGGTCCATCA | 8 | 859 |
| 1128524 | N/A | N/A | 24749 | 24768 | TTGCTCCAAGATGCGGGAAA | 28 | 860 |
| 1128556 | N/A | N/A | 24930 | 24949 | TAGCTTCCCTCATCGCCTAC | 0 | 861 |
| 1128588 | N/A | N/A | 25290 | 25309 | GGTCAAGAATGTAGATACCT | 33 | 862 |
| 1128620 | N/A | N/A | 25569 | 25588 | GCGATCGGCCTGGTTCATTG | 40 | 863 |
| 1128652 | N/A | N/A | 25952 | 25971 | CAGTTTCTGAGCCATACCCC | 25 | 864 |
| 1128684 | N/A | N/A | 26251 | 26270 | AGCCCTTTAGCTCCTGGCTA | 0 | 865 |

TABLE 13
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126252 | 8 | 27 | 2655 | 2674 | TGCAGTGAAACCTCGCAGCC | 0 | 866 |
| 1126284 | 134 | 153 | 2781 | 2800 | GTAGCTGGTGCCCGACGGGA | 55 | 867 |
| 1126316 | 234 | 253 | 2881 | 2900 | GTTTAAAGGCATGGCTGGCG | 44 | 868 |
| 1126348 | 411 | 430 | 4569 | 4588 | GAAGTAGTTGTCGCCCCATT | 62 | 869 |
| 1126380 | 648 | 667 | 8722 | 8741 | CGGCACTCCGATGTTGCAGG | 39 | 870 |
| 1126412 | 830 | 849 | 10062 | 10081 | GTGAAGATGGTTGCTACAGG | 53 | 871 |
| 1126444 | 976 | 995 | 10452 | 10471 | CGCAGTGGGCTGCCGCCCTT | 12 | 872 |
| 1126476 | 1160 | 1179 | 13259 | 13278 | TAAAAATGGCCCCGCACAAA | 10 | 873 |
| 1126508 | 1305 | 1324 | 14366 | 14385 | TGTCTGCTCGCTGCCGTTCA | 40 | 874 |
| 1126540 | 1439 | 1458 | 17872 | 17891 | TCATAAAGCTTCCTCCCGAA | 30 | 875 |
| 1126572 | 1617 | 1636 | 21335 | 21354 | GAGGCCGATTCGGCGGATGG | 21 | 876 |
| 1126604 | 1724 | 1743 | 21722 | 21741 | TGACAGCCACGGACAAACTC | 18 | 877 |
| 1126636 | 1909 | 1928 | 25314 | 25333 | AATCATCCAGGCTGCGGAAC | 0 | 878 |
| 1126668 | 1993 | 2012 | 25398 | 25417 | GCTCCGTGCGGTTCCGCTGG | 0 | 879 |
| 1126700 | 2166 | 2185 | 26424 | 26443 | CAGCGAGGGCGACGGTGGCA | 13 | 880 |
| 1126732 | 2302 | 2321 | 26560 | 26579 | GCCACTCTGGTGCACGGATG | 56 | 881 |
| 1126764 | 2511 | 2530 | 26769 | 26788 | TTAGGAGCAGCACCCCTCTG | 8 | 882 |
| 1126796 | 2736 | 2755 | 26994 | 27013 | TGGCACCACCGCAGAGTAAT | 67 | 883 |
| 1126828 | 2901 | 2920 | 27159 | 27178 | CTTGATCGCCCCATTCGCAG | 54 | 884 |
| 1126860 | 3048 | 3067 | 27306 | 27325 | CGGATGGAGGGATCCTCCAG | 6 | 885 |
| 1126892 | 3309 | 3328 | 27567 | 27586 | GAACCTAGTGGTTTCACAGT | 6 | 886 |
| 1126924 | 3558 | 3577 | 27816 | 27835 | CGGACACACTCCAATCACAC | 54 | 887 |
| 1126956 | N/A | N/A | 3069 | 3088 | CCGTCCTCCTACAACTCAGA | 61 | 888 |
| 1126988 | N/A | N/A | 3180 | 3199 | CTTTCATCTTAAGTCAAACG | 48 | 889 |
| 1127020 | N/A | N/A | 3335 | 3354 | CAGCTTCCCAGGTTTGCACG | 75 | 890 |
| 1127052 | N/A | N/A | 3381 | 3400 | TTATCCCCTAGACCATGCTG | 37 | 891 |
| 1127084 | N/A | N/A | 3542 | 3561 | TAGGAACTAATAGCTCAGGC | 90 | 892 |
| 1127116 | N/A | N/A | 3774 | 3793 | GCAAAGCAACCCCCCAGACC | 10 | 893 |
| 1127148 | N/A | N/A | 4094 | 4113 | TTAAATCGCAGCCAGGCTCC | 13 | 894 |
| 1127180 | N/A | N/A | 4308 | 4327 | GCAGTCCTCCTGGGTCTAAG | 60 | 895 |
| 1127212 | N/A | N/A | 4799 | 4818 | GTCCAAGCCTGCCTCGCTCT | 66 | 896 |
| 1127244 | N/A | N/A | 4930 | 4949 | GGCCTCTTGGATCTCCGTCT | 45 | 897 |
| 1127276 | N/A | N/A | 5412 | 5431 | CAATCACCGCCCACACAATG | 13 | 898 |
| 1127308 | N/A | N/A | 5791 | 5810 | GTCCCTTAGCCCTGCCGCCT | 48 | 899 |
| 1127340 | N/A | N/A | 5968 | 5987 | CTATTCTTAGGCCCCCAGCA | 25 | 900 |
| 1127372 | N/A | N/A | 6259 | 6278 | CCACGCCACACATTTCTAAC | 20 | 901 |
| 1127404 | N/A | N/A | 7658 | 7677 | CTTCCACGGGATGCTTCTTT | 63 | 902 |
| 1127436 | N/A | N/A | 8501 | 8520 | ATAGACTTAAGCCTCAGCTC | 56 | 903 |
| 1127468 | N/A | N/A | 8843 | 8862 | GGCTGATGGTCATTGTAGTT | 41 | 904 |
| 1127500 | N/A | N/A | 8971 | 8990 | AAGTTGGCATCGAGAGTAGA | 46 | 905 |
| 1127532 | N/A | N/A | 9060 | 9079 | ACAACTCCTAGAATTCCTCG | 33 | 906 |
| 1127564 | N/A | N/A | 9611 | 9630 | CTTGTCTCAAGGGTACGCTG | 53 | 907 |
| 1127596 | N/A | N/A | 9698 | 9717 | GGCCAACCATCTAAGTCCTG | 27 | 908 |
| 1127628 | N/A | N/A | 9825 | 9844 | AGCGGCCCACTGCAGCAATC | 3 | 909 |
| 1127660 | N/A | N/A | 9913 | 9932 | CGAGTGTTGGGAAAAGAATC | 21 | 910 |
| 1127692 | N/A | N/A | 10357 | 10376 | AGTCCGGTTAGAAGGATTGG | 0 | 911 |
| 1127724 | N/A | N/A | 12001 | 12020 | GACTTAGGATGGTCCAAGTT | 26 | 912 |
| 1127756 | N/A | N/A | 12198 | 12217 | TAAGACCAATCTACATTAGC | 0 | 913 |
| 1127788 | N/A | N/A | 12350 | 12369 | GAACATATAGAGCTAACCAG | 65 | 914 |
| 1127820 | N/A | N/A | 12630 | 12649 | TTAGGTTCACAAGCACAAGT | 74 | 915 |
| 1127852 | N/A | N/A | 13328 | 13347 | CCAGACCTAGATGGTTAGGC | 29 | 916 |
| 1127884 | N/A | N/A | 13574 | 13593 | AGCCCTCACCCGCTAGCCCT | 0 | 917 |
| 1127916 | N/A | N/A | 14186 | 14205 | CTATACTCCCCTCACGGCCC | 0 | 918 |
| 1127948 | N/A | N/A | 14468 | 14487 | AGCGAGGGCTGCTAACCAAA | 39 | 919 |
| 1127954 | N/A | N/A | 15654 | 15673 | CCGTCTACAGGATTTTCTAG | 96 | 83 |
| 1127981 | N/A | N/A | 15803 | 15822 | TTCGAGTTTGCTGTTCCTCA | 96 | 920 |
| 1128013 | N/A | N/A | 16767 | 16786 | GCACACAAGTAAAGCTAGCA | 62 | 921 |
| 1128045 | N/A | N/A | 16939 | 16958 | TCAACCTCAGAGGTTTGCGA | 50 | 922 |
| 1128077 | N/A | N/A | 17787 | 17806 | GCTGCCATTTACTGTTAGGC | 15 | 923 |
| 1128109 | N/A | N/A | 18187 | 18206 | GATCCCTTCATAAAGGGCTG | 0 | 924 |
| 1128141 | N/A | N/A | 18264 | 18283 | AGCCCACCCTAAGTGCCTCG | 1 | 925 |
| 1128173 | N/A | N/A | 19353 | 19372 | TAAAGTGGCCCGATAAAACT | 0 | 926 |
| 1128205 | N/A | N/A | 19561 | 19580 | GGGCCCCTAGACTCAATAGA | 0 | 927 |
| 1128237 | N/A | N/A | 19770 | 19789 | GGTTAGCCAAAAGCAGTTCA | 33 | 928 |
| 1128269 | N/A | N/A | 19857 | 19876 | GGGCCCGAGAAACTACAACT | 5 | 929 |
| 1128301 | N/A | N/A | 21103 | 21122 | TGTGGATTCCCACTAGTACA | 13 | 930 |
| 1128333 | N/A | N/A | 21605 | 21624 | ATTTAGCTAAGCCCTGAGAT | 3 | 931 |
| 1128365 | N/A | N/A | 21818 | 21837 | CTCTGACTAGGATGCCATGA | 28 | 932 |
| 1128397 | N/A | N/A | 21922 | 21941 | CAAAAGCACGCGCTGTAGCA | 24 | 933 |
| 1128429 | N/A | N/A | 22088 | 22107 | CGAGGAGACTTGGCCACACT | 34 | 934 |
| 1128461 | N/A | N/A | 24521 | 24540 | TGTACAGAGCATCTAGTACG | 0 | 935 |
| 1128493 | N/A | N/A | 24654 | 24673 | AGGATCGGGCCAAGGTCCAT | 8 | 936 |
| 1128525 | N/A | N/A | 24750 | 24769 | CTTGCTCCAAGATGCGGGAA | 21 | 937 |
| 1128557 | N/A | N/A | 24931 | 24950 | TTAGCTTCCCTCATCGCCTA | 0 | 938 |
| 1128589 | N/A | N/A | 25439 | 25458 | TACCCGGCCTAGGTATTTCC | 8 | 939 |
| 1128621 | N/A | N/A | 25570 | 25589 | CGCGATCGGCCTGGTTCATT | 36 | 940 |
| 1128653 | N/A | N/A | 25953 | 25972 | ACAGTTTCTGAGCCATACCC | 25 | 941 |
| 1128685 | N/A | N/A | 26341 | 26360 | GGTGAGCTGAGGACCTCACA | 0 | 942 |

TABLE 14
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126253 | 9 | 28 | 2656 | 2675 | CTGCAGTGAAACCTCGCAGC | 0 | 943 |
| 1126285 | 135 | 154 | 2782 | 2801 | AGTAGCTGGTGCCCGACGGG | 30 | 944 |
| 1126317 | 235 | 254 | 2882 | 2901 | GGTTTAAAGGCATGGCTGGC | 64 | 945 |
| 1126349 | 412 | 431 | 4570 | 4589 | GGAAGTAGTTGTCGCCCCAT | 46 | 946 |
| 1126381 | 649 | 668 | 8723 | 8742 | ACGGCACTCCGATGTTGCAG | 0 | 947 |
| 1126413 | 831 | 850 | 10063 | 10082 | GGTGAAGATGGTTGCTACAG | 75 | 948 |
| 1126445 | 978 | 997 | 10454 | 10473 | AGCGCAGTGGGCTGCCGCCC | 0 | 949 |
| 1126477 | 1161 | 1180 | 13260 | 13279 | ATAAAAATGGCCCCGCACAA | 0 | 950 |
| 1126509 | 1306 | 1325 | 14367 | 14386 | CTGTCTGCTCGCTGCCGTTC | 43 | 951 |
| 1126541 | 1440 | 1459 | 17873 | 17892 | TTCATAAAGCTTCCTCCCGA | 24 | 952 |
| 1126573 | 1618 | 1637 | 21336 | 21355 | AGAGGCCGATTCGGCGGATG | 9 | 953 |
| 1126605 | 1725 | 1744 | 21723 | 21742 | GTGACAGCCACGGACAAACT | 7 | 954 |
| 1126637 | 1910 | 1929 | 25315 | 25334 | GAATCATCCAGGCTGCGGAA | 18 | 955 |
| 1126669 | 1994 | 2013 | 25399 | 25418 | CGCTCCGTGCGGTTCCGCTG | 0 | 956 |
| 1126701 | 2167 | 2186 | 26425 | 26444 | ACAGCGAGGGCGACGGTGGC | 0 | 957 |
| 1126733 | 2303 | 2322 | 26561 | 26580 | GGCCACTCTGGTGCACGGAT | 41 | 958 |
| 1126765 | 2512 | 2531 | 26770 | 26789 | TTTAGGAGCAGCACCCCTCT | 0 | 959 |
| 1126797 | 2737 | 2756 | 26995 | 27014 | CTGGCACCACCGCAGAGTAA | 64 | 960 |
| 1126829 | 2902 | 2921 | 27160 | 27179 | ACTTGATCGCCCCATTCGCA | 55 | 961 |
| 1126861 | 3049 | 3068 | 27307 | 27326 | CCGGATGGAGGGATCCTCCA | 0 | 962 |
| 1126893 | 3314 | 3333 | 27572 | 27591 | ACCTAGAACCTAGTGGTTTC | 20 | 963 |
| 1126925 | 3559 | 3578 | 27817 | 27836 | CCGGACACACTCCAATCACA | 64 | 964 |
| 1126957 | N/A | N/A | 3073 | 3092 | AGCCCCGTCCTCCTACAACT | 20 | 965 |
| 1126989 | N/A | N/A | 3209 | 3228 | TAGGACCCAGAACCCGGGCT | 11 | 966 |
| 1127021 | N/A | N/A | 3339 | 3358 | CCGGCAGCTTCCCAGGTTTG | 52 | 967 |
| 1127053 | N/A | N/A | 3385 | 3404 | AGTTTTATCCCCTAGACCAT | 33 | 968 |
| 1127085 | N/A | N/A | 3543 | 3562 | CTAGGAACTAATAGCTCAGG | 78 | 969 |
| 1127117 | N/A | N/A | 3832 | 3851 | GTGTTCACGGGCCCCCTCTC | 24 | 970 |
| 1127149 | N/A | N/A | 4095 | 4114 | TTTAAATCGCAGCCAGGCTC | 19 | 971 |
| 1127181 | N/A | N/A | 4309 | 4328 | GGCAGTCCTCCTGGGTCTAA | 64 | 972 |
| 1127213 | N/A | N/A | 4802 | 4821 | TGTGTCCAAGCCTGCCTCGC | 13 | 973 |
| 1127245 | N/A | N/A | 4931 | 4950 | TGGCCTCTTGGATCTCCGTC | 38 | 974 |
| 1127277 | N/A | N/A | 5413 | 5432 | ACAATCACCGCCCACACAAT | 0 | 975 |
| 1127309 | N/A | N/A | 5884 | 5903 | GACCTGAATGGATTCCAGAC | 37 | 976 |
| 1127341 | N/A | N/A | 5969 | 5988 | GCTATTCTTAGGCCCCCAGC | 49 | 977 |
| 1127373 | N/A | N/A | 6260 | 6279 | GCCACGCCACACATTTCTAA | 56 | 978 |
| 1127405 | N/A | N/A | 7662 | 7681 | ATTCCTTCCACGGGATGCTT | 56 | 979 |
| 1127437 | N/A | N/A | 8502 | 8521 | TATAGACTTAAGCCTCAGCT | 52 | 980 |
| 1127469 | N/A | N/A | 8844 | 8863 | GGGCTGATGGTCATTGTAGT | 36 | 981 |
| 1127501 | N/A | N/A | 8972 | 8991 | GAAGTTGGCATCGAGAGTAG | 43 | 982 |
| 1127533 | N/A | N/A | 9078 | 9097 | ATATCACACTAGAGAACTAC | 0 | 983 |
| 1127565 | N/A | N/A | 9612 | 9631 | CCTTGTCTCAAGGGTACGCT | 39 | 984 |
| 1127597 | N/A | N/A | 9699 | 9718 | TGGCCAACCATCTAAGTCCT | 7 | 985 |
| 1127629 | N/A | N/A | 9826 | 9845 | CAGCGGCCCACTGCAGCAAT | 0 | 986 |
| 1127661 | N/A | N/A | 9916 | 9935 | CCACGAGTGTTGGGAAAAGA | 0 | 987 |
| 1127693 | N/A | N/A | 10358 | 10377 | CAGTCCGGTTAGAAGGATTG | 15 | 988 |
| 1127725 | N/A | N/A | 12002 | 12021 | GGACTTAGGATGGTCCAAGT | 8 | 989 |
| 1127757 | N/A | N/A | 12259 | 12278 | TGCCCAGTTCTGACCTAACC | 9 | 990 |
| 1127789 | N/A | N/A | 12351 | 12370 | GGAACATATAGAGCTAACCA | 90 | 991 |
| 1127821 | N/A | N/A | 12631 | 12650 | CTTAGGTTCACAAGCACAAG | 73 | 992 |
| 1127853 | N/A | N/A | 13329 | 13348 | CCCAGACCTAGATGGTTAGG | 0 | 993 |
| 1127885 | N/A | N/A | 13576 | 13595 | AAAGCCCTCACCCGCTAGCC | 3 | 994 |
| 1127917 | N/A | N/A | 14187 | 14206 | TCTATACTCCCCTCACGGCC | 9 | 995 |
| 1127949 | N/A | N/A | 14469 | 14488 | GAGCGAGGGCTGCTAACCAA | 25 | 996 |
| 1127954 | N/A | N/A | 15654 | 15673 | CCGTCTACAGGATTTTCTAG | 95 | 83 |
| 1127982 | N/A | N/A | 15804 | 15823 | CTTCGAGTTTGCTGTTCCTC | 94 | 997 |
| 1128014 | N/A | N/A | 16768 | 16787 | GGCACACAAGTAAAGCTAGC | 25 | 998 |
| 1128046 | N/A | N/A | 16940 | 16959 | ATCAACCTCAGAGGTTTGCG | 68 | 999 |
| 1128078 | N/A | N/A | 17836 | 17855 | TTGGCCGTGTCCCTGGAGGA | 18 | 1000 |
| 1128110 | N/A | N/A | 18188 | 18207 | AGATCCCTTCATAAAGGGCT | 0 | 1001 |
| 1128142 | N/A | N/A | 18267 | 18286 | AACAGCCCACCCTAAGTGCC | 0 | 1002 |
| 1128174 | N/A | N/A | 19354 | 19373 | CTAAAGTGGCCCGATAAAAC | 1 | 1003 |
| 1128206 | N/A | N/A | 19562 | 19581 | TGGGCCCCTAGACTCAATAG | 0 | 1004 |
| 1128238 | N/A | N/A | 19773 | 19792 | ATAGGTTAGCCAAAAGCAGT | 28 | 1005 |
| 1128270 | N/A | N/A | 19875 | 19894 | CCACTCTACTGAGTCTAGGG | 38 | 1006 |
| 1128302 | N/A | N/A | 21104 | 21123 | TTGTGGATTCCCACTAGTAC | 0 | 1007 |
| 1128334 | N/A | N/A | 21609 | 21628 | CCTTATTTAGCTAAGCCCTG | 0 | 1008 |
| 1128366 | N/A | N/A | 21819 | 21838 | GCTCTGACTAGGATGCCATG | 9 | 1009 |
| 1128398 | N/A | N/A | 21923 | 21942 | ACAAAAGCACGCGCTGTAGC | 17 | 1010 |
| 1128430 | N/A | N/A | 22089 | 22108 | ACGAGGAGACTTGGCCACAC | 29 | 1011 |
| 1128462 | N/A | N/A | 24524 | 24543 | CTGTGTACAGAGCATCTAGT | 15 | 1012 |
| 1128494 | N/A | N/A | 24655 | 24674 | TAGGATCGGGCCAAGGTCCA | 0 | 1013 |
| 1128526 | N/A | N/A | 24751 | 24770 | TCTTGCTCCAAGATGCGGGA | 20 | 1014 |
| 1128558 | N/A | N/A | 24933 | 24952 | ACTTAGCTTCCCTCATCGCC | 0 | 1015 |
| 1128590 | N/A | N/A | 25440 | 25459 | CTACCCGGCCTAGGTATTTC | 2 | 1016 |
| 1128622 | N/A | N/A | 25571 | 25590 | TCGCGATCGGCCTGGTTCAT | 18 | 1017 |
| 1128654 | N/A | N/A | 25954 | 25973 | GACAGTTTCTGAGCCATACC | 47 | 1018 |
| 1128686 | N/A | N/A | 26342 | 26361 | GGGTGAGCTGAGGACCTCAC | 7 | 1019 |

TABLE 15
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126254 | 10 | 29 | 2657 | 2676 | CCTGCAGTGAAACCTCGCAG | 8 | 1020 |
| 1126286 | 137 | 156 | 2784 | 2803 | CGAGTAGCTGGTGCCCGACG | 50 | 1021 |
| 1126318 | 236 | 255 | 2883 | 2902 | CGGTTTAAAGGCATGGCTGG | 41 | 1022 |
| 1126350 | 413 | 432 | 4571 | 4590 | AGGAAGTAGTTGTCGCCCCA | 56 | 1023 |
| 1126382 | 650 | 669 | 8724 | 8743 | CACGGCACTCCGATGTTGCA | 28 | 1024 |
| 1126414 | 833 | 852 | 10065 | 10084 | GTGGTGAAGATGGTTGCTAC | 0 | 1025 |
| 1126446 | 979 | 998 | 10455 | 10474 | GAGCGCAGTGGGCTGCCGCC | 3 | 1026 |
| 1126478 | 1163 | 1182 | 13262 | 13281 | CCATAAAAATGGCCCCGCAC | 0 | 1027 |
| 1126510 | 1307 | 1326 | 14368 | 14387 | ACTGTCTGCTCGCTGCCGTT | 28 | 1028 |
| 1126542 | 1441 | 1460 | 17874 | 17893 | ATTCATAAAGCTTCCTCCCG | 24 | 1029 |
| 1126574 | 1619 | 1638 | 21337 | 21356 | AAGAGGCCGATTCGGCGGAT | 0 | 1030 |
| 1126606 | 1726 | 1745 | 21724 | 21743 | GGTGACAGCCACGGACAAAC | 39 | 1031 |
| 1126638 | 1912 | 1931 | 25317 | 25336 | AGGAATCATCCAGGCTGCGG | 14 | 1032 |
| 1126670 | 2012 | 2031 | 25417 | 25436 | TCCAGAAGGTCGGAGAGGCG | 14 | 1033 |
| 1126702 | 2169 | 2188 | 26427 | 26446 | TGACAGCGAGGGCGACGGTG | 20 | 1034 |
| 1126734 | 2304 | 2323 | 26562 | 26581 | CGGCCACTCTGGTGCACGGA | 20 | 1035 |
| 1126766 | 2513 | 2532 | 26771 | 26790 | GTTTAGGAGCAGCACCCCTC | 21 | 1036 |
| 1126798 | 2742 | 2761 | 27000 | 27019 | AACCTCTGGCACCACCGCAG | 53 | 1037 |
| 1126830 | 2904 | 2923 | 27162 | 27181 | GGACTTGATCGCCCCATTCG | 42 | 1038 |
| 1126862 | 3051 | 3070 | 27309 | 27328 | AGCCGGATGGAGGGATCCTC | 22 | 1039 |
| 1126894 | 3315 | 3334 | 27573 | 27592 | GACCTAGAACCTAGTGGTTT | 0 | 1040 |
| 1126926 | 3561 | 3580 | 27819 | 27838 | CTCCGGACACACTCCAATCA | 26 | 1041 |
| 1126958 | N/A | N/A | 3074 | 3093 | TAGCCCCGTCCTCCTACAAC | 3 | 1042 |
| 1126990 | N/A | N/A | 3210 | 3229 | CTAGGACCCAGAACCCGGGC | 18 | 1043 |
| 1127022 | N/A | N/A | 3344 | 3363 | AAACCCCGGCAGCTTCCCAG | 50 | 1044 |
| 1127054 | N/A | N/A | 3386 | 3405 | GAGTTTTATCCCCTAGACCA | 61 | 1045 |
| 1127086 | N/A | N/A | 3546 | 3565 | CTCCTAGGAACTAATAGCTC | 5 | 1046 |
| 1127118 | N/A | N/A | 3833 | 3852 | GGTGTTCACGGGCCCCCTCT | 3 | 1047 |
| 1127150 | N/A | N/A | 4096 | 4115 | GTTTAAATCGCAGCCAGGCT | 30 | 1048 |
| 1127182 | N/A | N/A | 4367 | 4386 | AGCCCATAGTTTGGAGTAGG | 54 | 1049 |
| 1127214 | N/A | N/A | 4818 | 4837 | TATCACGCCTCCTTCCTGTG | 36 | 1050 |
| 1127246 | N/A | N/A | 4976 | 4995 | GCTGTCTTTCGATCTTGCTC | 16 | 1051 |
| 1127278 | N/A | N/A | 5414 | 5433 | GACAATCACCGCCCACACAA | 39 | 1052 |
| 1127310 | N/A | N/A | 5886 | 5905 | CGGACCTGAATGGATTCCAG | 29 | 1053 |
| 1127342 | N/A | N/A | 5970 | 5989 | GGCTATTCTTAGGCCCCCAG | 32 | 1054 |
| 1127374 | N/A | N/A | 6261 | 6280 | GGCCACGCCACACATTTCTA | 0 | 1055 |
| 1127406 | N/A | N/A | 7664 | 7683 | TAATTCCTTCCACGGGATGC | 29 | 1056 |
| 1127438 | N/A | N/A | 8504 | 8523 | TCTATAGACTTAAGCCTCAG | 44 | 1057 |
| 1127470 | N/A | N/A | 8894 | 8913 | GACCCTATGACTCCCAGAAT | 21 | 1058 |
| 1127502 | N/A | N/A | 8973 | 8992 | TGAAGTTGGCATCGAGAGTA | 49 | 1059 |
| 1127534 | N/A | N/A | 9081 | 9100 | GCAATATCACACTAGAGAAC | 58 | 1060 |
| 1127566 | N/A | N/A | 9613 | 9632 | CCCTTGTCTCAAGGGTACGC | 20 | 1061 |
| 1127598 | N/A | N/A | 9700 | 9719 | TTGGCCAACCATCTAAGTCC | 27 | 1062 |
| 1127630 | N/A | N/A | 9836 | 9855 | ACAGAGTGGGCAGCGGCCCA | 0 | 1063 |
| 1127662 | N/A | N/A | 9917 | 9936 | TCCACGAGTGTTGGGAAAAG | 0 | 1064 |
| 1127694 | N/A | N/A | 10359 | 10378 | GCAGTCCGGTTAGAAGGATT | 0 | 1065 |
| 1127726 | N/A | N/A | 12004 | 12023 | TAGGACTTAGGATGGTCCAA | 15 | 1066 |
| 1127758 | N/A | N/A | 12260 | 12279 | ATGCCCAGTTCTGACCTAAC | 29 | 1067 |
| 1127790 | N/A | N/A | 12352 | 12371 | TGGAACATATAGAGCTAACC | 68 | 1068 |
| 1127822 | N/A | N/A | 12632 | 12651 | GCTTAGGTTCACAAGCACAA | 51 | 1069 |
| 1127854 | N/A | N/A | 13330 | 13349 | CCCCAGACCTAGATGGTTAG | 0 | 1070 |
| 1127886 | N/A | N/A | 13577 | 13596 | CAAAGCCCTCACCCGCTAGC | 12 | 1071 |
| 1127918 | N/A | N/A | 14188 | 14207 | CTCTATACTCCCCTCACGGC | 15 | 1072 |
| 1127950 | N/A | N/A | 14470 | 14489 | GGAGCGAGGGCTGCTAACCA | 8 | 1073 |
| 1127954 | N/A | N/A | 15654 | 15673 | CCGTCTACAGGATTTTCTAG | 95 | 83 |
| 1127983 | N/A | N/A | 15826 | 15845 | CGTCTCAATCATGCCTCGGG | 91 | 1074 |
| 1128015 | N/A | N/A | 16772 | 16791 | CTTAGGCACACAAGTAAAGC | 72 | 1075 |
| 1128047 | N/A | N/A | 17029 | 17048 | CGTGCCTGACATCAGCTGTG | 36 | 1076 |
| 1128079 | N/A | N/A | 17837 | 17856 | GTTGGCCGTGTCCCTGGAGG | 46 | 1077 |
| 1128111 | N/A | N/A | 18189 | 18208 | TAGATCCCTTCATAAAGGGC | 7 | 1078 |
| 1128143 | N/A | N/A | 18337 | 18356 | GCTCATTGACCCCAGAATCC | 53 | 1079 |
| 1128175 | N/A | N/A | 19355 | 19374 | CCTAAAGTGGCCCGATAAAA | 15 | 1080 |
| 1128207 | N/A | N/A | 19563 | 19582 | TTGGGCCCCTAGACTCAATA | 15 | 1081 |
| 1128239 | N/A | N/A | 19774 | 19793 | TATAGGTTAGCCAAAAGCAG | 23 | 1082 |
| 1128271 | N/A | N/A | 19876 | 19895 | ACCACTCTACTGAGTCTAGG | 37 | 1083 |
| 1128303 | N/A | N/A | 21105 | 21124 | GTTGTGGATTCCCACTAGTA | 32 | 1084 |
| 1128335 | N/A | N/A | 21610 | 21629 | GCCTTATTTAGCTAAGCCCT | 10 | 1085 |
| 1128367 | N/A | N/A | 21820 | 21839 | AGCTCTGACTAGGATGCCAT | 0 | 1086 |
| 1128399 | N/A | N/A | 21924 | 21943 | CACAAAAGCACGCGCTGTAG | 11 | 1087 |
| 1128431 | N/A | N/A | 22090 | 22109 | TACGAGGAGACTTGGCCACA | 20 | 1088 |
| 1128463 | N/A | N/A | 24526 | 24545 | CCCTGTGTACAGAGCATCTA | 16 | 1089 |
| 1128495 | N/A | N/A | 24656 | 24675 | GTAGGATCGGGCCAAGGTCC | 19 | 1090 |
| 1128527 | N/A | N/A | 24752 | 24771 | CTCTTGCTCCAAGATGCGGG | 26 | 1091 |
| 1128559 | N/A | N/A | 24934 | 24953 | CACTTAGCTTCCCTCATCGC | 12 | 1092 |
| 1128591 | N/A | N/A | 25441 | 25460 | CCTACCCGGCCTAGGTATTT | 5 | 1093 |
| 1128623 | N/A | N/A | 25572 | 25591 | ATCGCGATCGGCCTGGTTCA | 5 | 1094 |
| 1128655 | N/A | N/A | 25985 | 26004 | CCCCTTATGTAGATAAGCAG | 17 | 1095 |
| 1128687 | N/A | N/A | 26353 | 26372 | GGTGCAAACCAGGGTGAGCT | 17 | 1096 |

TABLE 16
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126255 | 11 | 30 | 2658 | 2677 | CCCTGCAGTGAAACCTCGCA | 0 | 1097 |
| 1126287 | 161 | 180 | 2808 | 2827 | AATGCACCAGGTAGGGTGCG | 0 | 1098 |
| 1126319 | 237 | 256 | 2884 | 2903 | GCGGTTTAAAGGCATGGCTG | 51 | 1099 |
| 1126351 | 415 | 434 | 4573 | 4592 | CCAGGAAGTAGTTGTCGCCC | 64 | 1100 |
| 1126383 | 652 | 671 | 8726 | 8745 | ACCACGGCACTCCGATGTTG | 45 | 1101 |
| 1126415 | 871 | 890 | 10103 | 10122 | CACCGGCACACAGGTAGCGC | 5 | 1102 |
| 1126447 | 981 | 1000 | 10457 | 10476 | GTGAGCGCAGTGGGCTGCCG | 0 | 1103 |
| 1126479 | 1190 | 1209 | 13633 | 13652 | GTCTTGTCCAAGTTGAAGTC | 33 | 1104 |
| 1126511 | 1308 | 1327 | 14369 | 14388 | CACTGTCTGCTCGCTGCCGT | 27 | 1105 |
| 1126543 | 1443 | 1462 | 17876 | 17895 | GGATTCATAAAGCTTCCTCC | 27 | 1106 |
| 1126575 | 1621 | 1640 | 21339 | 21358 | TGAAGAGGCCGATTCGGCGG | 18 | 1107 |
| 1126607 | 1730 | 1749 | 21728 | 21747 | CCAAGGTGACAGCCACGGAC | 8 | 1108 |
| 1126639 | 1913 | 1932 | 25318 | 25337 | CAGGAATCATCCAGGCTGCG | 0 | 1109 |
| 1126671 | 2020 | 2039 | 25425 | 25444 | ATTTCCAGTCCAGAAGGTCG | 7 | 1110 |
| 1126703 | 2170 | 2189 | 26428 | 26447 | GTGACAGCGAGGGCGACGGT | 31 | 1111 |
| 1126735 | 2306 | 2325 | 26564 | 26583 | CGCGGCCACTCTGGTGCACG | 0 | 1112 |
| 1126767 | 2514 | 2533 | 26772 | 26791 | GGTTTAGGAGCAGCACCCCT | 33 | 1113 |
| 1126799 | 2743 | 2762 | 27001 | 27020 | AAACCTCTGGCACCACCGCA | 44 | 1114 |
| 1126831 | 2905 | 2924 | 27163 | 27182 | TGGACTTGATCGCCCCATTC | 42 | 1115 |
| 1126863 | 3052 | 3071 | 27310 | 27329 | GAGCCGGATGGAGGGATCCT | 27 | 1116 |
| 1126895 | 3316 | 3335 | 27574 | 27593 | GGACCTAGAACCTAGTGGTT | 28 | 1117 |
| 1126927 | 3562 | 3581 | 27820 | 27839 | TCTCCGGACACACTCCAATC | 48 | 1118 |
| 1126959 | N/A | N/A | 3075 | 3094 | GTAGCCCCGTCCTCCTACAA | 5 | 1119 |
| 1126991 | N/A | N/A | 3212 | 3231 | CCCTAGGACCCAGAACCCGG | 20 | 1120 |
| 1127023 | N/A | N/A | 3345 | 3364 | GAAACCCCGGCAGCTTCCCA | 56 | 1121 |
| 1127055 | N/A | N/A | 3387 | 3406 | GGAGTTTTATCCCCTAGACC | 80 | 1122 |
| 1127087 | N/A | N/A | 3547 | 3566 | ACTCCTAGGAACTAATAGCT | 20 | 1123 |
| 1127119 | N/A | N/A | 3842 | 3861 | ACCCGGCTGGGTGTTCACGG | 15 | 1124 |
| 1127151 | N/A | N/A | 4097 | 4116 | GGTTTAAATCGCAGCCAGGC | 33 | 1125 |
| 1127183 | N/A | N/A | 4368 | 4387 | GAGCCCATAGTTTGGAGTAG | 45 | 1126 |
| 1127215 | N/A | N/A | 4822 | 4841 | GTCCTATCACGCCTCCTTCC | 77 | 1127 |
| 1127247 | N/A | N/A | 4977 | 4996 | GGCTGTCTTTCGATCTTGCT | 48 | 1128 |
| 1127279 | N/A | N/A | 5415 | 5434 | TGACAATCACCGCCCACACA | 40 | 1129 |
| 1127311 | N/A | N/A | 5887 | 5906 | TCGGACCTGAATGGATTCCA | 61 | 1130 |
| 1127343 | N/A | N/A | 6034 | 6053 | AGCCTCGCTCTGAGGTCCCT | 0 | 1131 |
| 1127375 | N/A | N/A | 6262 | 6281 | CGGCCACGCCACACATTTCT | 0 | 1132 |
| 1127407 | N/A | N/A | 7665 | 7684 | CTAATTCCTTCCACGGGATG | 22 | 1133 |
| 1127439 | N/A | N/A | 8505 | 8524 | GTCTATAGACTTAAGCCTCA | 49 | 1134 |
| 1127471 | N/A | N/A | 8895 | 8914 | GGACCCTATGACTCCCAGAA | 31 | 1135 |
| 1127503 | N/A | N/A | 8974 | 8993 | ATGAAGTTGGCATCGAGAGT | 54 | 1136 |
| 1127535 | N/A | N/A | 9084 | 9103 | CCAGCAATATCACACTAGAG | 92 | 1137 |
| 1127567 | N/A | N/A | 9614 | 9633 | CCCCTTGTCTCAAGGGTACG | 0 | 1138 |
| 1127599 | N/A | N/A | 9701 | 9720 | TTTGGCCAACCATCTAAGTC | 2 | 1139 |
| 1127631 | N/A | N/A | 9845 | 9864 | CACTACTGCACAGAGTGGGC | 1 | 1140 |
| 1127663 | N/A | N/A | 9918 | 9937 | CTCCACGAGTGTTGGGAAAA | 19 | 1141 |
| 1127695 | N/A | N/A | 10360 | 10379 | AGCAGTCCGGTTAGAAGGAT | 11 | 1142 |
| 1127727 | N/A | N/A | 12005 | 12024 | TTAGGACTTAGGATGGTCCA | 4 | 1143 |
| 1127759 | N/A | N/A | 12262 | 12281 | GAATGCCCAGTTCTGACCTA | 25 | 1144 |
| 1127791 | N/A | N/A | 12357 | 12376 | AGTTCTGGAACATATAGAGC | 69 | 1145 |
| 1127823 | N/A | N/A | 12640 | 12659 | TATTACTTGCTTAGGTTCAC | 82 | 1146 |
| 1127855 | N/A | N/A | 13331 | 13350 | CCCCCAGACCTAGATGGTTA | 12 | 1147 |
| 1127887 | N/A | N/A | 13578 | 13597 | CCAAAGCCCTCACCCGCTAG | 14 | 1148 |
| 1127919 | N/A | N/A | 14189 | 14208 | TCTCTATACTCCCCTCACGG | 25 | 1149 |
| 1127951 | N/A | N/A | 14472 | 14491 | CCGGAGCGAGGGCTGCTAAC | 12 | 1150 |
| 1127954 | N/A | N/A | 15654 | 15673 | CCGTCTACAGGATTTTCTAG | 94 | 83 |
| 1127984 | N/A | N/A | 15844 | 15863 | CCATGGGACTCCCCACCGCG | 34 | 1151 |
| 1128016 | N/A | N/A | 16777 | 16796 | GACCACTTAGGCACACAAGT | 93 | 1152 |
| 1128048 | N/A | N/A | 17048 | 17067 | CTGGAAGGCTTAGGACCCAC | 51 | 1153 |
| 1128080 | N/A | N/A | 17838 | 17857 | TGTTGGCCGTGTCCCTGGAG | 48 | 1154 |
| 1128112 | N/A | N/A | 18190 | 18209 | ATAGATCCCTTCATAAAGGG | 0 | 1155 |
| 1128144 | N/A | N/A | 18859 | 18878 | TAGTGTGGGATCTGGCTCTG | 35 | 1156 |
| 1128176 | N/A | N/A | 19356 | 19375 | CCCTAAAGTGGCCCGATAAA | 0 | 1157 |
| 1128208 | N/A | N/A | 19564 | 19583 | TTTGGGCCCCTAGACTCAAT | 0 | 1158 |
| 1128240 | N/A | N/A | 19776 | 19795 | ATTATAGGTTAGCCAAAAGC | 25 | 1159 |
| 1128272 | N/A | N/A | 19877 | 19896 | CACCACTCTACTGAGTCTAG | 23 | 1160 |
| 1128304 | N/A | N/A | 21107 | 21126 | GAGTTGTGGATTCCCACTAG | 42 | 1161 |
| 1128336 | N/A | N/A | 21611 | 21630 | TGCCTTATTTAGCTAAGCCC | 15 | 1162 |
| 1128368 | N/A | N/A | 21821 | 21840 | TAGCTCTGACTAGGATGCCA | 20 | 1163 |
| 1128400 | N/A | N/A | 21925 | 21944 | TCACAAAAGCACGCGCTGTA | 4 | 1164 |
| 1128432 | N/A | N/A | 22091 | 22110 | TTACGAGGAGACTTGGCCAC | 5 | 1165 |
| 1128464 | N/A | N/A | 24544 | 24563 | GTCTCTCATTATTAGTATCC | 41 | 1166 |
| 1128496 | N/A | N/A | 24657 | 24676 | TGTAGGATCGGGCCAAGGTC | 45 | 1167 |
| 1128528 | N/A | N/A | 24753 | 24772 | GCTCTTGCTCCAAGATGCGG | 0 | 1168 |
| 1128560 | N/A | N/A | 24935 | 24954 | CCACTTAGCTTCCCTCATCG | 9 | 1169 |
| 1128592 | N/A | N/A | 25442 | 25461 | TCCTACCCGGCCTAGGTATT | 18 | 1170 |
| 1128624 | N/A | N/A | 25573 | 25592 | CATCGCGATCGGCCTGGTTC | 0 | 1171 |
| 1128656 | N/A | N/A | 25986 | 26005 | ACCCCTTATGTAGATAAGCA | 8 | 1172 |
| 1128688 | N/A | N/A | 26360 | 26379 | CGATGTGGGTGCAAACCAGG | 0 | 1173 |

TABLE 17
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126256 | 13 | 32 | 2660 | 2679 | GCCCCTGCAGTGAAACCTCG | 20 | 1174 |
| 1126288 | 162 | 181 | 2809 | 2828 | GAATGCACCAGGTAGGGTGC | 13 | 1175 |
| 1126320 | 239 | 258 | 2886 | 2905 | GTGCGGTTTAAAGGCATGGC | 42 | 1176 |
| 1126352 | 416 | 435 | 4574 | 4593 | ACCAGGAAGTAGTTGTCGCC | 44 | 1177 |
| 1126384 | 653 | 672 | 8727 | 8746 | TACCACGGCACTCCGATGTT | 17 | 1178 |
| 1126416 | 872 | 891 | 10104 | 10123 | GCACCGGCACACAGGTAGCG | 30 | 1179 |
| 1126448 | 982 | 1001 | 10458 | 10477 | CGTGAGCGCAGTGGGCTGCC | 4 | 1180 |
| 1126480 | 1195 | 1214 | 13638 | 13657 | ATAAGGTCTTGTCCAAGTTG | 54 | 1181 |
| 1126512 | 1311 | 1330 | 14372 | 14391 | AACCACTGTCTGCTCGCTGC | 59 | 1182 |
| 1126544 | 1450 | 1469 | 17883 | 17902 | CCAGTAAGGATTCATAAAGC | 11 | 1183 |
| 1126576 | 1622 | 1641 | 21340 | 21359 | TTGAAGAGGCCGATTCGGCG | 0 | 1184 |
| 1126608 | 1731 | 1750 | 21729 | 21748 | TCCAAGGTGACAGCCACGGA | 17 | 1185 |
| 1126640 | 1918 | 1937 | 25323 | 25342 | GCGAGCAGGAATCATCCAGG | 24 | 1186 |
| 1126672 | 2030 | 2049 | 25435 | 25454 | CGGCCTAGGTATTTCCAGTC | 5 | 1187 |
| 1126704 | 2229 | 2248 | 26487 | 26506 | CTCCAGCGGCCCGTTCCGGG | 5 | 1188 |
| 1126736 | 2307 | 2326 | 26565 | 26584 | GCGCGGCCACTCTGGTGCAC | 0 | 1189 |
| 1126768 | 2534 | 2553 | 26792 | 26811 | CAGTGCAGATCTGGAGCGGG | 57 | 1190 |
| 1126800 | 2744 | 2763 | 27002 | 27021 | AAAACCTCTGGCACCACCGC | 45 | 1191 |
| 1126832 | 2906 | 2925 | 27164 | 27183 | CTGGACTTGATCGCCCCATT | 52 | 1192 |
| 1126864 | 3054 | 3073 | 27312 | 27331 | AGGAGCCGGATGGAGGGATC | 29 | 1193 |
| 1126896 | 3341 | 3360 | 27599 | 27618 | GGTAAGGTTCCAGATCTAGA | 81 | 1194 |
| 1126928 | 3563 | 3582 | 27821 | 27840 | ATCTCCGGACACACTCCAAT | 58 | 1195 |
| 1126960 | N/A | N/A | 3076 | 3095 | AGTAGCCCCGTCCTCCTACA | 3 | 1196 |
| 1126992 | N/A | N/A | 3213 | 3232 | TCCCTAGGACCCAGAACCCG | 18 | 1197 |
| 1127024 | N/A | N/A | 3349 | 3368 | GTCTGAAACCCCGGCAGCTT | 45 | 1198 |
| 1127056 | N/A | N/A | 3388 | 3407 | TGGAGTTTTATCCCCTAGAC | 49 | 1199 |
| 1127088 | N/A | N/A | 3548 | 3567 | TACTCCTAGGAACTAATAGC | 42 | 1200 |
| 1127120 | N/A | N/A | 3843 | 3862 | TACCCGGCTGGGTGTTCACG | 11 | 1201 |
| 1127152 | N/A | N/A | 4098 | 4117 | GGGTTTAAATCGCAGCCAGG | 32 | 1202 |
| 1127184 | N/A | N/A | 4369 | 4388 | GGAGCCCATAGTTTGGAGTA | 38 | 1203 |
| 1127216 | N/A | N/A | 4823 | 4842 | TGTCCTATCACGCCTCCTTC | 64 | 1204 |
| 1127248 | N/A | N/A | 4978 | 4997 | AGGCTGTCTTTCGATCTTGC | 79 | 1205 |
| 1127280 | N/A | N/A | 5416 | 5435 | ATGACAATCACCGCCCACAC | 41 | 1206 |
| 1127312 | N/A | N/A | 5888 | 5907 | GTCGGACCTGAATGGATTCC | 36 | 1207 |
| 1127344 | N/A | N/A | 6057 | 6076 | CGACCTGGCTTTTTGCTGCC | 84 | 1208 |
| 1127376 | N/A | N/A | 6263 | 6282 | TCGGCCACGCCACACATTTC | 18 | 1209 |
| 1127408 | N/A | N/A | 7666 | 7685 | GCTAATTCCTTCCACGGGAT | 73 | 1210 |
| 1127440 | N/A | N/A | 8506 | 8525 | TGTCTATAGACTTAAGCCTC | 46 | 1211 |
| 1127472 | N/A | N/A | 8896 | 8915 | AGGACCCTATGACTCCCAGA | 49 | 1212 |
| 1127504 | N/A | N/A | 8977 | 8996 | TAAATGAAGTTGGCATCGAG | 66 | 1213 |
| 1127536 | N/A | N/A | 9086 | 9105 | GTCCAGCAATATCACACTAG | 70 | 1214 |
| 1127568 | N/A | N/A | 9615 | 9634 | ACCCCTTGTCTCAAGGGTAC | 1 | 1215 |
| 1127600 | N/A | N/A | 9703 | 9722 | TTTTTGGCCAACCATCTAAG | 40 | 1216 |
| 1127632 | N/A | N/A | 9846 | 9865 | CCACTACTGCACAGAGTGGG | 0 | 1217 |
| 1127664 | N/A | N/A | 9919 | 9938 | TCTCCACGAGTGTTGGGAAA | 0 | 1218 |
| 1127696 | N/A | N/A | 10361 | 10380 | CAGCAGTCCGGTTAGAAGGA | 0 | 1219 |
| 1127728 | N/A | N/A | 12007 | 12026 | GCTTAGGACTTAGGATGGTC | 87 | 1220 |
| 1127760 | N/A | N/A | 12285 | 12304 | GAACGGATACTAAGGGCTGG | 72 | 1221 |
| 1127792 | N/A | N/A | 12366 | 12385 | CTAGGGCAAAGTTCTGGAAC | 70 | 1222 |
| 1127824 | N/A | N/A | 12645 | 12664 | CCTAATATTACTTGCTTAGG | 10 | 1223 |
| 1127856 | N/A | N/A | 13332 | 13351 | TCCCCCAGACCTAGATGGTT | 20 | 1224 |
| 1127888 | N/A | N/A | 13587 | 13606 | CGGTAAAGCCCAAAGCCCTC | 16 | 1225 |
| 1127920 | N/A | N/A | 14190 | 14209 | GTCTCTATACTCCCCTCACG | 27 | 1226 |
| 1127952 | N/A | N/A | 15452 | 15471 | GTGAGTATCTCTGCTAATGA | 87 | 1227 |
| 1127954 | N/A | N/A | 15654 | 15673 | CCGTCTACAGGATTTTCTAG | 95 | 83 |
| 1127985 | N/A | N/A | 15845 | 15864 | CCCATGGGACTCCCCACCGC | 22 | 1228 |
| 1128017 | N/A | N/A | 16778 | 16797 | GGACCACTTAGGCACACAAG | 92 | 161 |
| 1128049 | N/A | N/A | 17050 | 17069 | CCCTGGAAGGCTTAGGACCC | 9 | 1229 |
| 1128081 | N/A | N/A | 17839 | 17858 | GTGTTGGCCGTGTCCCTGGA | 55 | 1230 |
| 1128113 | N/A | N/A | 18199 | 18218 | GACCTCAGAATAGATCCCTT | 26 | 1231 |
| 1128145 | N/A | N/A | 18860 | 18879 | CTAGTGTGGGATCTGGCTCT | 20 | 1232 |
| 1128177 | N/A | N/A | 19357 | 19376 | TCCCTAAAGTGGCCCGATAA | 22 | 1233 |
| 1128209 | N/A | N/A | 19565 | 19584 | TTTTGGGCCCCTAGACTCAA | 1 | 1234 |
| 1128241 | N/A | N/A | 19782 | 19801 | TTGGAAATTATAGGTTAGCC | 33 | 1235 |
| 1128273 | N/A | N/A | 19880 | 19899 | TACCACCACTCTACTGAGTC | 17 | 1236 |
| 1128305 | N/A | N/A | 21108 | 21127 | GGAGTTGTGGATTCCCACTA | 45 | 1237 |
| 1128337 | N/A | N/A | 21612 | 21631 | GTGCCTTATTTAGCTAAGCC | 6 | 1238 |
| 1128369 | N/A | N/A | 21822 | 21841 | CTAGCTCTGACTAGGATGCC | 33 | 1239 |
| 1128401 | N/A | N/A | 21926 | 21945 | ATCACAAAAGCACGCGCTGT | 16 | 1240 |
| 1128433 | N/A | N/A | 22092 | 22111 | CTTACGAGGAGACTTGGCCA | 21 | 1241 |
| 1128465 | N/A | N/A | 24545 | 24564 | GGTCTCTCATTATTAGTATC | 33 | 1242 |
| 1128497 | N/A | N/A | 24658 | 24677 | CTGTAGGATCGGGCCAAGGT | 24 | 1243 |
| 1128529 | N/A | N/A | 24754 | 24773 | TGCTCTTGCTCCAAGATGCG | 26 | 1244 |
| 1128561 | N/A | N/A | 24938 | 24957 | GACCCACTTAGCTTCCCTCA | 7 | 1245 |
| 1128593 | N/A | N/A | 25443 | 25462 | GTCCTACCCGGCCTAGGTAT | 21 | 1246 |
| 1128625 | N/A | N/A | 25574 | 25593 | CCATCGCGATCGGCCTGGTT | 17 | 1247 |
| 1128657 | N/A | N/A | 25987 | 26006 | CACCCCTTATGTAGATAAGC | 9 | 1248 |
| 1128689 | N/A | N/A | 26361 | 26380 | GCGATGTGGGTGCAAACCAG | 3 | 1249 |

TABLE 18
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126257 | 14 | 33 | 2661 | 2680 | CGCCCCTGCAGTGAAACCTC | 0 | 1250 |
| 1126289 | 163 | 182 | 2810 | 2829 | GGAATGCACCAGGTAGGGTG | 29 | 1251 |
| 1126321 | 240 | 259 | 2887 | 2906 | AGTGCGGTTTAAAGGCATGG | 38 | 1252 |
| 1126353 | 418 | 437 | 4576 | 4595 | CCACCAGGAAGTAGTTGTCG | 27 | 1253 |
| 1126385 | 654 | 673 | 8728 | 8747 | GTACCACGGCACTCCGATGT | 53 | 1254 |
| 1126417 | 874 | 893 | 10106 | 10125 | CGGCACCGGCACACAGGTAG | 51 | 1255 |
| 1126449 | 1009 | 1028 | 10485 | 10504 | CGGTGATCTGGGACACAGTA | 94† | 1256 |
| 1126481 | 1200 | 1219 | 13643 | 13662 | GAAGTATAAGGTCTTGTCCA | 64 | 1257 |
| 1126513 | 1312 | 1331 | 14373 | 14392 | CAACCACTGTCTGCTCGCTG | 44 | 1258 |
| 1126545 | 1472 | 1491 | 18000 | 18019 | TTCATGTCGGGAAGGCTCCC | 28 | 1259 |
| 1126577 | 1623 | 1642 | 21341 | 21360 | ATTGAAGAGGCCGATTCGGC | 2 | 1260 |
| 1126609 | 1758 | 1777 | 21756 | 21775 | CCAAGGCTCATAGTAGGAGG | 29 | 1261 |
| 1126641 | 1920 | 1939 | 25325 | 25344 | CTGCGAGCAGGAATCATCCA | 31 | 1262 |
| 1126673 | 2031 | 2050 | 25436 | 25455 | CCGGCCTAGGTATTTCCAGT | 6 | 1263 |
| 1126705 | 2231 | 2250 | 26489 | 26508 | TCCTCCAGCGGCCCGTTCCG | 6 | 1264 |
| 1126737 | 2308 | 2327 | 26566 | 26585 | GGCGCGGCCACTCTGGTGCA | 4 | 1265 |
| 1126769 | 2535 | 2554 | 26793 | 26812 | CCAGTGCAGATCTGGAGCGG | 36 | 1266 |
| 1126801 | 2745 | 2764 | 27003 | 27022 | TAAAACCTCTGGCACCACCG | 52 | 1267 |
| 1126833 | 2907 | 2926 | 27165 | 27184 | TCTGGACTTGATCGCCCCAT | 43 | 1268 |
| 1126865 | 3059 | 3078 | 27317 | 27336 | ATGACAGGAGCCGGATGGAG | 28 | 1269 |
| 1126897 | 3342 | 3361 | 27600 | 27619 | TGGTAAGGTTCCAGATCTAG | 62 | 1270 |
| 1126929 | 3564 | 3583 | 27822 | 27841 | CATCTCCGGACACACTCCAA | 49 | 1271 |
| 1126961 | N/A | N/A | 3077 | 3096 | TAGTAGCCCCGTCCTCCTAC | 0 | 1272 |
| 1126993 | N/A | N/A | 3239 | 3258 | CTAGGAGTCTCATTTTAGCG | 82 | 1273 |
| 1127025 | N/A | N/A | 3350 | 3369 | GGTCTGAAACCCCGGCAGCT | 37 | 1274 |
| 1127057 | N/A | N/A | 3390 | 3409 | TCTGGAGTTTTATCCCCTAG | 65 | 1275 |
| 1127089 | N/A | N/A | 3549 | 3568 | CTACTCCTAGGAACTAATAG | 27 | 1276 |
| 1127121 | N/A | N/A | 3844 | 3863 | ATACCCGGCTGGGTGTTCAC | 23 | 1277 |
| 1127153 | N/A | N/A | 4105 | 4124 | AGGAGCTGGGTTTAAATCGC | 33 | 1278 |
| 1127185 | N/A | N/A | 4371 | 4390 | TGGGAGCCCATAGTTTGGAG | 42 | 1279 |
| 1127217 | N/A | N/A | 4824 | 4843 | GTGTCCTATCACGCCTCCTT | 85 | 1280 |
| 1127249 | N/A | N/A | 4979 | 4998 | CAGGCTGTCTTTCGATCTTG | 86 | 1281 |
| 1127281 | N/A | N/A | 5417 | 5436 | AATGACAATCACCGCCCACA | 17 | 1282 |
| 1127313 | N/A | N/A | 5889 | 5908 | AGTCGGACCTGAATGGATTC | 80 | 1283 |
| 1127345 | N/A | N/A | 6058 | 6077 | CCGACCTGGCTTTTTGCTGC | 68 | 1284 |
| 1127377 | N/A | N/A | 6264 | 6283 | CTCGGCCACGCCACACATTT | 15 | 1285 |
| 1127409 | N/A | N/A | 7668 | 7687 | ATGCTAATTCCTTCCACGGG | 89 | 1286 |
| 1127441 | N/A | N/A | 8507 | 8526 | ATGTCTATAGACTTAAGCCT | 20 | 1287 |
| 1127473 | N/A | N/A | 8900 | 8919 | TTAAAGGACCCTATGACTCC | 8 | 1288 |
| 1127505 | N/A | N/A | 8978 | 8997 | GTAAATGAAGTTGGCATCGA | 71 | 1289 |
| 1127537 | N/A | N/A | 9124 | 9143 | GGGATTCATGAGCATCCAGG | 7 | 1290 |
| 1127569 | N/A | N/A | 9616 | 9635 | GACCCCTTGTCTCAAGGGTA | 4 | 1291 |
| 1127601 | N/A | N/A | 9706 | 9725 | GATTTTTTGGCCAACCATCT | 49 | 1292 |
| 1127633 | N/A | N/A | 9847 | 9866 | ACCACTACTGCACAGAGTGG | 9 | 1293 |
| 1127665 | N/A | N/A | 9923 | 9942 | ACTGTCTCCACGAGTGTTGG | 45 | 1294 |
| 1127697 | N/A | N/A | 10362 | 10381 | ACAGCAGTCCGGTTAGAAGG | 0 | 1295 |
| 1127729 | N/A | N/A | 12008 | 12027 | AGCTTAGGACTTAGGATGGT | 48 | 1296 |
| 1127761 | N/A | N/A | 12286 | 12305 | AGAACGGATACTAAGGGCTG | 41 | 1297 |
| 1127793 | N/A | N/A | 12387 | 12406 | AACTACAACCCCCATCAGCG | 4 | 1298 |
| 1127825 | N/A | N/A | 12681 | 12700 | GTGACACACCTACCCTGACT | 24 | 1299 |
| 1127857 | N/A | N/A | 13388 | 13407 | GAATACCCAGGTGCCCCCTC | 3 | 1300 |
| 1127889 | N/A | N/A | 13588 | 13607 | ACGGTAAAGCCCAAAGCCCT | 0 | 1301 |
| 1127921 | N/A | N/A | 14191 | 14210 | GGTCTCTATACTCCCCTCAC | 53 | 1302 |
| 1127953 | N/A | N/A | 15653 | 15672 | CGTCTACAGGATTTTCTAGA | 82 | 1303 |
| 1127954 | N/A | N/A | 15654 | 15673 | CCGTCTACAGGATTTTCTAG | 95 | 83 |
| 1127986 | N/A | N/A | 15866 | 15885 | TATGAGACATGTCACTCCAG | 66 | 1304 |
| 1128018 | N/A | N/A | 16781 | 16800 | AAAGGACCACTTAGGCACAC | 94 | 1305 |
| 1128050 | N/A | N/A | 17051 | 17070 | GCCCTGGAAGGCTTAGGACC | 1 | 1306 |
| 1128082 | N/A | N/A | 17840 | 17859 | CGTGTTGGCCGTGTCCCTGG | 50 | 1307 |
| 1128114 | N/A | N/A | 18202 | 18221 | GTGGACCTCAGAATAGATCC | 0 | 1308 |
| 1128146 | N/A | N/A | 18861 | 18880 | GCTAGTGTGGGATCTGGCTC | 22 | 1309 |
| 1128178 | N/A | N/A | 19358 | 19377 | GTCCCTAAAGTGGCCCGATA | 10 | 1310 |
| 1128210 | N/A | N/A | 19566 | 19585 | TTTTTGGGCCCCTAGACTCA | 23 | 1311 |
| 1128242 | N/A | N/A | 19788 | 19807 | GGACTATTGGAAATTATAGG | 28 | 1312 |
| 1128274 | N/A | N/A | 19882 | 19901 | CTTACCACCACTCTACTGAG | 0 | 1313 |
| 1128306 | N/A | N/A | 21109 | 21128 | AGGAGTTGTGGATTCCCACT | 22 | 1314 |
| 1128338 | N/A | N/A | 21613 | 21632 | AGTGCCTTATTTAGCTAAGC | 21 | 1315 |
| 1128370 | N/A | N/A | 21823 | 21842 | GCTAGCTCTGACTAGGATGC | 11 | 1316 |
| 1128402 | N/A | N/A | 21927 | 21946 | TATCACAAAAGCACGCGCTG | 16 | 1317 |
| 1128434 | N/A | N/A | 22093 | 22112 | TCTTACGAGGAGACTTGGCC | 0 | 1318 |
| 1128466 | N/A | N/A | 24556 | 24575 | GGGTAAGATAAGGTCTCTCA | 53 | 1319 |
| 1128498 | N/A | N/A | 24659 | 24678 | CCTGTAGGATCGGGCCAAGG | 25 | 1320 |
| 1128530 | N/A | N/A | 24836 | 24855 | CGTCGCTGAGCCCCCACAAC | 7 | 1321 |
| 1128562 | N/A | N/A | 24940 | 24959 | GAGACCCACTTAGCTTCCCT | 0 | 1322 |
| 1128594 | N/A | N/A | 25444 | 25463 | GGTCCTACCCGGCCTAGGTA | 0 | 1323 |
| 1128626 | N/A | N/A | 25575 | 25594 | ACCATCGCGATCGGCCTGGT | 57 | 1324 |
| 1128658 | N/A | N/A | 25988 | 26007 | CCACCCCTTATGTAGATAAG | 0 | 1325 |
| 1128690 | N/A | N/A | 26362 | 26381 | GGCGATGTGGGTGCAAACCA | 0 | 1326 |

TABLE 19
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126258 | 53 | 72 | 2700 | 2719 | CTAGGCAGAAGGAGGCCGCA | 6 | 1327 |
| 1126290 | 167 | 186 | 2814 | 2833 | CTAGGGAATGCACCAGGTAG | 60 | 1328 |
| 1126322 | 241 | 260 | 2888 | 2907 | AAGTGCGGTTTAAAGGCATG | 16 | 1329 |
| 1126354 | 422 | 441 | 4580 | 4599 | GGCCCCACCAGGAAGTAGTT | 1 | 1330 |
| 1126386 | 771 | 790 | 10003 | 10022 | CAACCACTCATGGAAGTGAG | 39 | 1331 |
| 1126418 | 875 | 894 | 10107 | 10126 | ACGGCACCGGCACACAGGTA | 45 | 1332 |
| 1126450 | 1010 | 1029 | 10486 | 10505 | GCGGTGATCTGGGACACAGT | 90† | 1333 |
| 1126482 | 1211 | 1230 | 13654 | 13673 | CCGGCGATAAAGAAGTATAA | 53 | 1334 |
| 1126514 | 1315 | 1334 | 14376 | 14395 | AGGCAACCACTGTCTGCTCG | 12 | 1335 |
| 1126546 | 1473 | 1492 | 18001 | 18020 | GTTCATGTCGGGAAGGCTCC | 37 | 1336 |
| 1126578 | 1624 | 1643 | 21342 | 21361 | TATTGAAGAGGCCGATTCGG | 0 | 1337 |
| 1126610 | 1759 | 1778 | 21757 | 21776 | CCCAAGGCTCATAGTAGGAG | 35 | 1338 |
| 1126642 | 1922 | 1941 | 25327 | 25346 | AGCTGCGAGCAGGAATCATC | 33 | 1339 |
| 1126674 | 2050 | 2069 | 26145 | 26164 | GGCGCGCAGACATATAGTAC | 13 | 1340 |
| 1126706 | 2232 | 2251 | 26490 | 26509 | TTCCTCCAGCGGCCCGTTCC | 27 | 1341 |
| 1126738 | 2310 | 2329 | 26568 | 26587 | TCGGCGCGGCCACTCTGGTG | 6 | 1342 |
| 1126770 | 2575 | 2594 | 26833 | 26852 | AGTGTTTGGCGGACTGGGTG | 54 | 1343 |
| 1126802 | 2809 | 2828 | 27067 | 27086 | GAATTTGTAAGCCACACGGG | 54 | 1344 |
| 1126834 | 2908 | 2927 | 27166 | 27185 | CTCTGGACTTGATCGCCCCA | 51 | 1345 |
| 1126866 | 3060 | 3079 | 27318 | 27337 | GATGACAGGAGCCGGATGGA | 51 | 1346 |
| 1126898 | 3343 | 3362 | 27601 | 27620 | GTGGTAAGGTTCCAGATCTA | 79 | 1347 |
| 1126930 | 3567 | 3586 | 27825 | 27844 | TCTCATCTCCGGACACACTC | 75 | 1348 |
| 1126962 | N/A | N/A | 3078 | 3097 | CTAGTAGCCCCGTCCTCCTA | 30 | 1349 |
| 1126994 | N/A | N/A | 3266 | 3285 | AGTCTCTCGGAAGCCTGCCT | 63 | 1350 |
| 1127026 | N/A | N/A | 3351 | 3370 | AGGTCTGAAACCCCGGCAGC | 49 | 1351 |
| 1127058 | N/A | N/A | 3430 | 3449 | CCGCTCAGGCCTCCTATCTC | 46 | 1352 |
| 1127090 | N/A | N/A | 3576 | 3595 | CGCATGTGTCCCAGGCCATG | 58 | 1353 |
| 1127122 | N/A | N/A | 3845 | 3864 | GATACCCGGCTGGGTGTTCA | 16 | 1354 |
| 1127154 | N/A | N/A | 4126 | 4145 | GTTCCCGAGTTCCCAGCTCA | 49 | 1355 |
| 1127186 | N/A | N/A | 4372 | 4391 | CTGGGAGCCCATAGTTTGGA | 48 | 1356 |
| 1127218 | N/A | N/A | 4825 | 4844 | GGTGTCCTATCACGCCTCCT | 23 | 1357 |
| 1127250 | N/A | N/A | 4981 | 5000 | ACCAGGCTGTCTTTCGATCT | 78 | 1358 |
| 1127282 | N/A | N/A | 5418 | 5437 | CAATGACAATCACCGCCCAC | 17 | 1359 |
| 1127314 | N/A | N/A | 5890 | 5909 | CAGTCGGACCTGAATGGATT | 61 | 1360 |
| 1127346 | N/A | N/A | 6059 | 6078 | GCCGACCTGGCTTTTTGCTG | 11 | 1361 |
| 1127378 | N/A | N/A | 6265 | 6284 | CCTCGGCCACGCCACACATT | 30 | 1362 |
| 1127410 | N/A | N/A | 7669 | 7688 | GATGCTAATTCCTTCCACGG | 95 | 1363 |
| 1127442 | N/A | N/A | 8508 | 8527 | GATGTCTATAGACTTAAGCC | 31 | 1364 |
| 1127474 | N/A | N/A | 8901 | 8920 | ATTAAAGGACCCTATGACTC | 24 | 1365 |
| 1127506 | N/A | N/A | 8979 | 8998 | AGTAAATGAAGTTGGCATCG | 81 | 1366 |
| 1127538 | N/A | N/A | 9146 | 9165 | GGGTCAAAACCAGGTCAGGC | 80 | 1367 |
| 1127570 | N/A | N/A | 9617 | 9636 | AGACCCCTTGTCTCAAGGGT | 8 | 1368 |
| 1127602 | N/A | N/A | 9707 | 9726 | GGATTTTTTGGCCAACCATC | 73 | 1369 |
| 1127634 | N/A | N/A | 9848 | 9867 | CACCACTACTGCACAGAGTG | 7 | 1370 |
| 1127666 | N/A | N/A | 9924 | 9943 | CACTGTCTCCACGAGTGTTG | 15 | 1371 |
| 1127698 | N/A | N/A | 10363 | 10382 | GACAGCAGTCCGGTTAGAAG | 9 | 1372 |
| 1127730 | N/A | N/A | 12009 | 12028 | CAGCTTAGGACTTAGGATGG | 61 | 1373 |
| 1127762 | N/A | N/A | 12287 | 12306 | GAGAACGGATACTAAGGGCT | 83 | 1374 |
| 1127794 | N/A | N/A | 12392 | 12411 | CGAAAAACTACAACCCCCAT | 33 | 1375 |
| 1127826 | N/A | N/A | 12683 | 12702 | TGGTGACACACCTACCCTGA | 13 | 1376 |
| 1127858 | N/A | N/A | 13389 | 13408 | CGAATACCCAGGTGCCCCCT | 11 | 1377 |
| 1127890 | N/A | N/A | 13589 | 13608 | CACGGTAAAGCCCAAAGCCC | 18 | 1378 |
| 1127922 | N/A | N/A | 14229 | 14248 | ATAGGACAGTCCCCACCTGG | 14 | 1379 |
| 1127954 | N/A | N/A | 15654 | 15673 | CCGTCTACAGGATTTTCTAG | 96 | 83 |
| 1127955 | N/A | N/A | 15655 | 15674 | TCCGTCTACAGGATTTTCTA | 87 | 1380 |
| 1127987 | N/A | N/A | 15867 | 15886 | CTATGAGACATGTCACTCCA | 54 | 1381 |
| 1128019 | N/A | N/A | 16828 | 16847 | GGCAATGCTAAGCCCCATTT | 91 | 1382 |
| 1128051 | N/A | N/A | 17140 | 17159 | GGTTTCTAGGATAGCAAGGA | 94 | 1383 |
| 1128083 | N/A | N/A | 17842 | 17861 | ACCGTGTTGGCCGTGTCCCT | 52 | 1384 |
| 1128115 | N/A | N/A | 18203 | 18222 | TGTGGACCTCAGAATAGATC | 48 | 1385 |
| 1128147 | N/A | N/A | 19198 | 19217 | TGTGTCCGCCTCGCTCTGTT | 36 | 1386 |
| 1128179 | N/A | N/A | 19359 | 19378 | CGTCCCTAAAGTGGCCCGAT | 9 | 1387 |
| 1128211 | N/A | N/A | 19567 | 19586 | TTTTTTGGGCCCCTAGACTC | 20 | 1388 |
| 1128243 | N/A | N/A | 19789 | 19808 | GGGACTATTGGAAATTATAG | 34 | 1389 |
| 1128275 | N/A | N/A | 19917 | 19936 | AGCAACTGTTCCATCATAGC | 91 | 1390 |
| 1128307 | N/A | N/A | 21111 | 21130 | CCAGGAGTTGTGGATTCCCA | 46 | 1391 |
| 1128339 | N/A | N/A | 21623 | 21642 | ACTCAGCCCAAGTGCCTTAT | 23 | 1392 |
| 1128371 | N/A | N/A | 21824 | 21843 | TGCTAGCTCTGACTAGGATG | 13 | 1393 |
| 1128403 | N/A | N/A | 21928 | 21947 | GTATCACAAAAGCACGCGCT | 46 | 1394 |
| 1128435 | N/A | N/A | 22094 | 22113 | GTCTTACGAGGAGACTTGGC | 18 | 1395 |
| 1128467 | N/A | N/A | 24577 | 24596 | GACTTAGTCTATTTTGATGG | 17 | 1396 |
| 1128499 | N/A | N/A | 24660 | 24679 | ACCTGTAGGATCGGGCCAAG | 34 | 1397 |
| 1128531 | N/A | N/A | 24868 | 24887 | CGCCTGTACCAAGTGCCAGG | 0 | 1398 |
| 1128563 | N/A | N/A | 24942 | 24961 | GAGAGACCCACTTAGCTTCC | 30 | 1399 |
| 1128595 | N/A | N/A | 25473 | 25492 | GATTTGCCAGGAGAGGATAG | 11 | 1400 |
| 1128627 | N/A | N/A | 25576 | 25595 | CACCATCGCGATCGGCCTGG | 26 | 1401 |
| 1128659 | N/A | N/A | 25989 | 26008 | TCCACCCCTTATGTAGATAA | 23 | 1402 |
| 1128691 | N/A | N/A | 26363 | 26382 | CGGCGATGTGGGTGCAAACC | 0 | 1403 |

TABLE 20
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126259 | 54 | 73 | 2701 | 2720 | CCTAGGCAGAAGGAGGCCGC | 14 | 1404 |
| 1126291 | 168 | 187 | 2815 | 2834 | TCTAGGGAATGCACCAGGTA | 77 | 1405 |
| 1126323 | 245 | 264 | 2892 | 2911 | GACAAAGTGCGGTTTAAAGG | 65 | 1406 |
| 1126355 | 424 | 443 | 4582 | 4601 | ACGGCCCCACCAGGAAGTAG | 35 | 1407 |
| 1126387 | 772 | 791 | 10004 | 10023 | CCAACCACTCATGGAAGTGA | 24 | 1408 |
| 1126419 | 878 | 897 | 10110 | 10129 | TCCACGGCACCGGCACACAG | 1 | 1409 |
| 1126451 | 1012 | 1031 | 10488 | 10507 | TGGCGGTGATCTGGGACACA | 93 | 1410 |
| 1126483 | 1213 | 1232 | 13656 | 13675 | GGCCGGCGATAAAGAAGTAT | 0 | 1411 |
| 1126515 | 1332 | 1351 | 14393 | 14412 | CGCTGGCATGATGAAGAAGG | 35 | 1412 |
| 1126547 | 1474 | 1493 | 18002 | 18021 | TGTTCATGTCGGGAAGGCTC | 25 | 1413 |
| 1126579 | 1625 | 1644 | 21343 | 21362 | CTATTGAAGAGGCCGATTCG | 10 | 1414 |
| 1126611 | 1765 | 1784 | 21763 | 21782 | TGTAGCCCCAAGGCTCATAG | 7 | 1415 |
| 1126643 | 1923 | 1942 | 25328 | 25347 | GAGCTGCGAGCAGGAATCAT | 3 | 1416 |
| 1126675 | 2051 | 2070 | 26146 | 26165 | TGGCGCGCAGACATATAGTA | 17 | 1417 |
| 1126707 | 2233 | 2252 | 26491 | 26510 | CTTCCTCCAGCGGCCCGTTC | 30 | 1418 |
| 1126739 | 2311 | 2330 | 26569 | 26588 | CTCGGCGCGGCCACTCTGGT | 19 | 1419 |
| 1126771 | 2576 | 2595 | 26834 | 26853 | GAGTGTTTGGCGGACTGGGT | 60 | 1420 |
| 1126803 | 2810 | 2829 | 27068 | 27087 | AGAATTTGTAAGCCACACGG | 89 | 1421 |
| 1126835 | 2911 | 2930 | 27169 | 27188 | CGGCTCTGGACTTGATCGCC | 35 | 1422 |
| 1126867 | 3062 | 3081 | 27320 | 27339 | GGGATGACAGGAGCCGGATG | 61 | 1423 |
| 1126899 | 3362 | 3381 | 27620 | 27639 | GGGATCAGTATGCAGTAACG | 90 | 1424 |
| 1126931 | 3570 | 3589 | 27828 | 27847 | TCATCTCATCTCCGGACACA | 90 | 217 |
| 1126963 | N/A | N/A | 3079 | 3098 | CCTAGTAGCCCCGTCCTCCT | 22 | 1425 |
| 1126995 | N/A | N/A | 3272 | 3291 | GTATCTAGTCTCTCGGAAGC | 55 | 1426 |
| 1127027 | N/A | N/A | 3352 | 3371 | TAGGTCTGAAACCCCGGCAG | 55 | 1427 |
| 1127059 | N/A | N/A | 3431 | 3450 | GCCGCTCAGGCCTCCTATCT | 22 | 1428 |
| 1127091 | N/A | N/A | 3577 | 3596 | ACGCATGTGTCCCAGGCCAT | 80 | 373 |
| 1127123 | N/A | N/A | 3846 | 3865 | CGATACCCGGCTGGGTGTTC | 36 | 1429 |
| 1127155 | N/A | N/A | 4132 | 4151 | ACTCAGGTTCCCGAGTTCCC | 61 | 1430 |
| 1127187 | N/A | N/A | 4373 | 4392 | ACTGGGAGCCCATAGTTTGG | 36 | 1431 |
| 1127219 | N/A | N/A | 4826 | 4845 | GGGTGTCCTATCACGCCTCC | 28 | 1432 |
| 1127251 | N/A | N/A | 4982 | 5001 | GACCAGGCTGTCTTTCGATC | 89 | 453 |
| 1127283 | N/A | N/A | 5419 | 5438 | ACAATGACAATCACCGCCCA | 0 | 1433 |
| 1127315 | N/A | N/A | 5891 | 5910 | ACAGTCGGACCTGAATGGAT | 64 | 1434 |
| 1127347 | N/A | N/A | 6060 | 6079 | CGCCGACCTGGCTTTTTGCT | 22 | 1435 |
| 1127379 | N/A | N/A | 6266 | 6285 | CCCTCGGCCACGCCACACAT | 0 | 1436 |
| 1127411 | N/A | N/A | 7670 | 7689 | TGATGCTAATTCCTTCCACG | 87 | 1437 |
| 1127443 | N/A | N/A | 8509 | 8528 | AGATGTCTATAGACTTAAGC | 34 | 1438 |
| 1127475 | N/A | N/A | 8902 | 8921 | GATTAAAGGACCCTATGACT | 22 | 1439 |
| 1127507 | N/A | N/A | 8983 | 9002 | CTAGAGTAAATGAAGTTGGC | 86 | 1440 |
| 1127539 | N/A | N/A | 9541 | 9560 | GCAGCCAGTTCCTCAATTAT | 18 | 1441 |
| 1127571 | N/A | N/A | 9618 | 9637 | AAGACCCCTTGTCTCAAGGG | 6 | 1442 |
| 1127603 | N/A | N/A | 9709 | 9728 | GAGGATTTTTTGGCCAACCA | 95 | 1443 |
| 1127635 | N/A | N/A | 9858 | 9877 | GTCAATCTGTCACCACTACT | 55 | 1444 |
| 1127667 | N/A | N/A | 9925 | 9944 | CCACTGTCTCCACGAGTGTT | 0 | 1445 |
| 1127699 | N/A | N/A | 10365 | 10384 | GGGACAGCAGTCCGGTTAGA | 9 | 1446 |
| 1127731 | N/A | N/A | 12010 | 12029 | CCAGCTTAGGACTTAGGATG | 51 | 1447 |
| 1127763 | N/A | N/A | 12288 | 12307 | TGAGAACGGATACTAAGGGC | 74 | 1448 |
| 1127795 | N/A | N/A | 12401 | 12420 | GTATATCAACGAAAAACTAC | 0 | 1449 |
| 1127827 | N/A | N/A | 12684 | 12703 | TTGGTGACACACCTACCCTG | 38 | 1450 |
| 1127859 | N/A | N/A | 13391 | 13410 | GCCGAATACCCAGGTGCCCC | 4 | 1451 |
| 1127891 | N/A | N/A | 13590 | 13609 | GCACGGTAAAGCCCAAAGCC | 10 | 1452 |
| 1127923 | N/A | N/A | 14231 | 14250 | TTATAGGACAGTCCCCACCT | 29 | 1453 |
| 1127954 | N/A | N/A | 15654 | 15673 | CCGTCTACAGGATTTTCTAG | 96 | 83 |
| 1127956 | N/A | N/A | 15656 | 15675 | TTCCGTCTACAGGATTTTCT | 78 | 1454 |
| 1127988 | N/A | N/A | 15868 | 15887 | TCTATGAGACATGTCACTCC | 29 | 1455 |
| 1128020 | N/A | N/A | 16829 | 16848 | TGGCAATGCTAAGCCCCATT | 71 | 1456 |
| 1128052 | N/A | N/A | 17141 | 17160 | AGGTTTCTAGGATAGCAAGG | 82 | 1457 |
| 1128084 | N/A | N/A | 17886 | 17905 | ACACCAGTAAGGATTCATAA | 24 | 1458 |
| 1128116 | N/A | N/A | 18213 | 18232 | CGTGCCCTTCTGTGGACCTC | 24 | 1459 |
| 1128148 | N/A | N/A | 19200 | 19219 | TATGTGTCCGCCTCGCTCTG | 35 | 1460 |
| 1128180 | N/A | N/A | 19425 | 19444 | CGCAGCTGCTGAGAGTTTGC | 7 | 1461 |
| 1128212 | N/A | N/A | 19568 | 19587 | GTTTTTTGGGCCCCTAGACT | 24 | 1462 |
| 1128244 | N/A | N/A | 19797 | 19816 | CTGCTTTAGGGACTATTGGA | 45 | 1463 |
| 1128276 | N/A | N/A | 19918 | 19937 | TAGCAACTGTTCCATCATAG | 72 | 1464 |
| 1128308 | N/A | N/A | 21114 | 21133 | TCACCAGGAGTTGTGGATTC | 18 | 1465 |
| 1128340 | N/A | N/A | 21624 | 21643 | GACTCAGCCCAAGTGCCTTA | 11 | 1466 |
| 1128372 | N/A | N/A | 21825 | 21844 | ATGCTAGCTCTGACTAGGAT | 23 | 1467 |
| 1128404 | N/A | N/A | 21929 | 21948 | TGTATCACAAAAGCACGCGC | 9 | 1468 |
| 1128436 | N/A | N/A | 22095 | 22114 | CGTCTTACGAGGAGACTTGG | 19 | 1469 |
| 1128468 | N/A | N/A | 24578 | 24597 | GGACTTAGTCTATTTTGATG | 19 | 1470 |
| 1128500 | N/A | N/A | 24661 | 24680 | AACCTGTAGGATCGGGCCAA | 30 | 1471 |
| 1128532 | N/A | N/A | 24869 | 24888 | GCGCCTGTACCAAGTGCCAG | 4 | 1472 |
| 1128564 | N/A | N/A | 24944 | 24963 | GAGAGAGACCCACTTAGCTT | 15 | 1473 |
| 1128596 | N/A | N/A | 25502 | 25521 | GCACCAAAGACCCCTAGCCA | 18 | 1474 |
| 1128628 | N/A | N/A | 25577 | 25596 | CCACCATCGCGATCGGCCTG | 31 | 1475 |
| 1128660 | N/A | N/A | 25990 | 26009 | CTCCACCCCTTATGTAGATA | 33 | 1476 |
| 1128692 | N/A | N/A | 26364 | 26383 | ACGGCGATGTGGGTGCAAAC | 23 | 1477 |

TABLE 21
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126260 | 55 | 74 | 2702 | 2721 | ACCTAGGCAGAAGGAGGCCG | 0 | 1478 |
| 1126292 | 169 | 188 | 2816 | 2835 | GTCTAGGGAATGCACCAGGT | 89 | 408 |
| 1126324 | 246 | 265 | 2893 | 2912 | GGACAAAGTGCGGTTTAAAG | 65 | 1479 |
| 1126356 | 429 | 448 | 4587 | 4606 | CGTGTACGGCCCCACCAGGA | 61 | 1480 |
| 1126388 | 774 | 793 | 10006 | 10025 | TGCCAACCACTCATGGAAGT | 23 | 1481 |
| 1126420 | 880 | 899 | 10112 | 10131 | AGTCCACGGCACCGGCACAC | 14 | 1482 |
| 1126452 | 1013 | 1032 | 10489 | 10508 | ATGGCGGTGATCTGGGACAC | 87† | 1483 |
| 1126484 | 1214 | 1233 | 13657 | 13676 | CGGCCGGCGATAAAGAAGTA | 0 | 1484 |
| 1126516 | 1333 | 1352 | 14394 | 14413 | GCGCTGGCATGATGAAGAAG | 15 | 1485 |
| 1126548 | 1475 | 1494 | 18003 | 18022 | TTGTTCATGTCGGGAAGGCT | 22 | 1486 |
| 1126580 | 1626 | 1645 | 21344 | 21363 | GCTATTGAAGAGGCCGATTC | 17 | 1487 |
| 1126612 | 1766 | 1785 | 21764 | 21783 | GTGTAGCCCCAAGGCTCATA | 30 | 1488 |
| 1126644 | 1924 | 1943 | 25329 | 25348 | TGAGCTGCGAGCAGGAATCA | 18 | 1489 |
| 1126676 | 2053 | 2072 | 26148 | 26167 | TGTGGCGCGCAGACATATAG | 29 | 1490 |
| 1126708 | 2234 | 2253 | 26492 | 26511 | TCTTCCTCCAGCGGCCCGTT | 20 | 1491 |
| 1126740 | 2313 | 2332 | 26571 | 26590 | CGCTCGGCGCGGCCACTCTG | 31 | 1492 |
| 1126772 | 2578 | 2597 | 26836 | 26855 | TGGAGTGTTTGGCGGACTGG | 30 | 1493 |
| 1126804 | 2811 | 2830 | 27069 | 27088 | TAGAATTTGTAAGCCACACG | 79 | 1494 |
| 1126836 | 2912 | 2931 | 27170 | 27189 | CCGGCTCTGGACTTGATCGC | 26 | 1495 |
| 1126868 | 3080 | 3099 | 27338 | 27357 | TGCTTGGCCAAAGTGTAGGG | 60 | 1496 |
| 1126900 | 3363 | 3382 | 27621 | 27640 | AGGGATCAGTATGCAGTAAC | 90 | 1497 |
| 1126932 | 3571 | 3590 | 27829 | 27848 | TTCATCTCATCTCCGGACAC | 73 | 1498 |
| 1126964 | N/A | N/A | 3080 | 3099 | CCCTAGTAGCCCCGTCCTCC | 45 | 1499 |
| 1126996 | N/A | N/A | 3273 | 3292 | GGTATCTAGTCTCTCGGAAG | 84 | 1500 |
| 1127028 | N/A | N/A | 3353 | 3372 | ATAGGTCTGAAACCCCGGCA | 62 | 1501 |
| 1127060 | N/A | N/A | 3432 | 3451 | CGCCGCTCAGGCCTCCTATC | 5 | 1502 |
| 1127092 | N/A | N/A | 3578 | 3597 | TACGCATGTGTCCCAGGCCA | 63 | 1503 |
| 1127124 | N/A | N/A | 3847 | 3866 | GCGATACCCGGCTGGGTGTT | 37 | 1504 |
| 1127156 | N/A | N/A | 4134 | 4153 | GGACTCAGGTTCCCGAGTTC | 8 | 1505 |
| 1127188 | N/A | N/A | 4374 | 4393 | GACTGGGAGCCCATAGTTTG | 13 | 1506 |
| 1127220 | N/A | N/A | 4827 | 4846 | TGGGTGTCCTATCACGCCTC | 12 | 1507 |
| 1127252 | N/A | N/A | 4983 | 5002 | TGACCAGGCTGTCTTTCGAT | 86 | 1508 |
| 1127284 | N/A | N/A | 5420 | 5439 | AACAATGACAATCACCGCCC | 14 | 1509 |
| 1127316 | N/A | N/A | 5892 | 5911 | CACAGTCGGACCTGAATGGA | 59 | 1510 |
| 1127348 | N/A | N/A | 6061 | 6080 | TCGCCGACCTGGCTTTTTGC | 28 | 1511 |
| 1127380 | N/A | N/A | 6268 | 6287 | CGCCCTCGGCCACGCCACAC | 36 | 1512 |
| 1127412 | N/A | N/A | 7803 | 7822 | TGTCCTGTGTCTATGCCCCC | 75 | 1513 |
| 1127444 | N/A | N/A | 8514 | 8533 | CAGGCAGATGTCTATAGACT | 82 | 1514 |
| 1127476 | N/A | N/A | 8903 | 8922 | GGATTAAAGGACCCTATGAC | 30 | 1515 |
| 1127508 | N/A | N/A | 8996 | 9015 | TATGCTTGGAAGACTAGAGT | 45 | 1516 |
| 1127540 | N/A | N/A | 9547 | 9566 | ATAGGAGCAGCCAGTTCCTC | 17 | 1517 |
| 1127572 | N/A | N/A | 9638 | 9657 | GGTCCTGGACATAGTTTCCA | 22 | 1518 |
| 1127604 | N/A | N/A | 9711 | 9730 | CTGAGGATTTTTTGGCCAAC | 81 | 1519 |
| 1127636 | N/A | N/A | 9859 | 9878 | GGTCAATCTGTCACCACTAC | 43 | 1520 |
| 1127668 | N/A | N/A | 9926 | 9945 | GCCACTGTCTCCACGAGTGT | 6 | 1521 |
| 1127700 | N/A | N/A | 10567 | 10586 | AGCTGCCTACCTCATTCACG | 7 | 1522 |
| 1127732 | N/A | N/A | 12011 | 12030 | CCCAGCTTAGGACTTAGGAT | 56 | 1523 |
| 1127764 | N/A | N/A | 12289 | 12308 | CTGAGAACGGATACTAAGGG | 83 | 1524 |
| 1127796 | N/A | N/A | 12443 | 12462 | CTTACAGGGACCCAATCATT | 8 | 1525 |
| 1127828 | N/A | N/A | 12686 | 12705 | CCTTGGTGACACACCTACCC | 0 | 1526 |
| 1127860 | N/A | N/A | 13392 | 13411 | GGCCGAATACCCAGGTGCCC | 2 | 1527 |
| 1127892 | N/A | N/A | 13591 | 13610 | GGCACGGTAAAGCCCAAAGC | 0 | 1528 |
| 1127924 | N/A | N/A | 14232 | 14251 | ATTATAGGACAGTCCCCACC | 27 | 1529 |
| 1127954 | N/A | N/A | 15654 | 15673 | CCGTCTACAGGATTTTCTAG | 96 | 83 |
| 1127957 | N/A | N/A | 15659 | 15678 | AAATTCCGTCTACAGGATTT | 37 | 1530 |
| 1127989 | N/A | N/A | 15869 | 15888 | CTCTATGAGACATGTCACTC | 45 | 1531 |
| 1128021 | N/A | N/A | 16845 | 16864 | GTACTTGTCCTGCAGATGGC | 92 | 1532 |
| 1128053 | N/A | N/A | 17142 | 17161 | TAGGTTTCTAGGATAGCAAG | 59 | 1533 |
| 1128085 | N/A | N/A | 17887 | 17906 | CACACCAGTAAGGATTCATA | 0 | 1534 |
| 1128117 | N/A | N/A | 18231 | 18250 | AAGTTGCACGAGTCAGCACG | 17 | 1535 |
| 1128149 | N/A | N/A | 19201 | 19220 | GTATGTGTCCGCCTCGCTCT | 25 | 1536 |
| 1128181 | N/A | N/A | 19428 | 19447 | AACCGCAGCTGCTGAGAGTT | 14 | 1537 |
| 1128213 | N/A | N/A | 19570 | 19589 | TAGTTTTTTGGGCCCCTAGA | 69 | 1538 |
| 1128245 | N/A | N/A | 19798 | 19817 | GCTGCTTTAGGGACTATTGG | 14 | 1539 |
| 1128277 | N/A | N/A | 19931 | 19950 | GGTGTATAGTTCTTAGCAAC | 59 | 1540 |
| 1128309 | N/A | N/A | 21115 | 21134 | ATCACCAGGAGTTGTGGATT | 18 | 1541 |
| 1128341 | N/A | N/A | 21655 | 21674 | GGTGGAAAATCACCTGGTAG | 0 | 1542 |
| 1128373 | N/A | N/A | 21828 | 21847 | GACATGCTAGCTCTGACTAG | 32 | 1543 |
| 1128405 | N/A | N/A | 21930 | 21949 | TTGTATCACAAAAGCACGCG | 34 | 1544 |
| 1128437 | N/A | N/A | 22120 | 22139 | AGGTGGAGTTGATGGGCACT | 29 | 1545 |
| 1128469 | N/A | N/A | 24579 | 24598 | CGGACTTAGTCTATTTTGAT | 28 | 1546 |
| 1128501 | N/A | N/A | 24662 | 24681 | CAACCTGTAGGATCGGGCCA | 34 | 1547 |
| 1128533 | N/A | N/A | 24885 | 24904 | ACACCGCCCTCTACTGGCGC | 0 | 1548 |
| 1128565 | N/A | N/A | 24945 | 24964 | GGAGAGAGACCCACTTAGCT | 20 | 1549 |
| 1128597 | N/A | N/A | 25503 | 25522 | TGCACCAAAGACCCCTAGCC | 15 | 1550 |
| 1128629 | N/A | N/A | 25578 | 25597 | GCCACCATCGCGATCGGCCT | 0 | 1551 |
| 1128661 | N/A | N/A | 25991 | 26010 | ACTCCACCCCTTATGTAGAT | 27 | 1552 |
| 1128693 | N/A | N/A | 26365 | 26384 | TACGGCGATGTGGGTGCAAA | 0 | 1553 |

TABLE 22
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126261 | 82 | 101 | 2729 | 2748 | ACCGCACCCCTGCCCCGAAG | 51 | 1554 |
| 1126293 | 170 | 189 | 2817 | 2836 | TGTCTAGGGAATGCACCAGG | 81 | 1555 |
| 1126325 | 247 | 266 | 2894 | 2913 | TGGACAAAGTGCGGTTTAAA | 69 | 1556 |
| 1126357 | 431 | 450 | 4589 | 4608 | TCCGTGTACGGCCCCACCAG | 32 | 1557 |
| 1126389 | 777 | 796 | 10009 | 10028 | GCCTGCCAACCACTCATGGA | 7 | 1558 |
| 1126421 | 881 | 900 | 10113 | 10132 | AAGTCCACGGCACCGGCACA | 18 | 1559 |
| 1126453 | 1016 | 1035 | 10492 | 10511 | TCGATGGCGGTGATCTGGGA | 71† | 1560 |
| 1126485 | 1217 | 1236 | 13660 | 13679 | TAGCGGCCGGCGATAAAGAA | 1 | 1561 |
| 1126517 | 1334 | 1353 | 14395 | 14414 | CGCGCTGGCATGATGAAGAA | 29 | 1562 |
| 1126549 | 1477 | 1496 | 18005 | 18024 | TCTTGTTCATGTCGGGAAGG | 20 | 1563 |
| 1126581 | 1627 | 1646 | 21345 | 21364 | TGCTATTGAAGAGGCCGATT | 30 | 1564 |
| 1126613 | 1767 | 1786 | 21765 | 21784 | TGTGTAGCCCCAAGGCTCAT | 16 | 1565 |
| 1126645 | 1959 | 1978 | 25364 | 25383 | CCGGCTCTGCTGACAGAAAC | 16 | 1566 |
| 1126677 | 2054 | 2073 | 26149 | 26168 | ATGTGGCGCGCAGACATATA | 0 | 1567 |
| 1126709 | 2235 | 2254 | 26493 | 26512 | GTCTTCCTCCAGCGGCCCGT | 9 | 1568 |
| 1126741 | 2314 | 2333 | 26572 | 26591 | ACGCTCGGCGCGGCCACTCT | 28 | 1569 |
| 1126773 | 2579 | 2598 | 26837 | 26856 | GTGGAGTGTTTGGCGGACTG | 63 | 1570 |
| 1126805 | 2827 | 2846 | 27085 | 27104 | CCTGCTCTGTATGCTGTAGA | 76 | 1571 |
| 1126837 | 2913 | 2932 | 27171 | 27190 | CCCGGCTCTGGACTTGATCG | 29 | 1572 |
| 1126869 | 3103 | 3122 | 27361 | 27380 | AGCCAAGTGGTTCTACCACC | 29 | 1573 |
| 1126901 | 3364 | 3383 | 27622 | 27641 | AAGGGATCAGTATGCAGTAA | 86 | 1574 |
| 1126933 | 3572 | 3591 | 27830 | 27849 | TTTCATCTCATCTCCGGACA | 75 | 1575 |
| 1126965 | N/A | N/A | 3081 | 3100 | CCCCTAGTAGCCCCGTCCTC | 15 | 1576 |
| 1126997 | N/A | N/A | 3274 | 3293 | AGGTATCTAGTCTCTCGGAA | 78 | 1577 |
| 1127029 | N/A | N/A | 3354 | 3373 | CATAGGTCTGAAACCCCGGC | 55 | 1578 |
| 1127061 | N/A | N/A | 3433 | 3452 | CCGCCGCTCAGGCCTCCTAT | 0 | 1579 |
| 1127093 | N/A | N/A | 3582 | 3601 | CCAGTACGCATGTGTCCCAG | 90 | 1580 |
| 1127125 | N/A | N/A | 3848 | 3867 | GGCGATACCCGGCTGGGTGT | 9 | 1581 |
| 1127157 | N/A | N/A | 4139 | 4158 | TACCCGGACTCAGGTTCCCG | 35 | 1582 |
| 1127189 | N/A | N/A | 4375 | 4394 | GGACTGGGAGCCCATAGTTT | 4 | 1583 |
| 1127221 | N/A | N/A | 4828 | 4847 | CTGGGTGTCCTATCACGCCT | 32 | 1584 |
| 1127253 | N/A | N/A | 4984 | 5003 | TTGACCAGGCTGTCTTTCGA | 77 | 1585 |
| 1127285 | N/A | N/A | 5421 | 5440 | TAACAATGACAATCACCGCC | 0 | 1586 |
| 1127317 | N/A | N/A | 5893 | 5912 | ACACAGTCGGACCTGAATGG | 51 | 1587 |
| 1127349 | N/A | N/A | 6062 | 6081 | CTCGCCGACCTGGCTTTTTG | 27 | 1588 |
| 1127381 | N/A | N/A | 6269 | 6288 | CCGCCCTCGGCCACGCCACA | 33 | 1589 |
| 1127413 | N/A | N/A | 7804 | 7823 | ATGTCCTGTGTCTATGCCCC | 84 | 1590 |
| 1127445 | N/A | N/A | 8515 | 8534 | GCAGGCAGATGTCTATAGAC | 77 | 1591 |
| 1127477 | N/A | N/A | 8905 | 8924 | TTGGATTAAAGGACCCTATG | 4 | 1592 |
| 1127509 | N/A | N/A | 8997 | 9016 | CTATGCTTGGAAGACTAGAG | 38 | 1593 |
| 1127541 | N/A | N/A | 9548 | 9567 | CATAGGAGCAGCCAGTTCCT | 21 | 1594 |
| 1127573 | N/A | N/A | 9669 | 9688 | GATTGGACAGACTGAAGTTT | 36 | 1595 |
| 1127605 | N/A | N/A | 9793 | 9812 | GGACCTGTAAATCCCTGAGG | 0 | 1596 |
| 1127637 | N/A | N/A | 9860 | 9879 | AGGTCAATCTGTCACCACTA | 27 | 1597 |
| 1127669 | N/A | N/A | 10133 | 10152 | CGTTCTCCAGGTTGTTGTAG | 78 | 1598 |
| 1127701 | N/A | N/A | 10574 | 10593 | CATAGACAGCTGCCTACCTC | 26 | 1599 |
| 1127733 | N/A | N/A | 12012 | 12031 | CCCCAGCTTAGGACTTAGGA | 31 | 1600 |
| 1127765 | N/A | N/A | 12290 | 12309 | ACTGAGAACGGATACTAAGG | 56 | 1601 |
| 1127797 | N/A | N/A | 12444 | 12463 | TCTTACAGGGACCCAATCAT | 9 | 1602 |
| 1127829 | N/A | N/A | 12687 | 12706 | CCCTTGGTGACACACCTACC | 25 | 1603 |
| 1127861 | N/A | N/A | 13393 | 13412 | AGGCCGAATACCCAGGTGCC | 8 | 1604 |
| 1127893 | N/A | N/A | 13592 | 13611 | AGGCACGGTAAAGCCCAAAG | 9 | 1605 |
| 1127925 | N/A | N/A | 14236 | 14255 | GAGAATTATAGGACAGTCCC | 27 | 1606 |
| 1127954 | N/A | N/A | 15654 | 15673 | CCGTCTACAGGATTTTCTAG | 96 | 83 |
| 1127958 | N/A | N/A | 15660 | 15679 | TAAATTCCGTCTACAGGATT | 30 | 1607 |
| 1127990 | N/A | N/A | 15878 | 15897 | CTACCCCTTCTCTATGAGAC | 57 | 1608 |
| 1128022 | N/A | N/A | 16849 | 16868 | ACTTGTACTTGTCCTGCAGA | 32 | 1609 |
| 1128054 | N/A | N/A | 17145 | 17164 | CCTTAGGTTTCTAGGATAGC | 66 | 1610 |
| 1128086 | N/A | N/A | 17890 | 17909 | ACTCACACCAGTAAGGATTC | 0 | 1611 |
| 1128118 | N/A | N/A | 18232 | 18251 | GAAGTTGCACGAGTCAGCAC | 28 | 1612 |
| 1128150 | N/A | N/A | 19202 | 19221 | TGTATGTGTCCGCCTCGCTC | 22 | 1613 |
| 1128182 | N/A | N/A | 19429 | 19448 | AAACCGCAGCTGCTGAGAGT | 21 | 1614 |
| 1128214 | N/A | N/A | 19572 | 19591 | TGTAGTTTTTTGGGCCCCTA | 15 | 1615 |
| 1128246 | N/A | N/A | 19799 | 19818 | GGCTGCTTTAGGGACTATTG | 35 | 1616 |
| 1128278 | N/A | N/A | 19960 | 19979 | GTGTGATAGGAAGTCCTGAG | 78 | 1617 |
| 1128310 | N/A | N/A | 21116 | 21135 | GATCACCAGGAGTTGTGGAT | 39 | 1618 |
| 1128342 | N/A | N/A | 21771 | 21790 | ACCCGGTGTGTAGCCCCAAG | 0 | 1619 |
| 1128374 | N/A | N/A | 21829 | 21848 | AGACATGCTAGCTCTGACTA | 66 | 1620 |
| 1128406 | N/A | N/A | 21941 | 21960 | TACATGAGCTATTGTATCAC | 38 | 1621 |
| 1128438 | N/A | N/A | 22147 | 22166 | ACTGGCTAATGTGAACCAAC | 31 | 1622 |
| 1128470 | N/A | N/A | 24583 | 24602 | TGTACGGACTTAGTCTATTT | 38 | 1623 |
| 1128502 | N/A | N/A | 24663 | 24682 | ACAACCTGTAGGATCGGGCC | 24 | 1624 |
| 1128534 | N/A | N/A | 24886 | 24905 | AACACCGCCCTCTACTGGCG | 11 | 1625 |
| 1128566 | N/A | N/A | 24966 | 24985 | GCACTCAGCTGCGGGAAGGC | 7 | 1626 |
| 1128598 | N/A | N/A | 25504 | 25523 | CTGCACCAAAGACCCCTAGC | 0 | 1627 |
| 1128630 | N/A | N/A | 25580 | 25599 | GAGCCACCATCGCGATCGGC | 25 | 1628 |
| 1128662 | N/A | N/A | 25993 | 26012 | TGACTCCACCCCTTATGTAG | 18 | 1629 |
| 1128694 | N/A | N/A | 26366 | 26385 | ATACGGCGATGTGGGTGCAA | 0 | 1630 |

TABLE 23
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126262 | 91 | 110 | 2738 | 2757 | TATTGCAAGACCGCACCCCT | 1 | 1631 |
| 1126294 | 171 | 190 | 2818 | 2837 | GTGTCTAGGGAATGCACCAG | 74 | 1632 |
| 1126326 | 248 | 267 | 2895 | 2914 | ATGGACAAAGTGCGGTTTAA | 67 | 1633 |
| 1126358 | 434 | 453 | 4592 | 4611 | TGCTCCGTGTACGGCCCCAC | 26 | 1634 |
| 1126390 | 779 | 798 | 10011 | 10030 | ACGCCTGCCAACCACTCATG | 15 | 1635 |
| 1126422 | 882 | 901 | 10114 | 10133 | GAAGTCCACGGCACCGGCAC | 0 | 1636 |
| 1126454 | 1017 | 1036 | 10493 | 10512 | CTCGATGGCGGTGATCTGGG | 79† | 1637 |
| 1126486 | 1218 | 1237 | 13661 | 13680 | ATAGCGGCCGGCGATAAAGA | 14 | 1638 |
| 1126518 | 1335 | 1354 | 14396 | 14415 | CCGCGCTGGCATGATGAAGA | 15 | 1639 |
| 1126550 | 1478 | 1497 | 18006 | 18025 | ATCTTGTTCATGTCGGGAAG | 68 | 1640 |
| 1126582 | 1628 | 1647 | 21346 | 21365 | CTGCTATTGAAGAGGCCGAT | 10 | 1641 |
| 1126614 | 1769 | 1788 | 21767 | 21786 | GGTGTGTAGCCCCAAGGCTC | 18 | 1642 |
| 1126646 | 1960 | 1979 | 25365 | 25384 | GCCGGCTCTGCTGACAGAAA | 19 | 1643 |
| 1126678 | 2055 | 2074 | 26150 | 26169 | CATGTGGCGCGCAGACATAT | 16 | 1644 |
| 1126710 | 2240 | 2259 | 26498 | 26517 | TCGCCGTCTTCCTCCAGCGG | 17 | 1645 |
| 1126742 | 2345 | 2364 | 26603 | 26622 | CGCTTGCTGCCGCTGGTGGA | 25 | 1646 |
| 1126774 | 2580 | 2599 | 26838 | 26857 | GGTGGAGTGTTTGGCGGACT | 60 | 1647 |
| 1126806 | 2829 | 2848 | 27087 | 27106 | GGCCTGCTCTGTATGCTGTA | 42 | 1648 |
| 1126838 | 2915 | 2934 | 27173 | 27192 | GCCCCGGCTCTGGACTTGAT | 23 | 1649 |
| 1126870 | 3104 | 3123 | 27362 | 27381 | CAGCCAAGTGGTTCTACCAC | 9 | 1650 |
| 1126902 | 3366 | 3385 | 27624 | 27643 | GAAAGGGATCAGTATGCAGT | 87 | 1651 |
| 1126934 | N/A | N/A | 2990 | 3009 | CACCCTTGTTAGCCACCTCC | 10 | 1652 |
| 1126966 | N/A | N/A | 3082 | 3101 | TCCCCTAGTAGCCCCGTCCT | 36 | 1653 |
| 1126998 | N/A | N/A | 3275 | 3294 | CAGGTATCTAGTCTCTCGGA | 84 | 1654 |
| 1127030 | N/A | N/A | 3355 | 3374 | TCATAGGTCTGAAACCCCGG | 39 | 1655 |
| 1127062 | N/A | N/A | 3438 | 3457 | TCCCACCGCCGCTCAGGCCT | 10 | 1656 |
| 1127094 | N/A | N/A | 3583 | 3602 | TCCAGTACGCATGTGTCCCA | 83 | 1657 |
| 1127126 | N/A | N/A | 3849 | 3868 | TGGCGATACCCGGCTGGGTG | 18 | 1658 |
| 1127158 | N/A | N/A | 4140 | 4159 | CTACCCGGACTCAGGTTCCC | 48 | 1659 |
| 1127190 | N/A | N/A | 4465 | 4484 | CCGAGGTCCATAGTTCTGGG | 45 | 1660 |
| 1127222 | N/A | N/A | 4829 | 4848 | TCTGGGTGTCCTATCACGCC | 55 | 1661 |
| 1127254 | N/A | N/A | 5303 | 5322 | GCATGTCCATTAGCCAAGGA | 53 | 1662 |
| 1127286 | N/A | N/A | 5455 | 5474 | GCTTGGCTGTTAGTAACAAT | 82 | 1663 |
| 1127318 | N/A | N/A | 5894 | 5913 | GACACAGTCGGACCTGAATG | 65 | 1664 |
| 1127350 | N/A | N/A | 6063 | 6082 | CCTCGCCGACCTGGCTTTTT | 0 | 1665 |
| 1127382 | N/A | N/A | 6984 | 7003 | CTATCCATAGGCCCATTTTG | 57 | 1666 |
| 1127414 | N/A | N/A | 7809 | 7828 | GTCACATGTCCTGTGTCTAT | 94 | 1667 |
| 1127446 | N/A | N/A | 8531 | 8550 | CGTGGTTCTCCCATTTGCAG | 49 | 1668 |
| 1127478 | N/A | N/A | 8906 | 8925 | CTTGGATTAAAGGACCCTAT | 35 | 1669 |
| 1127510 | N/A | N/A | 8998 | 9017 | CCTATGCTTGGAAGACTAGA | 10 | 1670 |
| 1127542 | N/A | N/A | 9549 | 9568 | CCATAGGAGCAGCCAGTTCC | 5 | 1671 |
| 1127574 | N/A | N/A | 9671 | 9690 | TAGATTGGACAGACTGAAGT | 12 | 1672 |
| 1127606 | N/A | N/A | 9794 | 9813 | GGGACCTGTAAATCCCTGAG | 30 | 1673 |
| 1127638 | N/A | N/A | 9862 | 9881 | AGAGGTCAATCTGTCACCAC | 35 | 1674 |
| 1127670 | N/A | N/A | 10154 | 10173 | ATCTGCCACGGTCCCAGCTC | 20 | 1675 |
| 1127702 | N/A | N/A | 10575 | 10594 | GCATAGACAGCTGCCTACCT | 49 | 1676 |
| 1127734 | N/A | N/A | 12013 | 12032 | TCCCCAGCTTAGGACTTAGG | 39 | 1677 |
| 1127766 | N/A | N/A | 12291 | 12310 | CACTGAGAACGGATACTAAG | 45 | 1678 |
| 1127798 | N/A | N/A | 12446 | 12465 | CTTCTTACAGGGACCCAATC | 22 | 1679 |
| 1127830 | N/A | N/A | 12688 | 12707 | TCCCTTGGTGACACACCTAC | 10 | 1680 |
| 1127862 | N/A | N/A | 13394 | 13413 | GAGGCCGAATACCCAGGTGC | 22 | 1681 |
| 1127894 | N/A | N/A | 13593 | 13612 | AAGGCACGGTAAAGCCCAAA | 13 | 1682 |
| 1127926 | N/A | N/A | 14245 | 14264 | CCTGTCAAGGAGAATTATAG | 28 | 1683 |
| 1127954 | N/A | N/A | 15654 | 15673 | CCGTCTACAGGATTTTCTAG | 94 | 83 |
| 1127959 | N/A | N/A | 15661 | 15680 | GTAAATTCCGTCTACAGGAT | 74 | 1684 |
| 1127991 | N/A | N/A | 16565 | 16584 | TCGGCCACTTTCTAGGTTCA | 71 | 1685 |
| 1128023 | N/A | N/A | 16850 | 16869 | GACTTGTACTTGTCCTGCAG | 22 | 1686 |
| 1128055 | N/A | N/A | 17146 | 17165 | CCCTTAGGTTTCTAGGATAG | 44 | 1687 |
| 1128087 | N/A | N/A | 17892 | 17911 | GCACTCACACCAGTAAGGAT | 0 | 1688 |
| 1128119 | N/A | N/A | 18233 | 18252 | TGAAGTTGCACGAGTCAGCA | 22 | 1689 |
| 1128151 | N/A | N/A | 19203 | 19222 | GTGTATGTGTCCGCCTCGCT | 41 | 1690 |
| 1128183 | N/A | N/A | 19494 | 19513 | GGGATCTAAAGCAGCTACAA | 5 | 1691 |
| 1128215 | N/A | N/A | 19575 | 19594 | AGTTGTAGTTTTTTGGGCCC | 0 | 1692 |
| 1128247 | N/A | N/A | 19808 | 19827 | AATGAGGTGGGCTGCTTTAG | 28 | 1693 |
| 1128279 | N/A | N/A | 19961 | 19980 | AGTGTGATAGGAAGTCCTGA | 67 | 1694 |
| 1128311 | N/A | N/A | 21120 | 21139 | AAGTGATCACCAGGAGTTGT | 16 | 1695 |
| 1128343 | N/A | N/A | 21772 | 21791 | CACCCGGTGTGTAGCCCCAA | 0 | 1696 |
| 1128375 | N/A | N/A | 21831 | 21850 | CAAGACATGCTAGCTCTGAC | 28 | 1697 |
| 1128407 | N/A | N/A | 21980 | 21999 | GTTCACTAGTCCTCAAAACC | 24 | 1698 |
| 1128439 | N/A | N/A | 22148 | 22167 | CACTGGCTAATGTGAACCAA | 28 | 1699 |
| 1128471 | N/A | N/A | 24584 | 24603 | TTGTACGGACTTAGTCTATT | 20 | 1700 |
| 1128503 | N/A | N/A | 24664 | 24683 | CACAACCTGTAGGATCGGGC | 58 | 1701 |
| 1128535 | N/A | N/A | 24893 | 24912 | ACGCCTCAACACCGCCCTCT | 13 | 1702 |
| 1128567 | N/A | N/A | 24967 | 24986 | TGCACTCAGCTGCGGGAAGG | 0 | 1703 |
| 1128599 | N/A | N/A | 25505 | 25524 | CCTGCACCAAAGACCCCTAG | 24 | 1704 |
| 1128631 | N/A | N/A | 25581 | 25600 | TGAGCCACCATCGCGATCGG | 5 | 1705 |
| 1128663 | N/A | N/A | 25994 | 26013 | CTGACTCCACCCCTTATGTA | 23 | 1706 |
| 1128695 | N/A | N/A | 26367 | 26386 | CATACGGCGATGTGGGTGCA | 0 | 1707 |

TABLE 24
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126263 | 92 | 111 | 2739 | 2758 | CTATTGCAAGACCGCACCCC | 2 | 1708 |
| 1126295 | 172 | 191 | 2819 | 2838 | GGTGTCTAGGGAATGCACCA | 0 | 1709 |
| 1126327 | 249 | 268 | 2896 | 2915 | CATGGACAAAGTGCGGTTTA | 46 | 1710 |
| 1126359 | 435 | 454 | 4593 | 4612 | CTGCTCCGTGTACGGCCCCA | 9 | 1711 |
| 1126391 | 801 | 820 | 10033 | 10052 | GGCACGACACAGGCAGAGTC | 15 | 1712 |
| 1126423 | 883 | 902 | 10115 | 10134 | AGAAGTCCACGGCACCGGCA | 26 | 1713 |
| 1126455 | 1019 | 1038 | 10495 | 10514 | GCCTCGATGGCGGTGATCTG | 82† | 1714 |
| 1126487 | 1221 | 1240 | 13664 | 13683 | CTCATAGCGGCCGGCGATAA | 18 | 1715 |
| 1126519 | 1337 | 1356 | 14398 | 14417 | GTCCGCGCTGGCATGATGAA | 1 | 1716 |
| 1126551 | 1479 | 1498 | 18007 | 18026 | CATCTTGTTCATGTCGGGAA | 30 | 1717 |
| 1126583 | 1629 | 1648 | 21347 | 21366 | ACTGCTATTGAAGAGGCCGA | 0 | 1718 |
| 1126615 | 1770 | 1789 | 21768 | 21787 | CGGTGTGTAGCCCCAAGGCT | 4 | 1719 |
| 1126647 | 1962 | 1981 | 25367 | 25386 | CCGCCGGCTCTGCTGACAGA | 0 | 1720 |
| 1126679 | 2056 | 2075 | 26151 | 26170 | CCATGTGGCGCGCAGACATA | 2 | 1721 |
| 1126711 | 2275 | 2294 | 26533 | 26552 | GGTCCTTGGCGGCCTCCTCG | 0 | 1722 |
| 1126743 | 2348 | 2367 | 26606 | 26625 | TTGCGCTTGCTGCCGCTGGT | 8 | 1723 |
| 1126775 | 2633 | 2652 | 26891 | 26910 | GCACGGCTTTGTGGATTCTG | 91 | 1724 |
| 1126807 | 2851 | 2870 | 27109 | 27128 | CCCGCATGCCGGGCCTGAGC | 16 | 1725 |
| 1126839 | 2916 | 2935 | 27174 | 27193 | TGCCCCGGCTCTGGACTTGA | 22 | 1726 |
| 1126871 | 3106 | 3125 | 27364 | 27383 | AGCAGCCAAGTGGTTCTACC | 50 | 1727 |
| 1126903 | 3402 | 3421 | 27660 | 27679 | GTTCTAGAACCCAGTGACCT | 11 | 1728 |
| 1126935 | N/A | N/A | 2991 | 3010 | TCACCCTTGTTAGCCACCTC | 40 | 1729 |
| 1126967 | N/A | N/A | 3085 | 3104 | GGTTCCCCTAGTAGCCCCGT | 35 | 1730 |
| 1126999 | N/A | N/A | 3276 | 3295 | CCAGGTATCTAGTCTCTCGG | 84 | 1731 |
| 1127031 | N/A | N/A | 3356 | 3375 | GTCATAGGTCTGAAACCCCG | 41 | 1732 |
| 1127063 | N/A | N/A | 3443 | 3462 | GGTCCTCCCACCGCCGCTCA | 28 | 1733 |
| 1127095 | N/A | N/A | 3584 | 3603 | TTCCAGTACGCATGTGTCCC | 76 | 1734 |
| 1127127 | N/A | N/A | 3850 | 3869 | CTGGCGATACCCGGCTGGGT | 8 | 1735 |
| 1127159 | N/A | N/A | 4141 | 4160 | ACTACCCGGACTCAGGTTCC | 13 | 1736 |
| 1127191 | N/A | N/A | 4467 | 4486 | GGCCGAGGTCCATAGTTCTG | 0 | 1737 |
| 1127223 | N/A | N/A | 4830 | 4849 | GTCTGGGTGTCCTATCACGC | 62 | 1738 |
| 1127255 | N/A | N/A | 5304 | 5323 | AGCATGTCCATTAGCCAAGG | 72 | 1739 |
| 1127287 | N/A | N/A | 5457 | 5476 | AAGCTTGGCTGTTAGTAACA | 68 | 1740 |
| 1127319 | N/A | N/A | 5895 | 5914 | GGACACAGTCGGACCTGAAT | 35 | 1741 |
| 1127351 | N/A | N/A | 6064 | 6083 | TCCTCGCCGACCTGGCTTTT | 12 | 1742 |
| 1127383 | N/A | N/A | 6986 | 7005 | CTCTATCCATAGGCCCATTT | 68 | 1743 |
| 1127415 | N/A | N/A | 7825 | 7844 | GCTTATAACCACATATGTCA | 74 | 1744 |
| 1127447 | N/A | N/A | 8532 | 8551 | CCGTGGTTCTCCCATTTGCA | 69 | 1745 |
| 1127479 | N/A | N/A | 8909 | 8928 | ATCCTTGGATTAAAGGACCC | 0 | 1746 |
| 1127511 | N/A | N/A | 8999 | 9018 | CCCTATGCTTGGAAGACTAG | 0 | 1747 |
| 1127543 | N/A | N/A | 9551 | 9570 | TGCCATAGGAGCAGCCAGTT | 17 | 1748 |
| 1127575 | N/A | N/A | 9672 | 9691 | TTAGATTGGACAGACTGAAG | 37 | 1749 |
| 1127607 | N/A | N/A | 9800 | 9819 | CGGACAGGGACCTGTAAATC | 47 | 1750 |
| 1127639 | N/A | N/A | 9864 | 9883 | GGAGAGGTCAATCTGTCACC | 47 | 1751 |
| 1127671 | N/A | N/A | 10160 | 10179 | TCCCCCATCTGCCACGGTCC | 0 | 1752 |
| 1127703 | N/A | N/A | 10577 | 10596 | TGGCATAGACAGCTGCCTAC | 8 | 1753 |
| 1127735 | N/A | N/A | 12015 | 12034 | GGTCCCCAGCTTAGGACTTA | 2 | 1754 |
| 1127767 | N/A | N/A | 12292 | 12311 | TCACTGAGAACGGATACTAA | 36 | 1755 |
| 1127799 | N/A | N/A | 12447 | 12466 | GCTTCTTACAGGGACCCAAT | 62 | 1756 |
| 1127831 | N/A | N/A | 12695 | 12714 | GGCCAGTTCCCTTGGTGACA | 13 | 1757 |
| 1127863 | N/A | N/A | 13395 | 13414 | GGAGGCCGAATACCCAGGTG | 13 | 1758 |
| 1127895 | N/A | N/A | 13594 | 13613 | CAAGGCACGGTAAAGCCCAA | 5 | 1759 |
| 1127927 | N/A | N/A | 14253 | 14272 | TACCCATTCCTGTCAAGGAG | 21 | 1760 |
| 1127954 | N/A | N/A | 15654 | 15673 | CCGTCTACAGGATTTTCTAG | 95 | 83 |
| 1127960 | N/A | N/A | 15662 | 15681 | AGTAAATTCCGTCTACAGGA | 87 | 1761 |
| 1127992 | N/A | N/A | 16566 | 16585 | CTCGGCCACTTTCTAGGTTC | 49 | 1762 |
| 1128024 | N/A | N/A | 16853 | 16872 | TAGGACTTGTACTTGTCCTG | 0 | 1763 |
| 1128056 | N/A | N/A | 17147 | 17166 | ACCCTTAGGTTTCTAGGATA | 36 | 1764 |
| 1128088 | N/A | N/A | 17893 | 17912 | GGCACTCACACCAGTAAGGA | 14 | 1765 |
| 1128120 | N/A | N/A | 18234 | 18253 | CTGAAGTTGCACGAGTCAGC | 19 | 1766 |
| 1128152 | N/A | N/A | 19298 | 19317 | CGTGTGCAGCAAGGCCCAAG | 18 | 1767 |
| 1128184 | N/A | N/A | 19495 | 19514 | AGGGATCTAAAGCAGCTACA | 45 | 1768 |
| 1128216 | N/A | N/A | 19576 | 19595 | GAGTTGTAGTTTTTTGGGCC | 1 | 1769 |
| 1128248 | N/A | N/A | 19809 | 19828 | TAATGAGGTGGGCTGCTTTA | 11 | 1770 |
| 1128280 | N/A | N/A | 19962 | 19981 | TAGTGTGATAGGAAGTCCTG | 18 | 1771 |
| 1128312 | N/A | N/A | 21122 | 21141 | CCAAGTGATCACCAGGAGTT | 22 | 1772 |
| 1128344 | N/A | N/A | 21775 | 21794 | ACTCACCCGGTGTGTAGCCC | 0 | 1773 |
| 1128376 | N/A | N/A | 21834 | 21853 | CCCCAAGACATGCTAGCTCT | 0 | 1774 |
| 1128408 | N/A | N/A | 21985 | 22004 | ATCCAGTTCACTAGTCCTCA | 22 | 1775 |
| 1128440 | N/A | N/A | 22150 | 22169 | AACACTGGCTAATGTGAACC | 4 | 1776 |
| 1128472 | N/A | N/A | 24585 | 24604 | CTTGTACGGACTTAGTCTAT | 15 | 1777 |
| 1128504 | N/A | N/A | 24665 | 24684 | TCACAACCTGTAGGATCGGG | 58 | 1778 |
| 1128536 | N/A | N/A | 24909 | 24928 | GTCTTAATCGCAATAGACGC | 0 | 1779 |
| 1128568 | N/A | N/A | 24968 | 24987 | GTGCACTCAGCTGCGGGAAG | 19 | 1780 |
| 1128600 | N/A | N/A | 25524 | 25543 | GTAGGATGAGACCCTGCACC | 0 | 1781 |
| 1128632 | N/A | N/A | 25582 | 25601 | ATGAGCCACCATCGCGATCG | 31 | 1782 |
| 1128664 | N/A | N/A | 25996 | 26015 | ATCTGACTCCACCCCTTATG | 13 | 1783 |
| 1128696 | N/A | N/A | 26368 | 26387 | GCATACGGCGATGTGGGTGC | 19 | 1784 |

TABLE 25
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126264 | 93 | 112 | 2740 | 2759 | CCTATTGCAAGACCGCACCC | 6 | 1785 |
| 1126296 | 173 | 192 | 2820 | 2839 | AGGTGTCTAGGGAATGCACC | 0 | 1786 |
| 1126328 | 250 | 269 | 2897 | 2916 | ACATGGACAAAGTGCGGTTT | 65 | 1787 |
| 1126360 | 438 | 457 | 4596 | 4615 | GCCCTGCTCCGTGTACGGCC | 6 | 1788 |
| 1126392 | 802 | 821 | 10034 | 10053 | GGGCACGACACAGGCAGAGT | 31 | 1789 |
| 1126424 | 884 | 903 | 10116 | 10135 | TAGAAGTCCACGGCACCGGC | 0 | 1790 |
| 1126456 | 1020 | 1039 | 10496 | 10515 | TGCCTCGATGGCGGTGATCT | 85† | 1791 |
| 1126488 | 1223 | 1242 | 13666 | 13685 | AACTCATAGCGGCCGGCGAT | 8 | 1792 |
| 1126520 | 1338 | 1357 | 14399 | 14418 | GGTCCGCGCTGGCATGATGA | 39 | 1793 |
| 1126552 | 1480 | 1499 | 18008 | 18027 | GCATCTTGTTCATGTCGGGA | 72 | 1794 |
| 1126584 | 1630 | 1649 | 21348 | 21367 | CACTGCTATTGAAGAGGCCG | 0 | 1795 |
| 1126616 | 1792 | 1811 | 24979 | 24998 | TTCCCATAACCGTGCACTCA | 27 | 1796 |
| 1126648 | 1969 | 1988 | 25374 | 25393 | TACGCTGCCGCCGGCTCTGC | 0 | 1797 |
| 1126680 | 2057 | 2076 | 26152 | 26171 | GCCATGTGGCGCGCAGACAT | 19 | 1798 |
| 1126712 | 2276 | 2295 | 26534 | 26553 | CGGTCCTTGGCGGCCTCCTC | 0 | 1799 |
| 1126744 | 2350 | 2369 | 26608 | 26627 | AGTTGCGCTTGCTGCCGCTG | 7 | 1800 |
| 1126776 | 2634 | 2653 | 26892 | 26911 | GGCACGGCTTTGTGGATTCT | 92 | 56 |
| 1126808 | 2853 | 2872 | 27111 | 27130 | GGCCCGCATGCCGGGCCTGA | 13 | 1801 |
| 1126840 | 2918 | 2937 | 27176 | 27195 | AGTGCCCCGGCTCTGGACTT | 26 | 1802 |
| 1126872 | 3107 | 3126 | 27365 | 27384 | GAGCAGCCAAGTGGTTCTAC | 66 | 1803 |
| 1126904 | 3404 | 3423 | 27662 | 27681 | GGGTTCTAGAACCCAGTGAC | 4 | 1804 |
| 1126936 | N/A | N/A | 2994 | 3013 | TGCTCACCCTTGTTAGCCAC | 24 | 1805 |
| 1126968 | N/A | N/A | 3086 | 3105 | GGGTTCCCCTAGTAGCCCCG | 16 | 1806 |
| 1127000 | N/A | N/A | 3277 | 3296 | CCCAGGTATCTAGTCTCTCG | 86 | 1807 |
| 1127032 | N/A | N/A | 3357 | 3376 | GGTCATAGGTCTGAAACCCC | 26 | 1808 |
| 1127064 | N/A | N/A | 3444 | 3463 | AGGTCCTCCCACCGCCGCTC | 44 | 1809 |
| 1127096 | N/A | N/A | 3585 | 3604 | ATTCCAGTACGCATGTGTCC | 85 | 1810 |
| 1127128 | N/A | N/A | 3851 | 3870 | CCTGGCGATACCCGGCTGGG | 8 | 1811 |
| 1127160 | N/A | N/A | 4142 | 4161 | GACTACCCGGACTCAGGTTC | 17 | 1812 |
| 1127192 | N/A | N/A | 4468 | 4487 | CGGCCGAGGTCCATAGTTCT | 11 | 1813 |
| 1127224 | N/A | N/A | 4831 | 4850 | TGTCTGGGTGTCCTATCACG | 50 | 1814 |
| 1127256 | N/A | N/A | 5305 | 5324 | TAGCATGTCCATTAGCCAAG | 70 | 1815 |
| 1127288 | N/A | N/A | 5458 | 5477 | CAAGCTTGGCTGTTAGTAAC | 45 | 1816 |
| 1127320 | N/A | N/A | 5897 | 5916 | AGGGACACAGTCGGACCTGA | 17 | 1817 |
| 1127352 | N/A | N/A | 6065 | 6084 | CTCCTCGCCGACCTGGCTTT | 9 | 1818 |
| 1127384 | N/A | N/A | 6987 | 7006 | CCTCTATCCATAGGCCCATT | 83 | 1819 |
| 1127416 | N/A | N/A | 7859 | 7878 | GCTACCCTTCTCCCACTCGC | 48 | 1820 |
| 1127448 | N/A | N/A | 8534 | 8553 | ATCCGTGGTTCTCCCATTTG | 63 | 1821 |
| 1127480 | N/A | N/A | 8910 | 8929 | AATCCTTGGATTAAAGGACC | 8 | 1822 |
| 1127512 | N/A | N/A | 9006 | 9025 | CAAGAATCCCTATGCTTGGA | 19 | 1823 |
| 1127544 | N/A | N/A | 9552 | 9571 | ATGCCATAGGAGCAGCCAGT | 21 | 1824 |
| 1127576 | N/A | N/A | 9674 | 9693 | GGTTAGATTGGACAGACTGA | 51 | 1825 |
| 1127608 | N/A | N/A | 9801 | 9820 | CCGGACAGGGACCTGTAAAT | 17 | 1826 |
| 1127640 | N/A | N/A | 9865 | 9884 | GGGAGAGGTCAATCTGTCAC | 33 | 1827 |
| 1127672 | N/A | N/A | 10162 | 10181 | AGTCCCCCATCTGCCACGGT | 23 | 1828 |
| 1127704 | N/A | N/A | 10578 | 10597 | TTGGCATAGACAGCTGCCTA | 0 | 1829 |
| 1127736 | N/A | N/A | 12016 | 12035 | TGGTCCCCAGCTTAGGACTT | 5 | 1830 |
| 1127768 | N/A | N/A | 12293 | 12312 | CTCACTGAGAACGGATACTA | 14 | 1831 |
| 1127800 | N/A | N/A | 12449 | 12468 | AAGCTTCTTACAGGGACCCA | 26 | 1832 |
| 1127832 | N/A | N/A | 13027 | 13046 | TTCCTATGAGGTCCCCCCTC | 7 | 1833 |
| 1127864 | N/A | N/A | 13396 | 13415 | AGGAGGCCGAATACCCAGGT | 30 | 1834 |
| 1127896 | N/A | N/A | 13595 | 13614 | ACAAGGCACGGTAAAGCCCA | 19 | 1835 |
| 1127928 | N/A | N/A | 14264 | 14283 | GGATCCTGCCATACCCATTC | 15 | 1836 |
| 1127954 | N/A | N/A | 15654 | 15673 | CCGTCTACAGGATTTTCTAG | 95 | 83 |
| 1127961 | N/A | N/A | 15663 | 15682 | AAGTAAATTCCGTCTACAGG | 77 | 1837 |
| 1127993 | N/A | N/A | 16567 | 16586 | TCTCGGCCACTTTCTAGGTT | 83 | 1838 |
| 1128025 | N/A | N/A | 16854 | 16873 | TTAGGACTTGTACTTGTCCT | 0 | 1839 |
| 1128057 | N/A | N/A | 17148 | 17167 | TACCCTTAGGTTTCTAGGAT | 59 | 1840 |
| 1128089 | N/A | N/A | 17951 | 17970 | AGAACGGAGTAATGAGAGGG | 0 | 1841 |
| 1128121 | N/A | N/A | 18235 | 18254 | ACTGAAGTTGCACGAGTCAG | 10 | 1842 |
| 1128153 | N/A | N/A | 19301 | 19320 | AGCCGTGTGCAGCAAGGCCC | 8 | 1843 |
| 1128185 | N/A | N/A | 19496 | 19515 | TAGGGATCTAAAGCAGCTAC | 40 | 1844 |
| 1128217 | N/A | N/A | 19611 | 19630 | CGTCAATGCCCCAGAAAGCC | 27 | 1845 |
| 1128249 | N/A | N/A | 19810 | 19829 | ATAATGAGGTGGGCTGCTTT | 12 | 1846 |
| 1128281 | N/A | N/A | 19963 | 19982 | CTAGTGTGATAGGAAGTCCT | 41 | 1847 |
| 1128313 | N/A | N/A | 21155 | 21174 | GACCACAAATGCACCGGACG | 35 | 1848 |
| 1128345 | N/A | N/A | 21776 | 21795 | CACTCACCCGGTGTGTAGCC | 2 | 1849 |
| 1128377 | N/A | N/A | 21835 | 21854 | GCCCCAAGACATGCTAGCTC | 22 | 1850 |
| 1128409 | N/A | N/A | 21987 | 22006 | ACATCCAGTTCACTAGTCCT | 8 | 1851 |
| 1128441 | N/A | N/A | 22428 | 22447 | AGCACAGCACTGCCGGAGTG | 12 | 1852 |
| 1128473 | N/A | N/A | 24586 | 24605 | TCTTGTACGGACTTAGTCTA | 8 | 1853 |
| 1128505 | N/A | N/A | 24666 | 24685 | CTCACAACCTGTAGGATCGG | 55 | 1854 |
| 1128537 | N/A | N/A | 24910 | 24929 | CGTCTTAATCGCAATAGACG | 11 | 1855 |
| 1128569 | N/A | N/A | 24969 | 24988 | CGTGCACTCAGCTGCGGGAA | 2 | 1856 |
| 1128601 | N/A | N/A | 25525 | 25544 | AGTAGGATGAGACCCTGCAC | 0 | 1857 |
| 1128633 | N/A | N/A | 25584 | 25603 | CCATGAGCCACCATCGCGAT | 6 | 1858 |
| 1128665 | N/A | N/A | 26020 | 26039 | GCACCCCATACAAATAATCC | 5 | 1859 |
| 1128697 | N/A | N/A | 26369 | 26388 | TGCATACGGCGATGTGGGTG | 0 | 1860 |

TABLE 26
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126265 | 94 | 113 | 2741 | 2760 | TCCTATTGCAAGACCGCACC | 28 | 1861 |
| 1126297 | 174 | 193 | 2821 | 2840 | GAGGTGTCTAGGGAATGCAC | 35 | 1862 |
| 1126329 | 251 | 270 | 2898 | 2917 | GACATGGACAAAGTGCGGTT | 56 | 1863 |
| 1126361 | 439 | 458 | 4597 | 4616 | CGCCCTGCTCCGTGTACGGC | 0 | 1864 |
| 1126393 | 803 | 822 | 10035 | 10054 | CGGGCACGACACAGGCAGAG | 24 | 1865 |
| 1126425 | 885 | 904 | 10117 | 10136 | GTAGAAGTCCACGGCACCGG | 14 | 1866 |
| 1126457 | 1022 | 1041 | 10498 | 10517 | TGTGCCTCGATGGCGGTGAT | 89† | 1867 |
| 1126489 | 1227 | 1246 | 13670 | 13689 | GGAGAACTCATAGCGGCCGG | 22 | 1868 |
| 1126521 | 1339 | 1358 | 14400 | 14419 | TGGTCCGCGCTGGCATGATG | 49 | 1869 |
| 1126553 | 1481 | 1500 | 18009 | 18028 | AGCATCTTGTTCATGTCGGG | 58 | 1870 |
| 1126585 | 1632 | 1651 | 21350 | 21369 | GGCACTGCTATTGAAGAGGC | 16 | 1871 |
| 1126617 | 1793 | 1812 | 24980 | 24999 | ATTCCCATAACCGTGCACTC | 0 | 1872 |
| 1126649 | 1970 | 1989 | 25375 | 25394 | ATACGCTGCCGCCGGCTCTG | 0 | 1873 |
| 1126681 | 2058 | 2077 | 26153 | 26172 | CGCCATGTGGCGCGCAGACA | 31 | 1874 |
| 1126713 | 2279 | 2298 | 26537 | 26556 | CGCCGGTCCTTGGCGGCCTC | 10 | 1875 |
| 1126745 | 2352 | 2371 | 26610 | 26629 | AGAGTTGCGCTTGCTGCCGC | 21 | 1876 |
| 1126777 | 2635 | 2654 | 26893 | 26912 | AGGCACGGCTTTGTGGATTC | 89 | 1877 |
| 1126809 | 2854 | 2873 | 27112 | 27131 | TGGCCCGCATGCCGGGCCTG | 17 | 1878 |
| 1126841 | 2919 | 2938 | 27177 | 27196 | AAGTGCCCCGGCTCTGGACT | 30 | 1879 |
| 1126873 | 3108 | 3127 | 27366 | 27385 | TGAGCAGCCAAGTGGTTCTA | 0 | 1880 |
| 1126905 | 3424 | 3443 | 27682 | 27701 | GAGCCTCGAGGTAAATGTGG | 60 | 1881 |
| 1126937 | N/A | N/A | 2996 | 3015 | CGTGCTCACCCTTGTTAGCC | 17 | 1882 |
| 1126969 | N/A | N/A | 3088 | 3107 | AAGGGTTCCCCTAGTAGCCC | 0 | 1883 |
| 1127001 | N/A | N/A | 3278 | 3297 | ACCCAGGTATCTAGTCTCTC | 87 | 372 |
| 1127033 | N/A | N/A | 3358 | 3377 | GGGTCATAGGTCTGAAACCC | 16 | 1884 |
| 1127065 | N/A | N/A | 3450 | 3469 | ATCCTCAGGTCCTCCCACCG | 32 | 1885 |
| 1127097 | N/A | N/A | 3586 | 3605 | TATTCCAGTACGCATGTGTC | 49 | 1886 |
| 1127129 | N/A | N/A | 3852 | 3871 | CCCTGGCGATACCCGGCTGG | 11 | 1887 |
| 1127161 | N/A | N/A | 4143 | 4162 | TGACTACCCGGACTCAGGTT | 44 | 1888 |
| 1127193 | N/A | N/A | 4469 | 4488 | CCGGCCGAGGTCCATAGTTC | 4 | 1889 |
| 1127225 | N/A | N/A | 4832 | 4851 | CTGTCTGGGTGTCCTATCAC | 68 | 1890 |
| 1127257 | N/A | N/A | 5308 | 5327 | TATTAGCATGTCCATTAGCC | 45 | 1891 |
| 1127289 | N/A | N/A | 5459 | 5478 | CCAAGCTTGGCTGTTAGTAA | 63 | 1892 |
| 1127321 | N/A | N/A | 5898 | 5917 | TAGGGACACAGTCGGACCTG | 17 | 1893 |
| 1127353 | N/A | N/A | 6066 | 6085 | GCTCCTCGCCGACCTGGCTT | 7 | 1894 |
| 1127385 | N/A | N/A | 6988 | 7007 | TCCTCTATCCATAGGCCCAT | 84 | 1895 |
| 1127417 | N/A | N/A | 7860 | 7879 | TGCTACCCTTCTCCCACTCG | 40 | 1896 |
| 1127449 | N/A | N/A | 8536 | 8555 | TCATCCGTGGTTCTCCCATT | 53 | 1897 |
| 1127481 | N/A | N/A | 8911 | 8930 | TAATCCTTGGATTAAAGGAC | 9 | 1898 |
| 1127513 | N/A | N/A | 9008 | 9027 | TCCAAGAATCCCTATGCTTG | 15 | 1899 |
| 1127545 | N/A | N/A | 9554 | 9573 | GAATGCCATAGGAGCAGCCA | 19 | 1900 |
| 1127577 | N/A | N/A | 9675 | 9694 | GGGTTAGATTGGACAGACTG | 53 | 1901 |
| 1127609 | N/A | N/A | 9802 | 9821 | ACCGGACAGGGACCTGTAAA | 2 | 1902 |
| 1127641 | N/A | N/A | 9886 | 9905 | CCCAGTAGGGACAAGGACTC | 0 | 1903 |
| 1127673 | N/A | N/A | 10163 | 10182 | CAGTCCCCCATCTGCCACGG | 4 | 1904 |
| 1127705 | N/A | N/A | 10580 | 10599 | CTTTGGCATAGACAGCTGCC | 21 | 1905 |
| 1127737 | N/A | N/A | 12017 | 12036 | ATGGTCCCCAGCTTAGGACT | 27 | 1906 |
| 1127769 | N/A | N/A | 12294 | 12313 | ACTCACTGAGAACGGATACT | 39 | 1907 |
| 1127801 | N/A | N/A | 12450 | 12469 | CAAGCTTCTTACAGGGACCC | 33 | 1908 |
| 1127833 | N/A | N/A | 13028 | 13047 | GTTCCTATGAGGTCCCCCCT | 44 | 1909 |
| 1127865 | N/A | N/A | 13397 | 13416 | AAGGAGGCCGAATACCCAGG | 38 | 1910 |
| 1127897 | N/A | N/A | 13596 | 13615 | CACAAGGCACGGTAAAGCCC | 0 | 1911 |
| 1127929 | N/A | N/A | 14266 | 14285 | TGGGATCCTGCCATACCCAT | 4 | 1912 |
| 1127954 | N/A | N/A | 15654 | 15673 | CCGTCTACAGGATTTTCTAG | 95 | 83 |
| 1127962 | N/A | N/A | 15668 | 15687 | CCCAAAAGTAAATTCCGTCT | 94 | 1913 |
| 1127994 | N/A | N/A | 16569 | 16588 | AATCTCGGCCACTTTCTAGG | 13 | 1914 |
| 1128026 | N/A | N/A | 16856 | 16875 | TATTAGGACTTGTACTTGTC | 54 | 1915 |
| 1128058 | N/A | N/A | 17149 | 17168 | CTACCCTTAGGTTTCTAGGA | 60 | 1916 |
| 1128090 | N/A | N/A | 17959 | 17978 | TGCATGTGAGAACGGAGTAA | 0 | 1917 |
| 1128122 | N/A | N/A | 18236 | 18255 | AACTGAAGTTGCACGAGTCA | 0 | 1918 |
| 1128154 | N/A | N/A | 19302 | 19321 | GAGCCGTGTGCAGCAAGGCC | 12 | 1919 |
| 1128186 | N/A | N/A | 19497 | 19516 | TTAGGGATCTAAAGCAGCTA | 30 | 1920 |
| 1128218 | N/A | N/A | 19679 | 19698 | GTACTGAAGTCCCTGGACCC | 0 | 1921 |
| 1128250 | N/A | N/A | 19811 | 19830 | CATAATGAGGTGGGCTGCTT | 17 | 1922 |
| 1128282 | N/A | N/A | 19965 | 19984 | AGCTAGTGTGATAGGAAGTC | 39 | 1923 |
| 1128314 | N/A | N/A | 21156 | 21175 | AGACCACAAATGCACCGGAC | 31 | 1924 |
| 1128346 | N/A | N/A | 21777 | 21796 | ACACTCACCCGGTGTGTAGC | 5 | 1925 |
| 1128378 | N/A | N/A | 21836 | 21855 | CGCCCCAAGACATGCTAGCT | 15 | 1926 |
| 1128410 | N/A | N/A | 21988 | 22007 | GACATCCAGTTCACTAGTCC | 0 | 1927 |
| 1128442 | N/A | N/A | 22429 | 22448 | GAGCACAGCACTGCCGGAGT | 14 | 1928 |
| 1128474 | N/A | N/A | 24587 | 24606 | ATCTTGTACGGACTTAGTCT | 7 | 1929 |
| 1128506 | N/A | N/A | 24667 | 24686 | TCTCACAACCTGTAGGATCG | 42 | 1930 |
| 1128538 | N/A | N/A | 24911 | 24930 | CCGTCTTAATCGCAATAGAC | 17 | 1931 |
| 1128570 | N/A | N/A | 24971 | 24990 | ACCGTGCACTCAGCTGCGGG | 0 | 1932 |
| 1128602 | N/A | N/A | 25526 | 25545 | AAGTAGGATGAGACCCTGCA | 16 | 1933 |
| 1128634 | N/A | N/A | 25586 | 25605 | AACCATGAGCCACCATCGCG | 23 | 1934 |
| 1128666 | N/A | N/A | 26026 | 26045 | ACTGCTGCACCCCATACAAA | 4 | 1935 |
| 1128698 | N/A | N/A | 26370 | 26389 | CTGCATACGGCGATGTGGGT | 0 | 1936 |

TABLE 27
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126266 | 95 | 114 | 2742 | 2761 | TTCCTATTGCAAGACCGCAC | 33 | 1937 |
| 1126298 | 177 | 196 | 2824 | 2843 | CCGGAGGTGTCTAGGGAATG | 20 | 1938 |
| 1126330 | 290 | 309 | 2937 | 2956 | TCGAATTCATCCTCCCAGTC | 59 | 1939 |
| 1126362 | 441 | 460 | 4599 | 4618 | CACGCCCTGCTCCGTGTACG | 10 | 1940 |
| 1126394 | 805 | 824 | 10037 | 10056 | GCCGGGCACGACACAGGCAG | 11 | 1941 |
| 1126426 | 886 | 905 | 10118 | 10137 | TGTAGAAGTCCACGGCACCG | 16 | 1942 |
| 1126458 | 1023 | 1042 | 10499 | 10518 | CTGTGCCTCGATGGCGGTGA | 91† | 1943 |
| 1126490 | 1229 | 1248 | 13672 | 13691 | TTGGAGAACTCATAGCGGCC | 36 | 1944 |
| 1126522 | 1340 | 1359 | 14401 | 14420 | TTGGTCCGCGCTGGCATGAT | 69 | 1945 |
| 1126554 | 1483 | 1502 | 18011 | 18030 | CCAGCATCTTGTTCATGTCG | 30 | 1946 |
| 1126586 | 1633 | 1652 | 21351 | 21370 | CGGCACTGCTATTGAAGAGG | 33 | 1947 |
| 1126618 | 1794 | 1813 | 24981 | 25000 | GATTCCCATAACCGTGCACT | 15 | 1948 |
| 1126650 | 1971 | 1990 | 25376 | 25395 | GATACGCTGCCGCCGGCTCT | 11 | 1949 |
| 1126682 | 2134 | 2153 | 26392 | 26411 | GTGGGTAGCGGTACCCCTGG | 16 | 1950 |
| 1126714 | 2281 | 2300 | 26539 | 26558 | TGCGCCGGTCCTTGGCGGCC | 2 | 1951 |
| 1126746 | 2353 | 2372 | 26611 | 26630 | CAGAGTTGCGCTTGCTGCCG | 40 | 1952 |
| 1126778 | 2636 | 2655 | 26894 | 26913 | AAGGCACGGCTTTGTGGATT | 85 | 1953 |
| 1126810 | 2855 | 2874 | 27113 | 27132 | GTGGCCCGCATGCCGGGCCT | 22 | 1954 |
| 1126842 | 2920 | 2939 | 27178 | 27197 | AAAGTGCCCCGGCTCTGGAC | 30 | 1955 |
| 1126874 | 3109 | 3128 | 27367 | 27386 | ATGAGCAGCCAAGTGGTTCT | 14 | 1956 |
| 1126906 | 3425 | 3444 | 27683 | 27702 | AGAGCCTCGAGGTAAATGTG | 64 | 1957 |
| 1126938 | N/A | N/A | 3036 | 3055 | ATCCACTACTGTCCTTCTCG | 67 | 1958 |
| 1126970 | N/A | N/A | 3089 | 3108 | CAAGGGTTCCCCTAGTAGCC | 20 | 1959 |
| 1127002 | N/A | N/A | 3279 | 3298 | GACCCAGGTATCTAGTCTCT | 56 | 1960 |
| 1127034 | N/A | N/A | 3359 | 3378 | AGGGTCATAGGTCTGAAACC | 36 | 1961 |
| 1127066 | N/A | N/A | 3464 | 3483 | TCTTAAGCAGCCAGATCCTC | 57 | 1962 |
| 1127098 | N/A | N/A | 3587 | 3606 | CTATTCCAGTACGCATGTGT | 60 | 1963 |
| 1127130 | N/A | N/A | 3853 | 3872 | GCCCTGGCGATACCCGGCTG | 28 | 1964 |
| 1127162 | N/A | N/A | 4144 | 4163 | CTGACTACCCGGACTCAGGT | 21 | 1965 |
| 1127194 | N/A | N/A | 4470 | 4489 | CCCGGCCGAGGTCCATAGTT | 13 | 1966 |
| 1127226 | N/A | N/A | 4836 | 4855 | GCATCTGTCTGGGTGTCCTA | 72 | 1967 |
| 1127258 | N/A | N/A | 5309 | 5328 | CTATTAGCATGTCCATTAGC | 28 | 1968 |
| 1127290 | N/A | N/A | 5462 | 5481 | TCTCCAAGCTTGGCTGTTAG | 64 | 1969 |
| 1127322 | N/A | N/A | 5899 | 5918 | TTAGGGACACAGTCGGACCT | 27 | 1970 |
| 1127354 | N/A | N/A | 6068 | 6087 | CCGCTCCTCGCCGACCTGGC | 41 | 1971 |
| 1127386 | N/A | N/A | 7001 | 7020 | CCTGCGACCTCTTTCCTCTA | 60 | 1972 |
| 1127418 | N/A | N/A | 8363 | 8382 | CGTGCCCCCTGCTTACTTTC | 70 | 1973 |
| 1127450 | N/A | N/A | 8537 | 8556 | CTCATCCGTGGTTCTCCCAT | 34 | 1974 |
| 1127482 | N/A | N/A | 8913 | 8932 | GCTAATCCTTGGATTAAAGG | 49 | 1975 |
| 1127514 | N/A | N/A | 9011 | 9030 | GATTCCAAGAATCCCTATGC | 39 | 1976 |
| 1127546 | N/A | N/A | 9555 | 9574 | AGAATGCCATAGGAGCAGCC | 30 | 1977 |
| 1127578 | N/A | N/A | 9676 | 9695 | AGGGTTAGATTGGACAGACT | 76 | 1978 |
| 1127610 | N/A | N/A | 9803 | 9822 | AACCGGACAGGGACCTGTAA | 0 | 1979 |
| 1127642 | N/A | N/A | 9887 | 9906 | CCCCAGTAGGGACAAGGACT | 21 | 1980 |
| 1127674 | N/A | N/A | 10164 | 10183 | GCAGTCCCCCATCTGCCACG | 30 | 1981 |
| 1127706 | N/A | N/A | 10581 | 10600 | GCTTTGGCATAGACAGCTGC | 0 | 1982 |
| 1127738 | N/A | N/A | 12025 | 12044 | GGTAACTGATGGTCCCCAGC | 80 | 1983 |
| 1127770 | N/A | N/A | 12295 | 12314 | TACTCACTGAGAACGGATAC | 41 | 1984 |
| 1127802 | N/A | N/A | 12468 | 12487 | GGATATATCCTCTACTTCCA | 21 | 1985 |
| 1127834 | N/A | N/A | 13100 | 13119 | ATCAATGTTGTGGTTGAATG | 35 | 1986 |
| 1127866 | N/A | N/A | 13398 | 13417 | TAAGGAGGCCGAATACCCAG | 16 | 1987 |
| 1127898 | N/A | N/A | 13597 | 13616 | CCACAAGGCACGGTAAAGCC | 14 | 1988 |
| 1127930 | N/A | N/A | 14268 | 14287 | ACTGGGATCCTGCCATACCC | 2 | 1989 |
| 1127954 | N/A | N/A | 15654 | 15673 | CCGTCTACAGGATTTTCTAG | 95 | 83 |
| 1127963 | N/A | N/A | 15669 | 15688 | GCCCAAAAGTAAATTCCGTC | 93 | 1990 |
| 1127995 | N/A | N/A | 16571 | 16590 | GCAATCTCGGCCACTTTCTA | 64 | 1991 |
| 1128027 | N/A | N/A | 16861 | 16880 | GCTATTATTAGGACTTGTAC | 90 | 469 |
| 1128059 | N/A | N/A | 17150 | 17169 | GCTACCCTTAGGTTTCTAGG | 53 | 1992 |
| 1128091 | N/A | N/A | 17960 | 17979 | ATGCATGTGAGAACGGAGTA | 1 | 1993 |
| 1128123 | N/A | N/A | 18237 | 18256 | CAACTGAAGTTGCACGAGTC | 0 | 1994 |
| 1128155 | N/A | N/A | 19303 | 19322 | GGAGCCGTGTGCAGCAAGGC | 0 | 1995 |
| 1128187 | N/A | N/A | 19499 | 19518 | AGTTAGGGATCTAAAGCAGC | 35 | 1996 |
| 1128219 | N/A | N/A | 19680 | 19699 | AGTACTGAAGTCCCTGGACC | 1 | 1997 |
| 1128251 | N/A | N/A | 19813 | 19832 | TTCATAATGAGGTGGGCTGC | 4 | 1998 |
| 1128283 | N/A | N/A | 19966 | 19985 | GAGCTAGTGTGATAGGAAGT | 47 | 1999 |
| 1128315 | N/A | N/A | 21159 | 21178 | AGGAGACCACAAATGCACCG | 34 | 2000 |
| 1128347 | N/A | N/A | 21778 | 21797 | TACACTCACCCGGTGTGTAG | 16 | 2001 |
| 1128379 | N/A | N/A | 21837 | 21856 | TCGCCCCAAGACATGCTAGC | 2 | 2002 |
| 1128411 | N/A | N/A | 21989 | 22008 | TGACATCCAGTTCACTAGTC | 30 | 2003 |
| 1128443 | N/A | N/A | 22557 | 22576 | CTCCTAATTATCATGCAAGC | 50 | 2004 |
| 1128475 | N/A | N/A | 24588 | 24607 | TATCTTGTACGGACTTAGTC | 12 | 2005 |
| 1128507 | N/A | N/A | 24668 | 24687 | CTCTCACAACCTGTAGGATC | 25 | 2006 |
| 1128539 | N/A | N/A | 24912 | 24931 | ACCGTCTTAATCGCAATAGA | 15 | 2007 |
| 1128571 | N/A | N/A | 25061 | 25080 | CGCTGACCGTAAGCTGAGGG | 44 | 2008 |
| 1128603 | N/A | N/A | 25530 | 25549 | TTACAAGTAGGATGAGACCC | 31 | 2009 |
| 1128635 | N/A | N/A | 25626 | 25645 | GCGATCCTTCCTGTTAGGCC | 65 | 2010 |
| 1128667 | N/A | N/A | 26027 | 26046 | CACTGCTGCACCCCATACAA | 14 | 2011 |
| 1128699 | N/A | N/A | 26371 | 26390 | CCTGCATACGGCGATGTGGG | 0 | 2012 |

TABLE 28
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126267 | 96 | 115 | 2743 | 2762 | CTTCCTATTGCAAGACCGCA | 8 | 2013 |
| 1126299 | 178 | 197 | 2825 | 2844 | CCCGGAGGTGTCTAGGGAAT | 22 | 2014 |
| 1126331 | 291 | 310 | 2938 | 2957 | GTCGAATTCATCCTCCCAGT | 77 | 2015 |
| 1126363 | 443 | 462 | 4601 | 4620 | CTCACGCCCTGCTCCGTGTA | 0 | 2016 |
| 1126395 | 806 | 825 | 10038 | 10057 | CGCCGGGCACGACACAGGCA | 0 | 2017 |
| 1126427 | 888 | 907 | 10120 | 10139 | GTTGTAGAAGTCCACGGCAC | 23 | 2018 |
| 1126459 | 1025 | 1044 | 10501 | 10520 | TGCTGTGCCTCGATGGCGGT | 77† | 2019 |
| 1126491 | 1230 | 1249 | 13673 | 13692 | GTTGGAGAACTCATAGCGGC | 58 | 2020 |
| 1126523 | 1342 | 1361 | 14403 | 14422 | TGTTGGTCCGCGCTGGCATG | 74 | 2021 |
| 1126555 | 1568 | 1587 | 21286 | 21305 | AGCATATTGTGGGTGCACAC | 40 | 2022 |
| 1126587 | 1634 | 1653 | 21352 | 21371 | TCGGCACTGCTATTGAAGAG | 18 | 2023 |
| 1126619 | 1819 | 1838 | 25006 | 25025 | CGGAGAGATTGGTGGAGATA | 36 | 2024 |
| 1126651 | 1973 | 1992 | 25378 | 25397 | ATGATACGCTGCCGCCGGCT | 7 | 2025 |
| 1126683 | 2135 | 2154 | 26393 | 26412 | CGTGGGTAGCGGTACCCCTG | 0 | 2026 |
| 1126715 | 2282 | 2301 | 26540 | 26559 | TTGCGCCGGTCCTTGGCGGC | 0 | 2027 |
| 1126747 | 2356 | 2375 | 26614 | 26633 | CCACAGAGTTGCGCTTGCTG | 59 | 2028 |
| 1126779 | 2637 | 2656 | 26895 | 26914 | AAAGGCACGGCTTTGTGGAT | 51 | 2029 |
| 1126811 | 2857 | 2876 | 27115 | 27134 | TGGTGGCCCGCATGCCGGGC | 0 | 2030 |
| 1126843 | 2921 | 2940 | 27179 | 27198 | GAAAGTGCCCCGGCTCTGGA | 27 | 2031 |
| 1126875 | 3110 | 3129 | 27368 | 27387 | AATGAGCAGCCAAGTGGTTC | 18 | 2032 |
| 1126907 | 3426 | 3445 | 27684 | 27703 | AAGAGCCTCGAGGTAAATGT | 59 | 2033 |
| 1126939 | N/A | N/A | 3044 | 3063 | GGGCCCCCATCCACTACTGT | 0 | 2034 |
| 1126971 | N/A | N/A | 3090 | 3109 | ACAAGGGTTCCCCTAGTAGC | 20 | 2035 |
| 1127003 | N/A | N/A | 3280 | 3299 | GGACCCAGGTATCTAGTCTC | 23 | 2036 |
| 1127035 | N/A | N/A | 3360 | 3379 | TAGGGTCATAGGTCTGAAAC | 45 | 2037 |
| 1127067 | N/A | N/A | 3482 | 3501 | AGCCCGTCCCATCTTTATTC | 37 | 2038 |
| 1127099 | N/A | N/A | 3588 | 3607 | CCTATTCCAGTACGCATGTG | 64 | 2039 |
| 1127131 | N/A | N/A | 3855 | 3874 | AGGCCCTGGCGATACCCGGC | 9 | 2040 |
| 1127163 | N/A | N/A | 4145 | 4164 | ACTGACTACCCGGACTCAGG | 11 | 2041 |
| 1127195 | N/A | N/A | 4471 | 4490 | CCCCGGCCGAGGTCCATAGT | 25 | 2042 |
| 1127227 | N/A | N/A | 4874 | 4893 | GCCCATGTCTGGGATCCACC | 0 | 2043 |
| 1127259 | N/A | N/A | 5310 | 5329 | GCTATTAGCATGTCCATTAG | 72 | 2044 |
| 1127291 | N/A | N/A | 5597 | 5616 | CCGATATTCCATGCTTCTCT | 84 | 2045 |
| 1127323 | N/A | N/A | 5900 | 5919 | CTTAGGGACACAGTCGGACC | 43 | 2046 |
| 1127355 | N/A | N/A | 6069 | 6088 | CCCGCTCCTCGCCGACCTGG | 21 | 2047 |
| 1127387 | N/A | N/A | 7005 | 7024 | GTCACCTGCGACCTCTTTCC | 63 | 2048 |
| 1127419 | N/A | N/A | 8364 | 8383 | CCGTGCCCCCTGCTTACTTT | 75 | 2049 |
| 1127451 | N/A | N/A | 8538 | 8557 | ACTCATCCGTGGTTCTCCCA | 39 | 2050 |
| 1127483 | N/A | N/A | 8919 | 8938 | GCCAGAGCTAATCCTTGGAT | 40 | 2051 |
| 1127515 | N/A | N/A | 9025 | 9044 | CTCACTTTGAGGCTGATTCC | 17 | 2052 |
| 1127547 | N/A | N/A | 9556 | 9575 | CAGAATGCCATAGGAGCAGC | 8 | 2053 |
| 1127579 | N/A | N/A | 9677 | 9696 | CAGGGTTAGATTGGACAGAC | 82 | 2054 |
| 1127611 | N/A | N/A | 9804 | 9823 | CAACCGGACAGGGACCTGTA | 0 | 2055 |
| 1127643 | N/A | N/A | 9888 | 9907 | TCCCCAGTAGGGACAAGGAC | 0 | 2056 |
| 1127675 | N/A | N/A | 10169 | 10188 | TACTCGCAGTCCCCCATCTG | 0 | 2057 |
| 1127707 | N/A | N/A | 10587 | 10606 | CAACTGGCTTTGGCATAGAC | 41 | 2058 |
| 1127739 | N/A | N/A | 12026 | 12045 | TGGTAACTGATGGTCCCCAG | 59 | 2059 |
| 1127771 | N/A | N/A | 12296 | 12315 | ATACTCACTGAGAACGGATA | 29 | 2060 |
| 1127803 | N/A | N/A | 12471 | 12490 | CTAGGATATATCCTCTACTT | 22 | 2061 |
| 1127835 | N/A | N/A | 13101 | 13120 | GATCAATGTTGTGGTTGAAT | 56 | 2062 |
| 1127867 | N/A | N/A | 13399 | 13418 | TTAAGGAGGCCGAATACCCA | 5 | 2063 |
| 1127899 | N/A | N/A | 13598 | 13617 | CCCACAAGGCACGGTAAAGC | 6 | 2064 |
| 1127931 | N/A | N/A | 14271 | 14290 | ACCACTGGGATCCTGCCATA | 35 | 2065 |
| 1127954 | N/A | N/A | 15654 | 15673 | CCGTCTACAGGATTTTCTAG | 95 | 83 |
| 1127964 | N/A | N/A | 15699 | 15718 | CGTCTCATATTCCAGTTCTG | 78 | 2066 |
| 1127996 | N/A | N/A | 16572 | 16591 | CGCAATCTCGGCCACTTTCT | 32 | 2067 |
| 1128028 | N/A | N/A | 16863 | 16882 | CAGCTATTATTAGGACTTGT | 89 | 2068 |
| 1128060 | N/A | N/A | 17151 | 17170 | AGCTACCCTTAGGTTTCTAG | 45 | 2069 |
| 1128092 | N/A | N/A | 17961 | 17980 | CATGCATGTGAGAACGGAGT | 0 | 2070 |
| 1128124 | N/A | N/A | 18238 | 18257 | GCAACTGAAGTTGCACGAGT | 0 | 2071 |
| 1128156 | N/A | N/A | 19304 | 19323 | TGGAGCCGTGTGCAGCAAGG | 5 | 2072 |
| 1128188 | N/A | N/A | 19502 | 19521 | TGAAGTTAGGGATCTAAAGC | 4 | 2073 |
| 1128220 | N/A | N/A | 19681 | 19700 | AAGTACTGAAGTCCCTGGAC | 0 | 2074 |
| 1128252 | N/A | N/A | 19816 | 19835 | ACATTCATAATGAGGTGGGC | 17 | 2075 |
| 1128284 | N/A | N/A | 19969 | 19988 | ACAGAGCTAGTGTGATAGGA | 75 | 2076 |
| 1128316 | N/A | N/A | 21188 | 21207 | CATGAGCCATGCGGACCCTG | 0 | 2077 |
| 1128348 | N/A | N/A | 21779 | 21798 | CTACACTCACCCGGTGTGTA | 2 | 2078 |
| 1128380 | N/A | N/A | 21838 | 21857 | CTCGCCCCAAGACATGCTAG | 0 | 2079 |
| 1128412 | N/A | N/A | 21990 | 22009 | GTGACATCCAGTTCACTAGT | 0 | 2080 |
| 1128444 | N/A | N/A | 22561 | 22580 | GCGACTCCTAATTATCATGC | 61 | 2081 |
| 1128476 | N/A | N/A | 24589 | 24608 | GTATCTTGTACGGACTTAGT | 37 | 2082 |
| 1128508 | N/A | N/A | 24671 | 24690 | TAACTCTCACAACCTGTAGG | 31 | 2083 |
| 1128540 | N/A | N/A | 24913 | 24932 | TACCGTCTTAATCGCAATAG | 0 | 2084 |
| 1128572 | N/A | N/A | 25141 | 25160 | CCAAGGGTGCAAGCTCCTCC | 23 | 2085 |
| 1128604 | N/A | N/A | 25531 | 25550 | GTTACAAGTAGGATGAGACC | 17 | 2086 |
| 1128636 | N/A | N/A | 25628 | 25647 | ATGCGATCCTTCCTGTTAGG | 38 | 2087 |
| 1128668 | N/A | N/A | 26048 | 26067 | CACCCTTTATGCAGATGAGC | 39 | 2088 |
| 1128700 | N/A | N/A | 26372 | 26391 | GCCTGCATACGGCGATGTGG | 0 | 2089 |

TABLE 29
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126268 | 98 | 117 | 2745 | 2764 | GGCTTCCTATTGCAAGACCG | 17 | 2090 |
| 1126300 | 180 | 199 | 2827 | 2846 | ACCCCGGAGGTGTCTAGGGA | 0 | 2091 |
| 1126332 | 292 | 311 | 2939 | 2958 | GGTCGAATTCATCCTCCCAG | 72 | 2092 |
| 1126364 | 446 | 465 | 4604 | 4623 | GTCCTCACGCCCTGCTCCGT | 39 | 2093 |
| 1126396 | 808 | 827 | 10040 | 10059 | GTCGCCGGGCACGACACAGG | 10 | 2094 |
| 1126428 | 889 | 908 | 10121 | 10140 | TGTTGTAGAAGTCCACGGCA | 0 | 2095 |
| 1126460 | 1029 | 1048 | 10505 | 10524 | CAAGTGCTGTGCCTCGATGG | 88† | 2096 |
| 1126492 | 1232 | 1251 | 13675 | 13694 | TTGTTGGAGAACTCATAGCG | 31 | 2097 |
| 1126524 | 1343 | 1362 | 14404 | 14423 | TTGTTGGTCCGCGCTGGCAT | 34 | 2098 |
| 1126556 | 1571 | 1590 | 21289 | 21308 | TCCAGCATATTGTGGGTGCA | 39 | 2099 |
| 1126588 | 1635 | 1654 | 21353 | 21372 | GTCGGCACTGCTATTGAAGA | 17 | 2100 |
| 1126620 | 1820 | 1839 | 25007 | 25026 | CCGGAGAGATTGGTGGAGAT | 44 | 2101 |
| 1126652 | 1974 | 1993 | 25379 | 25398 | GATGATACGCTGCCGCCGGC | 7 | 2102 |
| 1126684 | 2136 | 2155 | 26394 | 26413 | CCGTGGGTAGCGGTACCCCT | 0 | 2103 |
| 1126716 | 2283 | 2302 | 26541 | 26560 | GTTGCGCCGGTCCTTGGCGG | 0 | 2104 |
| 1126748 | 2357 | 2376 | 26615 | 26634 | TCCACAGAGTTGCGCTTGCT | 39 | 2105 |
| 1126780 | 2638 | 2657 | 26896 | 26915 | GAAAGGCACGGCTTTGTGGA | 68 | 2106 |
| 1126812 | 2858 | 2877 | 27116 | 27135 | TTGGTGGCCCGCATGCCGGG | 12 | 2107 |
| 1126844 | 2923 | 2942 | 27181 | 27200 | CTGAAAGTGCCCCGGCTCTG | 36 | 2108 |
| 1126876 | 3187 | 3206 | 27445 | 27464 | GAAGAGTAGGATCTGGTCCA | 14 | 2109 |
| 1126908 | 3427 | 3446 | 27685 | 27704 | GAAGAGCCTCGAGGTAAATG | 67 | 2110 |
| 1126940 | N/A | N/A | 3045 | 3064 | CGGGCCCCCATCCACTACTG | 20 | 2111 |
| 1126972 | N/A | N/A | 3091 | 3110 | CACAAGGGTTCCCCTAGTAG | 58 | 2112 |
| 1127004 | N/A | N/A | 3281 | 3300 | AGGACCCAGGTATCTAGTCT | 54 | 2113 |
| 1127036 | N/A | N/A | 3362 | 3381 | GTTAGGGTCATAGGTCTGAA | 91 | 2114 |
| 1127068 | N/A | N/A | 3483 | 3502 | CAGCCCGTCCCATCTTTATT | 5 | 2115 |
| 1127100 | N/A | N/A | 3589 | 3608 | CCCTATTCCAGTACGCATGT | 61 | 2116 |
| 1127132 | N/A | N/A | 3856 | 3875 | CAGGCCCTGGCGATACCCGG | 16 | 2117 |
| 1127164 | N/A | N/A | 4146 | 4165 | GACTGACTACCCGGACTCAG | 38 | 2118 |
| 1127196 | N/A | N/A | 4472 | 4491 | GCCCCGGCCGAGGTCCATAG | 16 | 2119 |
| 1127228 | N/A | N/A | 4876 | 4895 | TCGCCCATGTCTGGGATCCA | 21 | 2120 |
| 1127260 | N/A | N/A | 5311 | 5330 | CGCTATTAGCATGTCCATTA | 83 | 2121 |
| 1127292 | N/A | N/A | 5598 | 5617 | CCCGATATTCCATGCTTCTC | 76 | 2122 |
| 1127324 | N/A | N/A | 5901 | 5920 | CCTTAGGGACACAGTCGGAC | 52 | 2123 |
| 1127356 | N/A | N/A | 6083 | 6102 | AGTGGAGAAGCAGCCCCGCT | 0 | 2124 |
| 1127388 | N/A | N/A | 7012 | 7031 | CATATCTGTCACCTGCGACC | 58 | 2125 |
| 1127420 | N/A | N/A | 8365 | 8384 | TCCGTGCCCCCTGCTTACTT | 84 | 2126 |
| 1127452 | N/A | N/A | 8540 | 8559 | TCACTCATCCGTGGTTCTCC | 48 | 2127 |
| 1127484 | N/A | N/A | 8920 | 8939 | GGCCAGAGCTAATCCTTGGA | 13 | 2128 |
| 1127516 | N/A | N/A | 9032 | 9051 | CGCCAGCCTCACTTTGAGGC | 7 | 2129 |
| 1127548 | N/A | N/A | 9576 | 9595 | GGCAAAGGGACTACAACTCC | 38 | 2130 |
| 1127580 | N/A | N/A | 9678 | 9697 | GCAGGGTTAGATTGGACAGA | 65 | 2131 |
| 1127612 | N/A | N/A | 9805 | 9824 | CCAACCGGACAGGGACCTGT | 0 | 2132 |
| 1127644 | N/A | N/A | 9896 | 9915 | ATCGGCAATCCCCAGTAGGG | 9 | 2133 |
| 1127676 | N/A | N/A | 10170 | 10189 | ATACTCGCAGTCCCCCATCT | 31 | 2134 |
| 1127708 | N/A | N/A | 10588 | 10607 | GCAACTGGCTTTGGCATAGA | 74 | 2135 |
| 1127740 | N/A | N/A | 12028 | 12047 | AATGGTAACTGATGGTCCCC | 60 | 2136 |
| 1127772 | N/A | N/A | 12297 | 12316 | TATACTCACTGAGAACGGAT | 58 | 2137 |
| 1127804 | N/A | N/A | 12473 | 12492 | TCCTAGGATATATCCTCTAC | 6 | 2138 |
| 1127836 | N/A | N/A | 13110 | 13129 | GTTCATTCAGATCAATGTTG | 61 | 2139 |
| 1127868 | N/A | N/A | 13400 | 13419 | ATTAAGGAGGCCGAATACCC | 0 | 2140 |
| 1127900 | N/A | N/A | 13599 | 13618 | ACCCACAAGGCACGGTAAAG | 0 | 2141 |
| 1127932 | N/A | N/A | 14277 | 14296 | GTCAAGACCACTGGGATCCT | 40 | 2142 |
| 1127954 | N/A | N/A | 15654 | 15673 | CCGTCTACAGGATTTTCTAG | 95 | 83 |
| 1127965 | N/A | N/A | 15701 | 15720 | CCCGTCTCATATTCCAGTTC | 63 | 2143 |
| 1127997 | N/A | N/A | 16729 | 16748 | ACGGTCACTCCTCCACTTAA | 69 | 2144 |
| 1128029 | N/A | N/A | 16891 | 16910 | GTACACATGGCAAGCCTTCC | 54 | 2145 |
| 1128061 | N/A | N/A | 17152 | 17171 | CAGCTACCCTTAGGTTTCTA | 29 | 2146 |
| 1128093 | N/A | N/A | 17962 | 17981 | GCATGCATGTGAGAACGGAG | 0 | 2147 |
| 1128125 | N/A | N/A | 18239 | 18258 | AGCAACTGAAGTTGCACGAG | 6 | 2148 |
| 1128157 | N/A | N/A | 19305 | 19324 | GTGGAGCCGTGTGCAGCAAG | 11 | 2149 |
| 1128189 | N/A | N/A | 19543 | 19562 | GAATGACGGTAAGTTCTCAT | 53 | 2150 |
| 1128221 | N/A | N/A | 19686 | 19705 | ACCACAAGTACTGAAGTCCC | 30 | 2151 |
| 1128253 | N/A | N/A | 19827 | 19846 | CGATGGCAGAAACATTCATA | 38 | 2152 |
| 1128285 | N/A | N/A | 19970 | 19989 | GACAGAGCTAGTGTGATAGG | 62 | 2153 |
| 1128317 | N/A | N/A | 21189 | 21208 | TCATGAGCCATGCGGACCCT | 48 | 2154 |
| 1128349 | N/A | N/A | 21780 | 21799 | ACTACACTCACCCGGTGTGT | 14 | 2155 |
| 1128381 | N/A | N/A | 21839 | 21858 | CCTCGCCCCAAGACATGCTA | 25 | 2156 |
| 1128413 | N/A | N/A | 21991 | 22010 | TGTGACATCCAGTTCACTAG | 0 | 2157 |
| 1128445 | N/A | N/A | 22562 | 22581 | CGCGACTCCTAATTATCATG | 49 | 2158 |
| 1128477 | N/A | N/A | 24590 | 24609 | AGTATCTTGTACGGACTTAG | 45 | 2159 |
| 1128509 | N/A | N/A | 24729 | 24748 | GTCCATAGTTGGGAGATAAG | 38 | 2160 |
| 1128541 | N/A | N/A | 24914 | 24933 | CTACCGTCTTAATCGCAATA | 0 | 2161 |
| 1128573 | N/A | N/A | 25142 | 25161 | TCCAAGGGTGCAAGCTCCTC | 20 | 2162 |
| 1128605 | N/A | N/A | 25532 | 25551 | TGTTACAAGTAGGATGAGAC | 52 | 2163 |
| 1128637 | N/A | N/A | 25629 | 25648 | CATGCGATCCTTCCTGTTAG | 30 | 2164 |
| 1128669 | N/A | N/A | 26052 | 26071 | GCCACACCCTTTATGCAGAT | 41 | 2165 |
| 1128701 | N/A | N/A | 26373 | 26392 | GGCCTGCATACGGCGATGTG | 14 | 2166 |

TABLE 30
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126269 | 99 | 118 | 2746 | 2765 | CGGCTTCCTATTGCAAGACC | 27 | 2167 |
| 1126301 | 181 | 200 | 2828 | 2847 | GACCCCGGAGGTGTCTAGGG | 5 | 2168 |
| 1126333 | 296 | 315 | 2943 | 2962 | TCCAGGTCGAATTCATCCTC | 49 | 2169 |
| 1126365 | 508 | 527 | 4666 | 4685 | TGTTCATGGAATCCAGTGTC | 38 | 2170 |
| 1126397 | 809 | 828 | 10041 | 10060 | AGTCGCCGGGCACGACACAG | 4 | 2171 |
| 1126429 | 890 | 909 | 10122 | 10141 | TTGTTGTAGAAGTCCACGGC | 10 | 2172 |
| 1126461 | 1030 | 1049 | 10506 | 10525 | GCAAGTGCTGTGCCTCGATG | 75† | 2173 |
| 1126493 | 1273 | 1292 | 13716 | 13735 | AGTTGAGCCGAGCCAATGCC | 17 | 2174 |
| 1126525 | 1344 | 1363 | 14405 | 14424 | ATTGTTGGTCCGCGCTGGCA | 40 | 2175 |
| 1126557 | 1572 | 1591 | 21290 | 21309 | ATCCAGCATATTGTGGGTGC | 9 | 2176 |
| 1126589 | 1636 | 1655 | 21354 | 21373 | TGTCGGCACTGCTATTGAAG | 0 | 2177 |
| 1126621 | 1838 | 1857 | 25025 | 25044 | TCCATGAAGCAGCCGAAGCC | 0 | 2178 |
| 1126653 | 1975 | 1994 | 25380 | 25399 | GGATGATACGCTGCCGCCGG | 0 | 2179 |
| 1126685 | 2138 | 2157 | 26396 | 26415 | GGCCGTGGGTAGCGGTACCC | 9 | 2180 |
| 1126717 | 2284 | 2303 | 26542 | 26561 | TGTTGCGCCGGTCCTTGGCG | 12 | 2181 |
| 1126749 | 2359 | 2378 | 26617 | 26636 | TGTCCACAGAGTTGCGCTTG | 13 | 2182 |
| 1126781 | 2639 | 2658 | 26897 | 26916 | AGAAAGGCACGGCTTTGTGG | 68 | 2183 |
| 1126813 | 2860 | 2879 | 27118 | 27137 | ACTTGGTGGCCCGCATGCCG | 0 | 2184 |
| 1126845 | 2924 | 2943 | 27182 | 27201 | TCTGAAAGTGCCCCGGCTCT | 48 | 2185 |
| 1126877 | 3220 | 3239 | 27478 | 27497 | GATTCCCTGGAGGGAGATCT | 0 | 2186 |
| 1126909 | 3428 | 3447 | 27686 | 27705 | GGAAGAGCCTCGAGGTAAAT | 74 | 2187 |
| 1126941 | N/A | N/A | 3047 | 3066 | TCCGGGCCCCCATCCACTAC | 5 | 2188 |
| 1126973 | N/A | N/A | 3092 | 3111 | TCACAAGGGTTCCCCTAGTA | 33 | 2189 |
| 1127005 | N/A | N/A | 3283 | 3302 | TCAGGACCCAGGTATCTAGT | 67 | 2190 |
| 1127037 | N/A | N/A | 3364 | 3383 | CTGTTAGGGTCATAGGTCTG | 75 | 2191 |
| 1127069 | N/A | N/A | 3484 | 3503 | CCAGCCCGTCCCATCTTTAT | 19 | 2192 |
| 1127101 | N/A | N/A | 3590 | 3609 | CCCCTATTCCAGTACGCATG | 66 | 2193 |
| 1127133 | N/A | N/A | 3857 | 3876 | CCAGGCCCTGGCGATACCCG | 28 | 2194 |
| 1127165 | N/A | N/A | 4147 | 4166 | GGACTGACTACCCGGACTCA | 57 | 2195 |
| 1127197 | N/A | N/A | 4473 | 4492 | GGCCCCGGCCGAGGTCCATA | 2 | 2196 |
| 1127229 | N/A | N/A | 4877 | 4896 | TTCGCCCATGTCTGGGATCC | 32 | 2197 |
| 1127261 | N/A | N/A | 5312 | 5331 | ACGCTATTAGCATGTCCATT | 90 | 2198 |
| 1127293 | N/A | N/A | 5601 | 5620 | TTCCCCGATATTCCATGCTT | 47 | 2199 |
| 1127325 | N/A | N/A | 5903 | 5922 | TCCCTTAGGGACACAGTCGG | 44 | 2200 |
| 1127357 | N/A | N/A | 6189 | 6208 | CGTGTTTCTAAGACTTGCTG | 86 | 2201 |
| 1127389 | N/A | N/A | 7013 | 7032 | ACATATCTGTCACCTGCGAC | 56 | 2202 |
| 1127421 | N/A | N/A | 8367 | 8386 | TTTCCGTGCCCCCTGCTTAC | 64 | 2203 |
| 1127453 | N/A | N/A | 8542 | 8561 | CTTCACTCATCCGTGGTTCT | 52 | 2204 |
| 1127485 | N/A | N/A | 8921 | 8940 | AGGCCAGAGCTAATCCTTGG | 15 | 2205 |
| 1127517 | N/A | N/A | 9035 | 9054 | GCCCGCCAGCCTCACTTTGA | 8 | 2206 |
| 1127549 | N/A | N/A | 9582 | 9601 | AACATTGGCAAAGGGACTAC | 0 | 2207 |
| 1127581 | N/A | N/A | 9679 | 9698 | GGCAGGGTTAGATTGGACAG | 63 | 2208 |
| 1127613 | N/A | N/A | 9806 | 9825 | CCCAACCGGACAGGGACCTG | 2 | 2209 |
| 1127645 | N/A | N/A | 9897 | 9916 | AATCGGCAATCCCCAGTAGG | 11 | 2210 |
| 1127677 | N/A | N/A | 10171 | 10190 | CATACTCGCAGTCCCCCATC | 6 | 2211 |
| 1127709 | N/A | N/A | 10589 | 10608 | AGCAACTGGCTTTGGCATAG | 52 | 2212 |
| 1127741 | N/A | N/A | 12029 | 12048 | AAATGGTAACTGATGGTCCC | 63 | 2213 |
| 1127773 | N/A | N/A | 12298 | 12317 | TTATACTCACTGAGAACGGA | 68 | 2214 |
| 1127805 | N/A | N/A | 12478 | 12497 | GCTTCTCCTAGGATATATCC | 50 | 2215 |
| 1127837 | N/A | N/A | 13132 | 13151 | GTGAGGGTCCCATGTTTTAT | 23 | 2216 |
| 1127869 | N/A | N/A | 13401 | 13420 | AATTAAGGAGGCCGAATACC | 0 | 2217 |
| 1127901 | N/A | N/A | 13600 | 13619 | AACCCACAAGGCACGGTAAA | 5 | 2218 |
| 1127933 | N/A | N/A | 14352 | 14371 | CGTTCACCTGCGCAGAAAGA | 0 | 2219 |
| 1127954 | N/A | N/A | 15654 | 15673 | CCGTCTACAGGATTTTCTAG | 94 | 83 |
| 1127966 | N/A | N/A | 15702 | 15721 | CCCCGTCTCATATTCCAGTT | 56 | 2220 |
| 1127998 | N/A | N/A | 16730 | 16749 | CACGGTCACTCCTCCACTTA | 47 | 2221 |
| 1128030 | N/A | N/A | 16892 | 16911 | AGTACACATGGCAAGCCTTC | 34 | 2222 |
| 1128062 | N/A | N/A | 17154 | 17173 | CCCAGCTACCCTTAGGTTTC | 32 | 2223 |
| 1128094 | N/A | N/A | 17963 | 17982 | GGCATGCATGTGAGAACGGA | 14 | 2224 |
| 1128126 | N/A | N/A | 18240 | 18259 | CAGCAACTGAAGTTGCACGA | 0 | 2225 |
| 1128158 | N/A | N/A | 19306 | 19325 | GGTGGAGCCGTGTGCAGCAA | 24 | 2226 |
| 1128190 | N/A | N/A | 19544 | 19563 | AGAATGACGGTAAGTTCTCA | 31 | 2227 |
| 1128222 | N/A | N/A | 19687 | 19706 | GACCACAAGTACTGAAGTCC | 28 | 2228 |
| 1128254 | N/A | N/A | 19828 | 19847 | CCGATGGCAGAAACATTCAT | 0 | 2229 |
| 1128286 | N/A | N/A | 20025 | 20044 | TCCAACCCTAACAAAGGCCC | 6 | 2230 |
| 1128318 | N/A | N/A | 21190 | 21209 | CTCATGAGCCATGCGGACCC | 7 | 2231 |
| 1128350 | N/A | N/A | 21781 | 21800 | CACTACACTCACCCGGTGTG | 10 | 2232 |
| 1128382 | N/A | N/A | 21843 | 21862 | AGTGCCTCGCCCCAAGACAT | 0 | 2233 |
| 1128414 | N/A | N/A | 21997 | 22016 | GTGAACTGTGACATCCAGTT | 8 | 2234 |
| 1128446 | N/A | N/A | 22563 | 22582 | CCGCGACTCCTAATTATCAT | 28 | 2235 |
| 1128478 | N/A | N/A | 24591 | 24610 | CAGTATCTTGTACGGACTTA | 39 | 2236 |
| 1128510 | N/A | N/A | 24730 | 24749 | AGTCCATAGTTGGGAGATAA | 22 | 2237 |
| 1128542 | N/A | N/A | 24915 | 24934 | CCTACCGTCTTAATCGCAAT | 0 | 2238 |
| 1128574 | N/A | N/A | 25144 | 25163 | TCTCCAAGGGTGCAAGCTCC | 0 | 2239 |
| 1128606 | N/A | N/A | 25533 | 25552 | CTGTTACAAGTAGGATGAGA | 44 | 2240 |
| 1128638 | N/A | N/A | 25630 | 25649 | TCATGCGATCCTTCCTGTTA | 42 | 2241 |
| 1128670 | N/A | N/A | 26055 | 26074 | TTGGCCACACCCTTTATGCA | 7 | 2242 |
| 1128702 | N/A | N/A | 26374 | 26393 | GGGCCTGCATACGGCGATGT | 0 | 2243 |

TABLE 31
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126270 | 100 | 119 | 2747 | 2766 | TCGGCTTCCTATTGCAAGAC | 11 | 2244 |
| 1126302 | 183 | 202 | 2830 | 2849 | GGGACCCCGGAGGTGTCTAG | 0 | 2245 |
| 1126334 | 298 | 317 | 2945 | 2964 | TCTCCAGGTCGAATTCATCC | 55 | 2246 |
| 1126366 | 559 | 578 | 8633 | 8652 | GAGGGCCTCCCTCGATCAGC | 7 | 2247 |
| 1126398 | 810 | 829 | 10042 | 10061 | CAGTCGCCGGGCACGACACA | 3 | 2248 |
| 1126430 | 892 | 911 | 10124 | 10143 | GGTTGTTGTAGAAGTCCACG | 31 | 2249 |
| 1126462 | 1031 | 1050 | 10507 | 10526 | AGCAAGTGCTGTGCCTCGAT | 661 | 2250 |
| 1126494 | 1274 | 1293 | 13717 | 13736 | TAGTTGAGCCGAGCCAATGC | 9 | 2251 |
| 1126526 | 1346 | 1365 | 14407 | 14426 | AAATTGTTGGTCCGCGCTGG | 37 | 2252 |
| 1126558 | 1573 | 1592 | 21291 | 21310 | CATCCAGCATATTGTGGGTG | 13 | 2253 |
| 1126590 | 1637 | 1656 | 21355 | 21374 | CTGTCGGCACTGCTATTGAA | 10 | 2254 |
| 1126622 | 1849 | 1868 | 25036 | 25055 | CGATGTGTTCCTCCATGAAG | 24 | 2255 |
| 1126654 | 1976 | 1995 | 25381 | 25400 | TGGATGATACGCTGCCGCCG | 0 | 2256 |
| 1126686 | 2139 | 2158 | 26397 | 26416 | TGGCCGTGGGTAGCGGTACC | 8 | 2257 |
| 1126718 | 2285 | 2304 | 26543 | 26562 | ATGTTGCGCCGGTCCTTGGC | 7 | 2258 |
| 1126750 | 2360 | 2379 | 26618 | 26637 | GTGTCCACAGAGTTGCGCTT | 17 | 2259 |
| 1126782 | 2640 | 2659 | 26898 | 26917 | GAGAAAGGCACGGCTTTGTG | 60 | 2260 |
| 1126814 | 2861 | 2880 | 27119 | 27138 | AACTTGGTGGCCCGCATGCC | 4 | 2261 |
| 1126846 | 2926 | 2945 | 27184 | 27203 | ACTCTGAAAGTGCCCCGGCT | 22 | 2262 |
| 1126878 | 3225 | 3244 | 27483 | 27502 | AGGCGGATTCCCTGGAGGGA | 41 | 2263 |
| 1126910 | 3429 | 3448 | 27687 | 27706 | TGGAAGAGCCTCGAGGTAAA | 55 | 2264 |
| 1126942 | N/A | N/A | 3048 | 3067 | TTCCGGGCCCCCATCCACTA | 20 | 2265 |
| 1126974 | N/A | N/A | 3093 | 3112 | GTCACAAGGGTTCCCCTAGT | 50 | 2266 |
| 1127006 | N/A | N/A | 3287 | 3306 | CTGTTCAGGACCCAGGTATC | 41 | 2267 |
| 1127038 | N/A | N/A | 3365 | 3384 | GCTGTTAGGGTCATAGGTCT | 85 | 2268 |
| 1127070 | N/A | N/A | 3485 | 3504 | TCCAGCCCGTCCCATCTTTA | 47 | 2269 |
| 1127102 | N/A | N/A | 3591 | 3610 | TCCCCTATTCCAGTACGCAT | 79 | 2270 |
| 1127134 | N/A | N/A | 3951 | 3970 | TGACTAGGGACACCAGCATG | 13 | 2271 |
| 1127166 | N/A | N/A | 4148 | 4167 | AGGACTGACTACCCGGACTC | 46 | 2272 |
| 1127198 | N/A | N/A | 4485 | 4504 | CGTGTGAGGGCAGGCCCCGG | 10 | 2273 |
| 1127230 | N/A | N/A | 4878 | 4897 | ATTCGCCCATGTCTGGGATC | 46 | 2274 |
| 1127262 | N/A | N/A | 5328 | 5347 | CACCGCTAGAAGGTAGACGC | 54 | 2275 |
| 1127294 | N/A | N/A | 5602 | 5621 | CTTCCCCGATATTCCATGCT | 43 | 2276 |
| 1127326 | N/A | N/A | 5904 | 5923 | TTCCCTTAGGGACACAGTCG | 59 | 2277 |
| 1127358 | N/A | N/A | 6190 | 6209 | CCGTGTTTCTAAGACTTGCT | 92 | 2278 |
| 1127390 | N/A | N/A | 7014 | 7033 | CACATATCTGTCACCTGCGA | 74 | 2279 |
| 1127422 | N/A | N/A | 8368 | 8387 | TTTTCCGTGCCCCCTGCTTA | 43 | 2280 |
| 1127454 | N/A | N/A | 8543 | 8562 | TCTTCACTCATCCGTGGTTC | 26 | 2281 |
| 1127486 | N/A | N/A | 8945 | 8964 | GCCCTTGGATGTTCATGTCC | 15 | 2282 |
| 1127518 | N/A | N/A | 9043 | 9062 | TCGGGCAAGCCCGCCAGCCT | 0 | 2283 |
| 1127550 | N/A | N/A | 9584 | 9603 | CCAACATTGGCAAAGGGACT | 7 | 2284 |
| 1127582 | N/A | N/A | 9680 | 9699 | TGGCAGGGTTAGATTGGACA | 87 | 2285 |
| 1127614 | N/A | N/A | 9807 | 9826 | TCCCAACCGGACAGGGACCT | 2 | 2286 |
| 1127646 | N/A | N/A | 9898 | 9917 | GAATCGGCAATCCCCAGTAG | 0 | 2287 |
| 1127678 | N/A | N/A | 10172 | 10191 | GCATACTCGCAGTCCCCCAT | 45 | 2288 |
| 1127710 | N/A | N/A | 10633 | 10652 | CGGTTTATGCTATTTCAACT | 90 | 2289 |
| 1127742 | N/A | N/A | 12060 | 12079 | GGAGCTGTAGTTCTCTATTC | 14 | 2290 |
| 1127774 | N/A | N/A | 12299 | 12318 | CTTATACTCACTGAGAACGG | 58 | 2291 |
| 1127806 | N/A | N/A | 12575 | 12594 | GATCTGATCCACACTGGTGA | 36 | 2292 |
| 1127838 | N/A | N/A | 13155 | 13174 | GGTCACAATATCTGGGATTG | 51† | 2293 |
| 1127870 | N/A | N/A | 13402 | 13421 | GAATTAAGGAGGCCGAATAC | 4 | 2294 |
| 1127902 | N/A | N/A | 13601 | 13620 | GAACCCACAAGGCACGGTAA | 9 | 2295 |
| 1127934 | N/A | N/A | 14353 | 14372 | CCGTTCACCTGCGCAGAAAG | 7 | 2296 |
| 1127954 | N/A | N/A | 15654 | 15673 | CCGTCTACAGGATTTTCTAG | 95 | 83 |
| 1127967 | N/A | N/A | 15703 | 15722 | ACCCCGTCTCATATTCCAGT | 63 | 2297 |
| 1127999 | N/A | N/A | 16738 | 16757 | AACAGCCCCACGGTCACTCC | 54 | 2298 |
| 1128031 | N/A | N/A | 16907 | 16926 | GATGTCATGCCAAAAAGTAC | 17 | 2299 |
| 1128063 | N/A | N/A | 17155 | 17174 | GCCCAGCTACCCTTAGGTTT | 12 | 2300 |
| 1128095 | N/A | N/A | 17964 | 17983 | GGGCATGCATGTGAGAACGG | 0 | 2301 |
| 1128127 | N/A | N/A | 18245 | 18264 | GCCTCCAGCAACTGAAGTTG | 0 | 2302 |
| 1128159 | N/A | N/A | 19307 | 19326 | AGGTGGAGCCGTGTGCAGCA | 43 | 2303 |
| 1128191 | N/A | N/A | 19545 | 19564 | TAGAATGACGGTAAGTTCTC | 32 | 2304 |
| 1128223 | N/A | N/A | 19690 | 19709 | GTAGACCACAAGTACTGAAG | 63 | 2305 |
| 1128255 | N/A | N/A | 19829 | 19848 | CCCGATGGCAGAAACATTCA | 12 | 2306 |
| 1128287 | N/A | N/A | 20672 | 20691 | GCGATGCATCCGTTTTGGCC | 0 | 2307 |
| 1128319 | N/A | N/A | 21191 | 21210 | CCTCATGAGCCATGCGGACC | 13 | 2308 |
| 1128351 | N/A | N/A | 21782 | 21801 | CCACTACACTCACCCGGTGT | 0 | 2309 |
| 1128383 | N/A | N/A | 21844 | 21863 | CAGTGCCTCGCCCCAAGACA | 0 | 2310 |
| 1128415 | N/A | N/A | 22001 | 22020 | CAGTGTGAACTGTGACATCC | 5 | 2311 |
| 1128447 | N/A | N/A | 22565 | 22584 | GACCGCGACTCCTAATTATC | 16 | 2312 |
| 1128479 | N/A | N/A | 24592 | 24611 | CCAGTATCTTGTACGGACTT | 29 | 2313 |
| 1128511 | N/A | N/A | 24733 | 24752 | GAAAGTCCATAGTTGGGAGA | 8 | 2314 |
| 1128543 | N/A | N/A | 24916 | 24935 | GCCTACCGTCTTAATCGCAA | 1 | 2315 |
| 1128575 | N/A | N/A | 25145 | 25164 | GTCTCCAAGGGTGCAAGCTC | 14 | 2316 |
| 1128607 | N/A | N/A | 25534 | 25553 | ACTGTTACAAGTAGGATGAG | 13 | 2317 |
| 1128639 | N/A | N/A | 25805 | 25824 | GCAGTGGGTTTGAACAAGGC | 33 | 2318 |
| 1128671 | N/A | N/A | 26057 | 26076 | TCTTGGCCACACCCTTTATG | 26 | 2319 |
| 1128703 | N/A | N/A | 26375 | 26394 | TGGGCCTGCATACGGCGATG | 2 | 2320 |

TABLE 32
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126271 | 101 | 120 | 2748 | 2767 | CTCGGCTTCCTATTGCAAGA | 0 | 2321 |
| 1126303 | 184 | 203 | 2831 | 2850 | AGGGACCCCGGAGGTGTCTA | 0 | 2322 |
| 1126335 | 299 | 318 | 2946 | 2965 | TTCTCCAGGTCGAATTCATC | 0 | 2323 |
| 1126367 | 560 | 579 | 8634 | 8653 | AGAGGGCCTCCCTCGATCAG | 0 | 2324 |
| 1126399 | 811 | 830 | 10043 | 10062 | GCAGTCGCCGGGCACGACAC | 0 | 2325 |
| 1126431 | 961 | 980 | 10437 | 10456 | CCCTTTCCATGCAGTATCGG | 0 | 2326 |
| 1126463 | 1032 | 1051 | 10508 | 10527 | GAGCAAGTGCTGTGCCTCGA | 47† | 2327 |
| 1126495 | 1275 | 1294 | 13718 | 13737 | ATAGTTGAGCCGAGCCAATG | 0 | 2328 |
| 1126527 | 1347 | 1366 | 14408 | 14427 | GAAATTGTTGGTCCGCGCTG | 11 | 2329 |
| 1126559 | 1574 | 1593 | 21292 | 21311 | TCATCCAGCATATTGTGGGT | 14 | 2330 |
| 1126591 | 1638 | 1657 | 21356 | 21375 | CCTGTCGGCACTGCTATTGA | 0 | 2331 |
| 1126623 | 1850 | 1869 | 25037 | 25056 | GCGATGTGTTCCTCCATGAA | 45 | 2332 |
| 1126655 | 1977 | 1996 | 25382 | 25401 | CTGGATGATACGCTGCCGCC | 0 | 2333 |
| 1126687 | 2140 | 2159 | 26398 | 26417 | CTGGCCGTGGGTAGCGGTAC | 0 | 2334 |
| 1126719 | 2286 | 2305 | 26544 | 26563 | GATGTTGCGCCGGTCCTTGG | 6 | 2335 |
| 1126751 | 2362 | 2381 | 26620 | 26639 | CCGTGTCCACAGAGTTGCGC | 12 | 2336 |
| 1126783 | 2662 | 2681 | 26920 | 26939 | GATTATGCATATTCTGGAGC | 73 | 2337 |
| 1126815 | 2862 | 2881 | 27120 | 27139 | GAACTTGGTGGCCCGCATGC | 0 | 2338 |
| 1126847 | 2927 | 2946 | 27185 | 27204 | AACTCTGAAAGTGCCCCGGC | 11 | 2339 |
| 1126879 | 3226 | 3245 | 27484 | 27503 | CAGGCGGATTCCCTGGAGGG | 47 | 2340 |
| 1126911 | 3430 | 3449 | 27688 | 27707 | ATGGAAGAGCCTCGAGGTAA | 25 | 2341 |
| 1126943 | N/A | N/A | 3050 | 3069 | AGTTCCGGGCCCCCATCCAC | 28 | 2342 |
| 1126975 | N/A | N/A | 3094 | 3113 | GGTCACAAGGGTTCCCCTAG | 59 | 2343 |
| 1127007 | N/A | N/A | 3289 | 3308 | CCCTGTTCAGGACCCAGGTA | 0 | 2344 |
| 1127039 | N/A | N/A | 3366 | 3385 | TGCTGTTAGGGTCATAGGTC | 93 | 2345 |
| 1127071 | N/A | N/A | 3493 | 3512 | TTACAGTCTCCAGCCCGTCC | 46 | 2346 |
| 1127103 | N/A | N/A | 3592 | 3611 | TTCCCCTATTCCAGTACGCA | 75 | 2347 |
| 1127135 | N/A | N/A | 3952 | 3971 | CTGACTAGGGACACCAGCAT | 12 | 2348 |
| 1127167 | N/A | N/A | 4149 | 4168 | GAGGACTGACTACCCGGACT | 62 | 2349 |
| 1127199 | N/A | N/A | 4503 | 4522 | ACCCACTGTGGGCCCAAGCG | 6 | 2350 |
| 1127231 | N/A | N/A | 4879 | 4898 | CATTCGCCCATGTCTGGGAT | 37 | 2351 |
| 1127263 | N/A | N/A | 5329 | 5348 | GCACCGCTAGAAGGTAGACG | 62 | 2352 |
| 1127295 | N/A | N/A | 5603 | 5622 | TCTTCCCCGATATTCCATGC | 69 | 2353 |
| 1127327 | N/A | N/A | 5905 | 5924 | GTTCCCTTAGGGACACAGTC | 0 | 2354 |
| 1127359 | N/A | N/A | 6191 | 6210 | GCCGTGTTTCTAAGACTTGC | 73 | 2355 |
| 1127391 | N/A | N/A | 7015 | 7034 | TCACATATCTGTCACCTGCG | 81 | 2356 |
| 1127423 | N/A | N/A | 8369 | 8388 | ATTTTCCGTGCCCCCTGCTT | 11 | 2357 |
| 1127455 | N/A | N/A | 8545 | 8564 | TATCTTCACTCATCCGTGGT | 17 | 2358 |
| 1127487 | N/A | N/A | 8946 | 8965 | GGCCCTTGGATGTTCATGTC | 11 | 2359 |
| 1127519 | N/A | N/A | 9045 | 9064 | CCTCGGGCAAGCCCGCCAGC | 0 | 2360 |
| 1127551 | N/A | N/A | 9588 | 9607 | GCACCCAACATTGGCAAAGG | 19 | 2361 |
| 1127583 | N/A | N/A | 9681 | 9700 | CTGGCAGGGTTAGATTGGAC | 68 | 2362 |
| 1127615 | N/A | N/A | 9808 | 9827 | ATCCCAACCGGACAGGGACC | 0 | 2363 |
| 1127647 | N/A | N/A | 9899 | 9918 | AGAATCGGCAATCCCCAGTA | 17 | 2364 |
| 1127679 | N/A | N/A | 10173 | 10192 | GGCATACTCGCAGTCCCCCA | 33 | 2365 |
| 1127711 | N/A | N/A | 10634 | 10653 | TCGGTTTATGCTATTTCAAC | 89 | 2366 |
| 1127743 | N/A | N/A | 12093 | 12112 | TCAAATACAGGTCACCACCC | 0 | 2367 |
| 1127775 | N/A | N/A | 12300 | 12319 | CCTTATACTCACTGAGAACG | 54 | 2368 |
| 1127807 | N/A | N/A | 12576 | 12595 | GGATCTGATCCACACTGGTG | 17 | 2369 |
| 1127839 | N/A | N/A | 13291 | 13310 | CTTAAGACCTAGGTATATGC | 0 | 2370 |
| 1127871 | N/A | N/A | 13408 | 13427 | GCTCAAGAATTAAGGAGGCC | 19 | 2371 |
| 1127903 | N/A | N/A | 13602 | 13621 | AGAACCCACAAGGCACGGTA | 2 | 2372 |
| 1127935 | N/A | N/A | 14354 | 14373 | GCCGTTCACCTGCGCAGAAA | 4 | 2373 |
| 1127954 | N/A | N/A | 15654 | 15673 | CCGTCTACAGGATTTTCTAG | 94 | 83 |
| 1127968 | N/A | N/A | 15704 | 15723 | AACCCCGTCTCATATTCCAG | 60 | 2374 |
| 1128000 | N/A | N/A | 16740 | 16759 | ACAACAGCCCCACGGTCACT | 53 | 2375 |
| 1128032 | N/A | N/A | 16915 | 16934 | GACCCAATGATGTCATGCCA | 80 | 2376 |
| 1128064 | N/A | N/A | 17156 | 17175 | CGCCCAGCTACCCTTAGGTT | 8 | 2377 |
| 1128096 | N/A | N/A | 17990 | 18009 | GAAGGCTCCCACTGCAAGGC | 4 | 2378 |
| 1128128 | N/A | N/A | 18246 | 18265 | CGCCTCCAGCAACTGAAGTT | 20 | 2379 |
| 1128160 | N/A | N/A | 19308 | 19327 | GAGGTGGAGCCGTGTGCAGC | 19 | 2380 |
| 1128192 | N/A | N/A | 19546 | 19565 | ATAGAATGACGGTAAGTTCT | 30 | 2381 |
| 1128224 | N/A | N/A | 19692 | 19711 | ATGTAGACCACAAGTACTGA | 50 | 2382 |
| 1128256 | N/A | N/A | 19831 | 19850 | ATCCCGATGGCAGAAACATT | 7 | 2383 |
| 1128288 | N/A | N/A | 20674 | 20693 | AAGCGATGCATCCGTTTTGG | 35 | 2384 |
| 1128320 | N/A | N/A | 21192 | 21211 | TCCTCATGAGCCATGCGGAC | 9 | 2385 |
| 1128352 | N/A | N/A | 21783 | 21802 | CCCACTACACTCACCCGGTG | 20 | 2386 |
| 1128384 | N/A | N/A | 21845 | 21864 | CCAGTGCCTCGCCCCAAGAC | 10 | 2387 |
| 1128416 | N/A | N/A | 22072 | 22091 | CACTCGGCTGTATTTGTGAA | 15 | 2388 |
| 1128448 | N/A | N/A | 22566 | 22585 | TGACCGCGACTCCTAATTAT | 31 | 2389 |
| 1128480 | N/A | N/A | 24593 | 24612 | GCCAGTATCTTGTACGGACT | 51 | 2390 |
| 1128512 | N/A | N/A | 24734 | 24753 | GGAAAGTCCATAGTTGGGAG | 47 | 2391 |
| 1128544 | N/A | N/A | 24917 | 24936 | CGCCTACCGTCTTAATCGCA | 0 | 2392 |
| 1128576 | N/A | N/A | 25175 | 25194 | CGAGAGCCCCCCCATTTCCT | 18 | 2393 |
| 1128608 | N/A | N/A | 25536 | 25555 | CAACTGTTACAAGTAGGATG | 53 | 2394 |
| 1128640 | N/A | N/A | 25828 | 25847 | GTTCTGTTACTCTAGGATGG | 71 | 2395 |
| 1128672 | N/A | N/A | 26140 | 26159 | GCAGACATATAGTACTAGGG | 73 | 2396 |
| 1128704 | N/A | N/A | 26377 | 26396 | CCTGGGCCTGCATACGGCGA | 8 | 2397 |

TABLE 33
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126272 | 102 | 121 | 2749 | 2768 | GCTCGGCTTCCTATTGCAAG | 25 | 2398 |
| 1126304 | 185 | 204 | 2832 | 2851 | TAGGGACCCCGGAGGTGTCT | 0 | 2399 |
| 1126336 | 300 | 319 | 2947 | 2966 | GTTCTCCAGGTCGAATTCAT | 51 | 2400 |
| 1126368 | 562 | 581 | 8636 | 8655 | CCAGAGGGCCTCCCTCGATC | 0 | 2401 |
| 1126400 | 813 | 832 | 10045 | 10064 | AGGCAGTCGCCGGGCACGAC | 0 | 2402 |
| 1126432 | 962 | 981 | 10438 | 10457 | GCCCTTTCCATGCAGTATCG | 32 | 2403 |
| 1126464 | 1107 | 1126 | 13206 | 13225 | GTTCTGGAACTCATGCATGG | 91† | 2404 |
| 1126496 | 1276 | 1295 | 13719 | 13738 | GATAGTTGAGCCGAGCCAAT | 22 | 2405 |
| 1126528 | 1348 | 1367 | 14409 | 14428 | TGAAATTGTTGGTCCGCGCT | 8 | 2406 |
| 1126560 | 1576 | 1595 | 21294 | 21313 | AGTCATCCAGCATATTGTGG | 52 | 2407 |
| 1126592 | 1640 | 1659 | 21358 | 21377 | ACCCTGTCGGCACTGCTATT | 26 | 2408 |
| 1126624 | 1851 | 1870 | 25038 | 25057 | TGCGATGTGTTCCTCCATGA | 43 | 2409 |
| 1126656 | 1978 | 1997 | 25383 | 25402 | GCTGGATGATACGCTGCCGC | 1 | 2410 |
| 1126688 | 2141 | 2160 | 26399 | 26418 | GCTGGCCGTGGGTAGCGGTA | 14 | 2411 |
| 1126720 | 2287 | 2306 | 26545 | 26564 | GGATGTTGCGCCGGTCCTTG | 20 | 2412 |
| 1126752 | 2363 | 2382 | 26621 | 26640 | GCCGTGTCCACAGAGTTGCG | 13 | 2413 |
| 1126784 | 2665 | 2684 | 26923 | 26942 | GCTGATTATGCATATTCTGG | 80 | 2414 |
| 1126816 | 2863 | 2882 | 27121 | 27140 | AGAACTTGGTGGCCCGCATG | 33 | 2415 |
| 1126848 | 2929 | 2948 | 27187 | 27206 | CAAACTCTGAAAGTGCCCCG | 32 | 2416 |
| 1126880 | 3229 | 3248 | 27487 | 27506 | CTGCAGGCGGATTCCCTGGA | 7 | 2417 |
| 1126912 | 3431 | 3450 | 27689 | 27708 | GATGGAAGAGCCTCGAGGTA | 33 | 2418 |
| 1126944 | N/A | N/A | 3051 | 3070 | GAGTTCCGGGCCCCCATCCA | 54 | 2419 |
| 1126976 | N/A | N/A | 3095 | 3114 | AGGTCACAAGGGTTCCCCTA | 41 | 2420 |
| 1127008 | N/A | N/A | 3317 | 3336 | CGCAGAAATCCCATCCCCCC | 23 | 2421 |
| 1127040 | N/A | N/A | 3367 | 3386 | ATGCTGTTAGGGTCATAGGT | 92 | 2422 |
| 1127072 | N/A | N/A | 3494 | 3513 | ATTACAGTCTCCAGCCCGTC | 30 | 2423 |
| 1127104 | N/A | N/A | 3594 | 3613 | CCTTCCCCTATTCCAGTACG | 41 | 2424 |
| 1127136 | N/A | N/A | 3953 | 3972 | CCTGACTAGGGACACCAGCA | 38 | 2425 |
| 1127168 | N/A | N/A | 4150 | 4169 | TGAGGACTGACTACCCGGAC | 44 | 2426 |
| 1127200 | N/A | N/A | 4515 | 4534 | CGTGTAGATGCCACCCACTG | 19 | 2427 |
| 1127232 | N/A | N/A | 4880 | 4899 | CCATTCGCCCATGTCTGGGA | 44 | 2428 |
| 1127264 | N/A | N/A | 5330 | 5349 | AGCACCGCTAGAAGGTAGAC | 65 | 2429 |
| 1127296 | N/A | N/A | 5604 | 5623 | CTCTTCCCCGATATTCCATG | 65 | 2430 |
| 1127328 | N/A | N/A | 5906 | 5925 | GGTTCCCTTAGGGACACAGT | 16 | 2431 |
| 1127360 | N/A | N/A | 6192 | 6211 | TGCCGTGTTTCTAAGACTTG | 63 | 2432 |
| 1127392 | N/A | N/A | 7217 | 7236 | GTATCACATCCCGGCTAATT | 60 | 2433 |
| 1127424 | N/A | N/A | 8370 | 8389 | TATTTTCCGTGCCCCCTGCT | 46 | 2434 |
| 1127456 | N/A | N/A | 8549 | 8568 | CTCATATCTTCACTCATCCG | 30 | 2435 |
| 1127488 | N/A | N/A | 8947 | 8966 | GGGCCCTTGGATGTTCATGT | 10 | 2436 |
| 1127520 | N/A | N/A | 9046 | 9065 | TCCTCGGGCAAGCCCGCCAG | 13 | 2437 |
| 1127552 | N/A | N/A | 9596 | 9615 | CGCTGTCTGCACCCAACATT | 9 | 2438 |
| 1127584 | N/A | N/A | 9683 | 9702 | TCCTGGCAGGGTTAGATTGG | 26 | 2439 |
| 1127616 | N/A | N/A | 9809 | 9828 | AATCCCAACCGGACAGGGAC | 1 | 2440 |
| 1127648 | N/A | N/A | 9900 | 9919 | AAGAATCGGCAATCCCCAGT | 12 | 2441 |
| 1127680 | N/A | N/A | 10174 | 10193 | TGGCATACTCGCAGTCCCCC | 18 | 2442 |
| 1127712 | N/A | N/A | 10774 | 10793 | CTGGCGAATATTTTAGACAG | 28 | 2443 |
| 1127744 | N/A | N/A | 12094 | 12113 | CTCAAATACAGGTCACCACC | 14 | 2444 |
| 1127776 | N/A | N/A | 12310 | 12329 | GTAGTTCTCACCTTATACTC | 36 | 2445 |
| 1127808 | N/A | N/A | 12581 | 12600 | AACAGGGATCTGATCCACAC | 70 | 2446 |
| 1127840 | N/A | N/A | 13292 | 13311 | TCTTAAGACCTAGGTATATG | 2 | 2447 |
| 1127872 | N/A | N/A | 13409 | 13428 | CGCTCAAGAATTAAGGAGGC | 15 | 2448 |
| 1127904 | N/A | N/A | 13603 | 13622 | AAGAACCCACAAGGCACGGT | 20 | 2449 |
| 1127936 | N/A | N/A | 14355 | 14374 | TGCCGTTCACCTGCGCAGAA | 5 | 2450 |
| 1127954 | N/A | N/A | 15654 | 15673 | CCGTCTACAGGATTTTCTAG | 94 | 83 |
| 1127969 | N/A | N/A | 15705 | 15724 | CAACCCCGTCTCATATTCCA | 22 | 2451 |
| 1128001 | N/A | N/A | 16742 | 16761 | ACACAACAGCCCCACGGTCA | 48 | 2452 |
| 1128033 | N/A | N/A | 16916 | 16935 | GGACCCAATGATGTCATGCC | 80 | 2453 |
| 1128065 | N/A | N/A | 17157 | 17176 | GCGCCCAGCTACCCTTAGGT | 17 | 2454 |
| 1128097 | N/A | N/A | 17992 | 18011 | GGGAAGGCTCCCACTGCAAG | 8 | 2455 |
| 1128129 | N/A | N/A | 18248 | 18267 | CTCGCCTCCAGCAACTGAAG | 17 | 2456 |
| 1128161 | N/A | N/A | 19310 | 19329 | TAGAGGTGGAGCCGTGTGCA | 33 | 2457 |
| 1128193 | N/A | N/A | 19547 | 19566 | AATAGAATGACGGTAAGTTC | 39 | 2458 |
| 1128225 | N/A | N/A | 19695 | 19714 | CCAATGTAGACCACAAGTAC | 12 | 2459 |
| 1128257 | N/A | N/A | 19832 | 19851 | CATCCCGATGGCAGAAACAT | 0 | 2460 |
| 1128289 | N/A | N/A | 20675 | 20694 | CAAGCGATGCATCCGTTTTG | 27 | 2461 |
| 1128321 | N/A | N/A | 21193 | 21212 | TTCCTCATGAGCCATGCGGA | 0 | 2462 |
| 1128353 | N/A | N/A | 21784 | 21803 | GCCCACTACACTCACCCGGT | 0 | 2463 |
| 1128385 | N/A | N/A | 21902 | 21921 | GAACAGGTGATTACCATTGT | 24 | 2464 |
| 1128417 | N/A | N/A | 22073 | 22092 | ACACTCGGCTGTATTTGTGA | 35 | 2465 |
| 1128449 | N/A | N/A | 22567 | 22586 | CTGACCGCGACTCCTAATTA | 21 | 2466 |
| 1128481 | N/A | N/A | 24596 | 24615 | TGTGCCAGTATCTTGTACGG | 24 | 2467 |
| 1128513 | N/A | N/A | 24738 | 24757 | TGCGGGAAAGTCCATAGTTG | 7 | 2468 |
| 1128545 | N/A | N/A | 24918 | 24937 | TCGCCTACCGTCTTAATCGC | 2 | 2469 |
| 1128577 | N/A | N/A | 25176 | 25195 | CCGAGAGCCCCCCCATTTCC | 6 | 2470 |
| 1128609 | N/A | N/A | 25537 | 25556 | GCAACTGTTACAAGTAGGAT | 79 | 2471 |
| 1128641 | N/A | N/A | 25896 | 25915 | TTGGCCACACCCTTATGCAA | 12 | 2472 |
| 1128673 | N/A | N/A | 26141 | 26160 | CGCAGACATATAGTACTAGG | 30 | 2473 |
| 1128705 | N/A | N/A | 26378 | 26397 | CCCTGGGCCTGCATACGGCG | 0 | 2474 |

TABLE 34
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126273 | 103 | 122 | 2750 | 2769 | CGCTCGGCTTCCTATTGCAA | 5 | 2475 |
| 1126305 | 186 | 205 | 2833 | 2852 | GTAGGGACCCCGGAGGTGTC | 0 | 2476 |
| 1126337 | 319 | 338 | 2966 | 2985 | CCACTTCGAAGAGCACTGCG | 31 | 2477 |
| 1126369 | 563 | 582 | 8637 | 8656 | ACCAGAGGGCCTCCCTCGAT | 0 | 2478 |
| 1126401 | 814 | 833 | 10046 | 10065 | CAGGCAGTCGCCGGGCACGA | 6 | 2479 |
| 1126433 | 963 | 982 | 10439 | 10458 | CGCCCTTTCCATGCAGTATC | 86 | 2480 |
| 1126465 | 1115 | 1134 | 13214 | 13233 | GCATGGAGGTTCTGGAACTC | 98† | 2481 |
| 1126497 | 1277 | 1296 | 13720 | 13739 | AGATAGTTGAGCCGAGCCAA | 19 | 2482 |
| 1126529 | 1349 | 1368 | 14410 | 14429 | TTGAAATTGTTGGTCCGCGC | 34 | 2483 |
| 1126561 | 1579 | 1598 | 21297 | 21316 | AGGAGTCATCCAGCATATTG | 36 | 2484 |
| 1126593 | 1641 | 1660 | 21359 | 21378 | CACCCTGTCGGCACTGCTAT | 0 | 2485 |
| 1126625 | 1853 | 1872 | 25040 | 25059 | TCTGCGATGTGTTCCTCCAT | 62 | 2486 |
| 1126657 | 1979 | 1998 | 25384 | 25403 | CGCTGGATGATACGCTGCCG | 13 | 2487 |
| 1126689 | 2143 | 2162 | 26401 | 26420 | AGGCTGGCCGTGGGTAGCGG | 0 | 2488 |
| 1126721 | 2288 | 2307 | 26546 | 26565 | CGGATGTTGCGCCGGTCCTT | 0 | 2489 |
| 1126753 | 2365 | 2384 | 26623 | 26642 | TGGCCGTGTCCACAGAGTTG | 17 | 2490 |
| 1126785 | 2667 | 2686 | 26925 | 26944 | GCGCTGATTATGCATATTCT | 50 | 2491 |
| 1126817 | 2864 | 2883 | 27122 | 27141 | CAGAACTTGGTGGCCCGCAT | 54 | 2492 |
| 1126849 | 2952 | 2971 | 27210 | 27229 | GACCATCTGCTCTCAGTTAC | 72 | 2493 |
| 1126881 | 3234 | 3253 | 27492 | 27511 | GTCCTCTGCAGGCGGATTCC | 16 | 2494 |
| 1126913 | 3508 | 3527 | 27766 | 27785 | CAAGCGGTGCAGACACAGCA | 29 | 2495 |
| 1126945 | N/A | N/A | 3052 | 3071 | AGAGTTCCGGGCCCCCATCC | 40 | 2496 |
| 1126977 | N/A | N/A | 3164 | 3183 | AACGAACCATCCCTCTCCCA | 23 | 2497 |
| 1127009 | N/A | N/A | 3324 | 3343 | GTTTGCACGCAGAAATCCCA | 74 | 2498 |
| 1127041 | N/A | N/A | 3368 | 3387 | CATGCTGTTAGGGTCATAGG | 85 | 2499 |
| 1127073 | N/A | N/A | 3495 | 3514 | CATTACAGTCTCCAGCCCGT | 14 | 2500 |
| 1127105 | N/A | N/A | 3615 | 3634 | GACTAACTTAGGACTTCCCA | 53 | 2501 |
| 1127137 | N/A | N/A | 3954 | 3973 | CCCTGACTAGGGACACCAGC | 16 | 2502 |
| 1127169 | N/A | N/A | 4151 | 4170 | CTGAGGACTGACTACCCGGA | 4 | 2503 |
| 1127201 | N/A | N/A | 4516 | 4535 | CCGTGTAGATGCCACCCACT | 27 | 2504 |
| 1127233 | N/A | N/A | 4881 | 4900 | TCCATTCGCCCATGTCTGGG | 64 | 2505 |
| 1127265 | N/A | N/A | 5331 | 5350 | CAGCACCGCTAGAAGGTAGA | 51 | 2506 |
| 1127297 | N/A | N/A | 5663 | 5682 | TAATCCCTGATCTGCCCATC | 17 | 2507 |
| 1127329 | N/A | N/A | 5914 | 5933 | CTGTTCTGGGTTCCCTTAGG | 79 | 2508 |
| 1127361 | N/A | N/A | 6195 | 6214 | CACTGCCGTGTTTCTAAGAC | 53 | 2509 |
| 1127393 | N/A | N/A | 7218 | 7237 | GGTATCACATCCCGGCTAAT | 76 | 2510 |
| 1127425 | N/A | N/A | 8371 | 8390 | ATATTTTCCGTGCCCCCTGC | 53 | 2511 |
| 1127457 | N/A | N/A | 8608 | 8627 | CCCGAAATACACCTGGGATG | 0 | 2512 |
| 1127489 | N/A | N/A | 8948 | 8967 | TGGGCCCTTGGATGTTCATG | 0 | 2513 |
| 1127521 | N/A | N/A | 9047 | 9066 | TTCCTCGGGCAAGCCCGCCA | 7 | 2514 |
| 1127553 | N/A | N/A | 9598 | 9617 | TACGCTGTCTGCACCCAACA | 37 | 2515 |
| 1127585 | N/A | N/A | 9684 | 9703 | GTCCTGGCAGGGTTAGATTG | 36 | 2516 |
| 1127617 | N/A | N/A | 9810 | 9829 | CAATCCCAACCGGACAGGGA | 4 | 2517 |
| 1127649 | N/A | N/A | 9901 | 9920 | AAAGAATCGGCAATCCCCAG | 23 | 2518 |
| 1127681 | N/A | N/A | 10175 | 10194 | CTGGCATACTCGCAGTCCCC | 23 | 2519 |
| 1127713 | N/A | N/A | 10776 | 10795 | GTCTGGCGAATATTTTAGAC | 0 | 2520 |
| 1127745 | N/A | N/A | 12095 | 12114 | CCTCAAATACAGGTCACCAC | 26 | 2521 |
| 1127777 | N/A | N/A | 12313 | 12332 | GTTGTAGTTCTCACCTTATA | 31 | 2522 |
| 1127809 | N/A | N/A | 12582 | 12601 | TAACAGGGATCTGATCCACA | 61 | 2523 |
| 1127841 | N/A | N/A | 13296 | 13315 | CCTCTCTTAAGACCTAGGTA | 31 | 2524 |
| 1127873 | N/A | N/A | 13410 | 13429 | GCGCTCAAGAATTAAGGAGG | 0 | 2525 |
| 1127905 | N/A | N/A | 13605 | 13624 | TAAAGAACCCACAAGGCACG | 0 | 2526 |
| 1127937 | N/A | N/A | 14356 | 14375 | CTGCCGTTCACCTGCGCAGA | 0 | 2527 |
| 1127954 | N/A | N/A | 15654 | 15673 | CCGTCTACAGGATTTTCTAG | 94 | 83 |
| 1127970 | N/A | N/A | 15706 | 15725 | ACAACCCCGTCTCATATTCC | 46 | 2528 |
| 1128002 | N/A | N/A | 16743 | 16762 | CACACAACAGCCCCACGGTC | 46 | 2529 |
| 1128034 | N/A | N/A | 16921 | 16940 | GATGAGGACCCAATGATGTC | 67 | 2530 |
| 1128066 | N/A | N/A | 17158 | 17177 | CGCGCCCAGCTACCCTTAGG | 5 | 2531 |
| 1128098 | N/A | N/A | 17993 | 18012 | CGGGAAGGCTCCCACTGCAA | 25 | 2532 |
| 1128130 | N/A | N/A | 18250 | 18269 | GCCTCGCCTCCAGCAACTGA | 3 | 2533 |
| 1128162 | N/A | N/A | 19312 | 19331 | GGTAGAGGTGGAGCCGTGTG | 2 | 2534 |
| 1128194 | N/A | N/A | 19549 | 19568 | TCAATAGAATGACGGTAAGT | 22 | 2535 |
| 1128226 | N/A | N/A | 19696 | 19715 | CCCAATGTAGACCACAAGTA | 30 | 2536 |
| 1128258 | N/A | N/A | 19834 | 19853 | AGCATCCCGATGGCAGAAAC | 28 | 2537 |
| 1128290 | N/A | N/A | 20676 | 20695 | TCAAGCGATGCATCCGTTTT | 8 | 2538 |
| 1128322 | N/A | N/A | 21194 | 21213 | CTTCCTCATGAGCCATGCGG | 3 | 2539 |
| 1128354 | N/A | N/A | 21785 | 21804 | TGCCCACTACACTCACCCGG | 6 | 2540 |
| 1128386 | N/A | N/A | 21903 | 21922 | AGAACAGGTGATTACCATTG | 18 | 2541 |
| 1128418 | N/A | N/A | 22074 | 22093 | CACACTCGGCTGTATTTGTG | 27 | 2542 |
| 1128450 | N/A | N/A | 22568 | 22587 | GCTGACCGCGACTCCTAATT | 30 | 2543 |
| 1128482 | N/A | N/A | 24597 | 24616 | TTGTGCCAGTATCTTGTACG | 28 | 2544 |
| 1128514 | N/A | N/A | 24739 | 24758 | ATGCGGGAAAGTCCATAGTT | 40 | 2545 |
| 1128546 | N/A | N/A | 24919 | 24938 | ATCGCCTACCGTCTTAATCG | 6 | 2546 |
| 1128578 | N/A | N/A | 25177 | 25196 | CCCGAGAGCCCCCCCATTTC | 0 | 2547 |
| 1128610 | N/A | N/A | 25558 | 25577 | GGTTCATTGCTTAAAGGGAC | 22 | 2548 |
| 1128642 | N/A | N/A | 25897 | 25916 | TTTGGCCACACCCTTATGCA | 16 | 2549 |
| 1128674 | N/A | N/A | 26142 | 26161 | GCGCAGACATATAGTACTAG | 0 | 2550 |
| 1128706 | N/A | N/A | 26381 | 26400 | TACCCCTGGGCCTGCATACG | 9 | 2551 |

TABLE 35
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126274 | 121 | 140 | 2768 | 2787 | GACGGGAAGCTTGCAAGACG | 29 | 2552 |
| 1126306 | 188 | 207 | 2835 | 2854 | AGGTAGGGACCCCGGAGGTG | 18 | 2553 |
| 1126338 | 321 | 340 | 2968 | 2987 | GGCCACTTCGAAGAGCACTG | 28 | 2554 |
| 1126370 | 565 | 584 | 8639 | 8658 | CCACCAGAGGGCCTCCCTCG | 0 | 2555 |
| 1126402 | 815 | 834 | 10047 | 10066 | ACAGGCAGTCGCCGGGCACG | 0 | 2556 |
| 1126434 | 965 | 984 | 10441 | 10460 | GCCGCCCTTTCCATGCAGTA | 12 | 2557 |
| 1126466 | 1125 | 1144 | 13224 | 13243 | CTTGCTCTGAGCATGGAGGT | 921 | 2558 |
| 1126498 | 1278 | 1297 | 13721 | 13740 | CAGATAGTTGAGCCGAGCCA | 34 | 2559 |
| 1126530 | 1350 | 1369 | 14411 | 14430 | GTTGAAATTGTTGGTCCGCG | 51 | 2560 |
| 1126562 | 1580 | 1599 | 21298 | 21317 | GAGGAGTCATCCAGCATATT | 2 | 2561 |
| 1126594 | 1645 | 1664 | 21363 | 21382 | CCTTCACCCTGTCGGCACTG | 31 | 2562 |
| 1126626 | 1856 | 1875 | 25043 | 25062 | GGGTCTGCGATGTGTTCCTC | 58 | 2563 |
| 1126658 | 1981 | 2000 | 25386 | 25405 | TCCGCTGGATGATACGCTGC | 13 | 2564 |
| 1126690 | 2144 | 2163 | 26402 | 26421 | GAGGCTGGCCGTGGGTAGCG | 1 | 2565 |
| 1126722 | 2289 | 2308 | 26547 | 26566 | ACGGATGTTGCGCCGGTCCT | 15 | 2566 |
| 1126754 | 2366 | 2385 | 26624 | 26643 | GTGGCCGTGTCCACAGAGTT | 12 | 2567 |
| 1126786 | 2706 | 2725 | 26964 | 26983 | TGGCCTCTGGGAAGCGGTCC | 23 | 2568 |
| 1126818 | 2866 | 2885 | 27124 | 27143 | TCCAGAACTTGGTGGCCCGC | 40 | 2569 |
| 1126850 | 2953 | 2972 | 27211 | 27230 | GGACCATCTGCTCTCAGTTA | 78 | 2570 |
| 1126882 | 3235 | 3254 | 27493 | 27512 | TGTCCTCTGCAGGCGGATTC | 23 | 2571 |
| 1126914 | 3509 | 3528 | 27767 | 27786 | CCAAGCGGTGCAGACACAGC | 32 | 2572 |
| 1126946 | N/A | N/A | 3054 | 3073 | TCAGAGTTCCGGGCCCCCAT | 41 | 2573 |
| 1126978 | N/A | N/A | 3166 | 3185 | CAAACGAACCATCCCTCTCC | 31 | 2574 |
| 1127010 | N/A | N/A | 3325 | 3344 | GGTTTGCACGCAGAAATCCC | 58 | 2575 |
| 1127042 | N/A | N/A | 3370 | 3389 | ACCATGCTGTTAGGGTCATA | 90 | 2576 |
| 1127074 | N/A | N/A | 3496 | 3515 | TCATTACAGTCTCCAGCCCG | 27 | 2577 |
| 1127106 | N/A | N/A | 3617 | 3636 | CTGACTAACTTAGGACTTCC | 26 | 2578 |
| 1127138 | N/A | N/A | 3955 | 3974 | TCCCTGACTAGGGACACCAG | 9 | 2579 |
| 1127170 | N/A | N/A | 4153 | 4172 | AGCTGAGGACTGACTACCCG | 54 | 2580 |
| 1127202 | N/A | N/A | 4726 | 4745 | GGCAGGCTGTCCACCCGCTT | 19 | 2581 |
| 1127234 | N/A | N/A | 4882 | 4901 | GTCCATTCGCCCATGTCTGG | 76 | 2582 |
| 1127266 | N/A | N/A | 5332 | 5351 | CCAGCACCGCTAGAAGGTAG | 37 | 2583 |
| 1127298 | N/A | N/A | 5706 | 5725 | CTTTCCGTCTTCACCCTGGG | 64 | 2584 |
| 1127330 | N/A | N/A | 5915 | 5934 | GCTGTTCTGGGTTCCCTTAG | 89 | 2585 |
| 1127362 | N/A | N/A | 6196 | 6215 | CCACTGCCGTGTTTCTAAGA | 64 | 2586 |
| 1127394 | N/A | N/A | 7219 | 7238 | AGGTATCACATCCCGGCTAA | 52 | 2587 |
| 1127426 | N/A | N/A | 8372 | 8391 | AATATTTTCCGTGCCCCCTG | 77 | 2588 |
| 1127458 | N/A | N/A | 8609 | 8628 | GCCCGAAATACACCTGGGAT | 7 | 2589 |
| 1127490 | N/A | N/A | 8955 | 8974 | TAGAGCCTGGGCCCTTGGAT | 0 | 2590 |
| 1127522 | N/A | N/A | 9048 | 9067 | ATTCCTCGGGCAAGCCCGCC | 0 | 2591 |
| 1127554 | N/A | N/A | 9599 | 9618 | GTACGCTGTCTGCACCCAAC | 34 | 2592 |
| 1127586 | N/A | N/A | 9685 | 9704 | AGTCCTGGCAGGGTTAGATT | 64 | 2593 |
| 1127618 | N/A | N/A | 9811 | 9830 | GCAATCCCAACCGGACAGGG | 47 | 2594 |
| 1127650 | N/A | N/A | 9902 | 9921 | AAAAGAATCGGCAATCCCCA | 10 | 2595 |
| 1127682 | N/A | N/A | 10176 | 10195 | CCTGGCATACTCGCAGTCCC | 35 | 2596 |
| 1127714 | N/A | N/A | 10885 | 10904 | CAAATCATGCAATGGCACGA | 19 | 2597 |
| 1127746 | N/A | N/A | 12097 | 12116 | AGCCTCAAATACAGGTCACC | 32 | 2598 |
| 1127778 | N/A | N/A | 12315 | 12334 | GAGTTGTAGTTCTCACCTTA | 41 | 2599 |
| 1127810 | N/A | N/A | 12583 | 12602 | CTAACAGGGATCTGATCCAC | 42 | 2600 |
| 1127842 | N/A | N/A | 13303 | 13322 | ACGCCCTCCTCTCTTAAGAC | 17 | 2601 |
| 1127874 | N/A | N/A | 13426 | 13445 | TGAGCCCATTGTTCCAGCGC | 21 | 2602 |
| 1127906 | N/A | N/A | 13608 | 13627 | GCCTAAAGAACCCACAAGGC | 4 | 2603 |
| 1127938 | N/A | N/A | 14357 | 14376 | GCTGCCGTTCACCTGCGCAG | 19 | 2604 |
| 1127954 | N/A | N/A | 15654 | 15673 | CCGTCTACAGGATTTTCTAG | 96 | 83 |
| 1127971 | N/A | N/A | 15707 | 15726 | GACAACCCCGTCTCATATTC | 52 | 2605 |
| 1128003 | N/A | N/A | 16744 | 16763 | TCACACAACAGCCCCACGGT | 39 | 2606 |
| 1128035 | N/A | N/A | 16922 | 16941 | CGATGAGGACCCAATGATGT | 82 | 2607 |
| 1128067 | N/A | N/A | 17160 | 17179 | GCCGCGCCCAGCTACCCTTA | 15 | 2608 |
| 1128099 | N/A | N/A | 17994 | 18013 | TCGGGAAGGCTCCCACTGCA | 6 | 2609 |
| 1128131 | N/A | N/A | 18252 | 18271 | GTGCCTCGCCTCCAGCAACT | 11 | 2610 |
| 1128163 | N/A | N/A | 19313 | 19332 | GGGTAGAGGTGGAGCCGTGT | 0 | 2611 |
| 1128195 | N/A | N/A | 19550 | 19569 | CTCAATAGAATGACGGTAAG | 32 | 2612 |
| 1128227 | N/A | N/A | 19697 | 19716 | CCCCAATGTAGACCACAAGT | 30 | 2613 |
| 1128259 | N/A | N/A | 19835 | 19854 | CAGCATCCCGATGGCAGAAA | 11 | 2614 |
| 1128291 | N/A | N/A | 21090 | 21109 | TAGTACAACCTGTAAAGGTT | 0 | 2615 |
| 1128323 | N/A | N/A | 21195 | 21214 | ACTTCCTCATGAGCCATGCG | 34 | 2616 |
| 1128355 | N/A | N/A | 21802 | 21821 | ATGACCACGCTGTCCCCTGC | 14 | 2617 |
| 1128387 | N/A | N/A | 21905 | 21924 | GCAGAACAGGTGATTACCAT | 29 | 2618 |
| 1128419 | N/A | N/A | 22076 | 22095 | GCCACACTCGGCTGTATTTG | 14 | 2619 |
| 1128451 | N/A | N/A | 22570 | 22589 | GGGCTGACCGCGACTCCTAA | 18 | 2620 |
| 1128483 | N/A | N/A | 24606 | 24625 | GATCCTTTCTTGTGCCAGTA | 79 | 2621 |
| 1128515 | N/A | N/A | 24740 | 24759 | GATGCGGGAAAGTCCATAGT | 55 | 2622 |
| 1128547 | N/A | N/A | 24920 | 24939 | CATCGCCTACCGTCTTAATC | 12 | 2623 |
| 1128579 | N/A | N/A | 25178 | 25197 | GCCCGAGAGCCCCCCCATTT | 6 | 2624 |
| 1128611 | N/A | N/A | 25559 | 25578 | TGGTTCATTGCTTAAAGGGA | 43 | 2625 |
| 1128643 | N/A | N/A | 25920 | 25939 | CGAATTCTCAAGTGAGTCTC | 40 | 2626 |
| 1128675 | N/A | N/A | 26143 | 26162 | CGCGCAGACATATAGTACTA | 17 | 2627 |
| 1128707 | N/A | N/A | 26383 | 26402 | GGTACCCCTGGGCCTGCATA | 15 | 2628 |

TABLE 36
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126275 | 122 | 141 | 2769 | 2788 | CGACGGGAAGCTTGCAAGAC | 31 | 2629 |
| 1126307 | 189 | 208 | 2836 | 2855 | CAGGTAGGGACCCCGGAGGT | 8 | 2630 |
| 1126339 | 359 | 378 | 4517 | 4536 | ACCGTGTAGATGCCACCCAC | 38 | 2631 |
| 1126371 | 570 | 589 | 8644 | 8663 | GAGCACCACCAGAGGGCCTC | 7 | 2632 |
| 1126403 | 816 | 835 | 10048 | 10067 | TACAGGCAGTCGCCGGGCAC | 0 | 2633 |
| 1126435 | 966 | 985 | 10442 | 10461 | TGCCGCCCTTTCCATGCAGT | 27 | 2634 |
| 1126467 | 1131 | 1150 | 13230 | 13249 | TCGAGCCTTGCTCTGAGCAT | 27† | 2635 |
| 1126499 | 1280 | 1299 | 13723 | 13742 | AGCAGATAGTTGAGCCGAGC | 61 | 2636 |
| 1126531 | 1351 | 1370 | 14412 | 14431 | CGTTGAAATTGTTGGTCCGC | 60 | 2637 |
| 1126563 | 1581 | 1600 | 21299 | 21318 | TGAGGAGTCATCCAGCATAT | 27 | 2638 |
| 1126595 | 1646 | 1665 | 21364 | 21383 | ACCTTCACCCTGTCGGCACT | 17 | 2639 |
| 1126627 | 1886 | 1905 | 25291 | 25310 | CGGTCAAGAATGTAGATACC | 34 | 2640 |
| 1126659 | 1982 | 2001 | 25387 | 25406 | TTCCGCTGGATGATACGCTG | 15 | 2641 |
| 1126691 | 2145 | 2164 | 26403 | 26422 | CGAGGCTGGCCGTGGGTAGC | 0 | 2642 |
| 1126723 | 2291 | 2310 | 26549 | 26568 | GCACGGATGTTGCGCCGGTC | 26 | 2643 |
| 1126755 | 2429 | 2448 | 26687 | 26706 | CGCTCCTCGCCCAGGGAGCT | 3 | 2644 |
| 1126787 | 2721 | 2740 | 26979 | 26998 | GTAATGGCAGATTCCTGGCC | 37 | 2645 |
| 1126819 | 2867 | 2886 | 27125 | 27144 | TTCCAGAACTTGGTGGCCCG | 34 | 2646 |
| 1126851 | 2958 | 2977 | 27216 | 27235 | ATGGAGGACCATCTGCTCTC | 32 | 2647 |
| 1126883 | 3236 | 3255 | 27494 | 27513 | CTGTCCTCTGCAGGCGGATT | 10 | 2648 |
| 1126915 | 3510 | 3529 | 27768 | 27787 | ACCAAGCGGTGCAGACACAG | 60 | 2649 |
| 1126947 | N/A | N/A | 3056 | 3075 | ACTCAGAGTTCCGGGCCCCC | 42 | 2650 |
| 1126979 | N/A | N/A | 3167 | 3186 | TCAAACGAACCATCCCTCTC | 0 | 2651 |
| 1127011 | N/A | N/A | 3326 | 3345 | AGGTTTGCACGCAGAAATCC | 51 | 2652 |
| 1127043 | N/A | N/A | 3371 | 3390 | GACCATGCTGTTAGGGTCAT | 26 | 2653 |
| 1127075 | N/A | N/A | 3505 | 3524 | CCCAGGCATTCATTACAGTC | 0 | 2654 |
| 1127107 | N/A | N/A | 3618 | 3637 | TCTGACTAACTTAGGACTTC | 30 | 2655 |
| 1127139 | N/A | N/A | 3958 | 3977 | CTCTCCCTGACTAGGGACAC | 0 | 2656 |
| 1127171 | N/A | N/A | 4154 | 4173 | GAGCTGAGGACTGACTACCC | 42 | 2657 |
| 1127203 | N/A | N/A | 4727 | 4746 | TGGCAGGCTGTCCACCCGCT | 8 | 2658 |
| 1127235 | N/A | N/A | 4883 | 4902 | TGTCCATTCGCCCATGTCTG | 73 | 2659 |
| 1127267 | N/A | N/A | 5333 | 5352 | CCCAGCACCGCTAGAAGGTA | 0 | 2660 |
| 1127299 | N/A | N/A | 5731 | 5750 | TTTCTCCTTGGACAACAGCG | 94 | 70 |
| | | | 5765 | 5784 | | | |
| 1127331 | N/A | N/A | 5916 | 5935 | GGCTGTTCTGGGTTCCCTTA | 87 | 2661 |
| 1127363 | N/A | N/A | 6197 | 6216 | GCCACTGCCGTGTTTCTAAG | 85 | 2662 |
| 1127395 | N/A | N/A | 7220 | 7239 | AAGGTATCACATCCCGGCTA | 67 | 2663 |
| 1127427 | N/A | N/A | 8373 | 8392 | CAATATTTTCCGTGCCCCCT | 62 | 2664 |
| 1127459 | N/A | N/A | 8816 | 8835 | TGGGTGGAATGTGTCAGACG | 26 | 2665 |
| 1127491 | N/A | N/A | 8961 | 8980 | CGAGAGTAGAGCCTGGGCCC | 1 | 2666 |
| 1127523 | N/A | N/A | 9050 | 9069 | GAATTCCTCGGGCAAGCCCG | 10 | 2667 |
| 1127555 | N/A | N/A | 9602 | 9621 | AGGGTACGCTGTCTGCACCC | 2 | 2668 |
| 1127587 | N/A | N/A | 9686 | 9705 | AAGTCCTGGCAGGGTTAGAT | 58 | 2669 |
| 1127619 | N/A | N/A | 9812 | 9831 | AGCAATCCCAACCGGACAGG | 32 | 2670 |
| 1127651 | N/A | N/A | 9903 | 9922 | GAAAAGAATCGGCAATCCCC | 0 | 2671 |
| 1127683 | N/A | N/A | 10177 | 10196 | TCCTGGCATACTCGCAGTCC | 13 | 2672 |
| 1127715 | N/A | N/A | 10886 | 10905 | CCAAATCATGCAATGGCACG | 15 | 2673 |
| 1127747 | N/A | N/A | 12100 | 12119 | GAGAGCCTCAAATACAGGTC | 81 | 2674 |
| 1127779 | N/A | N/A | 12338 | 12357 | CTAACCAGTCAAAGGCCTTC | 8 | 2675 |
| 1127811 | N/A | N/A | 12584 | 12603 | GCTAACAGGGATCTGATCCA | 49 | 2676 |
| 1127843 | N/A | N/A | 13319 | 13338 | GATGGTTAGGCTCCCAACGC | 33 | 2677 |
| 1127875 | N/A | N/A | 13498 | 13517 | GTAATACAGAGGCATCACGG | 22 | 2678 |
| 1127907 | N/A | N/A | 13732 | 13751 | CTCACTCTGAGCAGATAGTT | 26 | 2679 |
| 1127939 | N/A | N/A | 14358 | 14377 | CGCTGCCGTTCACCTGCGCA | 8 | 2680 |
| 1127954 | N/A | N/A | 15654 | 15673 | CCGTCTACAGGATTTTCTAG | 96 | 83 |
| 1127972 | N/A | N/A | 15708 | 15727 | TGACAACCCCGTCTCATATT | 36 | 2681 |
| 1128004 | N/A | N/A | 16745 | 16764 | GTCACACAACAGCCCCACGG | 88 | 2682 |
| 1128036 | N/A | N/A | 16923 | 16942 | GCGATGAGGACCCAATGATG | 95 | 2683 |
| 1128068 | N/A | N/A | 17214 | 17233 | GCTGGTAATTGGCCACCTCG | 28 | 2684 |
| 1128100 | N/A | N/A | 17996 | 18015 | TGTCGGGAAGGCTCCCACTG | 0 | 2685 |
| 1128132 | N/A | N/A | 18253 | 18272 | AGTGCCTCGCCTCCAGCAAC | 16 | 2686 |
| 1128164 | N/A | N/A | 19343 | 19362 | CGATAAAACTACCACTCCCA | 0 | 2687 |
| 1128196 | N/A | N/A | 19552 | 19571 | GACTCAATAGAATGACGGTA | 55 | 2688 |
| 1128228 | N/A | N/A | 19698 | 19717 | TCCCCAATGTAGACCACAAG | 25 | 2689 |
| 1128260 | N/A | N/A | 19836 | 19855 | CCAGCATCCCGATGGCAGAA | 12 | 2690 |
| 1128292 | N/A | N/A | 21094 | 21113 | CCACTAGTACAACCTGTAAA | 7 | 2691 |
| 1128324 | N/A | N/A | 21258 | 21277 | AAGACTGCCGCTGCAGGAGC | 10 | 2692 |
| 1128356 | N/A | N/A | 21803 | 21822 | CATGACCACGCTGTCCCCTG | 14 | 2693 |
| 1128388 | N/A | N/A | 21907 | 21926 | TAGCAGAACAGGTGATTACC | 29 | 2694 |
| 1128420 | N/A | N/A | 22077 | 22096 | GGCCACACTCGGCTGTATTT | 10 | 2695 |
| 1128452 | N/A | N/A | 22626 | 22645 | ACTAGTACTATCTATCAATC | 14 | 2696 |
| 1128484 | N/A | N/A | 24620 | 24639 | GTCACAACCTGTAAGATCCT | 63 | 2697 |
| 1128516 | N/A | N/A | 24741 | 24760 | AGATGCGGGAAAGTCCATAG | 31 | 2698 |
| 1128548 | N/A | N/A | 24921 | 24940 | TCATCGCCTACCGTCTTAAT | 13 | 2699 |
| 1128580 | N/A | N/A | 25179 | 25198 | GGCCCGAGAGCCCCCCCATT | 0 | 2700 |
| 1128612 | N/A | N/A | 25560 | 25579 | CTGGTTCATTGCTTAAAGGG | 47 | 2701 |
| 1128644 | N/A | N/A | 25921 | 25940 | CCGAATTCTCAAGTGAGTCT | 64 | 2702 |
| 1128676 | N/A | N/A | 26214 | 26233 | CACTCACCGCATCCGCCTCG | 1 | 2703 |
| 1128708 | N/A | N/A | 26384 | 26403 | CGGTACCCCTGGGCCTGCAT | 12 | 2704 |

TABLE 37
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126308 | 190 | 209 | 2837 | 2856 | CCAGGTAGGGACCCCGGAGG | 0 | 2706 |
| 1126340 | 360 | 379 | 4518 | 4537 | CACCGTGTAGATGCCACCCA | 32 | 2707 |
| 1126372 | 633 | 652 | 8707 | 8726 | GCAGGTATCCCAGAGCTCTC | 35 | 2708 |
| 1126404 | 817 | 836 | 10049 | 10068 | CTACAGGCAGTCGCCGGGCA | 19 | 2709 |
| 1126436 | 967 | 986 | 10443 | 10462 | CTGCCGCCCTTTCCATGCAG | 20 | 2710 |
| 1126468 | 1132 | 1151 | 13231 | 13250 | TTCGAGCCTTGCTCTGAGCA | 15† | 2711 |
| 1126500 | 1281 | 1300 | 13724 | 13743 | GAGCAGATAGTTGAGCCGAG | 41 | 2712 |
| 1126532 | 1411 | 1430 | 17844 | 17863 | TCACCGTGTTGGCCGTGTCC | 17 | 2713 |
| 1126564 | 1607 | 1626 | 21325 | 21344 | CGGCGGATGGTGGTCAGGAT | 28 | 2714 |
| 1126596 | 1648 | 1667 | 21366 | 21385 | TCACCTTCACCCTGTCGGCA | 26 | 2715 |
| 1126628 | 1888 | 1907 | 25293 | 25312 | GCCGGTCAAGAATGTAGATA | 7 | 2716 |
| 1126660 | 1983 | 2002 | 25388 | 25407 | GTTCCGCTGGATGATACGCT | 19 | 2717 |
| 1126692 | 2147 | 2166 | 26405 | 26424 | ACCGAGGCTGGCCGTGGGTA | 13 | 2718 |
| 1126724 | 2292 | 2311 | 26550 | 26569 | TGCACGGATGTTGCGCCGGT | 4 | 2719 |
| 1126756 | 2431 | 2450 | 26689 | 26708 | TACGCTCCTCGCCCAGGGAG | 14 | 2720 |
| 1126788 | 2727 | 2746 | 26985 | 27004 | CGCAGAGTAATGGCAGATTC | 88 | 2721 |
| 1126820 | 2873 | 2892 | 27131 | 27150 | CGTGGTTTCCAGAACTTGGT | 84 | 365 |
| 1126852 | 2960 | 2979 | 27218 | 27237 | AAATGGAGGACCATCTGCTC | 48 | 2722 |
| 1126884 | 3277 | 3296 | 27535 | 27554 | TGGGAATAAGCCAGGTTAGG | 82 | 2723 |
| 1126916 | 3512 | 3531 | 27770 | 27789 | AAACCAAGCGGTGCAGACAC | 42 | 2724 |
| 1126948 | N/A | N/A | 3057 | 3076 | AACTCAGAGTTCCGGGCCCC | 10 | 2725 |
| 1126980 | N/A | N/A | 3169 | 3188 | AGTCAAACGAACCATCCCTC | 50 | 2726 |
| 1127012 | N/A | N/A | 3327 | 3346 | CAGGTTTGCACGCAGAAATC | 65 | 2727 |
| 1127044 | N/A | N/A | 3372 | 3391 | AGACCATGCTGTTAGGGTCA | 32 | 2728 |
| 1127076 | N/A | N/A | 3506 | 3525 | GCCCAGGCATTCATTACAGT | 0 | 2729 |
| 1127108 | N/A | N/A | 3619 | 3638 | CTCTGACTAACTTAGGACTT | 27 | 2730 |
| 1127140 | N/A | N/A | 4048 | 4067 | TGGCCAATCCAGCTTCTCAC | 31 | 2731 |
| 1127172 | N/A | N/A | 4190 | 4209 | CTGAAGGAATTAGGCACTGG | 81 | 2732 |
| 1127204 | N/A | N/A | 4728 | 4747 | ATGGCAGGCTGTCCACCCGC | 42 | 2733 |
| 1127236 | N/A | N/A | 4885 | 4904 | TCTGTCCATTCGCCCATGTC | 54 | 2734 |
| 1127268 | N/A | N/A | 5403 | 5422 | CCCACACAATGTGCTACCTT | 50 | 2735 |
| 1127300 | N/A | N/A | 5743 | 5762 | CCGCCTGATGGCTTTCTCCT | 56 | 2736 |
| | | | 5777 | 5796 | | | |
| 1127332 | N/A | N/A | 5925 | 5944 | AAGCGACTGGGCTGTTCTGG | 82 | 2737 |
| 1127364 | N/A | N/A | 6199 | 6218 | TAGCCACTGCCGTGTTTCTA | 69 | 2738 |
| 1127396 | N/A | N/A | 7222 | 7241 | ACAAGGTATCACATCCCGGC | 62 | 2739 |
| 1127428 | N/A | N/A | 8374 | 8393 | GCAATATTTTCCGTGCCCCC | 95 | 2740 |
| 1127460 | N/A | N/A | 8817 | 8836 | ATGGGTGGAATGTGTCAGAC | 49 | 2741 |
| 1127492 | N/A | N/A | 8962 | 8981 | TCGAGAGTAGAGCCTGGGCC | 17 | 2742 |
| 1127524 | N/A | N/A | 9052 | 9071 | TAGAATTCCTCGGGCAAGCC | 19 | 2743 |
| 1127556 | N/A | N/A | 9603 | 9622 | AAGGGTACGCTGTCTGCACC | 0 | 2744 |
| 1127588 | N/A | N/A | 9687 | 9706 | TAAGTCCTGGCAGGGTTAGA | 46 | 2745 |
| 1127620 | N/A | N/A | 9813 | 9832 | CAGCAATCCCAACCGGACAG | 21 | 2746 |
| 1127652 | N/A | N/A | 9904 | 9923 | GGAAAAGAATCGGCAATCCC | 14 | 2747 |
| 1127684 | N/A | N/A | 10179 | 10198 | TTTCCTGGCATACTCGCAGT | 21 | 2748 |
| 1127716 | N/A | N/A | 10889 | 10908 | CACCCAAATCATGCAATGGC | 6 | 2749 |
| 1127748 | N/A | N/A | 12111 | 12130 | GTATATAGTCAGAGAGCCTC | 58 | 2750 |
| 1127780 | N/A | N/A | 12339 | 12358 | GCTAACCAGTCAAAGGCCTT | 40 | 2751 |
| 1127812 | N/A | N/A | 12585 | 12604 | GGCTAACAGGGATCTGATCC | 47 | 2752 |
| 1127844 | N/A | N/A | 13320 | 13339 | AGATGGTTAGGCTCCCAACG | 47 | 2753 |
| 1127876 | N/A | N/A | 13499 | 13518 | GGTAATACAGAGGCATCACG | 38 | 2754 |
| 1127908 | N/A | N/A | 13733 | 13752 | CCTCACTCTGAGCAGATAGT | 29 | 2755 |
| 1127940 | N/A | N/A | 14359 | 14378 | TCGCTGCCGTTCACCTGCGC | 7 | 2756 |
| 1127954 | N/A | N/A | 15654 | 15673 | CCGTCTACAGGATTTTCTAG | 95 | 83 |
| 1127973 | N/A | N/A | 15709 | 15728 | CTGACAACCCCGTCTCATAT | 31 | 2757 |
| 1128005 | N/A | N/A | 16746 | 16765 | TGTCACACAACAGCCCCACG | 72 | 2758 |
| 1128037 | N/A | N/A | 16924 | 16943 | TGCGATGAGGACCCAATGAT | 89 | 2759 |
| 1128069 | N/A | N/A | 17217 | 17236 | CTGGCTGGTAATTGGCCACC | 11 | 2760 |
| 1128101 | N/A | N/A | 17998 | 18017 | CATGTCGGGAAGGCTCCCAC | 21 | 2761 |
| 1128133 | N/A | N/A | 18255 | 18274 | TAAGTGCCTCGCCTCCAGCA | 11 | 2762 |
| 1128165 | N/A | N/A | 19344 | 19363 | CCGATAAAACTACCACTCCC | 15 | 2763 |
| 1128197 | N/A | N/A | 19553 | 19572 | AGACTCAATAGAATGACGGT | 43 | 2764 |
| 1128229 | N/A | N/A | 19701 | 19720 | GGCTCCCCAATGTAGACCAC | 20 | 2765 |
| 1128261 | N/A | N/A | 19839 | 19858 | CTCCCAGCATCCCGATGGCA | 12 | 2766 |
| 1128293 | N/A | N/A | 21095 | 21114 | CCCACTAGTACAACCTGTAA | 25 | 2767 |
| 1128325 | N/A | N/A | 21259 | 21278 | AAAGACTGCCGCTGCAGGAG | 33 | 2768 |
| 1128357 | N/A | N/A | 21807 | 21826 | ATGCCATGACCACGCTGTCC | 23 | 2769 |
| 1128389 | N/A | N/A | 21908 | 21927 | GTAGCAGAACAGGTGATTAC | 31 | 2770 |
| 1128421 | N/A | N/A | 22078 | 22097 | TGGCCACACTCGGCTGTATT | 17 | 2771 |
| 1128453 | N/A | N/A | 22627 | 22646 | GACTAGTACTATCTATCAAT | 19 | 2772 |
| 1128485 | N/A | N/A | 24621 | 24640 | TGTCACAACCTGTAAGATCC | 51 | 2773 |
| 1128517 | N/A | N/A | 24742 | 24761 | AAGATGCGGGAAAGTCCATA | 28 | 2774 |
| 1128549 | N/A | N/A | 24922 | 24941 | CTCATCGCCTACCGTCTTAA | 9 | 2775 |
| 1128581 | N/A | N/A | 25181 | 25200 | GCGGCCCGAGAGCCCCCCCA | 17 | 2776 |
| 1128613 | N/A | N/A | 25561 | 25580 | CCTGGTTCATTGCTTAAAGG | 22 | 2777 |
| 1128645 | N/A | N/A | 25922 | 25941 | GCCGAATTCTCAAGTGAGTC | 34 | 2778 |
| 1128677 | N/A | N/A | 26217 | 26236 | GTCCACTCACCGCATCCGCC | 0 | 2779 |
| 1128709 | N/A | N/A | 26385 | 26404 | GCGGTACCCCTGGGCCTGCA | 4 | 2780 |

TABLE 38
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126277 | 125 | 144 | 2772 | 2791 | GCCCGACGGGAAGCTTGCAA | 24 | 2781 |
| 1126309 | 191 | 210 | 2838 | 2857 | TCCAGGTAGGGACCCCGGAG | 1 | 2782 |
| 1126341 | 362 | 381 | 4520 | 4539 | AGCACCGTGTAGATGCCACC | 0 | 2783 |
| 1126373 | 636 | 655 | 8710 | 8729 | GTTGCAGGTATCCCAGAGCT | 48 | 2784 |
| 1126405 | 819 | 838 | 10051 | 10070 | TGCTACAGGCAGTCGCCGGG | 10 | 2785 |
| 1126437 | 968 | 987 | 10444 | 10463 | GCTGCCGCCCTTTCCATGCA | 25 | 2786 |
| 1126469 | 1134 | 1153 | 13233 | 13252 | GATTCGAGCCTTGCTCTGAG | 56† | 2787 |
| 1126501 | 1282 | 1301 | 13725 | 13744 | TGAGCAGATAGTTGAGCCGA | 18 | 2788 |
| 1126533 | 1420 | 1439 | 17853 | 17872 | ACTTTTCCTTCACCGTGTTG | 65 | 2789 |
| 1126565 | 1609 | 1628 | 21327 | 21346 | TTCGGCGGATGGTGGTCAGG | 26 | 2790 |
| 1126597 | 1670 | 1689 | 21668 | 21687 | GAGAGGAACTCCGGGTGGAA | 44 | 2791 |
| 1126629 | 1889 | 1908 | 25294 | 25313 | CGCCGGTCAAGAATGTAGAT | 0 | 2792 |
| 1126661 | 1985 | 2004 | 25390 | 25409 | CGGTTCCGCTGGATGATACG | 6 | 2793 |
| 1126693 | 2157 | 2176 | 26415 | 26434 | CGACGGTGGCACCGAGGCTG | 14 | 2794 |
| 1126725 | 2293 | 2312 | 26551 | 26570 | GTGCACGGATGTTGCGCCGG | 0 | 2795 |
| 1126757 | 2432 | 2451 | 26690 | 26709 | TTACGCTCCTCGCCCAGGGA | 19 | 2796 |
| 1126789 | 2728 | 2747 | 26986 | 27005 | CCGCAGAGTAATGGCAGATT | 93 | 2797 |
| 1126821 | 2891 | 2910 | 27149 | 27168 | CCATTCGCAGGGACACCACG | 34 | 2798 |
| 1126853 | 2962 | 2981 | 27220 | 27239 | TGAAATGGAGGACCATCTGC | 49 | 2799 |
| 1126885 | 3278 | 3297 | 27536 | 27555 | TTGGGAATAAGCCAGGTTAG | 78 | 2800 |
| 1126917 | 3513 | 3532 | 27771 | 27790 | CAAACCAAGCGGTGCAGACA | 38 | 2801 |
| 1126949 | N/A | N/A | 3058 | 3077 | CAACTCAGAGTTCCGGGCCC | 4 | 2802 |
| 1126981 | N/A | N/A | 3170 | 3189 | AAGTCAAACGAACCATCCCT | 20 | 2803 |
| 1127013 | N/A | N/A | 3328 | 3347 | CCAGGTTTGCACGCAGAAAT | 87 | 2804 |
| 1127045 | N/A | N/A | 3373 | 3392 | TAGACCATGCTGTTAGGGTC | 35 | 2805 |
| 1127077 | N/A | N/A | 3535 | 3554 | TAATAGCTCAGGCCCTCAGC | 34 | 2806 |
| 1127109 | N/A | N/A | 3620 | 3639 | CCTCTGACTAACTTAGGACT | 25 | 2807 |
| 1127141 | N/A | N/A | 4055 | 4074 | CCCCTCATGGCCAATCCAGC | 16 | 2808 |
| 1127173 | N/A | N/A | 4193 | 4212 | GGTCTGAAGGAATTAGGCAC | 44 | 2809 |
| 1127205 | N/A | N/A | 4729 | 4748 | GATGGCAGGCTGTCCACCCG | 3 | 2810 |
| 1127237 | N/A | N/A | 4886 | 4905 | GTCTGTCCATTCGCCCATGT | 71 | 2811 |
| 1127269 | N/A | N/A | 5404 | 5423 | GCCCACACAATGTGCTACCT | 52 | 2812 |
| 1127301 | N/A | N/A | 5780 | 5799 | CTGCCGCCTGATGGCTTTCT | 25 | 2813 |
| 1127333 | N/A | N/A | 5926 | 5945 | GAAGCGACTGGGCTGTTCTG | 60 | 2814 |
| 1127365 | N/A | N/A | 6200 | 6219 | CTAGCCACTGCCGTGTTTCT | 55 | 2815 |
| 1127397 | N/A | N/A | 7223 | 7242 | CACAAGGTATCACATCCCGG | 72 | 2816 |
| 1127429 | N/A | N/A | 8375 | 8394 | TGCAATATTTTCCGTGCCCC | 91 | 2817 |
| 1127461 | N/A | N/A | 8826 | 8845 | GTTTCAGGTATGGGTGGAAT | 56 | 2818 |
| 1127493 | N/A | N/A | 8964 | 8983 | CATCGAGAGTAGAGCCTGGG | 61 | 2819 |
| 1127525 | N/A | N/A | 9053 | 9072 | CTAGAATTCCTCGGGCAAGC | 56 | 2820 |
| 1127557 | N/A | N/A | 9604 | 9623 | CAAGGGTACGCTGTCTGCAC | 29 | 2821 |
| 1127589 | N/A | N/A | 9688 | 9707 | CTAAGTCCTGGCAGGGTTAG | 20 | 2822 |
| 1127621 | N/A | N/A | 9814 | 9833 | GCAGCAATCCCAACCGGACA | 30 | 2823 |
| 1127653 | N/A | N/A | 9905 | 9924 | GGGAAAAGAATCGGCAATCC | 13 | 2824 |
| 1127685 | N/A | N/A | 10180 | 10199 | TTTTCCTGGCATACTCGCAG | 34 | 2825 |
| 1127717 | N/A | N/A | 10891 | 10910 | GCCACCCAAATCATGCAATG | 29 | 2826 |
| 1127749 | N/A | N/A | 12113 | 12132 | GAGTATATAGTCAGAGAGCC | 86 | 2827 |
| 1127781 | N/A | N/A | 12341 | 12360 | GAGCTAACCAGTCAAAGGCC | 46 | 2828 |
| 1127813 | N/A | N/A | 12586 | 12605 | TGGCTAACAGGGATCTGATC | 37 | 2829 |
| 1127845 | N/A | N/A | 13321 | 13340 | TAGATGGTTAGGCTCCCAAC | 47 | 2830 |
| 1127877 | N/A | N/A | 13502 | 13521 | TTAGGTAATACAGAGGCATC | 39 | 2831 |
| 1127909 | N/A | N/A | 13734 | 13753 | GCCTCACTCTGAGCAGATAG | 31 | 2832 |
| 1127941 | N/A | N/A | 14360 | 14379 | CTCGCTGCCGTTCACCTGCG | 10 | 2833 |
| 1127954 | N/A | N/A | 15654 | 15673 | CCGTCTACAGGATTTTCTAG | 96 | 83 |
| 1127974 | N/A | N/A | 15710 | 15729 | CCTGACAACCCCGTCTCATA | 17 | 2834 |
| 1128006 | N/A | N/A | 16752 | 16771 | TAGCAGTGTCACACAACAGC | 68 | 2835 |
| 1128038 | N/A | N/A | 16925 | 16944 | TTGCGATGAGGACCCAATGA | 87 | 2836 |
| 1128070 | N/A | N/A | 17218 | 17237 | TCTGGCTGGTAATTGGCCAC | 0 | 2837 |
| 1128102 | N/A | N/A | 18073 | 18092 | AGGTCCAATCCATACCTGCG | 11 | 2838 |
| 1128134 | N/A | N/A | 18256 | 18275 | CTAAGTGCCTCGCCTCCAGC | 16 | 2839 |
| 1128166 | N/A | N/A | 19345 | 19364 | CCCGATAAAACTACCACTCC | 21 | 2840 |
| 1128198 | N/A | N/A | 19554 | 19573 | TAGACTCAATAGAATGACGG | 45 | 2841 |
| 1128230 | N/A | N/A | 19702 | 19721 | TGGCTCCCCAATGTAGACCA | 15 | 2842 |
| 1128262 | N/A | N/A | 19840 | 19859 | ACTCCCAGCATCCCGATGGC | 11 | 2843 |
| 1128294 | N/A | N/A | 21096 | 21115 | TCCCACTAGTACAACCTGTA | 34 | 2844 |
| 1128326 | N/A | N/A | 21260 | 21279 | GAAAGACTGCCGCTGCAGGA | 34 | 2845 |
| 1128358 | N/A | N/A | 21808 | 21827 | GATGCCATGACCACGCTGTC | 7 | 2846 |
| 1128390 | N/A | N/A | 21912 | 21931 | CGCTGTAGCAGAACAGGTGA | 0 | 2847 |
| 1128422 | N/A | N/A | 22079 | 22098 | TTGGCCACACTCGGCTGTAT | 0 | 2848 |
| 1128454 | N/A | N/A | 22628 | 22647 | AGACTAGTACTATCTATCAA | 11 | 2849 |
| 1128486 | N/A | N/A | 24622 | 24641 | TTGTCACAACCTGTAAGATC | 41 | 2850 |
| 1128518 | N/A | N/A | 24743 | 24762 | CAAGATGCGGGAAAGTCCAT | 17 | 2851 |
| 1128550 | N/A | N/A | 24923 | 24942 | CCTCATCGCCTACCGTCTTA | 0 | 2852 |
| 1128582 | N/A | N/A | 25182 | 25201 | AGCGGCCCGAGAGCCCCCCC | 0 | 2853 |
| 1128614 | N/A | N/A | 25562 | 25581 | GCCTGGTTCATTGCTTAAAG | 10 | 2854 |
| 1128646 | N/A | N/A | 25923 | 25942 | GGCCGAATTCTCAAGTGAGT | 9 | 2855 |
| 1128678 | N/A | N/A | 26219 | 26238 | GGGTCCACTCACCGCATCCG | 0 | 2856 |
| 1128710 | N/A | N/A | 26387 | 26406 | TAGCGGTACCCCTGGGCCTG | 0 | 2857 |

TABLE 39
Reduction of GYS1 RNA by 5-10-5 MOE gapmers in A431 cells

| | SEQ | SEQ | SEQ | SEQ | | | |
|---|---|---|---|---|---|---|---|
| | ID | ID | ID | ID | | | |
| | NO: 1 | NO: 1 | NO: 2 | NO: 2 | | | SEQ |
| Compound | Start | Stop | Start | Stop | | % | ID |
| Number | Site | Site | Site | Site | Sequence (5′ to 3′) | Reduction | No. |
| 1126278 | 126 | 145 | 2773 | 2792 | TGCCCGACGGGAAGCTTGCA | 6 | 2858 |
| 1126310 | 192 | 211 | 2839 | 2858 | CTCCAGGTAGGGACCCCGGA | 8 | 2859 |
| 1126342 | 363 | 382 | 4521 | 4540 | CAGCACCGTGTAGATGCCAC | 12 | 2860 |
| 1126374 | 638 | 657 | 8712 | 8731 | ATGTTGCAGGTATCCCAGAG | 52 | 2861 |
| 1126406 | 820 | 839 | 10052 | 10071 | TTGCTACAGGCAGTCGCCGG | 3 | 2862 |
| 1126438 | 969 | 988 | 10445 | 10464 | GGCTGCCGCCCTTTCCATGC | 6 | 2863 |
| 1126470 | 1135 | 1154 | 13234 | 13253 | GGATTCGAGCCTTGCTCTGA | 52† | 2864 |
| 1126502 | 1283 | 1302 | 13726 | 13745 | CTGAGCAGATAGTTGAGCCG | 28 | 2865 |
| 1126534 | 1433 | 1452 | 17866 | 17885 | AGCTTCCTCCCGAACTTTTC | 35 | 2866 |
| 1126566 | 1610 | 1629 | 21328 | 21347 | ATTCGGCGGATGGTGGTCAG | 16 | 2867 |
| 1126598 | 1716 | 1735 | 21714 | 21733 | ACGGACAAACTCCTCATAGT | 10 | 2868 |
| 1126630 | 1891 | 1910 | 25296 | 25315 | ACCGCCGGTCAAGAATGTAG | 16 | 2869 |
| 1126662 | 1986 | 2005 | 25391 | 25410 | GCGGTTCCGCTGGATGATAC | 0 | 2870 |
| 1126694 | 2158 | 2177 | 26416 | 26435 | GCGACGGTGGCACCGAGGCT | 17 | 2871 |
| 1126726 | 2294 | 2313 | 26552 | 26571 | GGTGCACGGATGTTGCGCCG | 12 | 2872 |
| 1126758 | 2435 | 2454 | 26693 | 26712 | TAGTTACGCTCCTCGCCCAG | 11 | 2873 |
| 1126790 | 2729 | 2748 | 26987 | 27006 | ACCGCAGAGTAATGGCAGAT | 85 | 2874 |
| 1126822 | 2892 | 2911 | 27150 | 27169 | CCCATTCGCAGGGACACCAC | 4 | 2875 |
| 1126854 | 2963 | 2982 | 27221 | 27240 | TTGAAATGGAGGACCATCTG | 43 | 2876 |
| 1126886 | 3279 | 3298 | 27537 | 27556 | GTTGGGAATAAGCCAGGTTA | 82 | 2877 |
| 1126918 | 3515 | 3534 | 27773 | 27792 | TGCAAACCAAGCGGTGCAGA | 34 | 2878 |
| 1126950 | N/A | N/A | 3059 | 3078 | ACAACTCAGAGTTCCGGGCC | 29 | 2879 |
| 1126982 | N/A | N/A | 3171 | 3190 | TAAGTCAAACGAACCATCCC | 26 | 2880 |
| 1127014 | N/A | N/A | 3329 | 3348 | CCCAGGTTTGCACGCAGAAA | 85 | 2881 |
| 1127046 | N/A | N/A | 3375 | 3394 | CCTAGACCATGCTGTTAGGG | 32 | 2882 |
| 1127078 | N/A | N/A | 3536 | 3555 | CTAATAGCTCAGGCCCTCAG | 14 | 2883 |
| 1127110 | N/A | N/A | 3621 | 3640 | GCCTCTGACTAACTTAGGAC | 62 | 2884 |
| 1127142 | N/A | N/A | 4062 | 4081 | GGGAGACCCCCTCATGGCCA | 19 | 2885 |
| 1127174 | N/A | N/A | 4195 | 4214 | TTGGTCTGAAGGAATTAGGC | 91 | 2886 |
| 1127206 | N/A | N/A | 4730 | 4749 | CGATGGCAGGCTGTCCACCC | 6 | 2887 |
| 1127238 | N/A | N/A | 4887 | 4906 | TGTCTGTCCATTCGCCCATG | 78 | 2888 |
| 1127270 | N/A | N/A | 5405 | 5424 | CGCCCACACAATGTGCTACC | 10 | 2889 |
| 1127302 | N/A | N/A | 5781 | 5800 | CCTGCCGCCTGATGGCTTTC | 18 | 2890 |
| 1127334 | N/A | N/A | 5927 | 5946 | GGAAGCGACTGGGCTGTTCT | 72 | 2891 |
| 1127366 | N/A | N/A | 6202 | 6221 | TTCTAGCCACTGCCGTGTTT | 51 | 2892 |
| 1127398 | N/A | N/A | 7224 | 7243 | GCACAAGGTATCACATCCCG | 93 | 2893 |
| 1127430 | N/A | N/A | 8376 | 8395 | TTGCAATATTTTCCGTGCCC | 91 | 2894 |
| 1127462 | N/A | N/A | 8830 | 8849 | TGTAGTTTCAGGTATGGGTG | 70 | 2895 |
| 1127494 | N/A | N/A | 8965 | 8984 | GCATCGAGAGTAGAGCCTGG | 68 | 2896 |
| 1127526 | N/A | N/A | 9054 | 9073 | CCTAGAATTCCTCGGGCAAG | 31 | 2897 |
| 1127558 | N/A | N/A | 9605 | 9624 | TCAAGGGTACGCTGTCTGCA | 46 | 2898 |
| 1127590 | N/A | N/A | 9689 | 9708 | TCTAAGTCCTGGCAGGGTTA | 40 | 2899 |
| 1127622 | N/A | N/A | 9815 | 9834 | TGCAGCAATCCCAACCGGAC | 24 | 2900 |
| 1127654 | N/A | N/A | 9906 | 9925 | TGGGAAAAGAATCGGCAATC | 31 | 2901 |
| 1127686 | N/A | N/A | 10181 | 10200 | GTTTTCCTGGCATACTCGCA | 50 | 2902 |
| 1127718 | N/A | N/A | 11034 | 11053 | GCTAACATAAAGTGTTCTGG | 44 | 2903 |
| 1127750 | N/A | N/A | 12119 | 12138 | CCTCCAGAGTATATAGTCAG | 50 | 2904 |
| 1127782 | N/A | N/A | 12342 | 12361 | AGAGCTAACCAGTCAAAGGC | 76 | 2905 |
| 1127814 | N/A | N/A | 12611 | 12630 | TATAAGTCTCCTCAGCTACT | 34 | 2906 |
| 1127846 | N/A | N/A | 13322 | 13341 | CTAGATGGTTAGGCTCCCAA | 50 | 2907 |
| 1127878 | N/A | N/A | 13509 | 13528 | GGATGTCTTAGGTAATACAG | 54 | 2908 |
| 1127910 | N/A | N/A | 13786 | 13805 | CTCCCTAGTTATAAACTGCT | 24 | 2909 |
| 1127942 | N/A | N/A | 14362 | 14381 | TGCTCGCTGCCGTTCACCTG | 21 | 2910 |
| 1127954 | N/A | N/A | 15654 | 15673 | CCGTCTACAGGATTTTCTAG | 95 | 83 |
| 1127975 | N/A | N/A | 15711 | 15730 | TCCTGACAACCCCGTCTCAT | 24 | 2911 |
| 1128007 | N/A | N/A | 16754 | 16773 | GCTAGCAGTGTCACACAACA | 46 | 2912 |
| 1128039 | N/A | N/A | 16926 | 16945 | TTTGCGATGAGGACCCAATG | 81 | 2913 |
| 1128071 | N/A | N/A | 17219 | 17238 | CTCTGGCTGGTAATTGGCCA | 2 | 2914 |
| 1128103 | N/A | N/A | 18074 | 18093 | GAGGTCCAATCCATACCTGC | 24 | 2915 |
| 1128135 | N/A | N/A | 18257 | 18276 | CCTAAGTGCCTCGCCTCCAG | 22 | 2916 |
| 1128167 | N/A | N/A | 19346 | 19365 | GCCCGATAAAACTACCACTC | 29 | 2917 |
| 1128199 | N/A | N/A | 19555 | 19574 | CTAGACTCAATAGAATGACG | 41 | 2918 |
| 1128231 | N/A | N/A | 19706 | 19725 | GAAATGGCTCCCCAATGTAG | 26 | 2919 |
| 1128263 | N/A | N/A | 19842 | 19861 | CAACTCCCAGCATCCCGATG | 4 | 2920 |
| 1128295 | N/A | N/A | 21097 | 21116 | TTCCCACTAGTACAACCTGT | 29 | 2921 |
| 1128327 | N/A | N/A | 21537 | 21556 | ATGTGTTTGGCAGGACCAAT | 12 | 2922 |
| 1128359 | N/A | N/A | 21809 | 21828 | GGATGCCATGACCACGCTGT | 33 | 2923 |
| 1128391 | N/A | N/A | 21913 | 21932 | GCGCTGTAGCAGAACAGGTG | 31 | 2924 |
| 1128423 | N/A | N/A | 22080 | 22099 | CTTGGCCACACTCGGCTGTA | 12 | 2925 |
| 1128455 | N/A | N/A | 22630 | 22649 | ACAGACTAGTACTATCTATC | 40 | 2926 |
| 1128487 | N/A | N/A | 24646 | 24665 | GCCAAGGTCCATCATTCTAC | 69 | 2927 |
| 1128519 | N/A | N/A | 24744 | 24763 | CCAAGATGCGGGAAAGTCCA | 21 | 2928 |
| 1128551 | N/A | N/A | 24924 | 24943 | CCCTCATCGCCTACCGTCTT | 15 | 2929 |
| 1128583 | N/A | N/A | 25189 | 25208 | CTCTCCCAGCGGCCCGAGAG | 5 | 2930 |
| 1128615 | N/A | N/A | 25563 | 25582 | GGCCTGGTTCATTGCTTAAA | 14 | 2931 |
| 1128647 | N/A | N/A | 25924 | 25943 | GGGCCGAATTCTCAAGTGAG | 11 | 2932 |
| 1128679 | N/A | N/A | 26245 | 26264 | TTAGCTCCTGGCTAAGCAGA | 22 | 2933 |
| 1128711 | N/A | N/A | 26388 | 26407 | GTAGCGGTACCCCTGGGCCT | 24 | 2934 |

### Example 3: Effect of % Modified Oligonucleotides on Human GYS RNA In Vitro, Multiple Doses

Modified oligonucleotides selected from the examples above were tested at various doses in A431 cells. Cultured A431 cells at a density of 10,000 cells per well were treated using free uptake with various concentrations of modified oligonucleotide as specified in the tables below. After a treatment period of approximately 48 hours, total RNA was isolated from the cells and GYS1 RNA levels were measured by quantitative real-time RTPCR. Human GYS1 primer probe set RTS36346 was used to measure RNA levels, as described above. GYS1 RNA levels were normalized to total RNA content, as measured by RIBOGREEN®. Results are presented as percent reduction of GYS1 RNA relative to the amount of GYS1 RNA in untreated control cells (% reduction). As used herein, a value of ‘0’ indicates that treatment with the modified oligonucleotide did not inhibit GYS1 mRNA levels. Each table represents results from an individual assay plate. The half maximal inhibitory concentration (IC₅₀) of each modified oligonucleotide was calculated using a linear regression on a log/linear plot of the data in Excel. Modified oligonucleotides marked with a (†) symbol indicate that the modified oligonucleotide is complementary to the amplicon region of the primer probe set.

TABLE 40
Dose-dependent reduction of human GYS1 RNA
in A431 cells by modified oligonucleotides

| Compound | % Reduction | | | | |
|---|---|---|---|---|---|
| Number | 94 nM | 375 nM | 1500 nM | 6000 nM | IC₅₀ μM |
| 941366 | 16 | 23 | 41 | 50 | 5.7 |
| 941367 | 1 | 22 | 50 | 69 | 1.8 |
| 941452 | 12 | 29 | 51 | 67 | 1.6 |
| 941459 | 13 | 38 | 56 | 79 | 0.9 |
| 941553 | 59 | 78 | 88 | 88 | <0.1 |
| 941554 | 21 | 43 | 61 | 76 | 0.7 |
| 941555 | 14 | 37 | 57 | 70 | 1.1 |
| 941573 | 0 | 15 | 20 | 41 | >6.0 |
| 941583 | 17 | 21 | 44 | 61 | 2.6 |
| 941596 | 15 | 29 | 49 | 66 | 1.7 |
| 941597 | 36 | 58 | 77 | 85 | 0.2 |
| 941615 | 49 | 71 | 85 | 91 | <0.1 |
| 941637 | 24 | 53 | 81 | 90 | 0.4 |
| 941651 | 0 | 30 | 49 | 69 | 1.7 |
| 941667 | 3 | 15 | 41 | 57 | 3.5 |
| 941715 | 50 | 80 | 92 | 96 | <0.1 |
| 941717 | 0 | 25 | 51 | 72 | 1.6 |
| 941721 | 8 | 13 | 34 | 51 | >6.0 |
| 941722 | 25 | 59 | 83 | 91 | 0.3 |

TABLE 41
Dose-dependent reduction of human GYS1 RNA
in A431 cells by modified oligonucleotides

| Compound | % Reduction | | | | |
|---|---|---|---|---|---|
| Number | 94 nM | 375 nM | 1500 nM | 6000 nM | IC₅₀ μM |
| 941358 | 8 | 33 | 54 | 71 | 1.3 |
| 941370 | 0 | 11 | 41 | 63 | 2.8 |
| 941405 | 0 | 14 | 38 | 68 | 2.5 |
| 941466 | 0 | 15 | 38 | 54 | 4.3 |
| 941484 | 0 | 8 | 21 | 39 | >6.0 |
| 941485 | 16 | 30 | 48 | 60 | 2.1 |
| 941563 | 44 | 59 | 80 | 89 | 0.2 |
| 941574 | 0 | 12 | 31 | 57 | 4.4 |
| 941605 | 49 | 70 | 86 | 94 | <0.1 |
| 941611 | 38 | 54 | 71 | 84 | 0.3 |
| 941617 | 0 | 0 | 0 | 12 | >6.0 |
| 941634 | 6 | 9 | 25 | 41 | >6.0 |
| 941635 | 19 | 38 | 56 | 69 | 1.1 |
| 941652 | 7 | 26 | 58 | 75 | 1.3 |
| 941653 | 10 | 33 | 63 | 78 | 0.9 |
| 941715 | 44 | 80 | 93 | 96 | <0.1 |
| 941718 | 46 | 68 | 85 | 92 | 0.1 |
| 941724 | 1 | 0 | 0 | 0 | >6.0 |
| 941772 | 1 | 6 | 1 | 10 | >6.0 |

TABLE 42
Dose-dependent reduction of human GYS1 RNA
in A431 cells by modified oligonucleotides

| Compound | % Reduction | | | | |
|---|---|---|---|---|---|
| Number | 94 nM | 375 nM | 1500 nM | 6000 nM | IC₅₀ μM |
| 941359 | 32 | 54 | 77 | 90 | 0.3 |
| 941371 | 16 | 33 | 53 | 66 | 1.4 |
| 941378 | 8 | 25 | 47 | 59 | 2.5 |
| 941437 | 0 | 12 | 39 | 51 | 4.5 |
| 941438 | 20 | 37 | 59 | 75 | 0.9 |
| 941450 | 13 | 32 | 49 | 69 | 1.5 |
| 941462 | 8 | 20 | 23 | 33 | >6.0 |
| 941558 | 12 | 23 | 41 | 51 | 4.8 |
| 941581 | 0 | 9 | 14 | 44 | >6.0 |
| 941588 | 32 | 55 | 61 | 67 | 0.5 |
| 941612 | 24 | 45 | 61 | 76 | 0.7 |
| 941629 | 15 | 26 | 45 | 54 | 3.4 |
| 941630 | 44 | 68 | 84 | 89 | 0.1 |
| 941636 | 10 | 29 | 49 | 64 | 1.9 |
| 941648 | 0 | 24 | 51 | 71 | 1.7 |
| 941654 | 17 | 39 | 56 | 74 | 1.0 |
| 941714 | 27 | 44 | 62 | 78 | 0.6 |
| 941715 | 55 | 81 | 89 | 94 | <0.1 |
| 941726 | 26 | 55 | 78 | 89 | 0.3 |

TABLE 43
Dose-dependent reduction of human GYS1 RNA
in A431 cells by modified oligonucleotides

| Compound | % Reduction | | | | |
|---|---|---|---|---|---|
| Number | 63 nM | 250 nM | 1000 nM | 4000 nM | IC₅₀ μM |
| 1126888 | 50 | 74 | 80 | 84 | <0.1 |
| 1126889 | 13 | 54 | 73 | 80 | 0.4 |
| 1127015 | 36 | 59 | 79 | 85 | 0.2 |
| 1127016 | 27 | 40 | 68 | 79 | 0.4 |
| 1127017 | 33 | 52 | 75 | 87 | 0.2 |
| 1127113 | 48 | 68 | 83 | 90 | <0.1 |
| 1127241 | 11 | 51 | 74 | 84 | 0.4 |
| 1127335 | 13 | 39 | 64 | 76 | 0.6 |
| 1127369 | 32 | 55 | 71 | 85 | 0.2 |
| 1127399 | 37 | 57 | 77 | 85 | 0.2 |
| 1127431 | 25 | 52 | 74 | 86 | 0.3 |
| 1127432 | 50 | 80 | 91 | 96 | <0.1 |
| 1127433 | 50 | 74 | 90 | 94 | <0.1 |
| 1127463 | 30 | 61 | 78 | 84 | 0.2 |
| 1127464 | 47 | 69 | 85 | 92 | <0.1 |
| 1127879 | 24 | 48 | 71 | 80 | 0.3 |
| 1127954 | 40 | 76 | 91 | 95 | 0.1 |
| 1128009 | 36 | 49 | 68 | 78 | 0.2 |
| 1128040 | 27 | 54 | 78 | 91 | 0.2 |

TABLE 44
Dose-dependent reduction of human GYS1 RNA
in A431 cells by modified oligonucleotides

| Compound | % Reduction | | | | |
|---|---|---|---|---|---|
| Number | 63 nM | 250 nM | 1000 nM | 4000 nM | IC₅₀ μM |
| 1126795 | 26 | 42 | 62 | 69 | 0.5 |
| 1127018 | 16 | 40 | 50 | 71 | 0.8 |
| 1127019 | 39 | 58 | 74 | 82 | 0.1 |
| 1127020 | 10 | 43 | 63 | 71 | 0.6 |
| 1127084 | 20 | 54 | 74 | 88 | 0.3 |
| 1127114 | 29 | 53 | 78 | 89 | 0.2 |
| 1127402 | 27 | 58 | 75 | 87 | 0.2 |
| 1127403 | 35 | 46 | 67 | 78 | 0.3 |
| 1127434 | 26 | 46 | 70 | 81 | 0.3 |
| 1127466 | 87 | 95 | 97 | 97 | <0.1 |
| 1127467 | 47 | 76 | 87 | 91 | <0.1 |
| 1127498 | 36 | 72 | 83 | 89 | 0.1 |
| 1127499 | 48 | 72 | 84 | 90 | <0.1 |
| 1127882 | 36 | 56 | 73 | 80 | 0.2 |
| 1127954 | 36 | 75 | 90 | 95 | 0.1 |
| 1127979 | 49 | 76 | 89 | 94 | <0.1 |
| 1127980 | 36 | 60 | 83 | 88 | 0.1 |
| 1127981 | 53 | 76 | 90 | 95 | <0.1 |
| 1127982 | 41 | 77 | 90 | 95 | 0.1 |

TABLE 45
Dose-dependent reduction of human GYS1 RNA
in A431 cells by modified oligonucleotides

| Compound | % Reduction | | | | |
|---|---|---|---|---|---|
| Number | 63 nM | 250 nM | 1000 nM | 4000 nM | IC₅₀ μM |
| 1126896 | 37 | 57 | 72 | 77 | 0.2 |
| 1126993 | 12 | 48 | 76 | 79 | 0.4 |
| 1127055 | 23 | 35 | 63 | 75 | 0.5 |
| 1127085 | 10 | 11 | 41 | 65 | 1.8 |
| 1127215 | 11 | 25 | 59 | 66 | 1.0 |
| 1127217 | 20 | 39 | 64 | 81 | 0.5 |
| 1127248 | 33 | 48 | 66 | 77 | 0.3 |
| 1127249 | 38 | 56 | 80 | 88 | 0.1 |
| 1127344 | 27 | 44 | 67 | 76 | 0.4 |
| 1127409 | 27 | 45 | 74 | 83 | 0.3 |
| 1127535 | 33 | 58 | 82 | 88 | 0.2 |
| 1127952 | 22 | 48 | 68 | 82 | 0.4 |
| 1127953 | 19 | 57 | 75 | 83 | 0.3 |
| 1127954 | 35 | 76 | 90 | 95 | 0.1 |
| 1127983 | 27 | 64 | 81 | 88 | 0.2 |
| 1128015 | 28 | 36 | 56 | 62 | 0.8 |
| 1128016 | 45 | 70 | 83 | 90 | 0.1 |
| 1128017 | 19 | 52 | 79 | 88 | 0.3 |
| 1128018 | 43 | 75 | 91 | 96 | <0.1 |

TABLE 46
Dose-dependent reduction of human GYS1 RNA
in A431 cells by modified oligonucleotides

| Compound | % Reduction | | | | |
|---|---|---|---|---|---|
| Number | 63 nM | 250 nM | 1000 nM | 4000 nM | IC₅₀ μM |
| 1126803 | 39 | 61 | 81 | 88 | 0.1 |
| 1126899 | 56 | 78 | 89 | 91 | <0.1 |
| 1126900 | 27 | 65 | 83 | 88 | 0.2 |
| 1126931 | 43 | 65 | 80 | 86 | 0.1 |
| 1127251 | 39 | 67 | 84 | 89 | 0.1 |
| 1127313 | 7 | 35 | 50 | 52 | 1.8 |
| 1127410 | 46 | 73 | 89 | 95 | <0.1 |
| 1127411 | 19 | 44 | 73 | 86 | 0.4 |
| 1127506 | 20 | 41 | 66 | 80 | 0.4 |
| 1127507 | 22 | 50 | 75 | 85 | 0.3 |
| 1127538 | 6 | 38 | 62 | 71 | 0.7 |
| 1127603 | 41 | 73 | 88 | 95 | 0.1 |
| 1127954 | 36 | 78 | 92 | 95 | 0.1 |
| 1127955 | 32 | 48 | 72 | 83 | 0.3 |
| 1128019 | 30 | 63 | 82 | 87 | 0.2 |
| 1128021 | 34 | 74 | 89 | 94 | 0.1 |
| 1128051 | 52 | 76 | 86 | 91 | <0.1 |
| 1128052 | 6 | 37 | 62 | 76 | 0.7 |
| 1128275 | 37 | 44 | 70 | 67 | 0.3 |

TABLE 47
Dose-dependent reduction of human GYS1 RNA
in A431 cells by modified oligonucleotides

| Compound | % Reduction | | | | |
|---|---|---|---|---|---|
| Number | 63 nM | 250 nM | 1000 nM | 4000 nM | IC₅₀ μM |
| 1126292 | 0 | 29 | 59 | 78 | 0.8 |
| 1126293 | 6 | 27 | 49 | 77 | 0.9 |
| 1126901 | 24 | 43 | 72 | 81 | 0.4 |
| 1126902 | 29 | 54 | 77 | 82 | 0.2 |
| 1126996 | 32 | 50 | 67 | 72 | 0.3 |
| 1126997 | 28 | 47 | 68 | 74 | 0.3 |
| 1126998 | 28 | 49 | 68 | 83 | 0.3 |
| 1127093 | 9 | 35 | 68 | 79 | 0.6 |
| 1127094 | 5 | 41 | 65 | 79 | 0.6 |
| 1127252 | 19 | 42 | 69 | 80 | 0.4 |
| 1127253 | 21 | 35 | 59 | 71 | 0.7 |
| 1127286 | 8 | 36 | 62 | 67 | 0.8 |
| 1127413 | 8 | 28 | 59 | 71 | 0.9 |
| 1127414 | 38 | 63 | 84 | 92 | 0.1 |
| 1127444 | 20 | 54 | 69 | 75 | 0.4 |
| 1127604 | 13 | 32 | 51 | 57 | 1.5 |
| 1127669 | 37 | 53 | 66 | 76 | 0.2 |
| 1127954 | 48 | 80 | 91 | 95 | <0.1 |
| 1128278 | 33 | 49 | 70 | 79 | 0.3 |

TABLE 48
Dose-dependent reduction of human GYS1 RNA
in A431 cells by modified oligonucleotides

| Compound | % Reduction | | | | |
|---|---|---|---|---|---|
| Number | 63 nM | 250 nM | 1000 nM | 4000 nM | IC₅₀ μM |
| 1126775 | 48 | 74 | 84 | 87 | <0.1 |
| 1126776 | 48 | 71 | 83 | 88 | <0.1 |
| 1126777 | 41 | 65 | 78 | 84 | 0.1 |
| 1126778 | 5 | 39 | 62 | 77 | 0.6 |
| 1126999 | 32 | 39 | 73 | 81 | 0.3 |
| 1127000 | 19 | 45 | 68 | 77 | 0.4 |
| 1127001 | 47 | 66 | 80 | 88 | 0.1 |
| 1127096 | 9 | 35 | 60 | 71 | 0.8 |
| 1127291 | 21 | 53 | 74 | 84 | 0.3 |
| 1127384 | 0 | 24 | 46 | 70 | 1.3 |
| 1127385 | 24 | 46 | 73 | 87 | 0.3 |
| 1127954 | 47 | 80 | 92 | 96 | <0.1 |
| 1127960 | 31 | 62 | 83 | 92 | 0.2 |
| 1127961 | 36 | 42 | 67 | 81 | 0.3 |
| 1127962 | 49 | 72 | 88 | 95 | <0.1 |
| 1127963 | 21 | 48 | 81 | 92 | 0.3 |
| 1127993 | 16 | 45 | 64 | 73 | 0.5 |
| 1128027 | 37 | 65 | 83 | 90 | 0.1 |
| 1128028 | 32 | 70 | 84 | 91 | 0.1 |

TABLE 49
Dose-dependent reduction of human GYS1 RNA
in A431 cells by modified oligonucleotides

| Compound | % Reduction | | | | |
|---|---|---|---|---|---|
| Number | 63 nM | 250 nM | 1000 nM | 4000 nM | IC₅₀ μM |
| 1126331 | 0 | 20 | 53 | 63 | 1.4 |
| 1127036 | 28 | 57 | 78 | 87 | 0.2 |
| 1127037 | 33 | 47 | 68 | 78 | 0.3 |
| 1127038 | 29 | 52 | 73 | 84 | 0.3 |
| 1127039 | 57 | 80 | 90 | 94 | <0.1 |
| 1127040 | 53 | 81 | 90 | 94 | <0.1 |
| 1127102 | 37 | 46 | 66 | 78 | 0.3 |
| 1127260 | 21 | 42 | 73 | 78 | 0.4 |
| 1127261 | 34 | 60 | 79 | 86 | 0.2 |
| 1127292 | 23 | 35 | 59 | 69 | 0.7 |
| 1127357 | 23 | 52 | 70 | 84 | 0.3 |
| 1127358 | 31 | 61 | 81 | 89 | 0.2 |
| 1127391 | 33 | 44 | 63 | 80 | 0.3 |
| 1127420 | 24 | 46 | 67 | 72 | 0.4 |
| 1127579 | 3 | 7 | 48 | 77 | 1.2 |
| 1127582 | 40 | 64 | 76 | 83 | 0.1 |
| 1127954 | 58 | 84 | 92 | 96 | <0.1 |
| 1127964 | 29 | 55 | 71 | 70 | 0.3 |
| 1128032 | 18 | 40 | 64 | 81 | 0.5 |

TABLE 50
Dose-dependent reduction of human GYS1 RNA
in A431 cells by modified oligonucleotides

| Compound | % Reduction | | | | |
|---|---|---|---|---|---|
| Number | 63 nM | 250 nM | 1000 nM | 4000 nM | IC₅₀ μM |
| 1126433 | 0 | 0 | 13 | 12 | >4.0 |
| 1126784 | 13 | 45 | 63 | 76 | 0.5 |
| 1126850 | 24 | 46 | 63 | 72 | 0.5 |
| 1127041 | 24 | 53 | 68 | 77 | 0.3 |
| 1127042 | 48 | 67 | 84 | 89 | <0.1 |
| 1127299 | 40 | 63 | 85 | 92 | 0.1 |
| 1127329 | 42 | 62 | 76 | 81 | 0.1 |
| 1127330 | 28 | 62 | 76 | 86 | 0.2 |
| 1127331 | 44 | 62 | 78 | 82 | 0.1 |
| 1127363 | 22 | 49 | 71 | 81 | 0.3 |
| 1127426 | 31 | 47 | 59 | 72 | 0.4 |
| 1127428 | 48 | 75 | 89 | 95 | <0.1 |
| 1127954 | 53 | 80 | 92 | 96 | <0.1 |
| 1128004 | 34 | 60 | 81 | 89 | 0.2 |
| 1128033 | 20 | 43 | 65 | 74 | 0.5 |
| 1128035 | 18 | 43 | 67 | 82 | 0.4 |
| 1128036 | 44 | 71 | 90 | 95 | 0.1 |
| 1128483 | 31 | 49 | 63 | 70 | 0.4 |
| 1128609 | 27 | 48 | 68 | 75 | 0.3 |

TABLE 51
Dose-dependent reduction of human GYS1 RNA
in A431 cells by modified oligonucleotides

| Compound | % Reduction | | | | |
|---|---|---|---|---|---|
| Number | 63 nM | 250 nM | 1000 nM | 4000 nM | IC₅₀ μM |
| 1126788 | 23 | 30 | 58 | 66 | 0.8 |
| 1126789 | 48 | 67 | 82 | 79 | <0.1 |
| 1126790 | 24 | 36 | 62 | 75 | 0.5 |
| 1126820 | 21 | 30 | 57 | 63 | 1.0 |
| 1126884 | 5 | 32 | 64 | 67 | 0.8 |
| 1126885 | 22 | 39 | 60 | 70 | 0.6 |
| 1126886 | 0 | 37 | 66 | 81 | 0.7 |
| 1127013 | 8 | 37 | 63 | 75 | 0.6 |
| 1127014 | 22 | 45 | 65 | 79 | 0.4 |
| 1127172 | 6 | 15 | 44 | 50 | 3.1 |
| 1127174 | 29 | 53 | 80 | 86 | 0.2 |
| 1127332 | 28 | 42 | 59 | 59 | 0.7 |
| 1127398 | 42 | 65 | 81 | 90 | 0.1 |
| 1127429 | 15 | 50 | 71 | 79 | 0.4 |
| 1127430 | 50 | 61 | 78 | 85 | <0.1 |
| 1127954 | 45 | 78 | 92 | 96 | <0.1 |
| 1128037 | 0 | 35 | 61 | 75 | 0.8 |
| 1128038 | 34 | 53 | 75 | 82 | 0.2 |
| 1128039 | 9 | 51 | 71 | 77 | 0.4 |

### Example 4: Design of Modified Oligonucleotides Complementary to a GYS1 Nucleic Acid

Modified oligonucleotides were designed as indicated in the tables below.

The compounds in Table 52 are 5-10-5 MOE gapmers. The gapmers are 20 nucleosides in length, wherein the central gap segment consists of ten nucleosides comprising 2′-β-D-deoxyribosyl sugar moieties, the 5′ wing segment consists of five nucleosides comprising 2′-MOE modified sugar moieties, and the 3′ wing segment consists of five nucleosides comprising 2′-MOE modified sugar moieties. The sugar motif of the gapmers is (from 5′ to 3′): eeeeeddddddddddeeeee; wherein ‘d’ represents a 2′-β-D-deoxyribosyl sugar moiety, and ‘e’ represents a 2′-O(CH₂)₂OCH₃ ribosyl sugar moiety. The gapmers have an internucleoside linkage motif of (from 5′ to 3′): soooossssssssssooss; wherein “s” represents a phosphorothioate internucleoside linkage and “o” represents a phosphodiester internucleoside linkage. All cytosine residues are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. The modified oligonucleotide listed in the Tables below are 100% complementary to either SEQ ID NO: 1 (described herein above), or SEQ ID NO: 2 (described herein above). ‘N/A’ indicates that the modified oligonucleotide is not 100% complementary to that particular target nucleic acid sequence.

TABLE 52
5-10-5 MOE gapmers complementary to human GYS1

| | SEQ | SEQ | SEQ ID | SEQ ID | | |
|---|---|---|---|---|---|---|
| | ID NO: | ID NO: | NO: 2 | NO: 2 | | |
| Compound | 1 Start | 1 Stop | Start | Stop | | SEQ |
| Number | Site | Site | Site | Site | Sequence (5′ to 3′) | ID No. |
| 648138 | 290 | 309 | 2937 | 2956 | TCGAATTCATCCTCCCAGTC | 1939 |
| 648146 | 356 | 375 | N/A | N/A | GTGTAGATGCCACCCACCTT | 2935 |
| 648374 | N/A | N/A | 3639 | 3658 | CTCCCATCCTAGGGCCCAGC | 2936 |
| 648378 | N/A | N/A | 4509 | 4528 | GATGCCACCCACTGTGGGCC | 2937 |
| 648408 | N/A | N/A | 16869 | 16888 | AAATGTCAGCTATTATTAGG | 2938 |
| 648409 | N/A | N/A | 16872 | 16891 | CAGAAATGTCAGCTATTATT | 2939 |
| 648410 | N/A | N/A | 16875 | 16894 | TTCCAGAAATGTCAGCTATT | 2940 |
| 648411 | N/A | N/A | 16878 | 16897 | GCCTTCCAGAAATGTCAGCT | 2941 |
| 1251627 | N/A | N/A | 8379 | 8398 | CTGTTGCAATATTTTCCGTG | 673 |

The compound in Table 53 is a 5-10-5 MOE gapmer. The gapmer is 20 nucleosides in length, wherein the central gap segment consists of ten nucleosides comprising 2′-β-D-deoxyribosyl sugar moieties, the 5′ wing segment consists of five nucleosides comprising 2′-MOE modified sugar moieties, and the 3′ wing segment consists of five nucleosides comprising 2′-MOE modified sugar moieties. The sugar motif of the gapmer is (from 5′ to 3′): eeeeeddddddddddeeeee; wherein ‘d’ represents a 2′-β-D-deoxyribosyl sugar moiety, and ‘e’ represents a 2′-O(CH₂)₂OCH₃ ribosyl sugar moiety. The gapmer has an internucleoside linkage motif of (from 5′ to 3′): sooosssssssssssooos; wherein “s” represents a phosphorothioate internucleoside linkage and “o” represents a phosphodiester internucleoside linkage.

All cytosine residues are 5-methylcytosines. “Start site” indicates the 5′-most nucleoside of the target sequence to which the modified oligonucleotide is complementary. “Stop site” indicates the 3′-most nucleoside of the target sequence to which the modified oligonucleotide is complementary. The modified oligonucleotide listed in the Table below is 100% complementary to SEQ ID NO: 2 (described herein above). ‘N/A’ indicates that the modified oligonucleotide is not 100% complementary to that particular target nucleic acid sequence.

TABLE 53
5-10-5 MOE gapmer with complementary to human GYS1

| | SEQ | SEQ | SEQ | SEQ | | |
|---|---|---|---|---|---|---|
| | ID NO: | ID NO: | ID NO: | ID NO: | | |
| Compound | 1 Start | 1 Stop | 2 Start | 2 Stop | | SEQ |
| Number | Site | Site | Site | Site | Sequence (5′ to 3′) | ID No. |
| 1251628 | N/A | N/A | 8379 | 8398 | CTGTTGCAATATTTTCCGTG | 673 |

The compounds in Table 54 are 5-10-5 MOE gapmers. The gapmers are 20 nucleosides in length, wherein the central gap segment consists of ten nucleosides comprising 2′-β-D-deoxyribosyl sugar moieties, the 5′ wing segment consists of five nucleosides comprising 2′-MOE modified sugar moieties, and the 3′ wing segment consists of five nucleosides comprising 2′-MOE modified sugar moieties. The sugar motif of the gapmers is (from 5′ to 3′): eeeeeddddddddddeeeee; wherein ‘d’ represents a 2′-β-D-deoxyribosyl sugar moiety, and ‘e’ represents a 2′-O(CH₂)₂OCH₃ ribosyl sugar moiety. The gapmers have an internucleoside linkage motif of (from 5′ to 3′): sooosssssssssssooss; wherein “s” represents a phosphorothioate internucleoside linkage and “o” represents a phosphodiester internucleoside linkage. All cytosine residues are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. Each modified oligonucleotide listed in the Tables below is 100% complementary to SEQ ID NO: 1 (described herein above), or SEQ ID NO: 2 (described herein above). ‘N/A’ indicates that the modified oligonucleotide is not 100% complementary to that particular target nucleic acid sequence.

TABLE 54
5-10-5 MOE gapmers complementary to human GYS1

| | SEQ | SEQ | SEQ | SEQ | | |
|---|---|---|---|---|---|---|
| | ID | ID | ID | ID | | |
| | NO: | NO: | NO: | NO: | | |
| | 1 | 1 | 2 | 2 | | SEQ |
| Compound | Start | Stop | Start | Stop | | ID |
| Number | Site | Site | Site | Site | Sequence (5′ to 3′) | No. |
| 1251610 | N/A | N/A | 23519 | 23538 | TGCCCCTGACCTATAATTAT | 2942 |
| 1251611 | N/A | N/A | 15667 | 15686 | CCAAAAGTAAATTCCGTCTA | 2943 |
| 1251612 | N/A | N/A | 15666 | 15685 | CAAAAGTAAATTCCGTCTAC | 2944 |
| 1251613 | N/A | N/A | 15652 | 15671 | GTCTACAGGATTTTCTAGAA | 2945 |
| 1251614 | N/A | N/A | 15644 | 15663 | GATTTTCTAGAATACAAGCT | 2946 |
| 1251615 | N/A | N/A | 15658 | 15677 | AATTCCGTCTACAGGATTTT | 2947 |
| 1251616 | N/A | N/A | 15650 | 15669 | CTACAGGATTTTCTAGAATA | 2948 |
| 1251617 | N/A | N/A | 15665 | 15684 | AAAAGTAAATTCCGTCTACA | 2949 |
| 1251618 | N/A | N/A | 15670 | 15689 | TGCCCAAAAGTAAATTCCGT | 2950 |
| 1251619 | N/A | N/A | 15664 | 15683 | AAAGTAAATTCCGTCTACAG | 2951 |
| 1251620 | N/A | N/A | 15646 | 15665 | AGGATTTTCTAGAATACAAG | 2952 |
| 1251621 | N/A | N/A | 15651 | 15670 | TCTACAGGATTTTCTAGAAT | 2953 |
| 1251622 | N/A | N/A | 15657 | 15676 | ATTCCGTCTACAGGATTTTC | 2954 |
| 1251623 | N/A | N/A | 15671 | 15690 | TTGCCCAAAAGTAAATTCCG | 2955 |
| 1251624 | N/A | N/A | 15645 | 15664 | GGATTTTCTAGAATACAAGC | 2956 |
| 1270755 | N/A | N/A | 16887 | 16906 | ACATGGCAAGCCTTCCAGAA | 2957 |
| 1270756 | N/A | N/A | 16867 | 16886 | ATGTCAGCTATTATTAGGAC | 2958 |
| 1270757 | N/A | N/A | 12626 | 12645 | GTTCACAAGCACAAGTATAA | 2959 |
| 1270758 | N/A | N/A | 12597 | 12616 | GCTACTGCAAATGGCTAACA | 2960 |
| 1270759 | N/A | N/A | 16885 | 16904 | ATGGCAAGCCTTCCAGAAAT | 2961 |
| 1270760 | N/A | N/A | 12651 | 12670 | TTCTTGCCTAATATTACTTG | 2962 |
| 1270761 | N/A | N/A | 12643 | 12662 | TAATATTACTTGCTTAGGTT | 2963 |
| 1270762 | N/A | N/A | 12615 | 12634 | CAAGTATAAGTCTCCTCAGC | 2964 |
| 1270763 | N/A | N/A | 12650 | 12669 | TCTTGCCTAATATTACTTGC | 2965 |
| 1270764 | N/A | N/A | 12618 | 12637 | GCACAAGTATAAGTCTCCTC | 310 |
| 1270765 | N/A | N/A | 12610 | 12629 | ATAAGTCTCCTCAGCTACTG | 2966 |
| 1270766 | N/A | N/A | 16866 | 16885 | TGTCAGCTATTATTAGGACT | 2967 |
| 1270767 | N/A | N/A | 12628 | 12647 | AGGTTCACAAGCACAAGTAT | 2968 |
| 1270768 | N/A | N/A | 12607 | 12626 | AGTCTCCTCAGCTACTGCAA | 2969 |
| 1270769 | N/A | N/A | 16881 | 16900 | CAAGCCTTCCAGAAATGTCA | 2970 |
| 1270770 | N/A | N/A | 16883 | 16902 | GGCAAGCCTTCCAGAAATGT | 2971 |
| 1270771 | N/A | N/A | 12621 | 12640 | CAAGCACAAGTATAAGTCTC | 2972 |
| 1270772 | N/A | N/A | 12633 | 12652 | TGCTTAGGTTCACAAGCACA | 2973 |
| 1270773 | N/A | N/A | 16880 | 16899 | AAGCCTTCCAGAAATGTCAG | 2974 |
| 1270774 | N/A | N/A | 12609 | 12628 | TAAGTCTCCTCAGCTACTGC | 2975 |
| 1270775 | N/A | N/A | 12613 | 12632 | AGTATAAGTCTCCTCAGCTA | 2976 |
| 1270776 | N/A | N/A | 12653 | 12672 | CCTTCTTGCCTAATATTACT | 2977 |
| 1270777 | N/A | N/A | 12580 | 12599 | ACAGGGATCTGATCCACACT | 2978 |
| 1270778 | N/A | N/A | 16865 | 16884 | GTCAGCTATTATTAGGACTT | 2979 |
| 1270779 | N/A | N/A | 16882 | 16901 | GCAAGCCTTCCAGAAATGTC | 2980 |
| 1270780 | N/A | N/A | 12577 | 12596 | GGGATCTGATCCACACTGGT | 233 |
| 1270781 | N/A | N/A | 12599 | 12618 | CAGCTACTGCAAATGGCTAA | 2981 |
| 1270782 | N/A | N/A | 12600 | 12619 | TCAGCTACTGCAAATGGCTA | 2982 |
| 1270783 | N/A | N/A | 12591 | 12610 | GCAAATGGCTAACAGGGATC | 2983 |
| 1270784 | N/A | N/A | 12654 | 12673 | GCCTTCTTGCCTAATATTAC | 387 |
| 1270785 | N/A | N/A | 16886 | 16905 | CATGGCAAGCCTTCCAGAAA | 2984 |
| 1270786 | N/A | N/A | 12601 | 12620 | CTCAGCTACTGCAAATGGCT | 2985 |
| 1270787 | N/A | N/A | 12606 | 12625 | GTCTCCTCAGCTACTGCAAA | 2986 |
| 1270788 | N/A | N/A | 12649 | 12668 | CTTGCCTAATATTACTTGCT | 2987 |
| 1270789 | N/A | N/A | 12622 | 12641 | ACAAGCACAAGTATAAGTCT | 2988 |
| 1270790 | N/A | N/A | 12644 | 12663 | CTAATATTACTTGCTTAGGT | 2989 |
| 1270791 | N/A | N/A | 16855 | 16874 | ATTAGGACTTGTACTTGTCC | 392 |
| 1270792 | N/A | N/A | 12608 | 12627 | AAGTCTCCTCAGCTACTGCA | 2990 |
| 1270793 | N/A | N/A | 12648 | 12667 | TTGCCTAATATTACTTGCTT | 2991 |
| 1270794 | N/A | N/A | 12634 | 12653 | TTGCTTAGGTTCACAAGCAC | 2992 |
| 1270795 | N/A | N/A | 12623 | 12642 | CACAAGCACAAGTATAAGTC | 2993 |
| 1270796 | N/A | N/A | 12647 | 12666 | TGCCTAATATTACTTGCTTA | 2994 |
| 1311835 | N/A | N/A | 16879 | 16898 | AGCCTTCCAGAAATGTCAGC | 2995 |
| 1311836 | N/A | N/A | 16877 | 16896 | CCTTCCAGAAATGTCAGCTA | 2996 |
| 1311837 | N/A | N/A | 16876 | 16895 | CTTCCAGAAATGTCAGCTAT | 2997 |
| 1318941 | N/A | N/A | 10152 | 10171 | CTGCCACGGTCCCAGCTCAC | 2998 |
| 1318942 | N/A | N/A | 10145 | 10164 | GGTCCCAGCTCACGTTCTCC | 2999 |
| 1318943 | N/A | N/A | 10146 | 10165 | CGGTCCCAGCTCACGTTCTC | 3000 |
| 1318944 | N/A | N/A | 10148 | 10167 | CACGGTCCCAGCTCACGTTC | 3001 |
| 1318945 | N/A | N/A | 10149 | 10168 | CCACGGTCCCAGCTCACGTT | 3002 |
| 1318946 | N/A | N/A | 10147 | 10166 | ACGGTCCCAGCTCACGTTCT | 3003 |
| 1318948 | N/A | N/A | 10153 | 10172 | TCTGCCACGGTCCCAGCTCA | 3004 |
| 1318949 | N/A | N/A | 12655 | 12674 | TGCCTTCTTGCCTAATATTA | 3005 |
| 1318950 | N/A | N/A | 15698 | 15717 | GTCTCATATTCCAGTTCTGC | 3006 |
| 1318951 | N/A | N/A | 5761 | 5780 | TCCTTGGACAACAGCGTGCC | 3007 |
| 1318952 | N/A | N/A | 10139 | 10158 | AGCTCACGTTCTCCAGGTTG | 3008 |
| 1318953 | N/A | N/A | 5759 | 5778 | CTTGGACAACAGCGTGCCGC | 3009 |
| 1318954 | N/A | N/A | 5758 | 5777 | TTGGACAACAGCGTGCCGCC | 3010 |
| 1318955 | N/A | N/A | 10138 | 10157 | GCTCACGTTCTCCAGGTTGT | 3011 |
| 1318956 | N/A | N/A | 10143 | 10162 | TCCCAGCTCACGTTCTCCAG | 3012 |
| 1318957 | N/A | N/A | 5760 | 5779 | CCTTGGACAACAGCGTGCCG | 3013 |
| 1318958 | N/A | N/A | 5751 | 5770 | ACAGCGTGCCGCCTGATGGC | 3014 |
| 1318959 | N/A | N/A | 10140 | 10159 | CAGCTCACGTTCTCCAGGTT | 3015 |
| 1318960 | N/A | N/A | 5748 | 5767 | GCGTGCCGCCTGATGGCTTT | 3016 |
| 1318961 | N/A | N/A | 10144 | 10163 | GTCCCAGCTCACGTTCTCCA | 3017 |
| 1318962 | N/A | N/A | 5762 | 5781 | CTCCTTGGACAACAGCGTGC | 3018 |
| 1318963 | N/A | N/A | 5749 | 5768 | AGCGTGCCGCCTGATGGCTT | 3019 |
| 1318964 | N/A | N/A | 10135 | 10154 | CACGTTCTCCAGGTTGTTGT | 3020 |
| 1251609 | N/A | N/A | 5524 | 5543 | GTCTATTACTCTTGCCTGTG | 19 |
| 1318947 | N/A | N/A | 10150 | 10169 | GCCACGGTCCCAGCTCACGT | 3023 |

The compounds in Table 55 are 5-9-5 MOE gapmers. The gapmers are 19 nucleosides in length, wherein the central gap segment consists of nine nucleosides comprising 2′-β-D-deoxyribosyl sugar moieties, the 5′ wing segment consists of five nucleosides comprising 2′-MOE modified sugar moieties, and the 3′ wing segment consists of five nucleosides comprising 2′-MOE modified sugar moieties. The sugar motif of the gapmers is (from 5′ to 3′): eeeeedddddddddeeeee; wherein ‘d’ represents a 2′-β-D-deoxyribosyl sugar moiety, and ‘e’ represents a 2′-O(CH₂)₂OCH₃ ribosyl sugar moiety. The gapmers have an internucleoside linkage motif of (from 5′ to 3′): sooossssssssssooss; wherein “s” represents a phosphorothioate internucleoside linkage and “o” represents a phosphodiester internucleoside linkage. All cytosine residues are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. Each modified oligonucleotide listed in the Tables below is 100% complementary to SEQ ID NO: 1 (described herein above), or SEQ ID NO: 2 (described herein above). ‘N/A’ indicates that the modified oligonucleotide is not 100% complementary to that particular target nucleic acid sequence.

TABLE 55
5-9-5 MOE gapmers complementary to human GYS1

| | SEQ | SEQ | SEQ ID | SEQ ID | | |
|---|---|---|---|---|---|---|
| | ID NO: | ID NO: | NO: 2 | NO: 2 | | |
| Compound | 1 Start | 1 Stop | Start | Stop | | SEQ |
| Number | Site | Site | Site | Site | Sequence (5′ to 3′) | ID No. |
| 1251625 | N/A | N/A | 8379 | 8397 | TGTTGCAATATTTTCCGTG | 3021 |
| 1251626 | N/A | N/A | 8380 | 8398 | CTGTTGCAATATTTTCCGT | 3022 |

The compounds in Table 56 are 6-10-4 MOE gapmers. The gapmers are 20 nucleosides in length, wherein the central gap segment consists of ten nucleosides comprising 2′-β-D-deoxyribosyl sugar moieties, the 5′ wing segment consists of six nucleosides comprising 2′-MOE modified sugar moieties, and the 3′ wing segment consists of four nucleosides comprising 2′-MOE modified sugar moieties. The sugar motif of the gapmers is (from 5′ to 3′): eeeeeeddddddddddeeee; wherein ‘d’ represents a 2′-β-D-deoxyribosyl sugar moiety, and ‘e’ represents a 2′-O(CH₂)₂OCH₃ ribosyl sugar moiety. The gapmers have an internucleoside linkage motif of (from 5′ to 3′): sooooossssssssssoss; wherein “s” represents a phosphorothioate internucleoside linkage and “o” represents a phosphodiester internucleoside linkage. All cytosine residues are 5-methylcytosines.

“Start site” indicates the 5′-most nucleoside of the target sequence to which the modified oligonucleotide is complementary. “Stop site” indicates the 3′-most nucleoside of the target sequence to which the modified oligonucleotide is complementary. Each modified oligonucleotide listed in the Tables below is 100% complementary to SEQ ID NO: 1 (described herein above), or SEQ ID NO: 2 (described herein above). ‘N/A’ indicates that the modified oligonucleotide is not 100% complementary to that particular target nucleic acid sequence.

TABLE 56
6-10-4 MOE gapmers complementary to human GYS1

| | SEQ | SEQ | SEQ ID | SEQ ID | | |
|---|---|---|---|---|---|---|
| | ID NO: | ID NO: | NO: 2 | NO: 2 | | |
| Compound | 1 Start | 1 Stop | Start | Stop | | SEQ |
| Number | Site | Site | Site | Site | Sequence (5′ to 3′) | ID No. |
| 1311839 | N/A | N/A | 5743 | 5762 | CCGCCTGATGGCTTTCTCCT | 2736 |
| | | | 5777 | 5796 | | |
| 1311840 | 291 | 310 | 2938 | 2957 | GTCGAATTCATCCTCCCAGT | 2015 |
| 1311841 | 297 | 316 | 2944 | 2963 | CTCCAGGTCGAATTCATCCT | 333 |
| 1311842 | 360 | 379 | 4518 | 4537 | CACCGTGTAGATGCCACCCA | 2707 |
| 1311843 | 238 | 257 | 2885 | 2904 | TGCGGTTTAAAGGCATGGCT | 24 |
| 1311844 | 239 | 258 | 2886 | 2905 | GTGCGGTTTAAAGGCATGGC | 1176 |
| 1311845 | N/A | N/A | 4516 | 4535 | CCGTGTAGATGCCACCCACT | 2504 |
| 1311846 | N/A | N/A | 3275 | 3294 | CAGGTATCTAGTCTCTCGGA | 1654 |
| 1311847 | N/A | N/A | 4982 | 5001 | GACCAGGCTGTCTTTCGATC | 453 |
| 1311848 | N/A | N/A | 5311 | 5330 | CGCTATTAGCATGTCCATTA | 2121 |
| 1311849 | N/A | N/A | 5599 | 5618 | CCCCGATATTCCATGCTTCT | 377 |
| 1311850 | N/A | N/A | 7669 | 7688 | GATGCTAATTCCTTCCACGG | 1363 |
| 1311851 | N/A | N/A | 8374 | 8393 | GCAATATTTTCCGTGCCCCC | 2740 |
| 1311852 | N/A | N/A | 8375 | 8394 | TGCAATATTTTCCGTGCCCC | 2817 |
| 1311853 | N/A | N/A | 8376 | 8395 | TTGCAATATTTTCCGTGCCC | 2894 |
| 1311854 | N/A | N/A | 8378 | 8397 | TGTTGCAATATTTTCCGTGC | 597 |
| 1311855 | N/A | N/A | 8377 | 8396 | GTTGCAATATTTTCCGTGCC | 520 |
| 1311856 | N/A | N/A | 15656 | 15675 | TTCCGTCTACAGGATTTTCT | 1454 |
| 1311857 | N/A | N/A | 15654 | 15673 | CCGTCTACAGGATTTTCTAG | 83 |
| 1311858 | N/A | N/A | 15655 | 15674 | TCCGTCTACAGGATTTTCTA | 1380 |
| 1311859 | N/A | N/A | 7001 | 7020 | CCTGCGACCTCTTTCCTCTA | 1972 |
| 1311860 | N/A | N/A | 6987 | 7006 | CCTCTATCCATAGGCCCATT | 1819 |
| 1311861 | N/A | N/A | 6988 | 7007 | TCCTCTATCCATAGGCCCAT | 1895 |
| 1311862 | N/A | N/A | 7013 | 7032 | ACATATCTGTCACCTGCGAC | 2202 |
| 1311863 | N/A | N/A | 7012 | 7031 | CATATCTGTCACCTGCGACC | 2125 |
| 1311864 | N/A | N/A | 7014 | 7033 | CACATATCTGTCACCTGCGA | 2279 |
| 1311865 | N/A | N/A | 7217 | 7236 | GTATCACATCCCGGCTAATT | 2433 |
| 1311866 | N/A | N/A | 5746 | 5765 | GTGCCGCCTGATGGCTTTCT | 301 |
| 1311867 | N/A | N/A | 5731 | 5750 | TTTCTCCTTGGACAACAGCG | 70 |
| | | | 5765 | 5784 | | |
| 1311868 | N/A | N/A | 5747 | 5766 | CGTGCCGCCTGATGGCTTTC | 378 |
| 1311869 | N/A | N/A | 16878 | 16897 | GCCTTCCAGAAATGTCAGCT | 2941 |
| 1311870 | 289 | 308 | 2936 | 2955 | CGAATTCATCCTCCCAGTCC | 256 |
| 1311872 | 292 | 311 | 2939 | 2958 | GGTCGAATTCATCCTCCCAG | 2092 |
| 1311873 | 359 | 378 | 4517 | 4536 | ACCGTGTAGATGCCACCCAC | 2631 |
| 1311874 | N/A | N/A | 3324 | 3343 | GTTTGCACGCAGAAATCCCA | 2498 |
| 1311875 | N/A | N/A | 4509 | 4528 | GATGCCACCCACTGTGGGCC | 2937 |
| 1311876 | 356 | 375 | N/A | N/A | GTGTAGATGCCACCCACCTT | 2935 |
| 1311877 | 296 | 315 | 2943 | 2962 | TCCAGGTCGAATTCATCCTC | 2169 |
| 1311878 | 290 | 309 | 2937 | 2956 | TCGAATTCATCCTCCCAGTC | 1939 |
| 1318966 | N/A | N/A | 10138 | 10157 | GCTCACGTTCTCCAGGTTGT | 3011 |
| 1318967 | N/A | N/A | 10135 | 10154 | CACGTTCTCCAGGTTGTTGT | 3020 |
| 1318968 | N/A | N/A | 10139 | 10158 | AGCTCACGTTCTCCAGGTTG | 3008 |
| 1318969 | N/A | N/A | 10143 | 10162 | TCCCAGCTCACGTTCTCCAG | 3012 |
| 1318970 | N/A | N/A | 10144 | 10163 | GTCCCAGCTCACGTTCTCCA | 3017 |
| 1318971 | N/A | N/A | 10140 | 10159 | CAGCTCACGTTCTCCAGGTT | 3015 |
| 1318972 | N/A | N/A | 10147 | 10166 | ACGGTCCCAGCTCACGTTCT | 3003 |
| 1318973 | N/A | N/A | 10145 | 10164 | GGTCCCAGCTCACGTTCTCC | 2999 |
| 1318974 | N/A | N/A | 10150 | 10169 | GCCACGGTCCCAGCTCACGT | 3023 |
| 1318975 | N/A | N/A | 10146 | 10165 | CGGTCCCAGCTCACGTTCTC | 3000 |
| 1318976 | N/A | N/A | 10148 | 10167 | CACGGTCCCAGCTCACGTTC | 3001 |
| 1318977 | N/A | N/A | 10149 | 10168 | CCACGGTCCCAGCTCACGTT | 3002 |
| 1318978 | N/A | N/A | 10152 | 10171 | CTGCCACGGTCCCAGCTCAC | 2998 |
| 1318979 | N/A | N/A | 12654 | 12673 | GCCTTCTTGCCTAATATTAC | 387 |
| 1318980 | N/A | N/A | 12655 | 12674 | TGCCTTCTTGCCTAATATTA | 3005 |
| 1318981 | N/A | N/A | 15698 | 15717 | GTCTCATATTCCAGTTCTGC | 3006 |
| 1318982 | N/A | N/A | 10153 | 10172 | TCTGCCACGGTCCCAGCTCA | 3004 |
| 1318983 | N/A | N/A | 15699 | 15718 | CGTCTCATATTCCAGTTCTG | 2066 |
| 1318984 | N/A | N/A | 16876 | 16895 | CTTCCAGAAATGTCAGCTAT | 2997 |
| 1318985 | N/A | N/A | 16877 | 16896 | CCTTCCAGAAATGTCAGCTA | 2996 |
| 1318986 | N/A | N/A | 5762 | 5781 | CTCCTTGGACAACAGCGTGC | 3018 |
| 1318987 | N/A | N/A | 5751 | 5770 | ACAGCGTGCCGCCTGATGGC | 3014 |
| 1318988 | N/A | N/A | 5749 | 5768 | AGCGTGCCGCCTGATGGCTT | 3019 |
| 1318989 | N/A | N/A | 5758 | 5777 | TTGGACAACAGCGTGCCGCC | 3010 |
| 1318990 | N/A | N/A | 5748 | 5767 | GCGTGCCGCCTGATGGCTTT | 3016 |
| 1318991 | N/A | N/A | 5760 | 5779 | CCTTGGACAACAGCGTGCCG | 3013 |
| 1318992 | N/A | N/A | 5761 | 5780 | TCCTTGGACAACAGCGTGCC | 3007 |
| 1318993 | N/A | N/A | 5759 | 5778 | CTTGGACAACAGCGTGCCGC | 3009 |

### Example 5: Activity of Modified Oligonucleotides Complementary to Human GYS1 in Transgenic Mice

Modified oligonucleotides described above were tested in a human GYS1 transgenic mouse model FVB-Tg. The transgenic mouse was designed using the fosmid clone ABC9-43950100I15, which spans the entire genomic location of the human GYS1 gene (specifically, it spans chromosome 19 from positions 49468032 to 49508811 on assembly GRCh37.p2).

### Treatment

The GYS1 transgenic mice were divided into groups of 3-6 mice each. Each mouse received a single ICV bolus of 200 μg of modified oligonucleotide. A group of 3-4 mice received PBS as a negative control.

### RNA Analysis

Two weeks post treatment, mice were sacrificed and RNA was extracted from cortical brain tissue, and/or spinal cord for RT-PCR analysis to measure the amount of GYS1 RNA using human GYS1 primer probe set RTS36345 (forward sequence CGGCTCAACTATCTGCTCAG, designated herein as SEQ ID NO: 3024; reverse sequence GTGTCCCAAAGCTGTTTGC designated herein as SEQ ID NO: 3025; probe sequence CAACGTGGAAACCCTCAAAGGCC, designated herein as SEQ ID NO: 3026) or human GYS1 primer probe set RTS39670 (forward sequence ACTTTGTCCATGTCCTCACTG, designated herein as SEQ ID NO: 3027; reverse sequence CCTGTCACCTTCGCCTTC, designated herein as SEQ ID NO: 3028; and probe sequence ACCCACCTTGTTAGCCACCTCC, designated herein as 3029). Results are presented as percent reduction of GYS1 RNA relative to the amount of GYS1 RNA in the PBS control normalized to mouse cyclophilin A. As used herein, a value of ‘0’ indicates that treatment with the modified oligonucleotide did not inhibit GYS1 mRNA levels. Mouse cyclophilin A was amplified using primer probe set m_cyclo24 (forward sequence TCGCCGCTTGCTGCA, designated herein as SEQ ID NO: 3030; reverse sequence ATCGGCCGTGATGTCGA, designated herein as SEQ ID NO: 3031; probe sequence CCATGGTCAACCCCACCGTGTTC, designated herein as SEQ ID NO: 3032.

As shown in the tables below, treatment with modified oligonucleotides resulted in reduction of GYS1 RNA in comparison to the amount of GYS1 RNA in the PBS control (control designated as 0% reduction). Each table represents an individual study.

TABLE 57
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 941359 | 38 | 73 |
| 941597 | 2 | 42 |
| 941630 | 73 | 88 |
| 941694 | 72 | 73 |
| 941714 | 47 | 50 |
| 941718 | 64 | 67 |
| 941726 | 39 | 57 |
| 1126789 | 27 | 52 |
| 1127039 | 50 | 61 |
| 1127299 | 72 | 84 |
| 1127432 | 89 | 86 |
| 1127433 | 81 | 72 |
| 1127603 | 37 | 64 |
| 1127962 | 83 | 75 |
| 1127963 | 72 | 74 |
| 1127979 | 72 | 65 |
| 1127982 | 70 | 81 |
| 1128018 | 63 | 63 |

TABLE 58
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 941371 | 54 | 51 |
| 941438 | 69 | 30 |
| 941612 | 75 | 70 |
| 941686 | 44 | 69 |
| 1126433 | 60 | 48 |
| 1126450 | 34 | 16 |
| 1126452 | 32 | 32 |
| 1126458 | 20 | 28 |
| 1126464 | 9 | 31 |
| 1126775 | 45 | 47 |
| 1126888 | 34 | 22 |
| 1127040 | 64 | 52 |
| 1127084 | 38 | 0 |

TABLE 59
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 1127114 | 64 | 52 |
| 1127174 | 34 | 36 |
| 1127251 | 78 | 78 |
| 1127331 | 72 | 48 |
| 1127358 | 57 | 69 |
| 1127467 | 54 | 48 |
| 1127507 | 15 | 29 |
| 1127728 | 42 | 46 |
| 1127789 | 26 | 52 |
| 1127954 | 84 | 83 |
| 1128017 | 43 | 68 |
| 1128027 | 79 | 55 |
| 1128051 | 37 | 38 |

TABLE 60
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 648411 | 80 | 85 |
| 941366 | 72 | 83 |
| 941367 | 72 | 72 |
| 941554 | 0 | 32 |
| 941637 | 73 | 84 |
| 941695 | 82 | 85 |
| 1126456 | 42 | 76 |
| 1126457 | 55 | 57 |
| 1126460 | 0 | 22 |
| 1126466 | 38 | 45 |
| 1126788 | 0 | 8 |
| 1126790 | 0 | 34 |
| 1126884 | 0 | 18 |
| 1126886 | 0 | 26 |
| 1126898 | 0 | 20 |
| 1126901 | 0 | 0 |
| 1126993 | 28 | 59 |
| 1127016 | 32 | 43 |
| 1127041 | 26 | 71 |
| 1127054 | 30 | 75 |
| 1127055 | 20 | 61 |
| 1127057 | 50 | 66 |
| 1127096 | 68 | 69 |
| 1127155 | 34 | 59 |
| 1127246 | 0‡ | 50 |
| 1128040 | 14 | 22 |
| 1128052 | 0 | 32 |
| 1128275 | 10 | 38 |
| 1127954 | 74‡ | 83‡ |

‡Indicates that fewer than 3 samples were available

TABLE 61
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 1127249 | 80 | 73 |
| 1127252 | 55 | 61 |
| 1127261 | 70 | 70 |
| 1127344 | 54 | 60 |
| 1127357 | 61 | 75 |
| 1127398 | 56 | 76 |
| 1127409 | 46 | 36 |
| 1127410 | 61‡ | 83‡ |
| 1127429 | 94 | 90 |
| 1127430 | 76‡ | 90‡ |
| 1127431 | 91‡ | 88‡ |
| 1127463 | 37 | 38 |
| 1127506 | 46 | 38 |
| 1127535 | 62 | 60 |
| 1127579 | 11 | 10 |
| 1127711 | 63 | 68 |
| 1127720 | 60 | 41 |
| 1127721 | 49 | 44 |
| 1127788 | 53 | 34 |
| 1127822 | 20 | 37 |
| 1127823 | 47 | 50 |
| 1127952 | 58‡ | 65‡ |
| 1127960 | 67‡ | 60‡ |
| 1127981 | 69 | 77 |
| 1128016 | 81‡ | 70‡ |

‡Indicates that fewer than 3 samples were available

TABLE 62
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 941563 | 38 | 43 |
| 1126465 | 55‡ | 52‡ |
| 1126778 | 12 | 25 |
| 1126788 | 49 | 41 |
| 1126790 | 22 | 36 |
| 1126884 | 19‡ | 10‡ |
| 1126996 | 68‡ | 57‡ |
| 1127036 | 41 | 24 |
| 1127181 | 50‡ | 30‡ |
| 1127248 | 59 | 48 |
| 1127291 | 61 | 70 |
| 1127363 | 60 | 47 |
| 1127399 | 50 | 70 |
| 1127993 | 59 | 33 |
| 1128019 | 64 | 71 |
| 1128036 | 59‡ | 20‡ |
| 1128609 | 30 | 37 |

‡Indicates that fewer than 3 samples were available

TABLE 63
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 941428 | 18 | 6 |
| 941573 | 30‡ | 22‡ |
| 941679 | 35 | 35 |
| 941715 | 70 | 73 |
| 941722 | 27‡ | 12‡ |
| 1126449 | 26‡ | 7 |
| 1126931 | 30 | 23 |
| 1127001 | 65 | 73 |
| 1127015 | 73 | 69 |
| 1127042 | 68‡ | 70‡ |
| 1127239 | 26 | 39 |
| 1127242 | 47‡ | 41‡ |
| 1127402 | 80 | 72 |
| 1127411 | 64 | 77 |
| 1127413 | 66 | 69 |
| 1127434 | 74‡ | 74‡ |
| 1127498 | 32 | 30 |
| 1127604 | 47 | 47 |
| 1127710 | 32 | 52 |
| 1127954 | 69 | 75 |
| 1128028 | 41 | 63 |
| 1128038 | 40 | 55 |

‡Indicates that fewer than 3 samples were available

TABLE 64
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 941573 | 0 | 32 |
| 941679 | 35 | 30 |
| 941715 | 60 | 81 |
| 941721 | 47 | 51 |
| 1126449 | 46 | 59 |
| 1126931 | 0‡ | 12‡ |
| 1127015 | 55 | 65 |
| 1127242 | 47 | 46 |
| 1127411 | 75 | 81 |
| 1127413 | 60 | 80 |
| 1127498 | 39 | 29 |
| 1127604 | 65 | 47 |
| 1127710 | 57 | 57 |
| 1127954 | 74 | 70 |
| 1128028 | 62 | 72 |
| 1128036 | 63 | 42 |
| 1251609 | 64‡ | 56 |
| 1251611 | 65 | 66 |
| 1251612 | 29‡ | 35‡ |
| 1251613 | 74 | 73 |
| 1251614 | 44‡ | 28‡ |
| 1251615 | 57 | 58 |
| 1251617 | 48 | 38 |
| 1251619 | 30 | 21 |
| 1251620 | 42 | 38 |
| 1251624 | 79 | 57 |
| 1251625 | 64 | 61 |
| 1251626 | 76 | 73 |
| 1251627 | 78 | 69 |

‡Indicates that fewer than 3 samples were available

TABLE 65
Reduction of human GYS1 RNA in transgenic
mice compared to PBS control

| | |
|---|---|
| | GYS1 RNA |
| Compound | (% reduction) |

| Number | Cortex | Spinal cord |
|---|---|---|
| 941635 | 91 | 71 |
| 941653 | 75 | 74 |
| 1126292 | 59 | 72 |
| 1126293 | 43 | 54 |
| 1127250 | 68 | 57 |
| 1127253 | 72 | 66 |
| 1127286 | 57 | 61 |
| 1127418 | 73 | 80 |
| 1127419 | 96 | 86 |
| 1127420 | 94 | 77 |
| 1127421 | 53 | 58 |
| 1127422 | 36 | 39 |
| 1127423 | 68 | 50 |
| 1127424 | 63 | 49 |
| 1127425 | 50 | 37 |
| 1127426 | 54 | 69 |
| 1127427 | 68 | 64 |
| 1127817 | 67 | 68 |
| 1127953 | 63 | 56 |
| 1127955 | 86 | 87 |
| 1127956 | 91 | 83 |
| 1127957 | 43 | 53 |
| 1127958 | 44 | 36 |
| 1127959 | 48 | 51 |
| 1127961 | 71 | 56 |
| 1128004 | 48 | 55 |
| 1128009 | 88 | 69 |

TABLE 66
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 1251609 | 49 | 59 |
| 1251611 | 55 | 63 |
| 1251612 | 16 | 38 |
| 1251613 | 76‡ | 77‡ |
| 1251614 | 40 | 58 |
| 1251615 | 39 | 73 |
| 1251617 | 31 | 44 |
| 1251619 | 5 | 38 |
| 1251620 | 9‡ | 50‡ |
| 1251624 | 23 | 59 |
| 1251625 | 29 | 54 |
| 1251626 | 46 | 69 |
| 1251627 | 61 | 72 |
| 941358 | 27 | 56 |
| 941636 | 69 | 75 |
| 941652 | 44 | 47 |
| 1126291 | 44‡ | 52‡ |
| 1127292 | 62 | 78 |
| 1127385 | 75 | 84 |
| 1127391 | 78 | 76 |
| 1127444 | 55 | 63 |
| 1127538 | 35 | 55 |
| 1127747 | 66 | 55 |
| 1127762 | 21 | 49 |
| 1127879 | 47 | 74 |

‡Indicates that fewer than 3 samples were available

TABLE 67
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 941426 | 33 | 36 |
| 941427 | 42 | 39 |
| 941429 | 10 | 29 |
| 941430 | 24 | 40 |
| 941558 | 31 | 35 |
| 941648 | 58 | 77 |
| 941678 | 58 | 66 |
| 941690 | 49 | 72 |
| 941724 | 17 | 59 |
| 1126455 | 0 | 56 |
| 1126784 | 0 | 49 |
| 1126902 | 0 | 19 |
| 1127014 | 60‡ | 73‡ |
| 1127017 | 19 | 42 |
| 1127019 | 37 | 75 |
| 1127091 | 52‡ | 81‡ |
| 1127172 | 34 | 60 |
| 1127313 | 34‡ | 55‡ |
| 1127335 | 29 | 68 |
| 1127369 | 39 | 75 |
| 1127384 | 62 | 84 |
| 1128032 | 27 | 80 |
| 1128033 | 2 | 69 |
| 1128035 | 29 | 68 |
| 1128039 | 42 | 72 |
| 1270757 | 18 | 65 |
| 1270759 | 16‡ | 37‡ |
| 1270761 | 0 | 15 |
| 1270762 | 0 | 51 |
| 1270763 | 0 | 29 |
| 1270766 | 12 | 53 |
| 1270773 | 50 | 49 |

‡Indicates that fewer than 3 samples were available

TABLE 68
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 941717 | 84 | 35 |
| 941772 | 59‡ | 28‡ |
| 1126331 | 34 | 45 |
| 1126889 | 0 | 0 |
| 1126933 | 0‡ | 0‡ |
| 1126997 | 0‡ | 0‡ |
| 1127103 | 8 | 65 |
| 1127782 | 0 | 1 |
| 1127954 | 72 | 81 |
| 1251610 | 0 | 0 |
| 1251616 | 11 | 21 |
| 1251618 | 31 | 52 |
| 1251621 | 0 | 5 |
| 1251622 | 14 | 31 |
| 1251623 | 25 | 31 |
| 1251628 | 63 | 62 |
| 1270756 | 2 | 47 |
| 1270760 | 0 | 0 |
| 1270769 | 4 | 42 |
| 1270774 | 0 | 18 |
| 1270775 | 5 | 14 |
| 1270776 | 2 | 45 |
| 1270782 | 0 | 0 |
| 1270784 | 62 | 80 |
| 1270786 | 0 | 16 |
| 1270787 | 0 | 21 |
| 1270793 | 0 | 52 |
| 1270796 | 0 | 53 |

‡Indicates that fewer than 3 samples were available

TABLE 69
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 941424 | 64 | 29 |
| 941605 | 67 | 83 |
| 1126777 | 23 | 24 |
| 1126930 | 38 | 4 |
| 1127214 | 37‡ | 21‡ |
| 1127241 | 88 | 73 |
| 1127749 | 44‡ | 19‡ |
| 1128041 | 74 | 70 |
| 1128483 | 56 | 30 |
| 1270758 | 63 | 49 |
| 1270764 | 96‡ | 88‡ |
| 1270765 | 43 | 43 |
| 1270767 | 58 | 54 |
| 1270768 | 77 | 61 |
| 1270770 | 63 | 44 |
| 1270772 | 47 | 50 |
| 1270777 | 70 | 64 |
| 1270779 | 82 | 64 |
| 1270780 | 49 | 23 |
| 1270781 | 60 | 34 |
| 1270783 | 82 | 70 |
| 1270788 | 79 | 58 |
| 1270789 | 56 | 45 |
| 1270791 | 51 | 40 |

‡Indicates that fewer than 3 samples were available

TABLE 70
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 648374 | 38 | 38 |
| 941370 | 96 | 94 |
| 941555 | 21 | 8 |
| 941588 | 0 | 54 |
| 941617 | 19 | 69 |
| 941629 | 43‡ | 85‡ |
| 1126340 | 33 | 86 |
| 1126459 | 0 | 57 |
| 1126850 | 0 | 55 |
| 1127018 | 65 | 88 |
| 1127095 | 5 | 87 |
| 1127102 | 64 | 91 |
| 1127186 | 0 | 69 |
| 1127201 | 68 | 82 |
| 1127223 | 8 | 77 |
| 1127233 | 0 | 76 |
| 1127234 | 26 | 83 |
| 1127238 | 32 | 83 |
| 1127412 | 2 | 86 |
| 1127445 | 0 | 80 |
| 1127790 | 39‡ | 83‡ |
| 1270771 | 0 | 67 |
| 1270778 | 0 | 91 |
| 1270785 | 0 | 65 |
| 1270790 | 18 | 70 |
| 1270792 | 10‡ | 74‡ |
| 1270794 | 0 | 74 |
| 1270795 | 0 | 53 |
| 1270755 | 31 | 80 |

‡Indicates that fewer than 3 samples were available

TABLE 71
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 648408 | 57 | 43 |
| 648409 | 86 | 63 |
| 941654 | 29 | 40 |
| 941693 | 0 | 26 |
| 1126413 | 75 | 66 |
| 1126804 | 27 | 30 |
| 1127037 | 36 | 49 |
| 1127085 | 21‡ | 42‡ |
| 1127243 | 77 | 65 |
| 1127260 | 96 | 88 |
| 1127329 | 55 | 72 |
| 1127403 | 63 | 66 |
| 1127404 | 79 | 68 |
| 1127408 | 40 | 55 |
| 1127726 | 40 | 46 |
| 1127808 | 74 | 49 |
| 1127809 | 44 | 46 |
| 1127810 | 19 | 33 |
| 1127811 | 23 | 50 |
| 1127812 | 40 | 35 |
| 1127813 | 32 | 31 |
| 1127814 | 71 | 44 |
| 1127815 | 30‡ | 56‡ |
| 1127816 | 30 | 38 |
| 1127818 | 59‡ | 63‡ |
| 1127819 | 45 | 40 |
| 1127820 | 51‡ | 58‡ |
| 1127954 | 91 | 87 |

‡Indicates that fewer than 3 samples were available

TABLE 72
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 648410 | 76 | 54 |
| 941459 | 66 | 66 |
| 941462 | 7 | 26 |
| 941640 | 62 | 54 |
| 941641 | 71 | 59 |
| 941651 | 75 | 69 |
| 941725 | 43 | 47 |
| 1126323 | 30 | 33 |
| 1126341 | 68 | 50 |
| 1126454 | 33 | 44 |
| 1126461 | 24 | 22 |
| 1126805 | 10‡ | 16‡ |
| 1127020 | 77 | 63 |
| 1127200 | 64‡ | 69‡ |
| 1127300 | 78 | 70 |
| 1127393 | 56 | 49 |
| 1127405 | 70 | 60 |
| 1127406 | 22 | 52 |
| 1127407 | 0 | 18 |
| 1127578 | 21 | 17 |
| 1127669 | 88 | 82 |
| 1128006 | 71 | 74 |
| 1128007 | 77 | 74 |
| 1128008 | 69 | 50 |
| 1128013 | 88 | 78 |
| 1128014 | 79 | 70 |
| 1128026 | 45 | 45 |
| 1128029 | 49 | 38 |
| 1128036 | 70 | 58 |

‡Indicates that fewer than 3 samples were available

TABLE 73
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 941365 | 48 | 56 |
| 941450 | 43‡ | 72‡ |
| 1126290 | 26 | 61 |
| 1126294 | 32 | 78 |
| 1126315 | 56 | 70 |
| 1126316 | 51 | 75 |
| 1126317 | 42 | 74 |
| 1126318 | 37 | 67 |
| 1126319 | 30 | 71 |
| 1126320 | 54 | 74 |
| 1126321 | 55 | 74 |
| 1126322 | 51 | 64‡ |
| 1126324 | 26 | 67 |
| 1126325 | 47 | 75 |
| 1126326 | 38 | 76 |
| 1126327 | 47 | 68 |
| 1126328 | 40 | 71 |
| 1126329 | 43 | 70 |
| 1126795 | 0 | 48 |
| 1126885 | 0 | 47 |
| 1126933 | 0 | 41 |
| 1126998 | 52 | 77 |
| 1127013 | 17 | 76 |
| 1127038 | 38 | 73 |
| 1127428 | 95 | 98 |
| 1127821 | 67 | 72 |
| 1127964 | 81 | 82 |
| 1128010 | 0 | 57 |
| 1128011 | 0 | 55 |
| 1128012 | 0 | 57 |
| 1128015 | 49 | 77 |
| 1128278 | 11 | 46 |
| 1128284 | 28‡ | 43‡ |
| 1270764 | 79 | 88 |

‡Indicates that fewer than 3 samples were available

TABLE 74
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 648138 | 60 | 53 |
| 648146 | 52 | 51 |
| 941369 | 62 | 61 |
| 1126330 | 79 | 62 |
| 1126332 | 90 | 76 |
| 1126339 | 82 | 69 |
| 1127009 | 91 | 73 |
| 1127764 | 41‡ | 61‡ |
| 1128021 | 74 | 75 |
| 1311839 | 76 | 77 |
| 1311847 | 55 | 80 |
| 1311849 | 86 | 83 |
| 1311850 | 86 | 81 |
| 1311851 | 92 | 94 |
| 1311852 | 94 | 91 |
| 1311854 | 83 | 81 |
| 1311855 | 74 | 74 |
| 1311857 | 95 | 89 |
| 1311859 | 77 | 82 |
| 1311860 | 94 | 76 |
| 1311861 | 95 | 83 |
| 1311876 | 89‡ | 82‡ |
| 1311877 | 86 | 69 |
| 1311878 | 47‡ | 71‡ |
| 1318942 | 35 | 49 |
| 1318946 | 36 | 50 |
| 1318947 | 47‡ | 64‡ |

‡Indicates that fewer than 3 samples were available

TABLE 75
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 1311835 | 89 | 74 |
| 1311836 | 88 | 71 |
| 1311837 | 80 | 68 |
| 1311862 | 57 | 58 |
| 1311863 | 56 | 42 |
| 1311864 | 83 | 75 |
| 1311865 | 87 | 72 |
| 1311866 | 54‡ | 49‡ |
| 1311867 | 80 | 68 |
| 1311868 | 66 | 58 |
| 1311869 | 85 | 76 |
| 1311870 | 63 | 59 |
| 1311872 | 73 | 76 |
| 1311873 | 64 | 64 |
| 1311874 | 85 | 78 |
| 1311875 | 52 | 50 |
| 1318949 | 78 | 81 |
| 1318951 | 71 | 52 |
| 1318952 | 58‡ | 47‡ |
| 1318955 | 43 | 42 |
| 1318956 | 37 | 32 |
| 1318957 | 68 | 64 |
| 1318958 | 68 | 29 |
| 1318959 | 62‡ | 38‡ |

‡Indicates that fewer than 3 samples were available

TABLE 76
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 648378 | 27 | 26 |
| 1126333 | 64 | 60 |
| 1311840 | 68 | 68 |
| 1311841 | 73 | 72 |
| 1311842 | 71 | 54 |
| 1311843 | 43 | 51 |
| 1311844 | 51 | 45 |
| 1311845 | 41 | 53 |
| 1311846 | 55 | 60 |
| 1311848 | 78 | 72 |
| 1311853 | 79 | 78 |
| 1311856 | 94 | 87 |
| 1311858 | 94 | 89 |
| 1318941 | 31‡ | 18‡ |
| 1318943 | 15 | 6 |
| 1318944 | 0 | 0 |
| 1318945 | 9 | 21 |
| 1318948 | 23 | 0 |
| 1318950 | 91 | 90 |
| 1318953 | 61 | 42 |
| 1318954 | 53 | 43 |
| 1318960 | 36 | 55 |

‡Indicates that fewer than 3 samples were available

TABLE 77
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 1311843 | 33 | 47 |
| 1318961 | 5 | 3 |
| 1318962 | 51 | 47 |
| 1318964 | 58 | 38 |
| 1318966 | 45 | 41 |
| 1318967 | 28 | 38 |
| 1318968 | 31 | 6 |
| 1318969 | 8‡ | 0‡ |
| 1318970 | 4 | 0 |
| 1318971 | 17 | 5 |
| 1318972 | 0 | 0 |
| 1318974 | 24 | 13 |
| 1318975 | 27 | 13 |
| 1318976 | 0 | 11 |
| 1318977 | 0 | 0 |
| 1318978 | 23 | 4 |
| 1318979 | 83 | 72 |
| 1318980 | 72 | 55 |
| 1318981 | 85 | 82 |
| 1318983 | 90 | 72 |
| 1318986 | 66 | 25 |
| 1318989 | 26 | 3 |
| 1318992 | 38 | 0 |

‡Indicates that fewer than 3 samples were available

TABLE 78
Reduction of human GYS1 RNA in transgenic
mice compared to PBS control

| | |
|---|---|
| | GYS1 RNA |
| Compound | (% reduction) |

| Number | Cortex | Spinal Cord |
|---|---|---|
| 1318963 | 29 | 49 |
| 1318973 | 32 | 3 |
| 1318982 | 44 | 36 |
| 1318984 | 55 | 39 |
| 1318985 | 36 | 42 |
| 1318987 | 76 | 74 |
| 1318988 | 75 | 64 |
| 1318990 | 79 | 60 |
| 1318991 | 62 | 49 |
| 1318993 | 64 | 31 |

### Example 6: Potency of Modified Oligonucleotides Complementary to Human GYS1 in Transgenic Mice

Modified oligonucleotides described above were tested in the human GYS1 transgenic mouse model FVB-Tg (described herein above).

### Treatment

The GYS1 transgenic mice were divided into groups of 4 mice each. Each mouse received a single ICV bolus of modified oligonucleotide at the doses indicated in the tables below. A group of 4 mice received PBS as a negative control.

### RNA Analysis

Two weeks post treatment, mice were sacrificed, and RNA was extracted from the cortex, spinal cord, and hippocampus for RT-PCR analysis of RNA expression of GYS1 using Human GYS1 primer probe set RTS36345 (described herein above). Results are presented as percent reduction of GYS1 relative to the amount of GYS1 RNA in the PBS control, normalized to mouse cyclophilin A. Mouse cyclophilin A was amplified using primer probe set m_cyclo24 (described herein above). As used herein, a value of ‘0’ indicates that treatment with the modified oligonucleotide did not inhibit GYS1 mRNA levels.

As shown in the table below, treatment with modified oligonucleotides resulted in dose-responsive reduction of GYS1 RNA in comparison to the amount of GYS1 RNA in the PBS control (designated as 0% reduction). Dose response data were analyzed using Microsoft Excel (v14.4) and GraphPad Prism software (v 8.2.0, San Diego, CA). ED₅₀ values were calculated from log transformed dose or concentrations and individual animal GYS1 mRNA levels using the built in GraphPad formula “log(agonist) vs. response—Find ECanything”, with the following constraints: bottom=0, top=100, and F=50 for ED₅₀.

TABLE 79
Reduction of human GYS1 RNA in transgenic
mice compared to PBS control

GYS1 (% reduction)

| Compound | Dose | | ED₅₀ | Spinal | ED₅₀ |
|---|---|---|---|---|---|
| Number | (μg) | Cortex | (μg) | Cord | (μg) |
| 1127711 | 10 | 21 | 216 | 31 | 25 |
| | 30 | 26 | | 62 | |
| | 100 | 53‡ | | 67‡ | |
| | 300 | 50 | | 79 | |
| | 700 | 61 | | 81 | |
| 1127954 | 10 | 12 | 32 | 51 | 9 |
| | 30 | 51‡ | | 76‡ | |
| | 100 | 86 | | 86 | |
| | 300 | 84‡ | | 91‡ | |
| | 700 | 90 | | 94 | |
| 1127962 | 10 | 0 | 44 | 56 | 5 |
| | 30 | 29 | | 74 | |
| | 100 | 85‡ | | 73 | |
| | 300 | 90 | | 88 | |
| | 700 | 94 | | 95 | |
| 1127963 | 10 | 6 | 42 | 62 | 5 |
| | 30 | 49 | | 71 | |
| | 100 | 70 | | 88 | |
| | 300 | 90 | | 93 | |
| | 700 | 93 | | 93 | |

‡Indicates that fewer than 4 samples were available

TABLE 80
Reduction of human GYS1 RNA in transgenic
mice compared to PBS control

GYS1 (% reduction)

| Compound | Dose | | ED₅₀ | Spinal | ED₅₀ |
|---|---|---|---|---|---|
| Number | (μg) | Cortex | (μg) | Cord | (μg) |
| 648411 | 10 | 38‡ | 17 | 30‡ | 28 |
| | 30 | 60 | | 53 | |
| | 100 | 82 | | 73 | |
| | 300 | 80 | | 79 | |
| | 700 | 90 | | 85 | |
| 941366 | 10 | 0 | 187 | 26 | 155 |
| | 30 | 20 | | 16 | |
| | 100 | 57‡ | | 47‡ | |
| | 300 | 56 | | 62 | |
| | 700 | 64‡ | | 69‡ | |
| 941715 | 10 | 59 | 4 | 29 | 23 |
| | 30 | 75 | | 65 | |
| | 100 | 85 | | 68‡ | |
| | 300 | 85 | | 87 | |
| | 700 | 92 | | 89 | |
| 1127430 | 10 | 59 | 5 | 38 | 13 |
| | 30 | 76‡ | | 75 | |
| | 100 | 84 | | 79‡ | |
| | 300 | 91 | | 82 | |
| | 700 | 93‡ | | 88‡ | |
| 1127979 | 10 | 7 | 67 | 33 | 36 |
| | 30 | 42 | | 50 | |
| | 100 | 60 | | 64 | |
| | 300 | 76 | | 73 | |
| | 700 | 83 | | 77 | |
| 1128018 | 10 | 49‡ | 12 | 14 | 138 |
| | 30 | 65‡ | | 42‡ | |
| | 100 | 60 | | 26 | |
| | 300 | 85 | | 72 | |
| | 700 | 84 | | 76 | |

‡Indicates that fewer than 4 samples were available

TABLE 81
Reduction of human GYS1 RNA in transgenic
mice compared to PBS control

GYS1 (% reduction)

| Compound | Dose | | ED₅₀ | Spinal | ED₅₀ |
|---|---|---|---|---|---|
| Number | (μg) | Cortex | (μg) | Cord | (μg) |
| 1127432 | 10 | 28 | 36 | 4 | 52 |
| | 30 | 52 | | 49 | |
| | 100 | 62 | | 67 | |
| | 300 | 83 | | 81 | |
| | 700 | 84‡ | | 77‡ | |
| 1127981 | 10 | 21 | 19 | 32 | 19 |
| | 30 | 74 | | 65 | |
| | 100 | 86 | | 84 | |
| | 300 | 96 | | 91 | |
| | 700 | 96‡ | | 95‡ | |
| 1128016 | 10 | 34 | 132 | 17 | 83 |
| | 30 | 26 | | 40 | |
| | 100 | 41 | | 56 | |
| | 300 | 61 | | 69 | |
| | 700 | 73 | | 72 | |

‡Indicates that fewer than 4 samples were available

TABLE 82
Reduction of human GYS1 RNA in transgenic
mice compared to PBS control

GYS1 (% reduction)

| Compound | Dose | | ED₅₀ | Spinal | ED₅₀ |
|---|---|---|---|---|---|
| Number | (μg) | Cortex | (μg) | Cord | (μg) |
| 1318950 | 3 | 9 | 18 | 47 | 4 |
| | 10 | 27 | | 61 | |
| | 30 | 74 | | 71 | |
| | 100 | 85 | | 94 | |
| | 300 | 95 | | 95 | |
| 1311858 | 3 | 16 | 25 | 20 | 7 |
| | 10 | 17 | | 67 | |
| | 30 | 58 | | 82 | |
| | 100 | 87 | | 88 | |
| | 300 | 95‡ | | 95‡ | |
| 1311849 | 3 | 17 | 74 | 0 | 25 |
| | 10 | 0 | | 56 | |
| | 30 | 33 | | 52 | |
| | 100 | 60 | | 75 | |
| | 300 | 78 | | 76 | |
| 1311869 | 3 | 9 | 42 | 10 | 25 |
| | 10 | 22 | | 0 | |
| | 30 | 40 | | 69 | |
| | 100 | 71 | | 77 | |
| | 300 | 85 | | 81 | |

‡Indicates that fewer than 4 samples were available

TABLE 83
Reduction of human GYS1 RNA in transgenic
mice compared to PBS control

GYS1 (% reduction)

| Compound | Dose | | ED₅₀ | Spinal | ED₅₀ |
|---|---|---|---|---|---|
| Number | (μg) | Cortex | (μg) | Cord | (μg) |
| 1127260 | 3 | 12 | 20 | 10 | 53 |
| | 10 | 42 | | 33 | |
| | 30 | 57 | | 41 | |
| | 100 | 79 | | 63 | |
| | 300 | 84 | | 69 | |
| 1127955 | 3 | 50 | 5 | 24 | 18 |
| | 10 | 46 | | 39 | |
| | 30 | 83 | | 58 | |
| | 100 | 88 | | 79 | |
| | 300 | 96 | | 85 | |
| 1127956 | 3 | 17 | 19 | 22 | 32 |
| | 10 | 47 | | 40 | |
| | 30 | 55‡ | | 50‡ | |
| | 100 | 71 | | 55 | |
| | 300 | 93 | | 81 | |
| 1128013 | 3 | 26 | 28 | 7 | 44 |
| | 10 | 37 | | 35 | |
| | 30 | 52 | | 48 | |
| | 100 | 57 | | 62 | |
| | 300 | 85 | | 71 | |
| 1311856 | 3 | 29 | 6 | 35 | 6 |
| | 10 | 74 | | 59 | |
| | 30 | 66 | | 81 | |
| | 100 | 91 | | 84 | |
| | 300 | 91 | | 90 | |
| 1311848 | 3 | 36 | 11 | 29 | 14 |
| | 10 | 47 | | 57 | |
| | 30 | 62 | | 53 | |
| | 100 | 73‡ | | 70‡ | |
| | 300 | 86‡ | | 76‡ | |
| 1311853 | 3 | 28 | 14 | 16 | 18 |
| | 10 | 49 | | 40 | |
| | 30 | 51 | | 65 | |
| | 100 | 83‡ | | 79‡ | |
| | 300 | 89‡ | | 78‡ | |

‡Indicates that fewer than 4 samples were available

TABLE 84
Reduction of human GYS1 RNA in transgenic
mice compared to PBS control

GYS1 (% reduction)

| Compound | Dose | | ED₅₀ | Spinal | ED₅₀ |
|---|---|---|---|---|---|
| Number | (μg) | Cortex | (μg) | Cord | (μg) |
| 1127954 | 1 | 33‡ | 8 | 0‡ | 32 |
| | 3 | 25‡ | | 23‡ | |
| | 10 | 60 | | 18 | |
| | 30 | 58‡ | | 51‡ | |
| | 100 | 85 | | 78 | |
| | 300 | 94 | | 84 | |

‡Indicates that fewer than 4 samples were available

### Example 7: Activity of Modified Oligonucleotides Complementary to Human GYS1 in Transgenic Mice

Modified oligonucleotides selected from above were tested in the human GYS1 transgenic mouse model FVB-Tg (described herein above).

### Treatment

GYS1 transgenic mice were divided into groups of 4 mice each. Each mouse received a single ICV bolus of 200 g of modified oligonucleotide. A group of 4 mice received PBS as a negative control.

### RNA Analysis

Eight weeks post treatment, mice were sacrificed and RNA was extracted from cortical brain tissue, spinal cord, and hippocampus for RT-PCR analysis to measure the amount of GYS1 RNA using human GYS1 primer probe set RTS36345 (described herein above). Results are presented as percent reduction of GYS1 relative to the amount of GYS1 RNA in the PBS control normalized to mouse cyclophilin A. Mouse cyclophilin A was amplified using primer probe set m_cyclo24 (described herein above).

As shown in the table below, reduction of GYS1 RNA was observed 8 weeks after administration of the modified oligonucleotides (in comparison with the amount of GYS1 RNA in the PBS control designated as 0% reduction).

TABLE 85
Reduction of human GYS1 RNA in transgenic
mice compared to PBS control

| | |
|---|---|
| Compound | GYS1 RNA (% reduction) |

| Number | Cortex | Spinal Cord | Hippocampus |
|---|---|---|---|
| 941635 | 76 | 69 | 62 |
| 1127955 | 87 | 66 | 72 |
| 1127956 | 75 | 67 | 71 |
| 1128009 | 67 | 43 | 26 |

TABLE 86
Reduction of human GYS1 RNA in transgenic
mice compared to PBS control

| | |
|---|---|
| Compound | GYS1 RNA (% reduction) |

| Number | Cortex | Spinal Cord | Hippocampus |
|---|---|---|---|
| 648411 | 71‡ | 69‡ | 68‡ |
| 1127260 | 73 | 67 | 64 |
| 1127430 | 80‡ | 79‡ | 84‡ |
| 1127432 | 81‡ | 70‡ | 79‡ |
| 1127954 | 86 | 70 | 84 |
| 1127956 | 75 | 69 | 68 |
| 1127962 | 71 | 49 | 69 |
| 1127963 | 55 | 50 | 65 |
| 1251626 | 54 | 41 | 46 |
| 1311835 | 23 | 27 | 38 |
| 1311848 | 73 | 59 | 71 |
| 1311853 | 78‡ | 80‡ | 87‡ |
| 1311854 | 70‡ | 17‡ | 49‡ |
| 1311856 | 81‡ | 69‡ | 75‡ |
| 1311858 | 86 | 86 | 91 |

‡Indicates that fewer than 4 samples were available

1. (canceled)

2. (canceled)

3. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19, or 20 contiguous nucleobases complementary to:
an equal length portion of nucleobases 16915-16947 of SEQ ID NO: 2;
wherein the modified oligonucleotide comprises at least one modification selected from a modified sugar moiety and a modified internucleoside linkage.

4. An oligomeric compound comprising a modified oligonucleotide consisting of 12 to 30 linked nucleosides and having a nucleobase sequence comprising at least 8, at least 9, at least 10, at least 11, at least 12, at least 13, at least 14, at least 15, at least 16, at least 17, at least 18, at least 19 or 20 contiguous nucleobases of a sequence selected from:
SEQ ID NOs: 2376, 2453, 2530, 2607, 2683, 2759, 2836, 2913, 539, and 616;
wherein the modified oligonucleotide comprises at least one modification selected from a modified sugar moiety and a modified internucleoside linkage.

5. The oligomeric compound of claim 3, wherein the modified oligonucleotide has a nucleobase sequence that is at least 85%, at least 90%, at least 95%, or 100% complementary to the nucleobase sequence of any one of SEQ ID NOs: 1-10 and 14 when measured across the entire nucleobase sequence of the modified oligonucleotide.

6. (canceled)

7. (canceled)

8. The oligomeric compound of claim 3, wherein the modified oligonucleotide comprises at least one modified nucleoside comprising a bicyclic sugar moiety.

9. The oligomeric compound of claim 8, wherein the bicyclic sugar moiety has a 2′-4′ bridge selected from —O—CH₂—; and —O—CH(CH₃)—.

10. The oligomeric compound of claim 3, wherein the modified oligonucleotide comprises at least one modified nucleoside comprising a non-bicyclic modified sugar moiety.

11. The oligomeric compound of claim 10, wherein the non-bicyclic modified sugar moiety is a 2′-O(CH₂)₂OCH₃ ribosyl sugar moiety or a 2′-OMe modified sugar moiety.

12. The oligomeric compound of claim 3, wherein the modified oligonucleotide comprises at least one modified nucleoside comprising a sugar surrogate.

13. (canceled)

14. The oligomeric compound of claim 3, wherein the modified oligonucleotide is a gapmer.

15. The oligomeric compound of claim 3, wherein the modified oligonucleotide comprises at least one modified internucleoside linkage.

16. (canceled)

17. The oligomeric compound of claim 15, wherein at least one internucleoside linkage is a phosphorothioate internucleoside linkage.

18. The oligomeric compound of claim 15, wherein the modified oligonucleotide comprises at least one phosphodiester internucleoside linkage.

19. (canceled)

20. (canceled)

21. (canceled)

22. The oligomeric compound of claim 3, wherein the modified oligonucleotide comprises at least one modified nucleobase.

23. The oligomeric compound of claim 22, wherein the modified nucleobase is a 5-methylcytosine.

24.-30. (canceled)

31. The oligomeric compound of claim 3, wherein the modified oligonucleotide consists of 12-30, 12-22, 12-20,14-18, 14-20, 15-17, 15-25, 16-20, 18-22, or 18-20 linked nucleosides.

32. (canceled)

33. (canceled)

34. The oligomeric compound of claim 3, wherein the modified oligonucleotide consists of 16, 17, 18, 19, or 20 linked nucleosides.

35. The oligomeric compound of claim 3, wherein the modified oligonucleotide consists of 20 linked nucleosides.

36.-41. (canceled)

42. The oligomeric compound of claim 3, consisting of the modified oligonucleotide.

43. The oligomeric compound of claim 3, wherein the oligomeric compound comprises a conjugate group.

44.-59. (canceled)

60. An oligomeric duplex, comprising a first oligomeric compound and a second oligomeric compound comprising a second modified oligonucleotide, wherein the first oligomeric compound is an oligomeric compound of claim 3.

61.-101. (canceled)

102. A population of oligomeric compounds of claim 3, wherein all of the phosphorothioate internucleoside linkages of the modified oligonucleotide are stereorandom.

103. A pharmaceutical composition comprising an oligomeric compound of claim 3 and a pharmaceutically acceptable diluent.

104. The pharmaceutical composition of claim 103, wherein the pharmaceutically acceptable diluent is artificial cerebrospinal fluid (aCSF) or phosphate-buffered saline (PBS).

105. The pharmaceutical composition of claim 104, wherein the pharmaceutical composition consists essentially of the oligomeric compound and aCSF.

106. The pharmaceutical composition of claim 104, wherein the pharmaceutical composition consists essentially of the oligomeric compound and PBS.

107.-125. (canceled)
